(19) United States
(12) Patent Application Publication
(10) Pub. No.: US 2024/0199611 A1
(43) Pub. Date: Jun. 20, 2024

(54) ANTIBACTERIAL COMPOUNDS

(71) Applicant: JANSSEN SCIENCES IRELAND UNLIMITED COMPANY, Co. Cork (IE)

(72) Inventors: Jérôme Émile Georges GUILLEMONT, Andé (FR); Magali Madeleine Simone MOTTE, Louviers (FR); Maria Cristina VILLELLAS ARILLA, Turnhout (BE); Godelieve Maria J. LAMMENS, Geel (BE); Adeline Julie Dominique Marie RENÉ, Bois-Guillaume (FR); Matthieu Ludovic JEANTY, Le Vaudreuil (FR); Dirk Antonie LAMPRECHT, Turnhout (BE)

(21) Appl. No.: 18/550,323

(22) PCT Filed: Mar. 15, 2022
(86) PCT No.: PCT/EP2022/056588

§ 371 Date: Sep. 13, 2023

(30) Foreign Application Priority Data
Mar. 16, 2021 (EP) 21162835.9

(51) Int. Cl. C07D 471/04; A61K 31/53; A61P 31/06; C07D 487/04; C07D 487/14; C07D 491/147; C07D 498/14; C07D 513/04

(52) U.S. Cl. CPC C07D 471/04; A61K 31/53; A61P 31/06; C07D 487/04; C07D 487/14; C07D 491/147; C07D 498/14; C07D 513/04

(57) ABSTRACT

The present invention relates to the compounds (I) wherein the integers are as defined in the description, and where the compounds may be useful as medicaments, for instance for use in the treatment of tuberculosis.

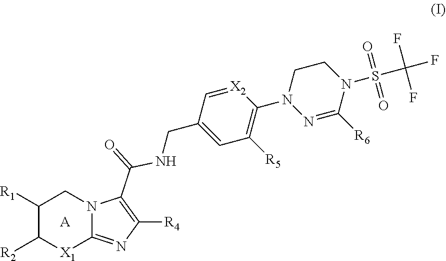

The present invention relates to novel compounds. The invention also relates to such compounds for use as a pharmaceutical and further for the use in the treatment of bacterial diseases, including diseases caused by pathogenic mycobacteria such as Mycobacterium tuberculosis. Such compounds may work by interfering with ATP synthase in M. tuberculosis, with the inhibition of cytochrome bc₁ activity as the primary mode of action. Hence, primarily, such compounds are antitubercular agents.

### BACKGROUND OF THE INVENTION

Mycobacterium tuberculosis is the causative agent of tuberculosis (TB), a serious and potentially fatal infection with a world-wide distribution. Estimates from the World Health Organization indicate that more than 8 million people contract TB each year, and 2 million people die from tuberculosis yearly. In the last decade, TB cases have grown 20% worldwide with the highest burden in the most impoverished communities. If these trends continue, TB incidence will increase by 41% in the next twenty years. Fifty years since the introduction of an effective chemotherapy, TB remains after AIDS, the leading infectious cause of adult mortality in the world. Complicating the TB epidemic is the rising tide of multi-drug-resistant strains, and the deadly symbiosis with HIV. People who are HIV-positive and infected with TB are 30 times more likely to develop active TB than people who are HIV-negative and TB is responsible for the death of one out of every three people with HIV/AIDS worldwide.

Existing approaches to treatment of tuberculosis all involve the combination of multiple agents. For example, the regimen recommended by the U.S. Public Health Service is a combination of isoniazid, rifampicin and pyrazinamide for two months, followed by isoniazid and rifampicin alone for a further four months. These drugs are continued for a further seven months in patients infected with HIV. For patients infected with multi-drug resistant strains of M. tuberculosis, agents such as ethambutol, streptomycin, kanamycin, amikacin, capreomycin, ethionamide, cycloserine, ciprofoxacin and ofloxacin are added to the combination therapies. There exists no single agent that is effective in the clinical treatment of tuberculosis, nor any combination of agents that offers the possibility of therapy of less than six months' duration.

There is a high medical need for new drugs that improve current treatment by enabling regimens that facilitate patient and provider compliance. Shorter regimens and those that require less supervision are the best way to achieve this. Most of the benefit from treatment comes in the first 2 months, during the intensive, or bactericidal, phase when four drugs are given together; the bacterial burden is greatly reduced, and patients become noninfectious. The 4- to 6-month continuation, or sterilizing, phase is required to eliminate persisting bacilli and to minimize the risk of relapse. A potent sterilizing drug that shortens treatment to 2 months or less would be extremely beneficial. Drugs that facilitate compliance by requiring less intensive supervision also are needed. Obviously, a compound that reduces both the total length of treatment and the frequency of drug administration would provide the greatest benefit.

Complicating the TB epidemic is the increasing incidence of multi-drug-resistant strains or MDR-TB. Up to four percent of all cases worldwide are considered MDR-TB—those resistant to the most effective drugs of the four-drug standard, isoniazid and rifampin. MDR-TB is lethal when untreated and cannot be adequately treated through the standard therapy, so treatment requires up to 2 years of “second-line” drugs. These drugs are often toxic, expensive and marginally effective. In the absence of an effective therapy, infectious MDR-TB patients continue to spread the disease, producing new infections with MDR-TB strains. There is a high medical need for a new drug with a new mechanism of action, which is likely to demonstrate activity against drug resistant, in particular MDR strains.

The term “drug resistant” as used hereinbefore or hereinafter is a term well understood by the person skilled in microbiology. A drug resistant Mycobacterium is a Mycobacterium which is no longer susceptible to at least one previously effective drug; which has developed the ability to withstand antibiotic attack by at least one previously effective drug. A drug resistant strain may relay that ability to withstand to its progeny. Said resistance may be due to random genetic mutations in the bacterial cell that alters its sensitivity to a single drug or to different drugs.

MDR tuberculosis is a specific form of drug resistant tuberculosis due to a bacterium resistant to at least isoniazid and rifampicin (with or without resistance to other drugs), which are at present the two most powerful anti-TB drugs. Thus, whenever used hereinbefore or hereinafter “drug resistant” includes multi drug resistant.

Another factor in the control of the TB epidemic is the problem of latent TB. In spite of decades of tuberculosis (TB) control programs, about 2 billion people are infected by M. tuberculosis, though asymptomatically. About 10% of these individuals are at risk of developing active TB during their lifespan. The global epidemic of TB is fuelled by infection of HIV patients with TB and rise of multi-drug resistant TB strains (MDR-TB). The reactivation of latent TB is a high risk factor for disease development and accounts for 32% deaths in HIV infected individuals. To control TB epidemic, the need is to discover new drugs that can kill dormant or latent bacilli. The dormant TB can get reactivated to cause disease by several factors like suppression of host immunity by use of immunosuppressive agents like antibodies against tumor necrosis factor α or interferon-γ. In case of HIV positive patients the only prophylactic treatment available for latent TB is two-three months regimens of rifampicin, pyrazinamide. The efficacy of the treatment regime is still not clear and furthermore the length of the treatments is an important constrain in resource-limited environments. Hence there is a drastic need to identify new drugs, which can act as chemoprophylatic agents for individuals harboring latent TB bacilli.

The tubercle bacilli enter healthy individuals by inhalation; they are phagocytosed by the alveolar macrophages of the lungs. This leads to potent immune response and formation of granulomas, which consist of macrophages infected with M. tuberculosis surrounded by T cells. After a period of 6-8 weeks the host immune response cause death of infected cells by necrosis and accumulation of caseous material with certain extracellular bacilli, surrounded by macrophages, epitheloid cells and layers of lymphoid tissue at the periphery. In case of healthy individuals, most of the mycobacteria are killed in these environments but a small proportion of bacilli still survive and are thought to exist in a non-replicating, hypometabolic state and are tolerant to killing by anti-TB drugs like isoniazid. These bacilli can remain in the altered physiological environments even for individual's lifetime without showing any clinical symptoms of disease. However, in 10% of the cases these latent bacilli may reactivate to cause disease. One of the hypothesis about development of these persistent bacteria is patho-physiological environment in human lesions namely, reduced oxygen tension, nutrient limitation, and acidic pH. These factors have been postulated to render these bacteria phenotypically tolerant to major anti-mycobacterial drugs.

In addition to the management of the TB epidemic, there is the emerging problem of resistance to first-line antibiotic agents. Some important examples include penicillin-resistant Streptococcus pneumoniae, vancomycin-resistant enterococci, methicillin-resistant Staphylococcus aureus, multi-resistant salmonellae.

The consequences of resistance to antibiotic agents are severe. Infections caused by resistant microbes fail to respond to treatment, resulting in prolonged illness and greater risk of death. Treatment failures also lead to longer periods of infectivity, which increase the numbers of infected people moving in the community and thus exposing the general population to the risk of contracting a resistant strain infection.

Hospitals are a critical component of the antimicrobial resistance problem worldwide. The combination of highly susceptible patients, intensive and prolonged antimicrobial use, and cross-infection has resulted in infections with highly resistant bacterial pathogens.

Self-medication with antimicrobials is another major factor contributing to resistance. Self-medicated antimicrobials may be unnecessary, are often inadequately dosed, or may not contain adequate amounts of active drug.

Patient compliance with recommended treatment is another major problem. Patients forget to take medication, interrupt their treatment when they begin to feel better, or may be unable to afford a full course, thereby creating an ideal environment for microbes to adapt rather than be killed.

Because of the emerging resistance to multiple antibiotics, physicians are confronted with infections for which there is no effective therapy. The morbidity, mortality, and financial costs of such infections impose an increasing burden for health care systems worldwide.

Therefore, there is a high need for new compounds to treat bacterial infections, especially mycobacterial infections including drug resistant and latent mycobacterial infections, and also other bacterial infections especially those caused by resistant bacterial strains.

Anti-infective compounds for treating tuberculosis have been disclosed in e.g. international patent application WO 2011/113606. Such a document is concerned with compounds that would prevent M. tuberculosis multiplication inside the host macrophage and relates to compounds with a bicyclic core, imidazopyridines, which are linked (e.g. via an amido moiety) to e.g. an optionally substituted benzyl group.

International patent application WO 2014/015167 also discloses compounds that are disclosed as being of potential use in the treatment of tuberculosis. Such compounds disclosed herein have a bicycle (a 5,5-fused bicycle) as an essential element, which is substituted by a linker group (e.g. an amido group), which itself may be attached to another bicycle or aromatic group. Such compounds in this document do not contain a series of more than three rings.

Journal article Nature Medicine, 19, 1157-1160 (2013) by Pethe et al “Discovery of Q203, a potent clinical candidate for the treatment of tuberculosis” identifies a specific compound that was tested against M. tuberculosis. This compound Q203 is depicted below.

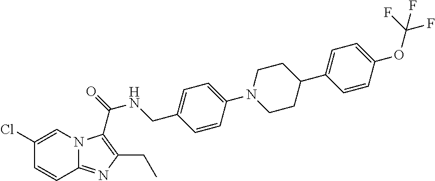 [SMILES: CCc1nc2ccc(Cl)cn2c1C(=O)NCc1ccc(N2CCC(c3ccc(OC(F)(F)F)cc3)CC2)cc1]

This clinical candidate is also discussed in journal article, J. Medicinal Chemistry, 2014, 57 (12), pp 5293-5305. It is stated to have activity against MDR tuberculosis, and have activity against the strain M. tuberculosis H37Rv at a MIC₅₀ of 0.28 nM inside macrophages. Positive control data (using known anti-TB compounds bedaquiline, isoniazid and moxifloxacin) are also reported. This document also suggests a mode of action, based on studies with mutants. It postulates that it acts by interfering with ATP synthase in M. tuberculosis, and that the inhibition of cytochrome bc₁ activity is the primary mode of action. Cytochrome bc₁ is an essential component of the electron transport chain required for ATP synthesis. It appeared that Q203 was highly active against both replicating and non-replicating bacteria.

International patent application WO 2015/014993 also discloses compounds as having activity against M. tuberculosis, as do international patent applications WO 2014/4015167, WO 2017/001660, WO 2017/001661, WO 2017/216281 and WO 2017/216283. International patent applications WO 2013/033070 and WO 2013/033167 disclose various compounds as kinase modulators.

The purpose of the present invention is to provide compounds for use in the treatment of bacterial diseases, particularly those diseases caused by pathogenic bacteria such as Mycobacterium tuberculosis (including the latent disease and including drug resistant M. tuberculosis strains). Such compounds may also be novel and may act by interfering with ATP synthase in M. tuberculosis, with the inhibition of cytochrome bc₁ activity being considered the primary mode of action.

### SUMMARY OF THE INVENTION

There is now provided a compound of formula (I)

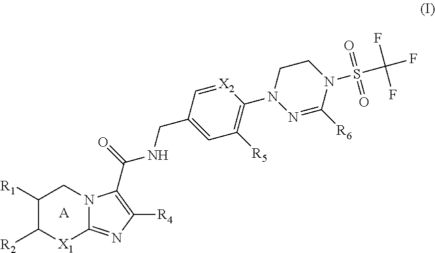

wherein
A is a 6-membered ring, which may be aromatic or non-aromatic,
X₁ represents ═N— or ═C(R₃)— (when aromatic) or —CH₂— (when non-aromatic);
X₂ represents ═N— or ═CH—;
R₁ is selected from H, —CH₃, F and Cl;
R₂ is selected from H and —CH₃;
R₃ is selected from H and F;
R₄ is selected from —CF₃, —CHF₂ and —C₂H₅;
R₅ is selected from H and F;
R₆ is selected from —CH₃, —C₂H₅, isopropyl, cyclopropyl, cyclobutyl, —C(═O)—OCH₃, —C(═O)—NH₂ and —C(═O)—N(CH₃)₂,
   or a pharmaceutically-acceptable salt thereof,
   which compounds may be referred to herein as “compounds of the invention”.

In compound of the invention, the integer R₆, in an embodiment can be defined as representing:
(i) C₁₋₄ alkyl (e.g. C₁₋₃ alkyl);
(ii) C₃₋₆ cycloalkyl (e.g. C₃₋₄ cycloalkyl); or
(iii) —C(O)OC₁₋₂ alkyl; or
(iv) —C(O)N(R^{a})(R^{b}) (in which R^{a} and R^{b} each independently represent hydrogen or C₁₋₂ alkyl).

Pharmaceutically-acceptable salts include acid addition salts and base addition salts. Such salts may be formed by conventional means, for example by reaction of a free acid or a free base form of a compound of formula I with one or more equivalents of an appropriate acid or base, optionally in a solvent, or in a medium in which the salt is insoluble, followed by removal of said solvent, or said medium, using standard techniques (e.g. in vacuo, by freeze-drying or by filtration). Salts may also be prepared by exchanging a counter-ion of a compound of the invention in the form of a salt with another counter-ion, for example using a suitable ion exchange resin.

The pharmaceutically acceptable acid addition salts as mentioned hereinabove are meant to comprise the therapeutically active non-toxic acid addition salt forms that the compounds of formula (I) are able to form. These pharmaceutically acceptable acid addition salts can conveniently be obtained by treating the base form with such appropriate acid. Appropriate acids comprise, for example, inorganic acids such as hydrohalic acids, e.g. hydrochloric or hydrobromic acid, sulfuric, nitric, phosphoric and the like acids; or organic acids such as, for example, acetic, propanoic, hydroxyacetic, lactic, pyruvic, oxalic (i.e. ethanedioic), malonic, succinic (i.e. butanedioic acid), maleic, fumaric, malic, tartaric, citric, methanesulfonic, ethanesulfonic, benzenesulfonic, p-toluenesulfonic, cyclamic, salicylic, p-aminosalicylic, pamoic and the like acids.

For the purposes of this invention solvates, prodrugs, N-oxides and stereoisomers of compounds of the invention are also included within the scope of the invention.

The term “prodrug” of a relevant compound of the invention includes any compound that, following oral or parenteral administration, is metabolised in vivo to form that compound in an experimentally-detectable amount, and within a predetermined time (e.g. within a dosing interval of between 6 and 24 hours (i.e. once to four times daily)).

For the avoidance of doubt, the term “parenteral” administration includes all forms of administration other than oral administration.

Prodrugs of compounds of the invention may be prepared by modifying functional groups present on the compound in such a way that the modifications are cleaved, in vivo when such prodrug is administered to a mammalian subject. The modifications typically are achieved by synthesising the parent compound with a prodrug substituent.

Prodrugs include compounds of the invention wherein a hydroxyl, amino, sulfhydryl, carboxy or carbonyl group in a compound of the invention is bonded to any group that may be cleaved in vivo to regenerate the free hydroxyl, amino, sulfhydryl, carboxy or carbonyl group, respectively.

Examples of prodrugs include, but are not limited to, esters and carbamates of hydroxy functional groups, esters groups of carboxyl functional groups, N-acyl derivatives and N-Mannich bases. General information on prodrugs may be found e.g. in Bundegaard, H. “Design of Prodrugs” p. 1-92, Elesevier, New York-Oxford (1985).

Compounds of the invention may contain double bonds and may thus exist as E (entgegen) and Z (zusammen) geometric isomers about each individual double bond. Positional isomers may also be embraced by the compounds of the invention. All such isomers (e.g. if a compound of the invention incorporates a double bond or a fused ring, the cis- and trans-forms, are embraced) and mixtures thereof are included within the scope of the invention (e.g. single positional isomers and mixtures of positional isomers may be included within the scope of the invention).

Compounds of the invention may also exhibit tautomerism. All tautomeric forms (or tautomers) and mixtures thereof are included within the scope of the invention. The term “tautomer” or “tautomeric form” refers to structural isomers of different energies which are interconvertible via a low energy barrier. For example, proton tautomers (also known as prototropic tautomers) include interconversions via migration of a proton, such as keto-enol and imine-enamine isomerisations. Valence tautomers include interconversions by reorganisation of some of the bonding electrons.

Compounds of the invention may also contain one or more asymmetric carbon atoms and may therefore exhibit optical and/or diastereoisomerism. Diastereoisomers may be separated using conventional techniques, e.g. chromatography or fractional crystallisation. The various stereoisomers may be isolated by separation of a racemic or other mixture of the compounds using conventional, e.g. fractional crystallisation or HPLC, techniques. Alternatively the desired optical isomers may be made by reaction of the appropriate optically active starting materials under conditions which will not cause racemisation or epimerisation (i.e. a ‘chiral pool’ method), by reaction of the appropriate starting material with a ‘chiral auxiliary’ which can subsequently be removed at a suitable stage, by derivatisation (i.e. a resolution, including a dynamic resolution), for example with a homochiral acid followed by separation of the diastereomeric derivatives by conventional means such as chromatography, or by reaction with an appropriate chiral reagent or chiral catalyst all under conditions known to the skilled person.

All stereoisomers (including but not limited to diastereoisomers, enantiomers and atropisomers) and mixtures thereof (e.g. racemic mixtures) are included within the scope of the invention.

In the structures shown herein, where the stereochemistry of any particular chiral atom is not specified, then all stereoisomers are contemplated and included as the compounds of the invention. Where stereochemistry is specified by a solid wedge or dashed line representing a particular configuration, then that stereoisomer is so specified and defined.

The compounds of the present invention may exist in unsolvated as well as solvated forms with pharmaceutically acceptable solvents such as water, ethanol, and the like, and it is intended that the invention embrace both solvated and unsolvated forms.

The present invention also embraces isotopically-labeled compounds of the present invention which are identical to those recited herein, but for the fact that one or more atoms are replaced by an atom having an atomic mass or mass number different from the atomic mass or mass number usually found in nature (or the most abundant one found in nature). All isotopes of any particular atom or element as specified herein are contemplated within the scope of the compounds of the invention. Exemplary isotopes that can be incorporated into compounds of the invention include isotopes of hydrogen, carbon, nitrogen, oxygen, phosphorus, sulfur, fluorine, chlorine and iodine, such as ²H, ³H, ¹¹C, ¹³C, ¹⁴C, ¹³N, ¹⁵O, ¹⁷O, ¹⁸O, ³²P, ³P, ³⁵S, ¹⁸F, ³⁶Cl, ¹²³I, and ¹²⁵I. Certain isotopically-labeled compounds of the present invention (e.g., those labeled with ³H and ¹⁴C) are useful in compound and for substrate tissue distribution assays. Tritiated (3H) and carbon-14 (¹⁴C) isotopes are useful for their ease of preparation and detectability. Further, substitution with heavier isotopes such as deuterium (i.e., ²H may afford certain therapeutic advantages resulting from greater metabolic stability (e.g., increased in vivo half-life or reduced dosage requirements) and hence may be preferred in some circumstances. Positron emitting isotopes such as ¹⁵O, ¹³N, ¹¹C and ¹⁸F are useful for positron emission tomography (PET) studies to examine substrate receptor occupancy. Isotopically labeled compounds of the present invention can generally be prepared by following procedures analogous to those disclosed in the description/Examples hereinbelow, by substituting an isotopically labeled reagent for a non-isotopically labeled reagent.

Unless otherwise specified, C_{1-q} alkyl groups (where q is the upper limit of the range) defined herein may be straight-chain or, when there is a sufficient number (i.e. a minimum of two or three, as appropriate) of carbon atoms, be branched-chain, and/or cyclic (so forming a C_{3-q}-cycloalkyl group). Such cycloalkyl groups may be monocyclic or bicyclic and may further be bridged. Further, when there is a sufficient number (i.e. a minimum of four) of carbon atoms, such groups may also be part cyclic. Such alkyl groups may also be saturated or, when there is a sufficient number (i.e. a minimum of two) of carbon atoms, be unsaturated (forming, for example, a C_{2-q} alkenyl or a C_{2-q} alkynyl group).

C_{3-q} cycloalkyl groups (where q is the upper limit of the range) that may be specifically mentioned may be monocyclic or bicyclic alkyl groups, which cycloalkyl groups may further be bridged (so forming, for example, fused ring systems such as three fused cycloalkyl groups). Such cycloalkyl groups may be saturated or unsaturated containing one or more double bonds (forming for example a cycloalkenyl group). Substituents may be attached at any point on the cycloalkyl group. Further, where there is a sufficient number (i.e. a minimum of four) such cycloalkyl groups may also be part cyclic.

The term “halo”, when used herein, preferably includes fluoro, chloro, bromo and iodo.

Aromatic groups may be aryl or heteroaryl. Ring A mentioned herein is a 6-membered aromatic group containing at least one nitrogen heteroatom. It may contain further heteroatom(s) including the one at the X₁ position (when X₁ represents ═N—).

Heteroatoms that may be mentioned include phosphorus, silicon, boron and, preferably, oxygen, nitrogen and sulfur.

For the avoidance of doubt, where it is stated herein that a group may be substituted by one or more substituents (e.g. selected from C₁₋₆ alkyl), then those substituents (e.g. alkyl groups) are independent of one another. That is, such groups may be substituted with the same substituent (e.g. same alkyl substituent) or different (e.g. alkyl) substituents.

All individual features (e.g. preferred features) mentioned herein may be taken in isolation or in combination with any other feature (including preferred feature) mentioned herein (hence, preferred features may be taken in conjunction with other preferred features, or independently of them).

The skilled person will appreciate that compounds of the invention that are the subject of this invention include those that are stable. That is, compounds of the invention include those that are sufficiently robust to survive isolation from e.g. a reaction mixture to a useful degree of purity.

In an embodiment of the invention, there is provided compounds of the invention as hereinbefore defined but in which X₂ represents N.

In an embodiment, there is also provided a compound of formula (II)

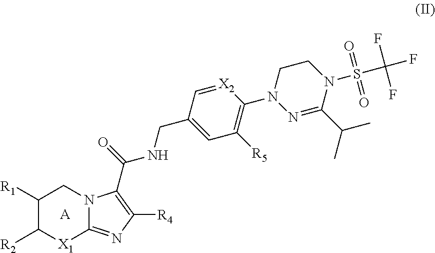

wherein
A is a 6-membered ring, which may be aromatic or non-aromatic,
X₁ represents ═N— or ═C(R₃)—;
X₂ represents ═N— or ═CH—;
R₁ is selected from H, —CH₃, and Cl;
R₂ is selected from H and —CH₃;
R₃ is selected from H and F;
R₄ is selected from —CF₃, —CHF₂ and —C₂H₅;
R₅ is selected from H and F,
   or a pharmaceutically-acceptable salt thereof.

In an embodiment, one of R₁ and R₂ represents hydrogen and the other represents a substituent other than hydrogen.

In an embodiment of the invention, preferred compounds include those of formula (IIa)

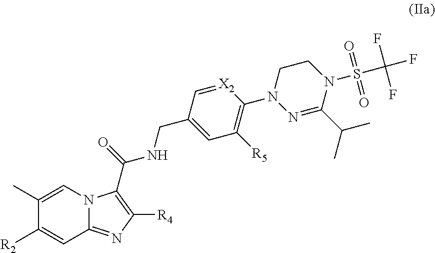

wherein
X₂ represents ═N— or ═CH—;
R₂ is selected from H and —CH₃;
R₄ is selected from —CHF₂ and —C₂H₅;
R₅ is selected from H and F,
   or a pharmaceutically-acceptable salt thereof.

In a further embodiment of the invention, preferred compounds include those of formula (IIa2)

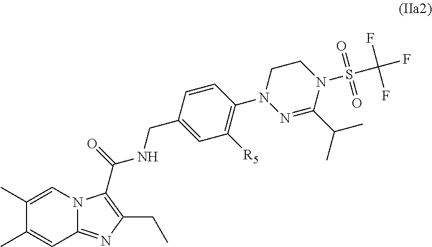

wherein
R₅ is selected from H and F,
   or a pharmaceutically-acceptable salt thereof.

In an embodiment of the invention, preferred compounds include those of formula (IIb)

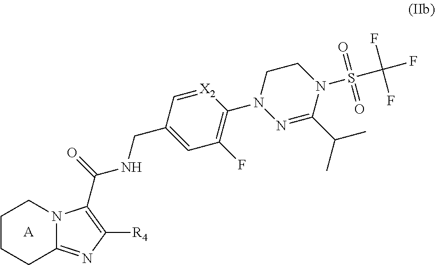

wherein
A is a 6-membered ring, which may be aromatic or non-aromatic,
X₂ represents ═N— or ═CH—;
R₄ is selected from —CF₃ and —C₂H₅,
   or a pharmaceutically-acceptable salt thereof.

In a further embodiment of the invention, preferred compounds include those of formula (IIb1)

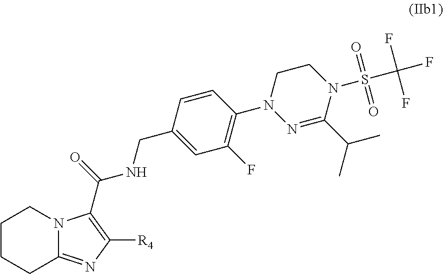

wherein
R₄ is selected from —CF₃ and —C₂H₅,
   or a pharmaceutically-acceptable salt thereof.

In a further embodiment of the invention, preferred compounds include those of formula (IIb21

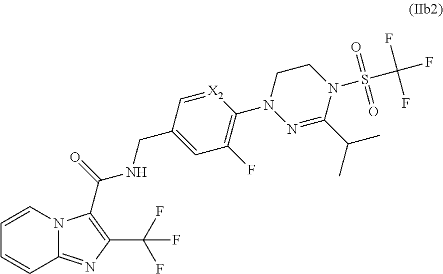

wherein
X₂ represents ═N— or ═CH—,
   or a pharmaceutically-acceptable salt thereof.

In an embodiment of the invention, preferred compounds include those of formula (IIc)

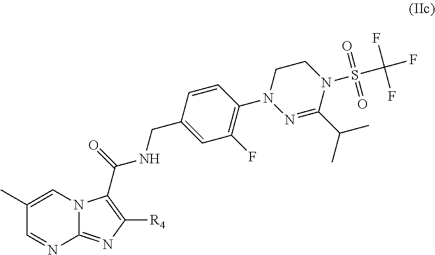

wherein
R₄ is selected from —CHF₂ and —C₂H₅,
   or a pharmaceutically-acceptable salt thereof.

In an embodiment of the invention, preferred compounds include those of formula (IId)

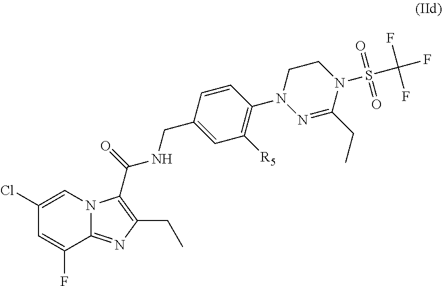

wherein
R₅ is selected from H and F,
   or a pharmaceutically-acceptable salt thereof.

In an embodiment, there is also provided a compound of formula (III)

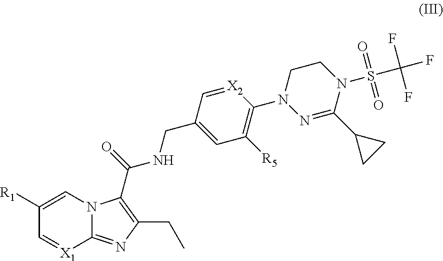

wherein
X₁ represents ═N— or ═C(R₃)—;
X₂ represents ═N— or ═CH—;
R₁ is selected from —CH₃, F, and Cl;
R₃ is selected from H and F;
R₅ is selected from H and F,
   or a pharmaceutically-acceptable salt thereof.

In an embodiment of the invention, preferred compounds include those of formula (IIIa)

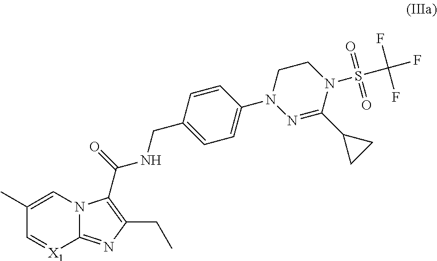

wherein
X₁ represents ═N— or ═CH—,
   or a pharmaceutically-acceptable salt thereof.

In an embodiment of the invention, preferred compounds include those of formula (IIIb)

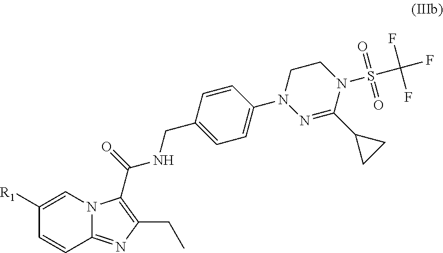

wherein
R₁ is selected from F, and Cl,
   or a pharmaceutically-acceptable salt thereof.

In an embodiment of the invention, preferred compounds include those of formula (IIIc)

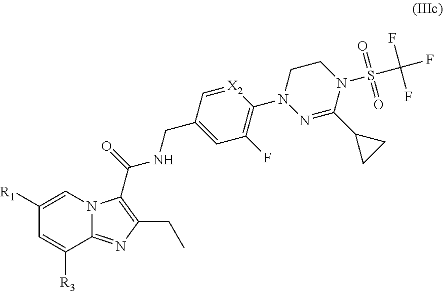

wherein
X₂ represents ═N— or ═CH—;
R₁ is selected from —CH₃ and Cl;
R₃ is selected from H and F,
   or a pharmaceutically-acceptable salt thereof.

In an embodiment, there is now also provided a compound of formula (IV),

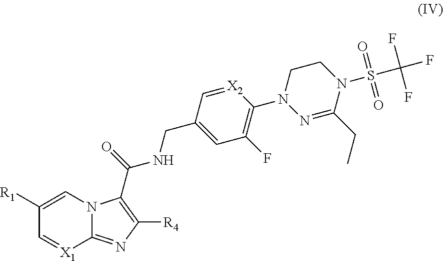

wherein
X₁ represents ═N— or ═CH—;
X₂ represents ═N— or ═CH—;
R₁ is selected from H and —CH₃;
R₄ is selected from —CF₃ and —C₂H₅,
   or a pharmaceutically-acceptable salt thereof.

In an embodiment of the invention, preferred compounds include those of formula (IVa)

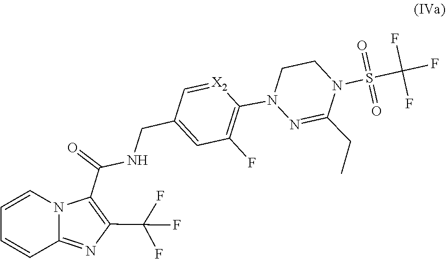

wherein
X₂ represents ═N— or ═CH—,
   or a pharmaceutically-acceptable salt thereof.

In an embodiment of the invention, preferred compounds include those of formula (IVb)

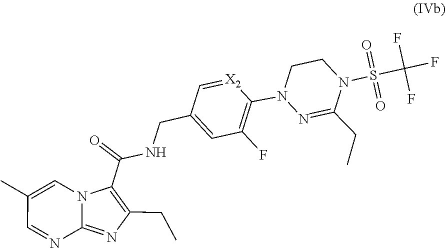

wherein
X₂ represents ═N— or ═CH—,
   or a pharmaceutically-acceptable salt thereof.

In an embodiment, there is now also provided a compound of formula (V),

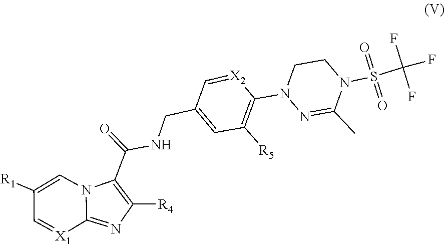

wherein
X₁ represents ═N— or ═CH—;
X₂ represents ═N— or ═CH—;
R₁ is selected from H and —CH₃;
R₄ is selected from —CF₃, —CHF₂ and —C₂H₅;
R₅ is selected from H and F,
   or a pharmaceutically-acceptable salt thereof.

In an embodiment of the invention, preferred compounds include those of formula (Va)

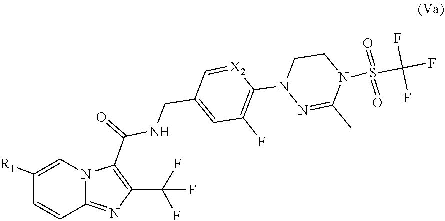

wherein
X₂ represents ═N— or ═CH—,
R₁ is selected from H and —CH₃,
   or a pharmaceutically-acceptable salt thereof.

In an embodiment of the invention, preferred compounds include those of formula (Vb)

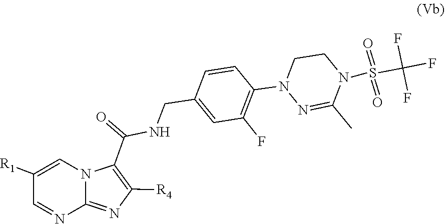

wherein
R₁ is selected from H and —CH₃;
R₄ is selected from —CF₃ and —CHF₂,
   or a pharmaceutically-acceptable salt thereof.

In an embodiment of the invention, preferred compounds include those of formula (Vc)

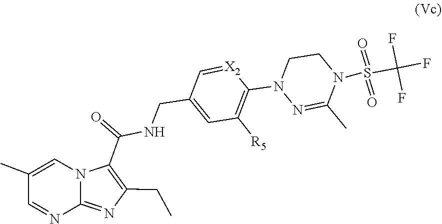

wherein
X₂ represents ═N— or ═CH—;
R₅ is selected from H and F,
   or a pharmaceutically-acceptable salt thereof.

In an embodiment, there is now also provided a compound of formula (VI),

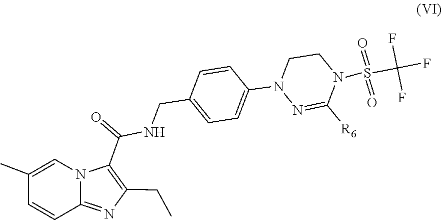

wherein
R₆ is selected from cyclopropyl, cyclobutyl, —C(═O)—OCH₃, —C(═O)—NH₂ and —C(═O)—N(CH₃)₂,
   or a pharmaceutically-acceptable salt thereof.

In an embodiment of the invention, preferred compounds include those of formula (VIa)

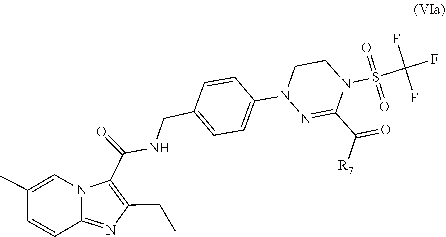

wherein
R₇ is selected from —OCH₃, —NH₂ and —N(CH₃)₂,
   or a pharmaceutically-acceptable salt thereof.

In another embodiment of the invention, preferred compounds include those of formula (VIa) wherein
R₇ is selected from —NH₂ and —N(CH₃)₂.
   or a pharmaceutically-acceptable salt thereof.

### Pharmacology

The compounds according to the invention have surprisingly been shown to be suitable for the treatment of a bacterial infection including a mycobacterial infection, particularly those diseases caused by pathogenic mycobacteria such as Mycobacterium tuberculosis (including the latent and drug resistant form thereof). The present invention thus also relates to compounds of the invention as defined hereinabove, for use as a medicine, in particular for use as a medicine for the treatment of a bacterial infection including a mycobacterial infection.

Such compounds of the invention may act by interfering with ATP synthase in M. tuberculosis, with the inhibition of cytochrome bc₁ activity being the primary mode of action. Cytochrome bc₁ is an essential component of the electron transport chain required for ATP synthesis.

Further, the present invention also relates to the use of a compound of the invention, as well as any of the pharmaceutical compositions thereof as described hereinafter for the manufacture of a medicament for the treatment of a bacterial infection including a mycobacterial infection.

Accordingly, in another aspect, the invention provides a method of treating a patient suffering from, or at risk of, a bacterial infection, including a mycobacterial infection, which comprises administering to the patient a therapeutically effective amount of a compound or pharmaceutical composition according to the invention.

The compounds of the present invention also show activity against resistant bacterial strains.

Whenever used hereinbefore or hereinafter, that the compounds can treat a bacterial infection it is meant that the compounds can treat an infection with one or more bacterial strains.

The invention also relates to a composition comprising a pharmaceutically acceptable carrier and, as active ingredient, a therapeutically effective amount of a compound according to the invention. The compounds according to the invention may be formulated into various pharmaceutical forms for administration purposes. As appropriate compositions there may be cited all compositions usually employed for systemically administering drugs. To prepare the pharmaceutical compositions of this invention, an effective amount of the particular compound, optionally in addition salt form, as the active ingredient is combined in intimate admixture with a pharmaceutically acceptable carrier, which carrier may take a wide variety of forms depending on the form of preparation desired for administration. These pharmaceutical compositions are desirable in unitary dosage form suitable, in particular, for administration orally or by parenteral injection. For example, in preparing the compositions in oral dosage form, any of the usual pharmaceutical media may be employed such as, for example, water, glycols, oils, alcohols and the like in the case of oral liquid preparations such as suspensions, syrups, elixirs, emulsions and solutions; or solid carriers such as starches, sugars, kaolin, diluents, lubricants, binders, disintegrating agents and the like in the case of powders, pills, capsules and tablets.

Because of their ease in administration, tablets and capsules represent the most advantageous oral dosage unit forms in which case solid pharmaceutical carriers are obviously employed. For parenteral compositions, the carrier will usually comprise sterile water, at least in large part, though other ingredients, for example, to aid solubility, may be included. Injectable solutions, for example, may be prepared in which the carrier comprises saline solution, glucose solution or a mixture of saline and glucose solution. Injectable suspensions may also be prepared in which case appropriate liquid carriers, suspending agents and the like may be employed. Also included are solid form preparations which are intended to be converted, shortly before use, to liquid form preparations.

Depending on the mode of administration, the pharmaceutical composition will preferably comprise from 0.05 to 99% by weight, more preferably from 0.1 to 70% by weight, even more preferably from 0.1 to 50% by weight of the active ingredient(s), and, from 1 to 99.95% by weight, more preferably from 30 to 99.9% by weight, even more preferably from 50 to 99.9% by weight of a pharmaceutically acceptable carrier, all percentages being based on the total weight of the composition.

The pharmaceutical composition may additionally contain various other ingredients known in the art, for example, a lubricant, stabilising agent, buffering agent, emulsifying agent, viscosity-regulating agent, surfactant, preservative, flavouring or colorant.

It is especially advantageous to formulate the aforementioned pharmaceutical compositions in unit dosage form for ease of administration and uniformity of dosage. Unit dosage form as used herein refers to physically discrete units suitable as unitary dosages, each unit containing a predetermined quantity of active ingredient calculated to produce the desired therapeutic effect in association with the required pharmaceutical carrier. Examples of such unit dosage forms are tablets (including scored or coated tablets), capsules, pills, powder packets, wafers, suppositories, injectable solutions or suspensions and the like, and segregated multiples thereof. The daily dosage of the compound according to the invention will, of course, vary with the compound employed, the mode of administration, the treatment desired and the mycobacterial disease indicated. However, in general, satisfactory results will be obtained when the compound according to the invention is administered at a daily dosage not exceeding 1 gram, e.g. in the range from 10 to 50 mg/kg body weight.

Given the fact that the compounds of formula (Ia) or Formula (Ib) are active against bacterial infections, the present compounds may be combined with other antibacterial agents in order to effectively combat bacterial infections.

Therefore, the present invention also relates to a combination of (a) a compound according to the invention, and (b) one or more other antibacterial agents.

The present invention also relates to a combination of (a) a compound according to the invention, and (b) one or more other antibacterial agents, for use as a medicine.

The present invention also relates to the use of a combination or pharmaceutical composition as defined directly above for the treatment of a bacterial infection.

A pharmaceutical composition comprising a pharmaceutically acceptable carrier and, as active ingredient, a therapeutically effective amount of (a) a compound according to the invention, and (b) one or more other antibacterial agents, is also comprised by the present invention.

The weight ratio of (a) the compound according to the invention and (b) the other antibacterial agent(s) when given as a combination may be determined by the person skilled in the art. Said ratio and the exact dosage and frequency of administration depends on the particular compound according to the invention and the other antibacterial agent(s) used, the particular condition being treated, the severity of the condition being treated, the age, weight, gender, diet, time of administration and general physical condition of the particular patient, the mode of administration as well as other medication the individual may be taking, as is well known to those skilled in the art. Furthermore, it is evident that the effective daily amount may be lowered or increased depending on the response of the treated subject and/or depending on the evaluation of the physician prescribing the compounds of the instant invention. A particular weight ratio for the present compound of the invention and another antibacterial agent may range from 1/10 to 10/1, more in particular from 1/5 to 5/1, even more in particular from 1/3 to 3/1.

The compounds according to the invention and the one or more other antibacterial agents may be combined in a single preparation or they may be formulated in separate preparations so that they can be administered simultaneously, separately or sequentially. Thus, the present invention also relates to a product containing (a) a compound according to the invention, and (b) one or more other antibacterial agents, as a combined preparation for simultaneous, separate or sequential use in the treatment of a bacterial infection.

The other antibacterial agents which may be combined with the compounds of the invention are for example antibacterial agents known in the art. For example, the compounds of the invention may be combined with antibacterial agents known to interfere with the respiratory chain of Mycobacterium tuberculosis, including for example direct inhibitors of the ATP synthase (e.g. bedaquiline, bedaquiline fumarate or any other compounds that may have be disclosed in the prior art, e.g. compounds disclosed in WO2004/011436), inhibitors of ndh2 (e.g. clofazimine) and inhibitors of cytochrome bd. Additional mycobacterial agents which may be combined with the compounds of the invention are for example rifampicin (=rifampin); isoniazid; pyrazinamide; amikacin; ethionamide; ethambutol; streptomycin; para-aminosalicylic acid; cycloserine; capreomycin; kanamycin; thioacetazone; PA-824; delamanid; quinolones/fluoroquinolones such as for example moxifloxacin, gatifloxacin, ofloxacin, ciprofloxacin, sparfloxacin; macrolides such as for example clarithromycin, amoxycillin with clavulanic acid; rifamycins; rifabutin; rifapentin; as well as others, which are currently being developed (but may not yet be on the market; see e.g. http://www.newtbdrugs.org/pipeline.php).

Compounds of the invention (including forms and compositions/combinations comprising compounds of the invention) may have the advantage that they may be more efficacious than, be less toxic than, be longer acting than, be more potent than, produce fewer side effects than, be more easily absorbed than, and/or have a better pharmacokinetic profile (e.g. higher oral bioavailability and/or lower clearance) than, and/or have other useful pharmacological, physical, or chemical properties over, compounds known in the prior art, whether for use in the above-stated indications or otherwise. For instance compounds of the invention may advantages associated with: lower cardiotoxicity; no reactive metabolite formation (e.g. that may cause toxicity issues, e.g. genotoxicity); no formation of degradants (e.g. that are undesired or may elicit unwanted side-effects); and/or faster oral absorption and improved bioavailability.

### General Preparation

The compounds according to the invention can generally be prepared by a succession of steps, each of which may be known to the skilled person or described herein.

### Experimental Part

Compounds of formula I may be prepared in accordance with the techniques employed in the examples hereinafter (and those methods know by those skilled in the art), for example by using the following techniques.

Compounds of formula (I) or (Ia) may be prepared by:
(i) reaction of a compound of formula (XIV),

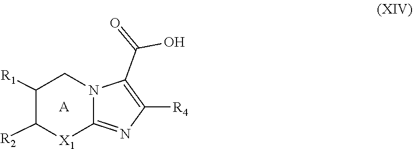

in which the integers are hereinbefore defined, with a compound of formula (XV) or (XVa), respectively,

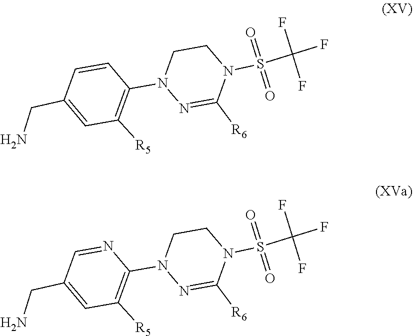

wherein the integers are as hereinbefore defined, which reaction may be performed in the presence of a suitable coupling reagent, for instance selected from diisopropylethylamine (DIPEA), 1-[bis(dimethylamino)methylene]-1H-1,2,3-triazolo[4,5-b]pyridinium-3-oxid hexafluorophosphate (HATU), 1-(3-dimethylaminopropyl)-3-ethylcarbodiimide (EDCI), 1-hydroxybenzotriazole (HOBt), O-(benzotriazole-1-yl)-N,N,N′,N′-tetramethyluronium tetrafluoroborate (TBTU), or a combination thereof, unders suitable conditions such as those described in the examples hereinafter; for example, in the presence of a suitable coupling reagent (e.g. 1,1′-carbonyldiimidazole, N,N-dicyclohexylcarbodiimide, 1-(3-dimethylaminopropyl)-3-ethylcarbodiimide (or hydrochloride thereof) or N,N-disuccinimidyl carbonate), optionally in the presence of a suitable base (e.g. sodium hydride, sodium bicarbonate, potassium carbonate, pyridine, triethylamine, dimethylaminopyridine, diisopropylamine, sodium hydroxide, potassium tert-butoxide and/or lithium diisopropylamide (or variants thereof) and an appropriate solvent (e.g. tetrahydrofuran, pyridine, toluene, dichloromethane, chloroform, acetonitrile, dimethylformamide, trifluoromethylbenzene, dioxane or triethylamine). Alternatively, the carboxylic acid group of the compound of formula (XIV) may first be converted under standard conditions to the corresponding acyl chloride (e.g. in the presence of POCl₃, PCl₅, SOCl₂ or oxalyl chloride), which acyl chloride is then reacted with a compound of formula (XV), for example under similar conditions to those mentioned above;
(ii) coupling of a compound of formula (XVII) or (XVIIa), respectively,

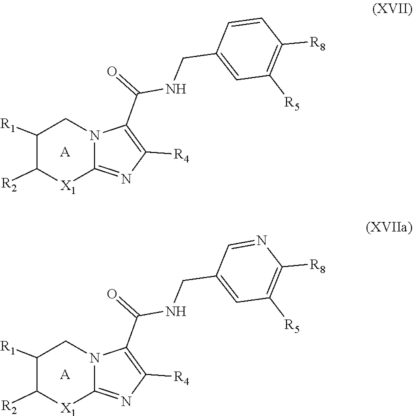

wherein the integers are as hereinbefore defined, and R₈ represents a suitable group, e.g. a suitable leaving group such as chloro, bromo, iodo or a sulfonate group (for example a type of group that may be deployed for a coupling), with a compound of formula (XVI),

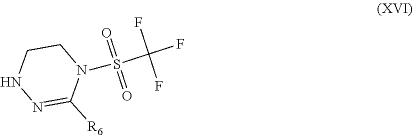

under standard conditions, for example optionally in the presence of an appropriate metal catalyst (or a salt or complex thereof) such as Pd(dba)₂, Pd(OAc)₂, Cu, Cu(OAc)₂, CuI, NiCl₂ or the like, with an optional additive such as Ph₃P, X-phos or the like, in the presence of an appropriate base (e.g. t-BuONa, or the like) in a suitable solvent (e.g. dioxane or the like) under reaction conditions known to those skilled in the art;
(iii) reaction of a compound of formula (XVIII) or (XVIIIa), respectively,

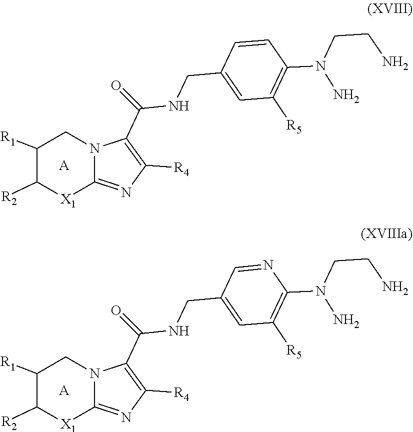

wherein the integers are as hereinbefore defined, reaction with a compound of formula (XIX)

R₆C(OCH₃)₃ (XIX)
or the like, wherein R₆ is as hereinbefore defined, under reaction conditions such as those herein described, for instance in the examples, followed by reaction with a compound of formula (XIXA)

LG¹-S(O)₂CF₃ (XIXA)
wherein LG¹ represents a suitable leaving group e.g. chloro, bromo, iodo or a sulfonate group. Hence in an embodiment, there is provided a process for the preparation of a compound of formula (I), which comprises reaction of a corresponding compound in which the —S(O)₂CF₃ group is not present (i.e. a hydrogen is present in its place) with a compound of formula (XIXA) as hereinbefore defined.

It will be appreciated by those skilled in the art that some compounds of formula (I) (e.g. those in which R₆ represents —C(O)OC₁₋₂ alkyl) may be converted to other compounds of formula (I) (e.g. those in which R₆ represents —C(O)N(R^{a})(R^{b}) by reaction with HN(R^{a})(R^{b})).

It is evident that in the foregoing and in the following reactions, the reaction products may be isolated from the reaction medium and, if necessary, further purified according to methodologies generally known in the art, such as extraction, crystallization and chromatography. It is further evident that reaction products that exist in more than one enantiomeric form, may be isolated from their mixture by known techniques, in particular preparative chromatography, such as preparative HPLC, chiral chromatography. Individual diastereoisomers or individual enantiomers can also be obtained by Supercritical Fluid Chromatography (SCF).

The starting materials and the intermediates are compounds that are either commercially available or may be prepared according to conventional reaction procedures generally known in the art.

### EXAMPLES

### 1. General Information

Melting Points

Melting points were recorded using a differential scanning calorimeter DSC 1 Mettler Toledo. Melting points were measured with a temperature gradient of 10° C. per min from 25 to 350° C. Values are peak values. Unless indicated, this method is used.

An alternative method is with open capilliary tubes on a Mettler Toledo MP50, which may be indicated at “MT”. With this method, melting points are measured with a temperature gradient of 10° C./minute. Maximum temperature is 300° C. The melting point data is read from a digital display and checked from a video recording system.

¹H NMR

¹H NMR spectra were recorded on a Bruker Avance DRX 400 spectrometer or Bruker Advance III 400 spectrometer using internal deuterium lock and equipped with reverse double-resonance (¹H, 13C, SEI) probe head with z gradients and operating at 400 MHz for proton and 100 MHz for carbon and a Bruker Avance 500 MHz spectrometer equipped with a Bruker 5 mm BBFO probe head with z gradients and operating at 500 MHz for proton and 125 MHz for carbon.

NMR spectra were recorded at ambient temperature unless otherwise stated.

Data are reported as follow: chemical shift in parts per million (ppm) relative to TMS (δ=0 ppm) on the scale, integration, multiplicity (s=singulet, d=doublet, t=triplet, q=quartet, quin=quintuplet, sex=sextuplet, m=multiplet, b=broad, or a combination of these), coupling constant(s) J in Hertz (Hz).

HPLC-LCMS

Analytical Methods

LCMS

The mass of some compounds was recorded with LCMS (liquid chromatography mass spectrometry). The methods used are described below.

General Procedure LCMS Methods A and B

The High Performance Liquid Chromatography (HPLC) measurement was performed using a LC pump, a diode-array (DAD) or a UV detector and a column as specified in the respective methods. If necessary, additional detectors were included (see table of methods below). Flow from the column was brought to the Mass Spectrometer (MS) which was configured with an atmospheric pressure ion source. It is within the knowledge of the skilled person to set the tune parameters (e.g. scanning range, dwell time . . . ) in order to obtain ions allowing the identification of the compound's nominal monoisotopic molecular weight (MW). Data acquisition was performed with appropriate software.

Compounds are described by their experimental retention times (Rt) and ions. If not specified differently in the table of data, the reported molecular ion corresponds to the [M+H]⁺ (protonated molecule) and/or [M−H]⁻ (deprotonated molecule). In case the compound was not directly ionizable the type of adduct is specified (i.e. [M+NH₄]⁺, [M+HCOO]⁻, etc. . . . ). For molecules with multiple isotopic patterns (Br, Cl . . . ), the reported value is the one obtained for the lowest isotope mass. All results were obtained with experimental uncertainties that are commonly associated with the method used. Hereinafter, “SQD” means Single Quadrupole Detector, “RT” room temperature, “BEH” bridged ethylsiloxane/silica hybrid, “HSS” High Strength Silica, “DAD” Diode Array Detector, “MSD” Mass Selective Detector.

TABLE
LCMS Method codes (Flow expressed in mL/min; column temperature (T) in ° C.; Run time in minutes).

| Method | | | | | Flow | Run |
|---|---|---|---|---|---|---|
| code | Instrument | Column | Mobile phase | gradient | Column T | time |
| A | Waters: Acquity | Waters: BEH C18 | A: 95% CH₃COONH₄ | 84.2% A for 0.49 min, | 0.343 | 6.2 |
| | UPLC ® - DAD | (1.7 μm, 2.1 × | 7 mM/5% CH₃CN | to 10.5% A in 2.18 min, | 40 | |
| | and Quattro | 100 mm) | B: CH₃CN | held for 1.94 min, back | | |
| | Micro ™ | | | to 84.2% A in 0.73 min, | | |
| | | | | held for 0.73 min. | | |
| B | Waters: Acquity ® | Waters: BEH C18 | A: 95% CH₃COONH₄ | 84.2% A to 10.5% A in 2.18 | 0.343 | 6.1 |
| | H-Class - DAD | (1.7 μm, 2.1 × | 7 mM/5% CH₃CN | min, held for 1.96 min, back | 40 | |
| | and SQD2 ™ | 100 mm) | B: CH₃CN | to 84.2% A in 0.73 min, held | | |
| | | | | for 0.73 min. | | |
| C | Waters: Acquity | Waters: BEH C18 | A: 95% CH₃COONH₄ | From 85% A to 10% A in | 0.35 | 6.1 |
| | UPLC ® H-Class - | (1.7 μm, 2.1 × | 7 mM/5% CH₃CN, | 2.1 min, held for 2 min, | 40 | |
| | DAD and QDa | 100 mm) | B: CH₃CN | back to 85% A in 0.8 min, | | |
| | | | | held for 0.7 min. | | |
| D | Agilent 1100 HPLC | YMC-pack ODS-AQ | A: 0.1% HCOOH in | From 95% A to 5% A in | 2.6 | 6.2 |
| | DAD LC/MS G1956A | C18 (50 × 4.6 mm, | H2O B: CH3CN | 4.8 min, held for 1.0 min, | 35 | |
| | | 3 μm) | | to 95% A in 0.2 min. | | |
| E | Waters: Acquity | Waters BEH ® C18 | A: 95% CH₃COONH₄ | From 95% A/5% B to 5% | 0.5 | 3.3 |
| | UPLC ® H-Class - | (1.7 μm, 2.1 × 50 mm) | 7 mM/5% CH₃CN, | A in 1 min, held for 1.6 min, | 40 | |
| | DAD and QDa | | B: CH₃CN | back to 95% A/5% B in 0.2 | | |
| | | | | min, held for 0.5 min. | | |

When a compound is a mixture of isomers which give different peaks in the LCMS method, only the retention time of the main component is given in the LCMS table.

### 2. Abbreviations (and Formulae)

AcOH Acetic acid
AcCl Acetyl chloride
BINAP 2,2′-bis(diphenylphosphino)-1,1′-binaphthyl
BrettPhos 2-(Dicyclohexylphosphino)3,6-dimethoxy-2′,4′,6′-triisopropyl-1,1′-biphenyl
BrettPhos Pd G3 [(2-Di-cyclohexylphosphino-3,6-dimethoxy-2′,4′,6′-triisopropyl-1,1′-biphenyl)-2-(2′-amino-1,1′-biphenyl)]palladium(II) methanesulfonate methanesulfonate
CBr₄ Tetrabromomethane
CbzCl Benzyl chloroformate
CH₃CN/ACN Acetonitrile
Cs₂CO₃ Cesium carbonate
CSA Camphor-O-sulfonic acid
DCE Dichloroethane
DCM or CH₂Cl₂ Dichloromethane
DIPEA N,N-Diisopropylethylamine
DMAP 4-(Dimethylamino)pyridine
DME 1,2-Dimethoxyethane
DMF Dimethylformamide
DMF-DMA N,N-dimethylformamide dimethyl acetal
DMSO Methyl sulfoxide
EDCI·HCl N-(3-Dimethylaminopropyl)-N′-ethylcarbodiimide hydrochloride
Et₂O Diethylether
Et₃N or TEA Triethylamine
EtOAc Ethyl acetate
EtOH Ethanol
h hour
H₂ Dihydrogen gas
HATU Hexafluorophosphate Azabenzotriazole Tetramethyl Uronium
HCl Hydrochloric acid
HFIP Hexafluoroisopropanol
HOBT·H₂O 1-Hydroxybenzotriazole hydrate
i-PrOH Isopropyl alcohol
K₂CO₃ Potassium carbonate
KHSO₄ Potassium bisulfate
LiOH Lithium hydroxide
LiHMDS Lithium bis(trimethylsilyl)amide
MeOH Methanol
MeTHF/2-MeTHF Methyltetrahydrofurane
MgSO₄ Magnesium sulfate
min Minute
N₂ Nitrogen
NaCl Sodium Chloride
NaHCO₃ Sodium Bicarbonate
NaOH Sodium hydroxide
NBS 1-bromopyrrolidine-2,5-dione
NH₃ Ammonia
NH₄Cl Ammonium, chloride
NH₄HCO₃ Ammonium bicarbonate
NMR Nuclear Magnetic Resonance
Pd/C Palladium on carbon
PdCl₂(PPh₃)₂ Dichlorobis(triphenylphosphine)palladium(II)
Pd(OAc)₂ Palladium(II) acetate
Pd₂dba₃ Tris(dibenzylideneacetone)dipalladium(O)
Pd(PPh₃)₄ Palladium-tetrakis(triphenylphosphine)
PIDA (Diacetoxyiodo)benzene
POCl₃ Phosphorous Oxychloride
Ra-Ni/Ni Raney Raney®-Nickel
rt/RT Room temperature
RuPhos 2-Dicyclohexylphosphino-2′,6′-diisopropoxybiphenyl
RuPhos Pd G3 (2-Dicyclohexylphosphino-2′,6′-diisopropoxy-1,1′-biphenyl)[2-(2′-amino-1,1′-biphenyl)] palladium(II) methanesulfonate
t-AmylOH tert-Amyl alcohol
SiOH Silica Gel
TBTU O-(benzotriazole-1-yl)-N,N,N′,N′-tetramethyluronium tetrafluoroborate
Tf₂O Trifluoromethanesulfonic Anhydride
TFA Trifluoroactetic acid
THF Tetrahydrofuran
TMSCl Trimethylsilyl chloride
TsOH or PTSA p-Toluensulfonic acid
XantPhos 4,5-Bis(diphenylphosphino)-9,9-dimethylxanthene

### Reference Examples

### 3. Procedures

Synthesis of Compound 1

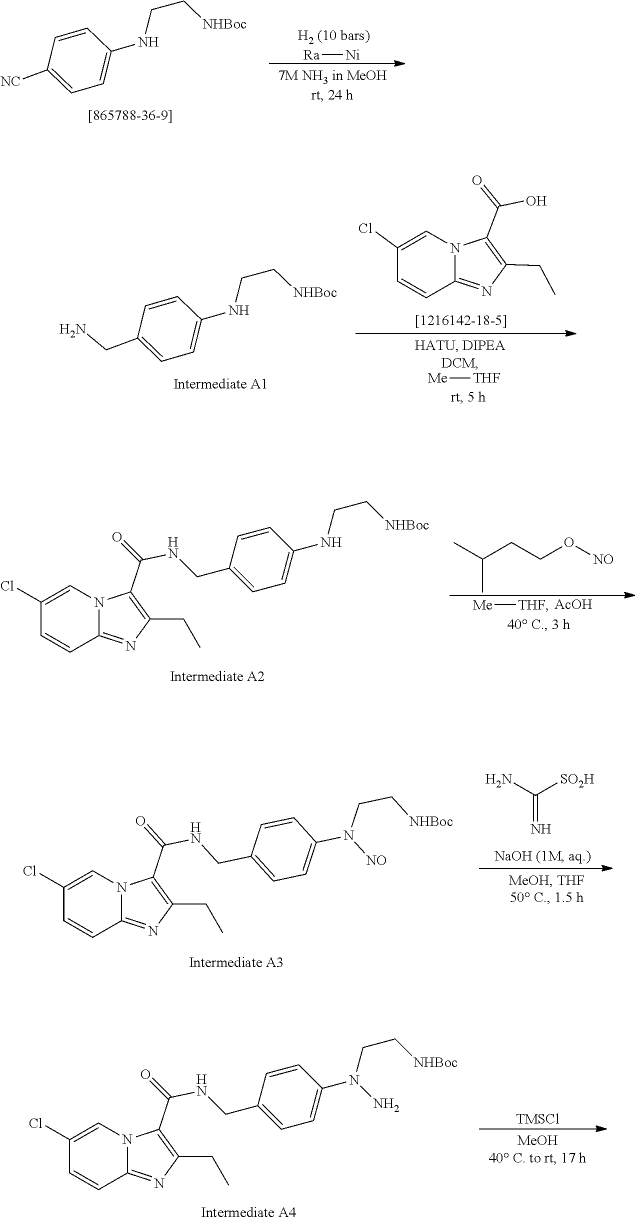

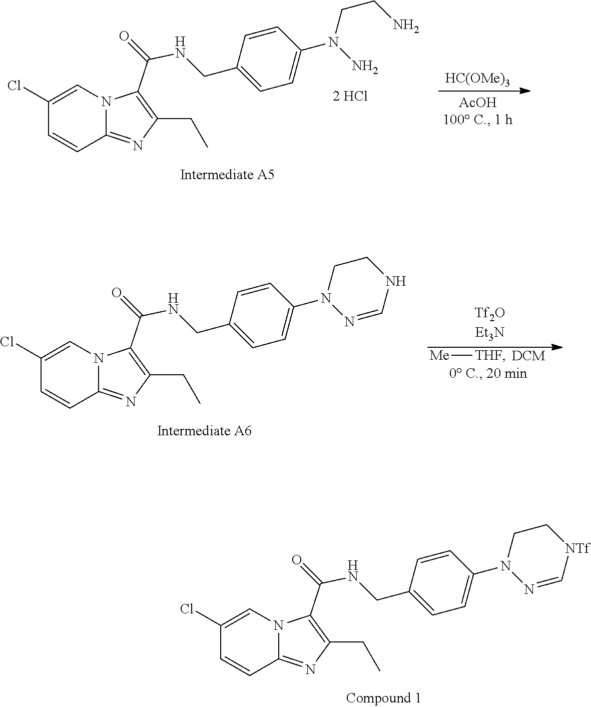

Preparation of Intermediate A1

In an 1 L autoclave, a mixture of N-Boc-[2-[(4-cyanophenyl)amino]ethyl] [865788-36-9] (50.0 g, 191 mmol) and Raney Nickel (2.25 g, 38.2 mmol) in a 7M solution of NH₃ in MeOH (600 mL) was hydrogenated at room temperature under 10 bars of H₂ for 24 h. The reaction mixture was filtered through a pad of Celite® and washed with a mixture of DCM and MeOH (9/1). The filtrate was evaporated in vacuo to afford 50.2 g of intermediate A1 as a greenish oil (99%).

Preparation of Intermediate A2

A 2 L flask was charged with 6-chloro-2-ethylimidazo[1,2-a]pyridine-3-carboxylic acid [1216142-18-5] (15.0 g, 66.8 mmol), intermediate A1 (18.6 g, 70.1 mmol) and DIPEA (17.3 mL, 100 mmol) in DCM (600 mL) and Me-THF (100 mL). The reaction mixture was stirred for 10 min at room temperature, then HATU (27.9 g, 73.4 mmol) was added portionwise over 5 minutes and the reaction mixture was stirred at room temperature for 5 h. The mixture was diluted with DCM (1 L) and water (800 mL). The organic layer was separated and washed with water (400 mL), dried over MgSO₄, filtered and evaporated in vacuo. The residue was solubilized in a minimum amount of warm EtOAc. The solution was cooled to room temperature, and then to 0° C. The suspension was collected by filtration and the solid was washed with cold EtOAc, then with Et₂O before being dried under vacuum to afford 21.7 g of intermediate A2 as an off-white solid (69%).

Preparation of Intermediate A3

Intermediate A2 (5.00 g, 10.6 mmol) was solubilized at 40° C. in Me-THF (80 mL) and acetic acid (6.1 mL, 106 mmol). Isopentyl nitrite (7.12 mL, 53.0 mmol) was added dropwise and the reaction mixture was stirred at 40° C. for 3 h. The solution was diluted in EtOAc and water, washed with NaHCO₃ (sat., aq.) (twice) and brine, dried over MgSO₄ and evaporated in vacuo. The residue was triturated in Et₂O. the product was collected by filtration, washed with Et₂O and dried under vacuum to give 4.26 g of intermediate A3 as a beige solid (80%).

Preparation of Intermediate A4

A solution of intermediate A3 (5.00 g, 9.98 mmol) in THE (100 mL) and MeOH (65 mL) was treated with a NaOH (1M, aq., 100 mL). Formamidinesulfinic acid (5.40 g, 49.9 mmol) was added and the reaction mixture was stirred at 50° C. for 1.5 h. The reaction mixture was diluted in DCM and K₂CO₃ (10%, aq.) was added. The layers were separated. The aqueous phase was extracted with DCM and MeOH (95/5). The combined organic extracts were dried over MgSO₄, filtered and evaporated in vacuo to give 4.67 g of intermediate A4 as a white solid (Quant.).

Preparation of Intermediate A5

To a solution of intermediate A4 (4.67 g, 9.59 mmol) in MeOH (96 mL) was added dropwise TMSCl (9.73 mL, 76.7 mmol). The reaction mixture was stirred at 40° C. for 1.5 h and at room temperature for another 17 h. The mixture was concentrated in vacuo. The residue was triturated in Et₂O. the solid was collected by filtration, washed with Et₂O, and dried under vacuum to afford 4.76 g of intermediate A5 as a pale yellow solid (Quant.).

Preparation of Intermediate A6

A mixture of intermediate A5 (4.76 g, 10.4 mmol) and trimethyl orthoformate (3.40 mL, 31.1 mmol) in acetic acid (52 mL) was stirred for 1 h at 100° C. The reaction mixture was concentrated in vacuo. The residue was diluted in DCM and K₂CO₃ (10%, aq.) was added. The aqueous layer was extracted with DCM and MeOH (95/5) twice. The combined organic extracts were dried over MgSO₄, filtered and evaporated in vacuo to give 3.44 g of intermediate A6 as a beige solid (83%).

Preparation of Compound 1

A solution of intermediate A6 (80 mg, 0.202 mmol) in DCM (6 mL) and Me-THF (3 mL) was treated with Et₃N (70 μL, 0.50 mmol). The mixture was cooled to 0° C. and a solution of Tf₂O (1M in DCM, 302 μL, 0.302 mmol) was added dropwise. The reaction mixture was stirred at 0° C. for 20 min. MeOH (0.3 mL) was added, followed by K₂CO₃ (10%, aq., 5 mL) and DCM. The layers were separated. The organic phase was dried over MgSO₄, filtered and evaporated in vacuo. The crude mixture was purified by preparative LC (irregular SiOH 15-40 μm, 12 g, dry loading (Celite®), mobile phase: heptane/EtOAc, gradient from 70:30 to 0:100). The residue (62 mg) was dissolved in warm EtOAc (3 mL) and allowed to cool down to room temperature. The supernatent was removed. The solid was triturated in Et₂O. The product was collected by filtration and dried under vacuum to afford 42 mg of compound 1 as a white solid (36%).

¹H NMR (400 MHz, DMSO-d₆) δ ppm 9.07 (s, 1H), 8.47 (br s, 1H), 7.67 (d, J=8.1 Hz, 1H), 7.46 (br d, J=9.1 Hz, 1H), 7.30 (br d, J=8.1 Hz, 2H), 7.20 (br d, J=7.6 Hz, 2H), 4.49 (br d, J=5.1 Hz, 2H), 4.41 (s, 2H), 4.18 (s, 2H), 3.39-3.31 (m, 1H), 2.98 (q, J=7.4 Hz, 2H), 2.63-2.58 (m, 2H), 2.34-2.29 (m, 2H), 1.26 (br t, J=7.3 Hz, 3H)

¹H NMR (400 MHz, DMSO-d₆) δ ppm 9.12 (s, 1H) 8.71 (m, 1H) 7.79 (d, J=9.4 Hz, 1H) 7.68 (d, J=8.8 Hz, 1H) 7.26-7.37 (m, 3H) 7.19 (d, J=8.7 Hz, 2H) 4.48 (d, J=5.9 Hz, 2H) 4.08 (t, J=4.5 Hz, 2H) 3.83 (t, J=4.8 Hz, 2H) 3.01 (q, J=7.6 Hz, 2H) 1.27 (t, J=7.5 Hz, 3H)

Synthesis of Compound 2

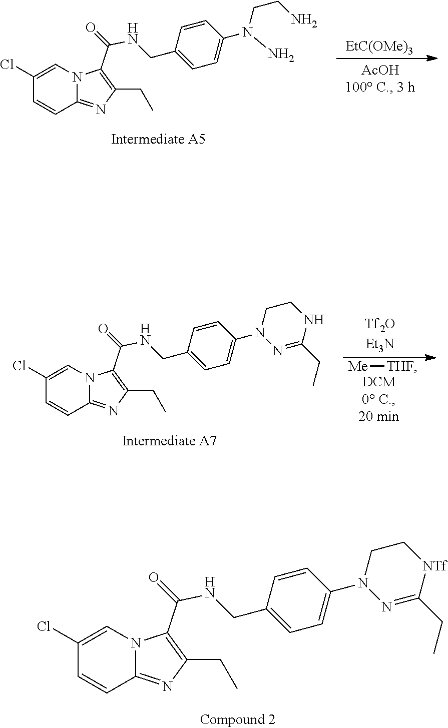

Preparation of Intermediate A7

A mixture of intermediate A5 (300 mg, 0.652 mmol) and trimethyl orthopropionate (0.102 mL, 0.718 mmol) in acetic acid (6 mL) was stirred for 1 h at 100° C. Additional amount of trimethylorthopropionate (0.102 mL, 0.718 mmol) was added and the reaction mixture was stirred for at 100° C. for another 2 h. The reaction mixture was diluted in DCM and NaOH (3M, aq.). The layers were separated and the organic phase was dried over MgSO₄, filtered and evaporated in vacuo to give 138 mg of intermediate A7 as a foam (50%).

Preparation of Compound 2

A solution of intermediate A7 (138 mg, 0.325 mmol) in DCM (4 mL) was treated with Et₃N (113 μL, 0.812 mmol). The mixture was cooled to 0° C. and a solution of Tf₂O in DCM (1M in DCM, 357 μL, 0.357 mmol) was added dropwise. The reaction mixture was stirred at 0° C. for 20 min. The reaction was quenched with MeOH (0.2 mL) and pyridine (0.1 mL). Celite® was added and the mixture was evaporated in vacuo. The residue was purified by preparative LC (irregular SiOH 15-40 μm, 24 g, dry loading (Celite®), mobile phase: heptane/EtOAc, gradient from 70:30 to 0:100). A second purification was performed by reverse phase (stationary phase: YMC-actus Triaroom temperature C18 10 μm 30*150 mm, mobile phase: NH₄HCO₃ (0.2% in water)/MeCN, gradient from 40:60 to 10:90) to give 60 mg of compound 2 as a white solid (33%).

¹H NMR (400 MHz, DMSO-d₆) δ ppm 9.07 (d, J=1.6 Hz, 1H) 8.43 (t, J=5.9 Hz, 1H) 7.66 (d, J=9.5 Hz, 1H) 7.45 (dd, J=9.5, 2.1 Hz, 1H) 7.32 (d, J=8.7 Hz, 2H) 7.18 (d, J=8.8 Hz, 2H) 4.46 (d, J=5.9 Hz, 2H) 3.91-4.02 (m, 2H) 3.79-3.90 (m, 2H) 2.98 (q, J=7.5 Hz, 2H) 2.61 (q, J=7.3 Hz, 2H) 1.26 (t, J=7.5 Hz, 3H) 1.18 (t, J=7.3 Hz, 3H).

Synthesis of Compound 3

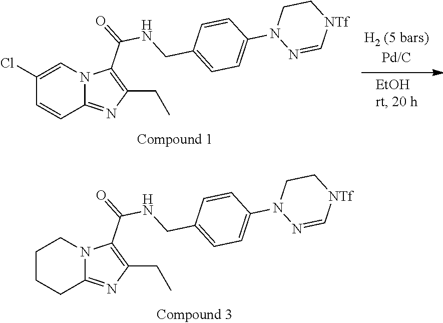

In a pressure vessel reactor, a mixture of compound 1 (250 mg, 0.473 mmol) and Pd/C (54 mg, 50.5 μmol) in EtOH (15 mL) was stirred at room temperature under 5 bar of H₂ for 20 h. The mixture was filtered over a pad of Celite®. The filtered cake was washed with EtOH and DCM, and the filtrate was evaporated in vacuo. The residue was combined with another batch to give 250 mg of a crude mixture. The residue was purified by reverse phase (Stationary phase: YMC-actus Triaroom temperature C18 10 μm 30*150 mm, mobile phase: NH₄HCO₃ (0.2% in water)/MeCN, gradient from 55:45 to 30:70). The residue was triturated in Et₂O, and the solvent was removed under reduced pressure to give 165 mg of compound 3 as a white solid (58%).

¹H NMR (400 MHz, DMSO-d₆) δ ppm 8.16 (t, J=6.1 Hz, 1H) 7.28 (s, 1H) 7.26 (d, J=8.6 Hz, 2H) 7.16 (d, J=8.6 Hz, 2H) 4.35 (d, J=6.1 Hz, 2H) 4.07 (t, J=4.6 Hz, 2H) 3.97 (t, J=5.7 Hz, 2H) 3.77-3.87 (m, 2H) 2.68-2.75 (t, J=6.4 Hz, 2H) 2.60 (q, J=7.5 Hz, 2H) 1.73-1.90 (m, 4H) 1.09 (t, J=7.5 Hz, 3H).

Synthesis of Compound 4

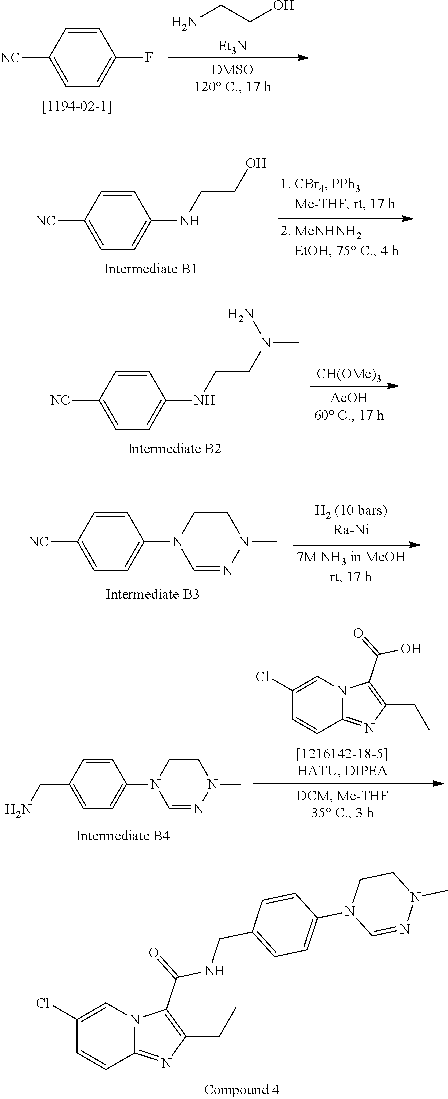

Preparation of Intermediate B1

A flask (equipped with a findenser) was charged with 4-fluorobenzonitrile [1194-02-1](1.00 g, 8.26 mmol), DMSO (5.9 mL) and ethanolamine (0.757 g, 12.4 mmol). Et₃N (1.72 mL, 12.4 mmol) was added and the reaction mixture was stirred at 120° C. for 17 h. The mixture was poured into brine. The layers were separated and the aqueous phase was extracted with EtOAc. The combined organic extracts were washed with brine (3 times), dried over MgSO₄, filtered and evaporated in vacuo to afford intermediate B1 as pale-yellow oil (Quant.).

Preparation of Intermediate B2

A solution of the intermediate B1 (2.00 g, 12.3 mmol) and triphenylphosphine (4.21 g, 16.0 mmol) in Me-THF (100 mL) was treated with CBr₄ (5.32 g, 16.0 mmol). The reaction mixture was stirred at room temperature for 17 h. The mixture was evaporated in vacuo. The residue was solubilized in EtOH (40 mL) and treated with methylhydrazine (5.19 mL, 98.6 mmol). The reaction mixture was stirred at 75° C. for 4 h and concentrated in vacuo. The residue was diluted with DCM and HCl (3M, aq.) was added. The layers were separated and the organic phase was washed with water. The combined aqueous extracts were basified by the addition of K₂CO₃. The aqueous phase was extracted with DCM (twice). The combined organic layers were dried over MgSO₄, filtered and evaporated in vacuo to afford 2.54 g of compound B2 as an orange oil (Quant.).

Preparation of Intermediate B3

A solution of intermediate B2 (2.15 g, 11.3 mmol) and trimethyl orthoformate (3.71 mL, 33.9 mmol) in acetic acid (60 mL) was stirred at 60° C. for 17 h. The yellow solution was cooled to room temperature. Water (150 mL) and EtOAc (150 mL) were added. K₂CO₃ was added portionwise until basification of the aqueous layer. The organic layer was separated, washed with water, and brine, dried over MgSO₄, filtered and evaporated in vacuo to give 1.50 g of intermediate B3 as an orange solid (66%).

Preparation of Intermediate B4

In an autoclave, a mixture of intermediate B3 (1.5 g, 7.49 mmol) and Raney Nickel (440 mg, 7.49 mmol) in a 7M solution of NH₃ in MeOH (64 mL) was hydrogenated at room temperature under 5 bars of H₂ for 17 h. The reaction mixture was filtered through a pad of Celite®, and washed with a mixture of DCM and MeOH (9/1). The filtrate was evaporated in vacuo to afford 1.53 g of intermediate B4 as a grey solid (Quant.).

Preparation of Compound 4

6-Chloro-2-ethylimidazo[1,2-a]pyridine-3-carboxylic acid [1216142-18-5] (600 mg, 2.67 mmol) was solubilized in Me-THF (30 mL), and DCM (15 mL) and DIPEA (0.736 mL, 4.27 mmol) was added. After complete solubilization, intermediate B4 (627 mg, 3.07 mmol) was added followed by HATU (1.17 g, 3.07 mmol). The reaction mixture was stirred for 3 h at 35° C. EtOAc and water was added. The organic layer was separated and washed with water, then brine. The combined organic extracts were dried over MgSO₄, filtered and evaporated in vacuo. The residue was solubilized in a minimum amount of warm EtOAc. The solution was cooled to room temperature and the suspension was filtered. The solid was washed with EtOAc, then with EtOH and Et₂O. The solid was collected by filtration and dried under vacuum to afford 210 mg of an off-white solid. The solid was combined with the filtrate and evaporated in vacuo. The residue was purified by preparative LC (irregular SiOH 15-40 μm, 80 g, mobile phase: DCM/(DCM/MeOH/NH₃ aq., 18/20/2), gradient from 90:10 to 60:40). The residue was crystallized from EtOAc, washed with Et₂O and dried under vacuum to afford 317 mg of compound 4.

¹H NMR (400 MHz, DMSO-d₆) δ ppm 9.07 (d, J=1.47 Hz, 1H) 8.45 (t, J=5.81 Hz, 1H) 7.67 (d, J=9.66 Hz, 1H) 7.46 (dd, J=9.41, 2.08 Hz, 1H) 7.30-7.36 (m, 3H) 7.11 (d, J=8.56 Hz, 2H) 4.47 (d, J=5.87 Hz, 2H) 3.70 (t, J=5.01 Hz, 2H) 3.17 (d, J=5.14 Hz, 1H) 2.88-3.01 (m, 4H) 2.54-2.65 (m, 4H) 1.26 (t, J=7.52 Hz, 3H).

Synthesis of Compound 5

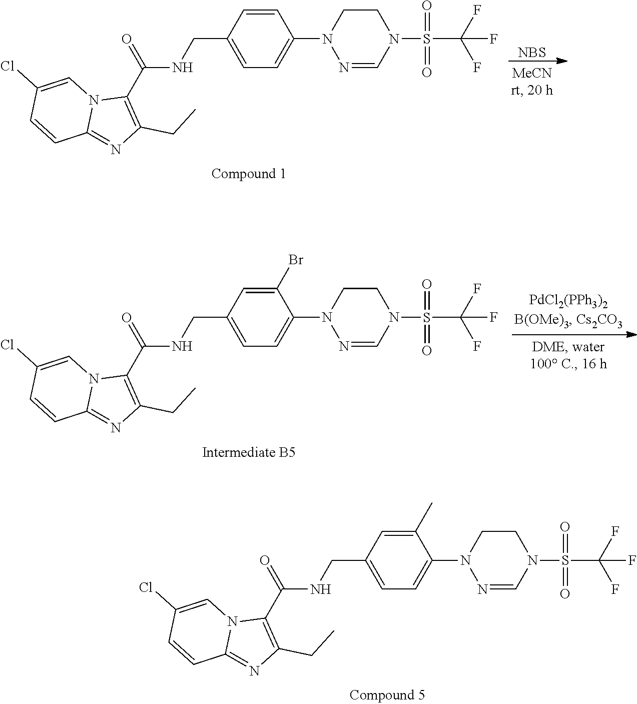

Preparation of Intermediate B5

NBS (204 mg, 1.15 mmol) was added to a solution of Compound 1 (600 mg, 1.13 mmol) in MeCN (9.5 mL) and the reaction mixture was stirred at room temperature for 20 h. The mixture was diluted with EtOAc and water. The layers were separated. The organic phase was washed NaHCO₃ (sat., aq.), dried over MgSO₄, filtered and the solvent was removed under reduced pressure to give 700 mg of intermediate B5 as a brown residue.

Preparation of Compound 5

A mixture of intermediate B5 (250 mg, 0.234 mmol), trimethylboroxine (131 μL, 0.938 mmol) and Cs₂CO₃ (229 mg, 0.703 mmol) in DME (3.6 mL) and water (3.6 mL) was purged with N₂. PdCl₂(PPh₃)₂ (32.9 mg, 0.0469 mmol) was added and the mixture was purged again with N₂. The reaction mixture was stirred at 100° C. for 16 h. Water and EtOAc were added. The layers were separated and the aqueous phase was extracted with EtOAc. The combined organic extracts were washed with brine, dried over MgSO₄, filtered and evaporated to dryness in vacuo. The residue was purified by preparative LC (irregular SiOH 15-40 μm, 24 g, dry loading (Celite®), mobile phase: DCM/MeOH, gradient from 99:1 to 95:5). A second purification was performed via reverse phase (stationary phase: YMC-actus Triaroom temperature C18 10 μm 30*150 mm, mobile phase NH₄HCO₃ (0.2% in water/MeCN, gradient from 55:45 to 35:65) to give 14 mg of a white residue which was solubilized in MeCN, extended with water and freeze-dried to give 12 mg of compound 5 as a white powder (7%).

¹H NMR (400 MHz, DMSO-d₆) δ ppm 9.07 (d, J=1.34 Hz, 1H) 8.48 (t, J=5.99 Hz, 1H) 7.67 (d, J=9.41 Hz, 1H) 7.46 (dd, J=9.54, 2.08 Hz, 1H) 7.29 (s, 1H) 7.22 (s, 1H) 7.21 (d, J=7.74 Hz, 2H) 7.12-7.17 (m, 1H) 4.49 (d, J=6.11 Hz, 2H) 4.10 (br d, J=4.28 Hz, 2H) 3.38-3.54 (m, 4H) 3.00 (q, J=7.42 Hz, 2H) 2.67-2.69 (m, 1H) 2.52-2.56 (m, 5H) 2.33-2.45 (m, 2H) 2.25 (s, 3H) 1.19-1.33 (m, 3H).

Synthesis of Compound 6

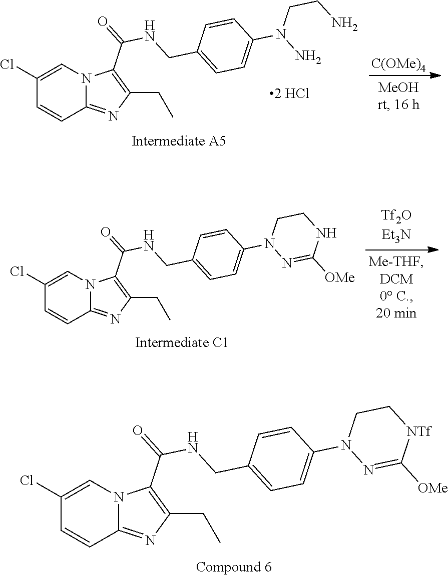

Preparation of Intermediate C1

In a sealed tube, a mixture of intermediate A5 (300 mg, 0.652 mmol) and molecular sieves 3 Å in MeOH (4.3 mL) was stirred at room temperature for 10 min. Tetramethyl orthocarbonate (347 μL, 2.61 mmol) was added and the reaction mixture was stirred at room temperature for 16 h. Water and DCM were added. The layers were separated and the organic phase was dried over MgSO₄, filtered and evaporated in vacuo to dryness. The residue was purified by preparative LC (irregular SiOH 15-40 μm, 24 g, dry loading (Celite®), mobile phase: heptane/EtOAc, gradient from 60:40 to 0:100) to give 77 mg of intermediate C1 as a white solid (24%).

Preparation of Compound 6

To a solution of intermediate C1 (48 mg, 0.112 mmol) in anhydrous DCM (1.3 mL) at room temperature was added Et₃N (23.4 μL, 0.169 mmol) and the mixture was stirred at room temperature for 10 min. The mixture was cooled at 0° C. and a solution of Tf₂O in DCM (1M in DCM, 112 μL, 0.112 mmol) was added dropwise. The mixture was stirred warming to room temperature for 1 h. A solution of Tf₂O in DCM (1M in DCM, 112 μL, 0.112 mmol) was added and the mixture was stirred at room temperature for another 1 h. NaHCO₃ (sat., aq.) and DCM were added. The layers were separated, and the organic phase was washed with NaHCO₃ (twice) and brine. The combined organic extracts were dried over MgSO₄, filtered and concentrated in vacuo. The residue was purified by preparative LC (irregular SiOH 15-40 μm, 24 g, dry loading (Celite®), mobile phase: heptane/EtOAc, gradient from 50:50 to 0:100). A second purification was performed via reverse phase (stationary phase: YMC-actus Triaroom temperature C18 10 μm 30*150 mm, mobile phase: NH₄HCO₃ (0.2% in water)/MeCN, gradient from 45:55 to 25:75) to give 33 mg of compound 6 as a white solid (37%).

¹H NMR (500 MHz, DMSO-d₆) δ ppm 9.07 (d, J=1.58 Hz, 1H) 8.39 (t, J=5.83 Hz, 1H) 7.66 (d, J=9.46 Hz, 1H) 7.44 (dd, J=9.46, 2.21 Hz, 1H) 7.29 (d, J=8.51 Hz, 2H) 7.15 (d, J=8.83 Hz, 2H) 4.46 (d, J=5.99 Hz, 2H) 4.06-4.14 (m, 2H) 3.85 (s, 3H) 3.71-3.77 (m, 2H) 3.32-3.46 (m, 2H) 3.17 (d, J=5.36 Hz, 1H) 2.97 (q, J=7.36 Hz, 2H) 2.52-2.58 (m, 6H) 1.26 (t, J=7.57 Hz, 3H).

Synthesis of Compound 7

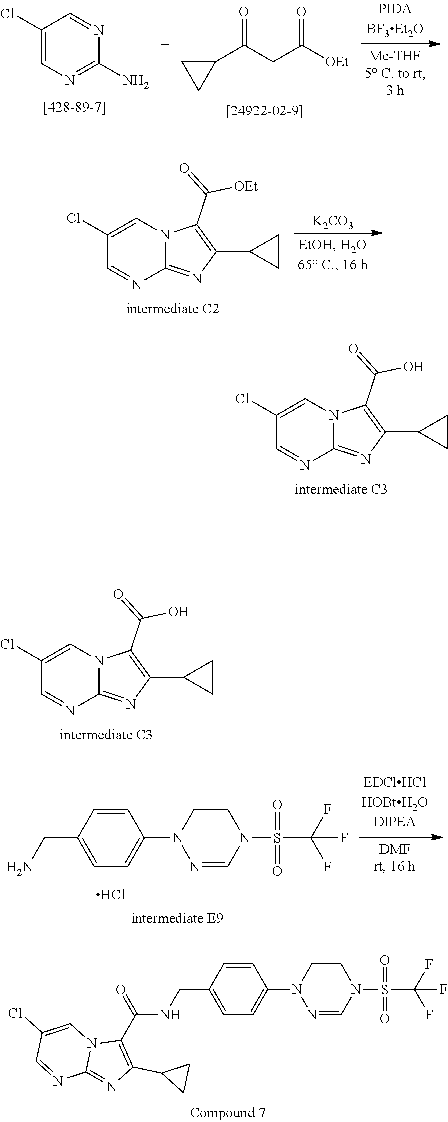

Preparation of Intermediate C2

To a solution of 2-amino-5-chloropyrimidine [428-89-7] (500 mg, 3.86 mmol) in Me-THF (40 mL) at 5° C. were added ethyl 3-cyclopropyl-3-oxopropanoate [24922-02-9](0.603 g, 3.86 mmol) and (diacetoxyiodo)benzene (1.24 g, 3.86 mmol). Boron trifluoride etherate (50 μL, 0.191 mmol) was added dropwise, and the reaction mixture was stirred at 5° C. for 30 min, then at room temperature for 1 h. Extra amounts of ethyl 3-cyclopropyl-3-oxopropanoate (0.301 g, 1.93 mmol) (diacetoxyiodo)benzene (0.622 g, 1.93 mmol) and boron trifluoride etherate (50 μL, 0.191 mmol) were added. The mixture was purged with N₂ and stirred at room temperature for 1 h. Extra amounts of ethyl 3-cyclopropyl-3-oxopropanoate (0.301 g, 1.93 mmol), (diacetoxyiodo)benzene (0.622 g, 1.93 mmol) and boron trifluoride etherate (50 μL, 0.191 mmol) were added again. The mixture was purged with N₂ and stirred at room temperature for another 1 h.

EtOAc and water were added. The layers were separated, and the organic phase was dried over MgSO₄, filtered and concentrated in vacuo. The crude mixture was purified by preparative LC (irregular SiOH 15-40 μm, 80 g, dry loading (Celite®), mobile phase: heptane/EtOAc, 80:20, 65:35). The residue was triturated in pentane. The solid was collected by filtration and dried under vacuum to give 598 mg of intermediate C2 as a white solid (58%).

Preparation of Intermediate C3

To a solution of the intermediate C2 (125 mg, 0.47 mmol) in EtOH (2.2 mL) and water (2.2 mL) was added K₂CO₃ (196 mg, 1.42 mmol). The reaction mixture was stirred at 65° C. for 16 h. The mixture was cooled to room temperature and the reaction was quenched with HCl (1M in water) until pH-3. The mixture was evaporated in vacuo to afford 294 mg of intermediate C3 as a white solid. The crude product was used as such in the next step.

Preparation of Compound 7

To a solution of intermediate C3 (294 mg, 0.472 mmol) in DMF (4.5 mL) were added EDCI·HCl (110 mg, 0.574 mmol), HOBt·H₂O (76 mg, 0.496 mmol), DIPEA (0.245 mL, 1.42 mmol) and intermediate E9 (185 mg, 0.516 mmol). The reaction mixture was stirred at room temperature for 16 h evaporated in vacuo. The residue was taken-up in EtOAc, washed with NaHCO₃ (sat., aq.) and brine. The organic layer was dried over MgSO₄, filtered and evaporated in vacuo. The crude mixture was purified by preparative LC (irregular SiOH 15-40 μm, 24 g Büchi, dry loading (Celite®), mobile phase: heptane/(EtOAc/MeOH, 9:1), gradient from 90:10 to 40:60) to afford a light yellow solid. The solid was crystallized from EtOAc and sonicated in pentane. The solid was collected by filtration and dried under vacuum to obtain 121 mg of compound 7 as a white solid (47%).

¹H NMR (400 MHz, DMSO-d₆) δ ppm 9.40 (d, J=1.8 Hz, 1H) 8.58-8.75 (m, 2H) 7.34 (d, J=8.1 Hz, 2H) 7.29 (s, 1H) 7.19 (d, J=8.4 Hz, 2H) 4.50 (d, J=5.6 Hz, 2H) 4.08 (s, 2H) 3.83 (s, 2H) 2.38-2.46 (m, 1H) 1.03-1.13 (m, 4H).

Synthesis of Compound 8

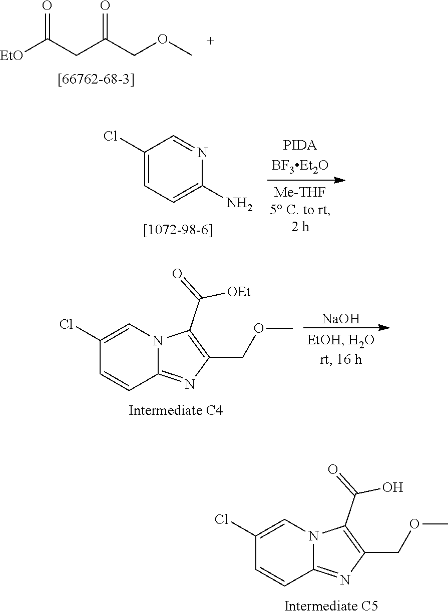

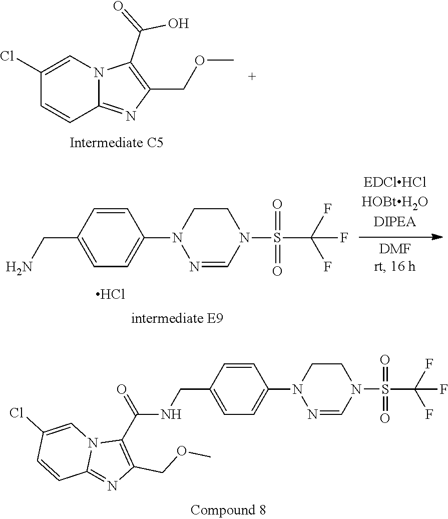 [SMILES: COCc1nc2ccc(Cl)cn2c1C(=O)O ; NCc1ccc(N2CCN(S(=O)(=O)C(F)(F)F)C=N2)cc1 ; Cl ; COCc1nc2ccc(Cl)cn2c1C(=O)NCc1ccc(N2CCN(S(=O)(=O)C(F)(F)F)C=N2)cc1]

Preparation of Intermediate C4

To a solution of 2-amino-5-chloropyridine [1072-98-6] (3.00 g, 23.3 mmol) in Me-THF (100 mL) were added iodobenzene diacetate (7.50 g, 23.3 mmol) and and ethyl-4-methoxy-3-oxobutanoate [66762-68-3] (6.00 g, 34.8 mmol). Then boron trifluoride etherate (0.30 mL, 1.15 mmol) was added dropwise. The solution was stirred at 5° C. for 1 h. The mixture was warmed to room temperature and stirred for another 1 h. EtOAc and NaHCO₃ (sat., aq.) were added. The layers were separated, and the aqueous layer was extracted with EtOAc. The combined organic extracts were washed with brine (twice), dried over MgSO₄, filtered and evaporated to give a brown liquid. The crude mixture was purified by preparative LC (irregular SiOH 15-40 μm, 120 g, dry loading (Celite®), mobile phase: heptane/EtOAc, gradient from 90:10 to 40:60) to afford 2.44 g of the intermediate C4 as a yellow solid (39%).

Preparation of Intermediate C5

To a solution of intermediate C4 (1.44 g, 5.36 mmol) in EtOH (11.5 mL) and water (11.5 mL) was added NaOH (650 mg, 16.3 mmol) and the reaction mixture was stirred at room temperature overnight. The reaction was quenched with HCl (3N in water) until pH˜3. The mixture was filtered to afford 996 mg of the intermediate C5 as an off-white solid (77%).

Preparation of Compound 8

To a mixture of intermediate C5 (125 mg, 0.519 mmol) and DIPEA (270 μL, 1.57 mmol) in DMF (5 mL) at room temperature were added EDCI·HCl (125 mg, 0.652 mmol) and HOBt·H₂O (85 mg, 0.555 mmol). Intermediate E9 (205 mg, 0.571 mmol) was added and the resulting mixture was stirred for 16 h. NaHCO₃ (1%, aq.) and EtOAc were added and the layers were separated. The organic layer was washed with brine (3 times), dried over MgSO₄, filtered and concentrated in vacuo until dryness to give an orange solid which was purified by preparative LC (irregular SiOH 15-40 μm, 24 g, dry loading (Celite®), mobile phase: heptane/(EtOAc/MeOH, 9:1), gradient from 75:20 to 30:70) to obtain a white solid. The residue was purified by reverse phase (spherical C18, 25 μm, 40 g YMC-ODS-25, dry loading (Celite®), mobile phase: NH₄HCO₃ (0.2% in water)/MeCN, gradient from 60:40 to 0:100) to give 233 mg of compound 8 as a white solid (71%).

¹H NMR (400 MHz, CDCl₃-d) 6 ppm 9.68 (dd, J=2.0, 0.8 Hz, 1H) 8.51 (t, J=4.7 Hz, 1H) 7.56 (d, J=9.4 Hz, 1H) 7.31-7.36 (m, 3H) 7.18 (d, J=7.9 Hz, 2H) 7.11 (s, 1H) 4.75 (s, 2H) 4.59 (d, J=5.5 Hz, 2H) 4.06 (t, J=4.7 Hz, 2H) 3.79 (t, J=4.7 Hz, 2H) 3.28 (s, 3H)

Synthesis of Compound 9

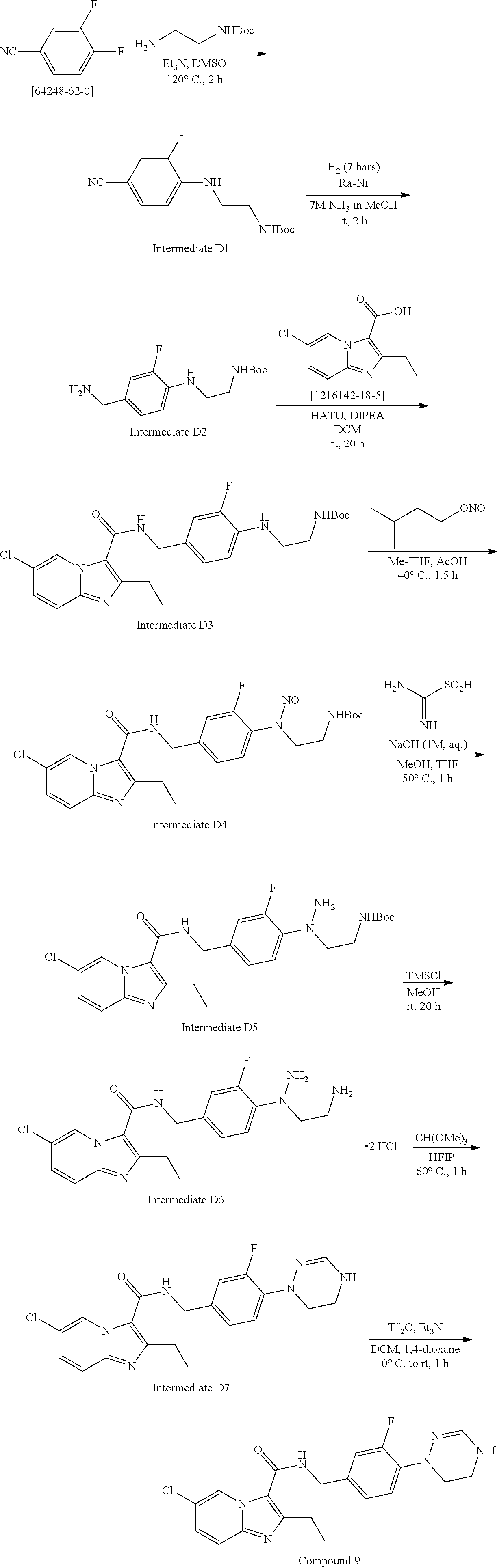

Preparation of Intermediate D1

A mixture of 3,4-difluorobenzonitrile [64248-62-0] (3.67 g, 26.4 mmol), N-Boc-1,2-diaminoethane (5.50 g, 34.3 mmol) and Et₃N (14.7 mL, 105 mmol) in DMSO (47 mL) was stirred at 120° C. for 2 h. The reaction mixture was cooled down and diluted with EtOAc and water. The layers were separated and the aqueous phase was extracted with EtOAc (twice). The combined organic layers were washed with brine (3 times), dried over MgSO₄, filtered and evaporated in vacuo. The residue was purified by preparative LC (irregular SiOH 15-40 μm, 80 g, liquid injection (DCM), mobile phase: heptane/EtOAc, gradient from 100:0 to 50:50) to give 5.02 g of intermediate D1 as a white solid (68%).

Preparation of Intermediate D2

In an autoclave, to a solution of intermediate D1 (2.00 g, 7.16 mmol) in a 7M solution of NH₃ in MeOH (70 mL), purged with nitrogen, was added Raney-Nickel (3.39 g, 57.7 mmol). The reaction mixture was hydrogenated under 7 bars at room temperature for 2 h. The mixture was filtered through a pad of Celite® and rinsed with MeOH. The filtrate was concentrated in vacuo to give 2.11 g of the intermediate D2 as a white solid (Quant.).

Preparation of Intermediate D3

HATU (2.57 g, 6.77 mmol) was added to a mixture of 6-chloro-2-ethylimidazo[1,2-a]pyridine-3-carboxylic acid [1216142-18-5] (1.52 g, 6.77 mmol) and DIPEA (4.7 mL, 27.1 mmol) in DCM (126 mL). The reaction mixture was stirred at room temperature for 10 min and then intermediate D2 (2.11 g, 7.45 mmol) was added and the reaction mixture was stirred at room temperature for 20 h. The reaction mixture was diluted with DCM and water. The aqueous layer was extracted with DCM (twice). The combined organic layers were washed with brine (twice), dried over MgSO₄, filtered and evaporated in vacuo. The residue was purified by preparative LC (irregular SiOH 15-40 μm, 120 g, liquid injection (DCM), mobile phase: heptane/EtOAc, gradient from 50:50 to 0:100) to give 2.76 g of intermediate D3 as a pale brown solid (83%).

Preparation of Intermediate D4

Intermediate D3 (1.5 g, 3.06 mmol) was solubilized at 40° C. in Me-THF (23.2 mL) and AcOH (1.75 mL). Isopentyl nitrite (2.06 mL, 15.3 mmol) was added dropwise over 10 min and the reaction mixture was stirred at 40° C. for 1 h. The solution was diluted in EtOAc and NaHCO₃ (sat., qa.). The layers were separated and the organic layer was washed with NaHCO₃ (sat., aq.) (twice), and brine, dried over MgSO₄ and evaporated in vacuo to give 1.74 g of intermediate D4 as a pale-yellow oil.

Preparation of Intermediate D5

A solution of intermediate D4 (1.59 g, 3.06 mmol) in THE (47 mL) and MeOH (32 mL) was treated with NaOH (1M, aq., 37 mL). Thisurea dioxide (formamidinesulfonic acid) (1.66 g, 15.3 mmol) was added and the reaction mixture was stirred at 50° C. for 1 h (using findeser equipment). The reaction mixture was diluted with DCM and K₂CO₃ (10%, aq.) was added. The layers were separated, and the organic layer was dried over MgSO₄, filtered and the solvent was removed under reduced pressure to give 1.44 g of intermediate D5 as a yellow oil.

Preparation of Intermediate D6

A solution of intermediate A5 (1.55 g, 3.06 mmol) in MeOH (34 mL) was treated with TMSCl (3.88 mL, 30.6 mmol) and the reaction mixture was stirred at room temperature for 20 h. The solvent was removed under reduced pressure and the resulting solid was triturated in Et₂O. The solvent was evaporated to give 1.51 g of intermediate D6 as a pale-yellow solid (Quant.).

Preparation of Intermediate D7

Trimethyl orthoformate (0.618 mL, 5.65 mmol) was added to a suspension of intermediate D6 (900 mg, 1.88 mmol) in HFIP (18 mL) and the reaction mixture was stirred at 60° C. for 1 h. The reaction mixture was cooled down to room temperature, diluted with EtOAc and then basified with NaHCO₃ (sat., aq.). The layers were separated, and the aqueous layer was extracted with EtOAc. The combined organic layers were driedₒᵥₑᵣ MgSO₄, filtered and the solvent was removed under reduced pressure. The residue was purified by preparative LC (irregular SiOH 15-40 μm, 24 g, liquid injection (DCM), mobile phase: DCM/MeOH, gradient from 100:0 to 90:10) to give 202 mg of the intermediate D7 as an off-white solid (33%).

Preparation of Compound 9

Et₃N (0.169 mL, 1.22 mmol) was added to a solution of intermediate D7 (202 mg, 0.487 mmol) in DCM (9 mL) and 1,4-dioxane (6 mL). The solution was cooled to 5° C. and a solution of Tf₂O in DCM (1M in DCM, 0.487 mL, 0.487 mmol) was added dropwise over 5 min. The reaction mixture was diluted with DCM and with NaHCO₃ (sat., aq.). The layers were separated. The organic layer was washed with brine, dried over MgSO₄, filtered and the solvent was removed under reduced pressure. The residue was purified by preparative LC (irregular SiOH 15-40 μm, 12 g, liquid injection (DCM), mobile phase: heptane/EtOAc, gradient from 70:30 to 0:100) to give 183 mg of a yellow solid. The solid was triturated _{and} sonicated in EtOAc. The suspension was filtered off. The solid and the filtrate were combined. The residue was triturated in Et₂O and sonicated, filtered off, washed with Et₂O and collected to give 125 mg of compound 9 as a white solid (47%).

¹H NMR (400 MHz, DMSO-d₆) δ ppm 9.09 (d, J=1.5 Hz, 1H) 8.48 (t, J=5.9 Hz, 1H) 7.67 (d, J=9.5 Hz, 1H) 7.47 (dd, J=9.5, 2.0 Hz, 1H) 7.30-7.41 (m, 2H) 7.16-7.30 (m, 2H) 4.50 (d, J=5.9 _{Hz}, 2H) 4.10 (br t, J=4.2 Hz, 2H) 3.65 (t, J=4.6 Hz, 2H) 3.00 (q, J=7.5 Hz, 2H) 1.27 (t, J=7.5 Hz, 3H).

Synthesis of Compound 10

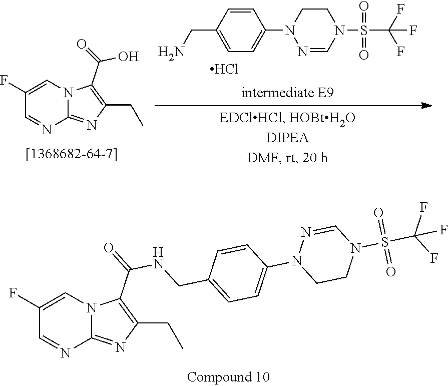 [SMILES: CCc1nc2ncc(F)cn2c1C(=O)O ; NCc1ccc(N2CCN(S(=O)(=O)C(F)(F)F)C=N2)cc1 ; Cl ; CCc1nc2ncc(F)cn2c1C(=O)NCc1ccc(N2CCN(S(=O)(=O)C(F)(F)F)C=N2)cc1]

To a solution of 2-ethyl-6-fluoroimidazo[1,2-a]pyridine-3-carboxylic acid [1368682-64-7] (82 mg 0.393 mmol) in DMF (4.5 mL) were added EDCI·HCl (91 mg, 0.474 mmol), HOBt·H₂O (63 mg, 0.415 mmol) and DIPEA (203 μL, 1.18 mmol). The mixture was stirred at room temperature for 15 min. Intermediate B9 (155 mg, 0.432 mmol) was added and the reaction mixture was stirred at room temperature for 20 h. The solvent was removed under reduced pressure and the residue was diluted with EtOAc and water. The layers were separated and the aqueous layer was extracted with EtOAc. The combined organic layers were washed with brine (twice), dried over MgSO₄, filtered and the solvent was removed under reduced pressure. The residue was purified by preparative LC (irregular SiOH 15-40 μm, 12 g, liquid injection (DCM), mobile phase: DCM/MeOH, gradient from 100:0 to 90:10). A second purification was performed by reverse phase (stationary phase: YMC-actus Triart C18 10 μm 30*150 mm, mobile phase: NH₄HCO₃ (0.2% in water)/MeCN, gradient from 50:50 to 25:75). The residue was solubilized in MeCN and MeOH (50:50), extended with water and freeze-dried to give 44 mg of compound 10 as a white solid (22%).

¹H NMR (400 MHz, DMSO-d₆) δ ppm 9.40 (dd, J=4.8, 2.9 Hz, 1H) 8.82 (d, J=3.1 Hz, 1H) 8.51 (t, J=5.7 Hz, 1H) 7.26-7.35 (m, 3H) 7.18 (d, J=8.7 Hz, 2H) 4.48 (d, J=5.7 Hz, 2H) 4.08 (t, J=4.6 Hz, 2H) 3.82 (t, J=4.8 Hz, 2H) 3.02 (q, J=7.5 Hz, 2H) 1.27 (t, J=7.5 Hz, 3H).

Synthesis of Compound 11

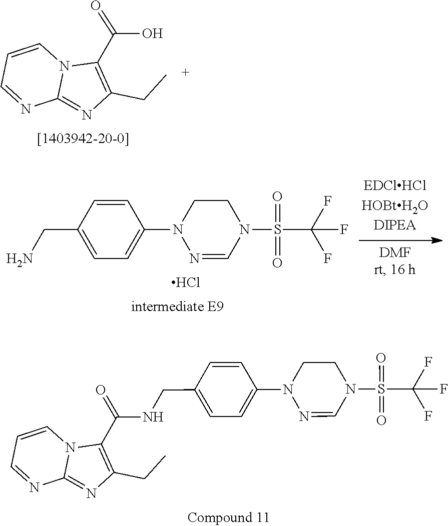 [SMILES: CCc1nc2ncccn2c1C(=O)O ; NCc1ccc(N2CCN(S(=O)(=O)C(F)(F)F)C=N2)cc1 ; Cl ; CCc1nc2ncccn2c1C(=O)NCc1ccc(N2CCN(S(=O)(=O)C(F)(F)F)C=N2)cc1]

To a mixture of 2-ethyl-imidazo[1,2-a]pyrimidine-3-carboxylic acid [1403942-20-0](125 mg, 0.654 mmol) and DIPEA (228 μL, 1.32 mmol) in DMF (6.5 mL) at room temperature were added EDCI·HCl (150 mg, 0.782 mmol) and HOBt·H₂O (105 mg, 0.686 mmol). Intermediate E9 (230 mg, 0.714 mmol) was added and the resulting mixture was stirred for 16 h. NaHCO₃ (1%, aq.) and EtOAc were added. The layers were separated, and the organic layer was washed with brine (twice), dried over MgSO₄, filtered and concentrated in vacuo until dryness. The residue was purified by preparative LC (irregular SiOH 15-40 μm, 24 g, dry loading (Celite®), mobile phase: heptane/(EtOAc/MeOH, 9/1), gradient from 60:40 to 10:90). The residue was crystallized from EtOAc and collected by filtration to give 170 mg of compound 11 as a white solid (52%).

¹H NMR (400 MHz, DMSO-d₆) δ ppm 9.30 (dd, J=7.0, 2.0 Hz, 1H) 8.61 (dd, J=4.2, 2.0 Hz, 1H) 8.48 (t, J=5.9 Hz, 1H) 7.27-7.35 (m, 3H) 7.13-7.21 (m, 3H) 4.47 (d, J=6.0 Hz, 2H) 4.05-4.11 (m, 2H) 3.83 (t, J=4.8 Hz, 2H) 3.01 (q, J=7.5 Hz, 2H) 1.27 (t, J=7.5 Hz, 3H).

Synthesis of Compound 12

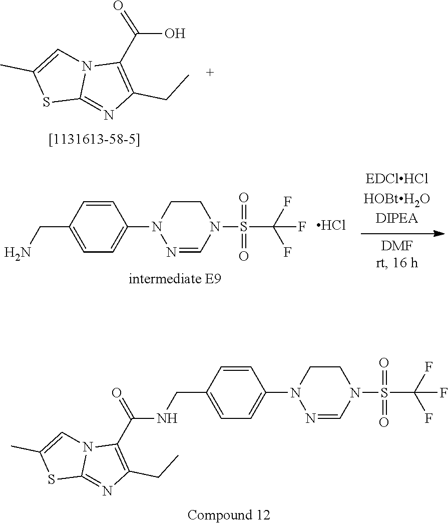 [SMILES: CCc1nc2sc(C)cn2c1C(=O)O ; NCc1ccc(N2CCN(S(=O)(=O)C(F)(F)F)C=N2)cc1 ; Cl ; CCc1nc2sc(C)cn2c1C(=O)NCc1ccc(N2CCN(S(=O)(=O)C(F)(F)F)C=N2)cc1]

To a mixture of 6-ethyl-2-methyl-imidazo[2,1-b]thiazole-5-carboxylic acid [1131613-58-5] (150 mg, 0.608 mmol) and DIPEA (345 μL, 2.00 mmol) in DMF (6.5 mL) were added EDCI·HCl (140 mg, 0.730 mmol) and HOBt·H₂O (100 mg, 0.653 mmol). The mixture was stirred at room temperature for 15 min. Then intermediate E9 (240 mg, 0.669 mmol) was added and the resulting mixture was stirred for 16 h. The mixture was evaporated in vacuo. NaHCO₃ (1%, aq.) and EtOAc were added and the layers were separated. The organic layer was washed with brine, dried over MgSO₄ and concentrated to dryness. The residue was purified by preparative LC (irregular SiOH 15-40 μm, 24 g, dry loading (Celite®), mobile phase: heptane/(EtOAc/MeOH, 9/1), gradient from 95:5 to 50:50). A second purification was performed by reverse phase (spherical C18, 25 μm, 40 g YMC-ODS-25, dry loading (Celite®), mobile phase: NH₄HCO₃ (0.2% in water)/MeCN, gradient from 60:40 to 5:95) to give 206 mg of compound 12 as a white solid (66%).

¹H NMR (500 MHz, DMSO-d₆) δ ppm 8.05 (t, J=6.0 Hz, 1H) 7.87 (s, 1H) 7.24-7.30 (m, 3H) 7.17 (d, J=8.5 Hz, 2H) 4.41 (d, J=6.0 Hz, 2H) 4.04-4.10 (m, 2H) 3.81 (br t, J=4.7 Hz, 2H) 2.86 (q, J=7.6 Hz, 2H) 2.41 (s, 3H) 1.20 (t, J=7.6 Hz, 3H).

Synthesis of Compound 13 and Compound 14

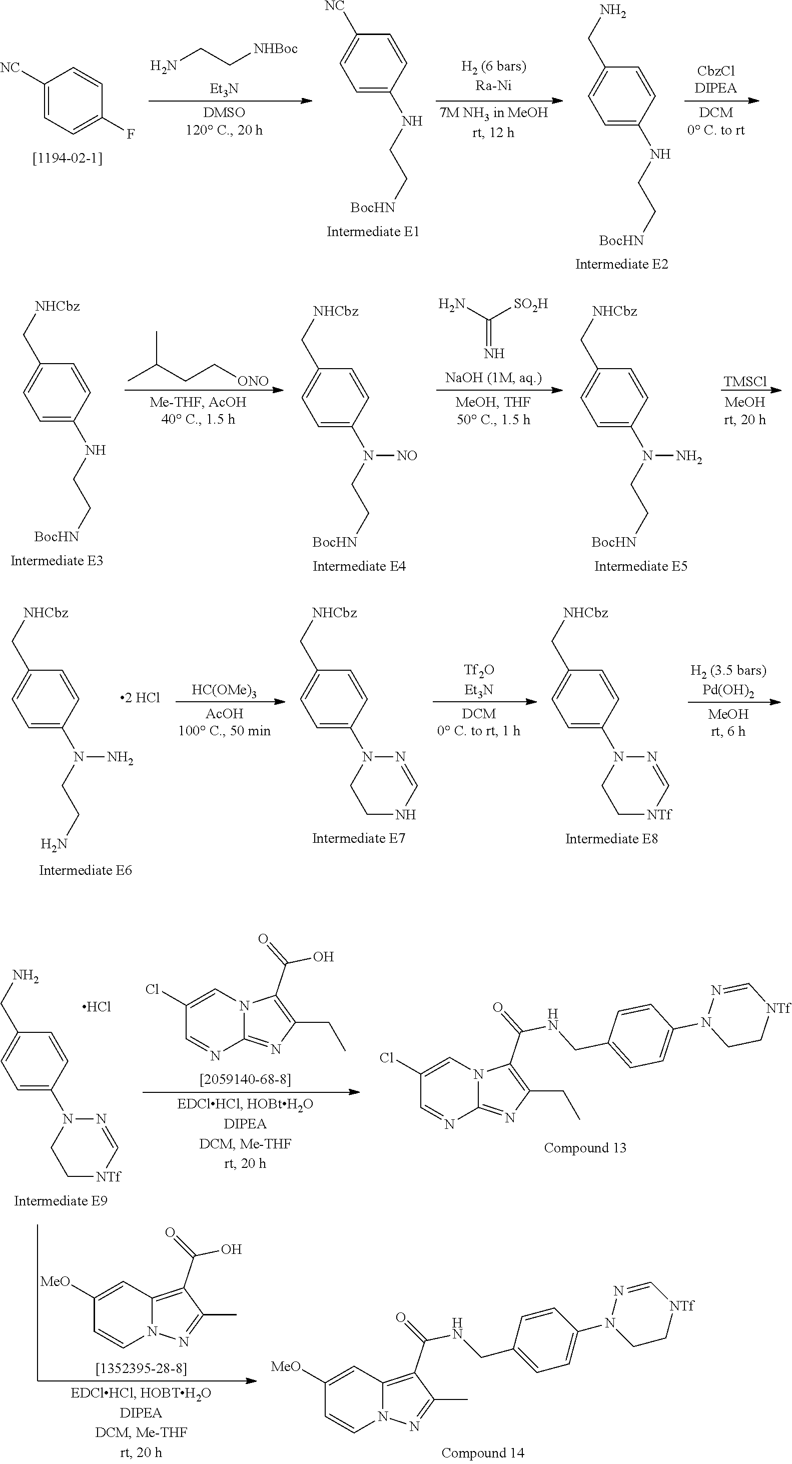

Preparation of Intermediate E1

The reaction was performed on 2 batches. Herein is reported the procedure for one batch. Herein, where “Tf” is used, for avoidance of doubt, it represents —S(O)₂CH₃. Further, Intermediate E9 may be prepared and/or employed as the HCl salt. A 1 L flask equipped with a findenser was charged with 4-fluorobenzonitrile [1194-02-1] (20 g, 165 mmol), DMSO (320 mL) and N-boc-1,2-diaminoethane (39.7 g, 248 mmol). Et₃N (92 mL, 661 mmol) was added and the reaction mixture was stirred at 120° C. for 20 h. The two batches were combined and poured in a mixture of crushed ice and water (1 L). Brine (1 kg) was added and the mixture was stirred at room temperature for 30 min. EtOAc (1 L) was added. The layers were separated and the aqueous layer was extracted with EtOAc (2×500 mL). The combined organic layers were washed with brine (2×1 L), dried over MgSO₄, filtered and evaporated in vacuo. The residue was triturated in pentane (500 mL). The solid was collected by filtration, washed with cold Et₂O, and dried under vacuum to give 48.28 g of intermediate E1 as a white solid (46%, 92% purity).

Preparation of Intermediate E2

In an 1 L autoclave, a mixture of intermediate E1 (41.5 g, 159 mmol) and Raney-Nickel (4.66 g, 79.4 mmol) in a 7M solution of NH₃ in MeOH (500 mL) was hydrogenated at room temperature under 6 bars of H₂ for 12 h. The reaction mixture was filtered through a pad of Celite®, washed with a mixture of DCM and MeOH (9/1) and the filtrate was evaporated in vacuo to afford 41.8 g of intermediate E2 as a green oil (99%).

Preparation of Intermediate E3

Under N₂ at 0° C., benzylchloroformate (0.592 mL, 4.15 mmol) was added dropwise to a mixture of intermediate E2 (1 g, 3.8 mmol) and DIPEA (0.78 mL, 4.52 mmol) in DCM (38 mL). The reaction mixture was stirred at room temperature for 16 h and diluted with DCM. The mixture was washed with NaHCO₃ (sat., aq.), dried over MgSO₄, filtered and the solvent was removed under reduced pressure to give 1.11 g of intermediate E3 as a white solid (74%).

Preparation of Intermediate E4

Intermediate E3 (1.11 g, 2.78 mmol) was solubilized at 40° C. in Me-THF (21 mL) and AcOH (1.6 mL). Isopentylnitrite (1.87 mL, 13.9 mmol) was added dropwise over 15 min and the reaction mixture was stirred at 40° C. for 1.5 h. The solution was diluted with EtOAc and NaHCO₃ (sat., aq.). The layers were separated and the organic phase was washed with NaHCO₃ (sat., aq., twice), brine, dried over MgSO₄ and evaporated in vacuo to give 1.23 g of intermediate E4 as a pale-yellow solid (Quant.).

Preparation of Intermediate E5

A solution of intermediate E4 (1.24 g, 2.89 mmol) in THE (29 mL) and MeOH (19 mL) was treated with NaOH (1M, aq., 29 mL). Thiourea dioxide (formamidinesulfonic acid) (1.56 g, 14.5 mmol) was then added and the reaction mixture was stirred at 50° C. for 1.5 h. The reaction mixture was diluted with DCM and K₂CO₃ (10%, aq.) was added. The layers were separated. The aqueous layer was extracted with DCM and MeOH (95/5). The combined organic layers were dried over MgSO₄, filtered and evaporated in vacuo to give 970 mg of intermediate E5 as a pale-yellow oil (81%).

Preparation of Intermediate E6

To a solution of intermediate E5 (970 mg, 2.34 mmol) in MeOH (23 mL) was added dropwise TMSCl (2.4 mL, 18.7 mmol). The reaction mixture was stirred at room temperature for 20 h and concentrated in vacuo to give 710 mg of intermediate E6 as a brown solid (78%).

Preparation of Intermediate E7

A mixture of intermediate E6 (0.71 g, 1.83 mmol) and trimethyl orthoformate (0.602 mL, 5.50 mmol) in AcOH (9.2 mL) was stirred for 50 min at 100° C. The reaction mixture was concentrated in vacuo. The residue was diluted in a solution of DCM and K₂CO₃ (10%, aq.). The layers were separated and the aqueous layer was extracted with DCM and MeOH (95/5) (twice). The combined organic layers were dried over MgSO₄, filtered and evaporated in vacuo. The residue was purified by preparative LC (irregular SiOH 15-40 μm, 40 g, liquid injection (DCM), mobile phase: DCM/MeOH, gradient from 100:0 to 90:10) to give 273 mg of intermediate E7 as a yellow residue (46%).

Preparation of Intermediate E8

Et₃N (0.292 mL, 2.10 mmol) was added to a solution of intermediate E7 (273 mg, 0.842 mmol) in DCM (12 mL). The solution was then cooled to 5° C. and a solution of Tf₂O (1M in DCM, 1.0 mL, 1.0 mmol) was added dropwise over 5 min. The reaction mixture was stirred for 1 h and diluted with DCM and NaHCO₃ (sat., aq.). The layers were separated. The aqueous layer was extracted with DCM (twice). The combined organic layers were dried over MgSO₄, filtered and the solvent was removed under reduced pressure. The residue was purified by preparative LC (irregular SiOH 15-40 μm, 12 g, dry loading (Celite®), mobile phase: heptane/EtOAc, gradient from 100:0 to 0:100) to give 105 mg of intermediate E8 as a white solid (27%).

Preparation of Intermediate E9

In a steal bomb, a mixture of intermediate E8 (85 mg, 0.186 mmol) and Pd(OH)₂ (21 mg, 0.075 mmol) in MeOH (8.5 mL) was hydrogenated at room temperature under 10 bars of H₂ for 6 h. The mixture was filtered on a pad of Celite® and the filtrate was evaporated in vacuo to give 65 mg of intermediate E9 as a white residue (Quant.).

Preparation of Compound 13

To a mixture of 6-chloro-2-ethyl-imidazo[1,2-a]pyrimidine-3-carboxylic acid [2059140-68-8] (46 mg, 0.202 mmol) and DIPEA (0.070 mL, 0.403 mmol) in DCM (3 mL) and Me-THF (3 mL) were added EDCI·HCl (39 mg, 0.202 mmol), HOBt·H₂O (31 mg, 0.202 mmol) and intermediate E9 (65 mg, 0.202 mmol). The reaction mixture was stirred at room temperature for 20 h. The reaction mixture was diluted with DCM and washed with NaHCO₃ (sat., aq.). The organic layer was dried over MgSO₄, filtered and the solvent was removed under reduced pressure. The residue was purified by preparative LC (irregular SiOH 15-40 μm, 12 g, liquid injection (DCM), mobile phase: DCM/MeOH, gradient from 100:0 to 90:10). The solid (70 mg) was triturated and sonicated in Et₂O and the solvent was removed under reduced pressure. The residue (68 mg) was purified by reverse phase (stationary phase: YMC-actus Triart C18 10 μm 30*150 mm, mobile phase: NH₄HCO₃ (0.2% in water)/MeCN, gradient from 55:45 to 35:65) to give 42 mg of compound 13 as a white solid (39%).

¹H NMR (400 MHz, DMSO-d₆) δ ppm 9.40 (d, J=2.69 Hz, 1H) 8.68 (d, J=2.57 Hz, 1H) 8.55 (t, J=5.87 Hz, 1H) 7.32 (m, J=8.68 Hz, 2H) 7.28 (s, 1H) 7.19 (m, J=8.68 Hz, 2H) 4.47 (d, J=5.87 Hz, 2H) 4.08 (t, J=4.58 Hz, 2H) 3.83 (t, J=4.77 Hz, 2H) 3.01 (q, J=7.46 Hz, 2H) 1.29 (t, J=7.46 Hz, 3H).

Preparation of Compound 14

Compound 14 was prepared following the procedure reported for the synthesis of compound 13 starting from intermediate E9 and 5-methoxy-2-methylpyrazolo[1,5-a]pyridine-3-carboxylic acid [1352395-28-8] affording 32 mg as white fluffy solid (40%).

¹H NMR (400 MHz, DMSO-d₆) δ ppm 8.50 (d, J=7.46 Hz, 1H) 7.86 (t, J=5.99 Hz, 1H) 7.25-7.33 (m, 3H) 7.24 (d, J=2.69 Hz, 1H) 7.18 (d, J=8.68 Hz, 2H) 6.63 (dd, J=7.46, 2.81 Hz, 1H) 4.43 (d, J=5.99 Hz, 2H) 4.08 (t, J=4.59 Hz, 2H) 3.85 (s, 3H) 3.79-3.83 (m, 2H).

Synthesis of Compound 15

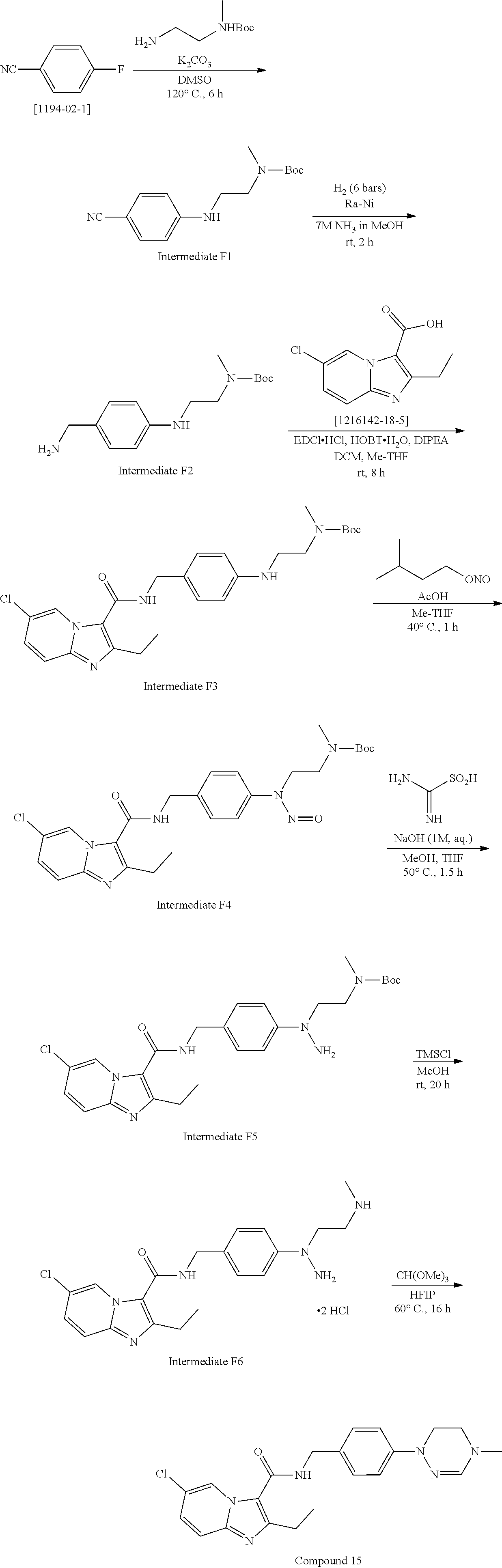

Preparation of Intermediate F1

A mixture of 4-fluorobenzonitrile [1194-02-1] (10.0 g, 82.6 mmol), N-boc-N-methylethylenediamine (20.2 mL, 116 mmol) and K₂CO₃ (13.7 g, 99.1 mmol) in anhydrous DMSO (40 mL) was heated at 120° C. for 6 h. The reaction mixture was poured in brine and EtOAc was added. The layers were separated and the aqueous layer was extracted with EtOAc. The combined organic layers were washed with water and brine, dried over MgSO₄, filtered and evaporated in vacuo. The crude mixture was purified by preparative LC (irregular SiOH 15-40 μm, 330 g, liquid injection (DCM), mobile phase: heptane/EtOAc, gradient from 90:10 to 30:70) to give 18.04 g of intermediate F1 as a colorless oil (80%).

Preparation of Intermediate F2

In a 1 L autoclave, a mixture of intermediate F1 (17.0 g, 61.7 mmol) and Raney-Nickel (14.5 g, 247 mmol) in MeOH (330 mL) was stirred at room temperature for 2 h under 6 bars of H₂. The mixture was filtered on a pad of Celite®, washed with MeOH and the filtrate was evaporated in vacuo to give 17.25 g of intermediate F2 as a blue/green oil (Quant.).

Preparation of Intermediate F3

To a mixture of 6-chloro-2-ethylimidazo[1,2-a]pyridine-3-carboxylic acid [1216142-18-5] (2.35 g, 10.0 mmol), intermediate F2 (3.07 g, 11.0 mmol) and DIPEA (3.45 mL, 20.0 mmol) in DCM (70 mL) and Me-THF (70 mL) were added EDCI·HCl (2.30 g, 12.0 mmol) and HOBt·H₂O (1.62 g, 12.0 mmol). The reaction mixture was stirred at room temperature for 8 h. The mixture was evaporated and the crude mixture was purified by preparative LC (irregular SiOH 15-40 μm, 220 g, dry loading (Celite®), mobile phase: heptane/EtOAc, gradient from 70:30 to EtOAc 0:100) to give 3.703 g of intermediate F3 as a brown foam (76%).

Preparation of Intermediate F4

Intermediate F3 (3.54 g, 7.28 mmol) was solubilized in Me-THF (62 mL) and AcOH (4.17 mL, 72.8 mmol). Isopentyl nitrite (4.89 mL, 36.4 mmol) was added dropwise and the reaction mixture was stirred at 40° C. for 1 h. The resulting solution was diluted in EtOAc. The organic layer was washed with K₂CO₃ (10%, aq.) (twice) and brine, dried over MgSO₄ and evaporated in vacuo. The residue was purified by preparative LC (irregular SiOH 15-40 μm, 80 g, dry loading (Celite®), mobile phase: heptane/EtOAc, gradient from 50:50 to 0:100) to give 3.54 g of intermediate F4 as an orange paste (94%).

Preparation of Intermediate F5

A solution of intermediate F4 (1.13 g, 2.19 mmol) in THF (22 mL) and MeOH (14 mL) was treated with NaOH (1M aq., 22 mL, 22 mmol). Formamidinesulfonic acid (1.19 g, 11.0 mmol) was added and the reaction mixture was stirred at 50° C. for 1.5 h. The reaction mixture was diluted in DCM and K₂CO₃ (10% aq.) was added. The aqueous layer was extracted with DCM and MeOH (95/5) (twice). The combined organic layers were dried over MgSO₄, filtered and evaporated in vacuo to give 970 mg of intermediate F5 as a yellow foam (91% purity, 80%).

Preparation of Intermediate F6

A solution of intermediate F5 (932 mg, 1.69 mmol) in MeOH (18 mL) was treated with TMSCl (2.15 mL, 16.9 mmol). The reaction mixture was stirred at room temperature for 20 h and evaporated in vacuo. The solid was triturated in Et₂O. The supernatant was removed and the yellow powder was dried under vacuum to give 915 mg of intermediate F6 (Quant.).

Preparation of Compound 15

To a solution of intermediate F6 (270 mg, 0.570 mmol) in HFIP (4.86 mL) was added trimethyl orthoformate (187 μL, 1.71 mmol) and the reaction mixture was stirred at 60° C. for 16 h. The reaction mixture was diluted with EtOAc and quenched with K₂CO₃ (10%, aq.). The organic layer was washed with H₂O (once) and brine (once), dried over MgSO₄, filtered and evaporated in vacuo. The crude mixture was purified by preparative LC (irregular SiOH 15-40 μm, 12 g, dry loading (Celite®), mobile phase: DCM/(DCM/MeOH, 80:20), gradient from 95:5 to 75:25). The residue was heated under reflux in EtOH for 20 min. The solution was cooled to room temperature and at 0° C. The mixture was filtered. The solid was rinsed with cold EtOH and dried under vacuum at 60° C. for 7 h to give 51 mg of compound 15 as a beige downy solid (22%).

¹H NMR (400 MHz, DMSO-d₆) δ ppm 9.03 (s, 1H) 8.40 (t, J=5.8 Hz, 1H) 7.66 (d, J=9.4 Hz, 1H) 7.45 (dd, J=9.5, 2.08 Hz, 1H) 7.18 (d, J=8.7 Hz, 2H) 7.10 (d, J=8.7 Hz, 2H) 6.70 (s, 1H) 4.42 (d, J=5.8 Hz, 2H) 3.51 (t, J=5.2 Hz, 2H) 3.34 (t, J=5.2 Hz, 2H) 2.96 (q, J=7.6 Hz, 2H) 2.83 (s, 3H) 1.25 (t, J=7.5 Hz, 3H).

Synthesis of Compound 16

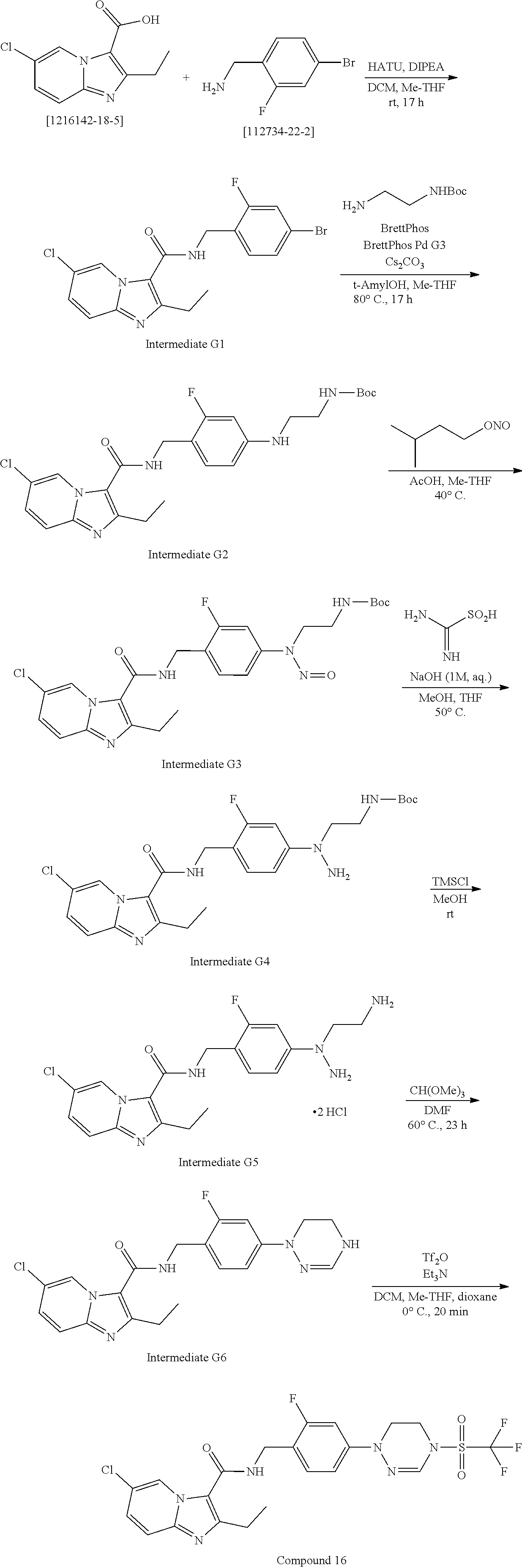

Preparation of Intermediate G1

A flask was charged with 6-chloro-2-ethylimidazo[1,2-a]pyridine-3-carboxylic acid [1216142-18-5] (1.00 g, 4.45 mmol), 4-bromo-2-fluorobenzylamine [112734-22-2](0.954 g, 4.67 mmol), Me-THF (15 mL), DCM (15 mL) and DIPEA (1.23 mL, 7.12 mmol). HATU (1.86 g, 4.90 mmol) was added portion wise and the reaction mixture was stirred at room temperature for 17 h. The mixture was diluted with EtOAc and water. The layers were separated and the organic layer was washed with brine (twice), dried over MgSO₄, filtered and evaporated in vacuo. The residue was solubilized in warm EtOAc. The solution was cooled to room temperature and to 0° C. The suspension was filtered off and the solid was washed with cold EtOAc and then with Et₂O. The solid was dried in vacuo to afford 773 mg of intermediate G1 as an off-white solid (42%).

Preparation of Intermediate G2

A mixture of intermediate G1 (740 mg, 1.80 mmol), N-boc-ethylenediamine (375 mg, 2.34 mmol) and Cs₂CO₃ (1.06 g, 3.24 mmol) in tert-Amyl alcohol (24 mL) and Me-THF (16 mL) was purged with N₂. Brettphos Pd G3 (82 mg, 0.090 mmol) and Brettphos (97 mg, 0.18 mmol) were added. The reaction mixture was purged again with N₂ and stirred for 17 h at 80° C. The reaction mixture was cooled to room temperature. Celite® was added and the mixture was evaporated in vacuo. The residue was purified by preparative LC (irregular SiOH 15-40 μm, 40 g, mobile phase: heptane/EtOAc, gradient from 50:50 0:100) to give 444 mg of intermediate G2 as a pale-yellow foam (50%).

Preparation of Intermediate G3

Intermediate G3 was prepared following the synthesis reported for the synthesis of intermediate F4 starting from intermediate G2 and affording 408 mg as a yellow solid (87%).

Preparation of Intermediate G4

Intermediate G4 was prepared following the procedure reported for the synthesis of intermediate F5 starting from intermediate G3 and affording 362 mg as a beige solid (94%).

Preparation of Intermediate G5

Intermediate G5 was prepared following the procedure reported for the synthesis of intermediate F6 starting from intermediate G4 and affording 343 mg as a yellow powder (Quant.).

Preparation of Intermediate G6

A mixture of intermediate G5 (283 mg, 0.592 mmol) and trimethyl orthoformate (194 μL, 1.78 mmol) in anhydrous DMF (3.7 mL) was stirred for 23 h at 60° C. Additional amount of anhydrous DMF (3.7 mL) and trimethyl orthoformate (194 μL, 1.78 mmol) were added at room temperature and the reaction mixture was stirred at 60° C. for another 1.5 h. The reaction mixture was diluted with DCM and quenched with K₂CO₃ (10%, aq.). The layers were separated and the aqueous layer was extracted with DCM and MeOH (95/5) (twice). The combined organic layers were washed with water and brine, dried over MgSO₄, filtered and evaporated in vacuo. The crude mixture was purified by preparative LC (irregular SiOH 15-40 μm, 12 g, dry loading (Celite®), mobile phase: DCM/(DCM/MeOH, 80/20), gradient from 95:5 to 70:30) to give 156 mg of intermediate G6 as a white solid (63%).

Preparation of Compound 16

Under N₂ atmosphere, a mixture of intermediate G6 (143 mg, 0.345 mmol) and Et₃N (240 μL, 1.72 mmol) in anhydrous DCM (5 mL), anhydrous Me-THF (5 mL) and anhydrous 1,4-dioxane (5 mL) was heated at 40° C. The reaction mixture was cooled to 0° C. and trifluoromethanesulfonic anhydride (0.517 mL, 0.517 mmol) was added dropwise. The mixture was stirred at 0° C. for 20 min and diluted with DCM. A small quantity of MeOH was added and K₂CO₃ (10%, aq.) was added. The layers were separated and the aqueous layer was extracted with DCM (twice). The combined organic layers were washed with water and brine, dried over MgSO₄, filtered and evaporated in vacuo. The crude mixture was purified by preparative LC (irregular SiOH 15-40 μm, 12 g, dry loading (Celite®), mobile phase: DCM/(DCM/MeOH, 80:20), gradient from 100:0 to 80/20). The residue was purified by reverse phase (stationary phase: YMC-actus Triart C18 10 μm 30*150 mm, mobile phase: NH₄HCO₃ (0.2% in water)/MeCN, gradient from 55:45 to 25:75) to give 84 mg of compound 16 as a white solid (45%).

¹H NMR (500 MHz, DMSO-d₆) δ ppm 9.05 (s, 1H) 8.40 (t, J=5.8 Hz, 1H) 7.66 (d, J=9.5 Hz, 1H) 7.45 (dd, J=9.5, 2.1 Hz, 1H) 7.36 (t, J=8.5 Hz, 1H) 7.02 (m, 2H) 7.32 (s, 1H) 4.50 (d, J=5.8 Hz, 2H) 4.07 (t, J=4.7 Hz, 2H) 3.86 (t, J=4.7 Hz, 2H) 2.96 (q, J=7.5 Hz, 2H) 1.25 (t, J=7.5 Hz, 3H).

Synthesis of Compound 17

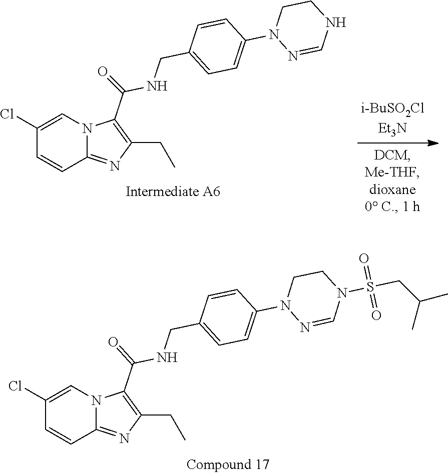

Preparation of Compound 17

Under N₂ atmosphere, a mixture of intermediate A6 (180 mg, 0.454 mmol) and Et₃N (315 μL, 2.27 mmol) in anhydrous Me-THF (7 mL), anhydrous 1,4-dioxane (7 mL) and anhydrous DCM (7 mL) was cooled to 0° C. Isobutanesulfonyl chloride (88.8 μL, 0.680 mmol) was added dropwise. The reaction mixture was stirred for 1 h at 0° C. and diluted with DCM and quenched with K₂CO₃ (10%, aq.). The layers were separated and the aqueous layer was extracted with DCM and MeOH (95/5) (twice). The combined organic layers were dried over MgSO₄, filtered and evaporated in vacuo. The solid was purified by preparative LC (irregular SiOH 15-40 μm, 12 g, dry loading (Celite®), mobile phase: DCM/(DCM:MeOH, 80:20), gradient from 100:0 to 95:5) to give 124 mg of compound 17 as a slightly yellow solid (53%).

¹H NMR (500 MHz, CDCl₃) δ ppm 9.51-9.54 (m, 1H) 7.51-7.55 (m, 1H) 7.32 (d, J=8.7 Hz, 2H) 7.29 (dd, J=9.5, 2.0 Hz, 1H) 7.23 (s, 1H) 7.18 (d, J=8.7 Hz, 2H) 6.03 (br t, 1H) 3.71 (t, J=4.6 Hz, 2H) 3.00 (d, J=6.6 Hz, 2H) 2.95 (q, J=7.6, 2H) 2.32 (m, 1H) 1.39 (t, J=7.6 Hz, 3H) 1.15 (s, 3H) 1.14 (s, 3H).

Synthesis of Compound 18

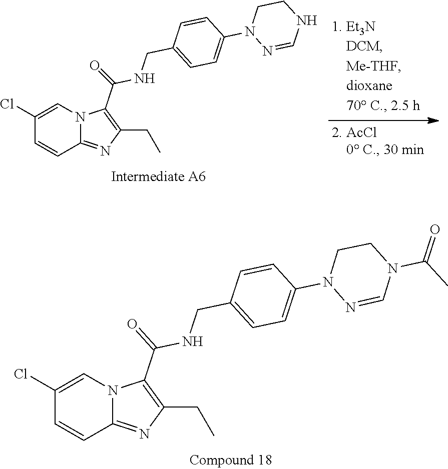

Under N₂ atmosphere a mixture of intermediate A6 (300 mg, 0.756 mmol) and Et₃N (0.525 mL, 3.78 mmol) in anhydrous DCM (11.5 mL), anhydrous Me-THF (11.5 mL) and anhydrous 1,4-dioxane (11.5 mL) was stirred for 2.5 h at 70° C. The mixture was cooled to room temperature and then to 0° C. Acetyl chloride (53.9 μL, 0.756 mmol) was added dropwise and the reaction mixture was stirred for 30 min at 0° C. The reaction mixture was diluted with DCM and quenched with MeOH and K₂CO₃ (10%, aq.). The layers were separated and the aqueous layer was extracted with DCM and MeOH (95/5) (twice). The combined organic layers were washed with brine, dried over MgSO₄, filtered and evaporated in vacuo. The residue was purified by preparative LC (irregular SiOH 15-40 μm, 12 g, dry loading (Celite®), mobile phase: DCM/(DCM/MeOH, 80/20), gradient from 95:5 to 85:15) to give 180 mg of compound 18 as a white solid (54%).

¹H NMR (500 MHz, DMSO-d₆) δ ppm rotamers: 9.08 (d, J=1.3 Hz, 1H) 8.17 (br t, J=5.4 Hz, 1H) 7.62 (d, J=9.8 Hz, 1H) 7.58 (br s, 1H) 7.41 (dd, J=9.5, 2.2 Hz, 1H) 7.30 (d, J=8.8 Hz, 2H) 7.20 (d, J=8.5 Hz, 2H) 4.49 (d, J=6.0 Hz, 2H) 3.86 (br s, 2H) 3.66 (t, J=5.0 Hz, 2H) 2.99 (q, J=7.6 Hz, 2H) 2.25 (s, 3H) 1.28 (t, J=7.6 Hz, 3H).

Synthesis of Compound 19

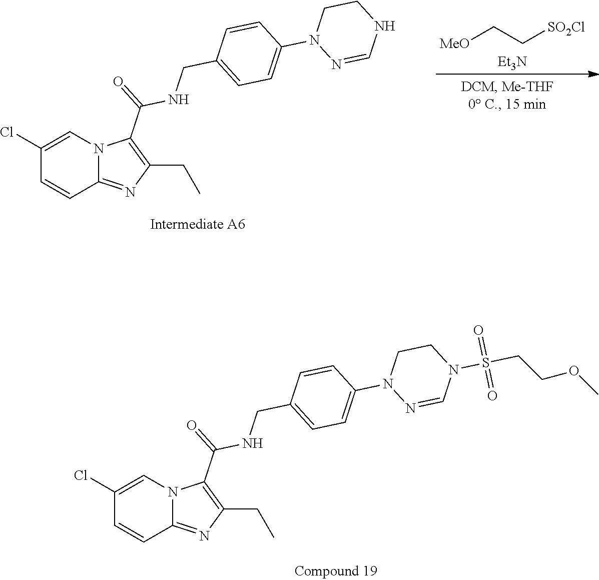 [SMILES: "Et3N" ; CCc1nc2ccc(Cl)cn2c1C(=O)NCc1ccc(N2CCNC=N2)cc1 ; COCCS(=O)(=O)Cl ; CCc1nc2ccc(Cl)cn2c1C(=O)NCc1ccc(N2CCN(S(=O)(=O)CCOC)C=N2)cc1]

To a mixture of intermediate A6 (100 mg, 0.252 mmol) and Et₃N (0.175 mL, 1.26 mmol) in anhydrous DCM (2.7 mL) and anhydrous Me-THF (2.7 mL) was added 2-methoxy-1-ethanesulfonyl chloride (88.3 μL, 0.756 mmol) at 0° C. and the reaction mixture was stirred at 0° C. for 15 min. The reaction was quenched with a small amount of MeOH and K₂CO₃ (10%, aq.) was added. The layers were separated and the aqueous layer was extracted with DCM (twice). The combined organic layers were washed with water (twice) and brine, dried over MgSO₄, filtered and evaporated in vacuo. The residue was purified by preparative LC (irregular SiOH 15-40 μm, 12 g, dry loading (Celite®), mobile phase: heptane/EtAOc, gradient from 55:45 to 0:100, then EtOAc/MeOH 99:1). The solid was triturated in MeCN, the supernatant was removed and the solid was dried under vacuum to give 53 mg of compound 19 as a white solid (41%).

¹H NMR (400 MHz, DMSO-d₆) δ ppm 9.06 (d, J=1.5 Hz, 1H) 8.43 (t, J=5.9 Hz, 1H) 7.66 (d, J=9.5 Hz, 1H) 7.45 (dd, J=9.5, 2.1 Hz, 1H) 7.28 (d, J=8.7 Hz, 2H) 7.17 (d, J=8.7 Hz, 2H) 7.14 (s, 1H) 4.45 (d, J=5.9 Hz, 2H) 3.84 (t, J=4.3 Hz, 2H) 3.63-3.75 (m, 6H) 3.24 (s, 3H) 2.97 (q, J=7.5 Hz, 2H) 1.25 (t, J=7.5 Hz, 3H) 1.09 (t, J=7.0 Hz, 1H).

Synthesis of Compound 20

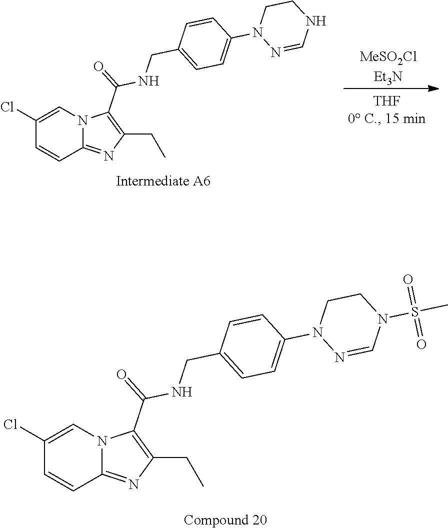 [SMILES: "MeSO2ClEt3N" ; CCc1nc2ccc(Cl)cn2c1C(=O)NCc1ccc(N2CCNC=N2)cc1 ; CCc1nc2ccc(Cl)cn2c1C(=O)NCc1ccc(N2CCN(S(C)(=O)=O)C=N2)cc1]

A mixture of intermediate A6 (120 mg, 0.302 mmol) and Et₃N (210 μL, 1.51 mmol) in anhydrous THE (6 mL) was cooled to 0° C. Methanesulfonyl chloride (46.8 μL, 0.605 mmol) was added dropwise and the reaction mixture was stirred at 0° C. for 15 min. Additional amount of methanesulfonyl chloride (23.4 μL, 0.302 mmol) was added dropwise at 0° C. and the reaction mixture was stirred for another 30 min at 0° C. The reaction mixture was diluted with DCM and quenched with a small amount of MeOH and K₂CO₃ (10%, aq.) was added. The layers were separated and the aqueous layer was extracted with DCM (twice). The combined organic layers were washed with water and brine, dried over MgSO₄, filtered and evaporated in vacuo. The residue was purified by preparative LC (irregular SiOH 15-40 μm, 12 g, dry loading (Celite®), mobile phase: heptane/EtOAc, gradient from 30:70 to 0:100, then EtOAc/MeOH 99:1). The solid was triturated in EtOAc and the supernatant was removed to give 68 mg of compound 20 as a white solid (47%).

¹H NMR (400 MHz, DMSO-d₆) δ ppm 9.06 (d, J=1.6 Hz, 1H) 8.43 (t, J=5.8 Hz, 1H) 7.66 (d, J=9.5 Hz, 1H) 7.45 (dd, J=9.4, 2.08 Hz, 1H) 7.28 (d, J=8.6 Hz, 2H) 7.19 (s, 1H) 7.17 (d, J=8.8 Hz, 2H) 4.46 (d, J=5.9 Hz, 2H) 3.86 (t, J=5.1 Hz, 2H) 3.70 (t, J=5.1 Hz, 2H) 3.27 (s, 3H) 2.97 (d, J=7.5 Hz, 2H) 1.99 (s, 1H) 1.25 (t, J=7.5 Hz, 3H).

Synthesis of Compound 21

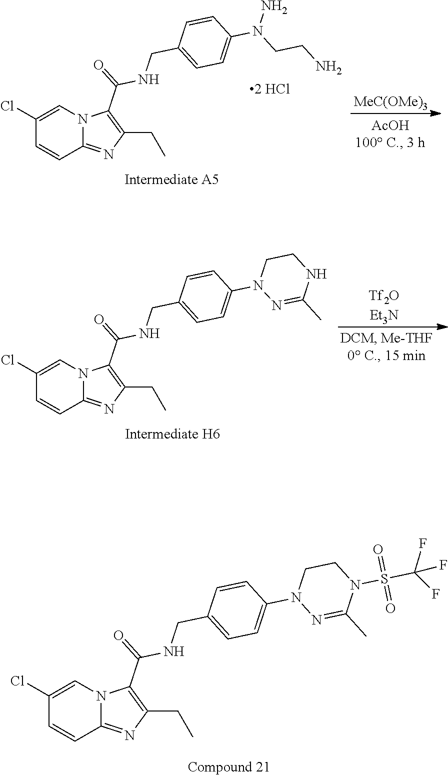 [SMILES: "MeC(OMe)3" ; CCc1nc2ccc(Cl)cn2c1C(=O)NCc1ccc(N(N)CCN)cc1 ; CCc1nc2ccc(Cl)cn2c1C(=O)NCc1ccc(N2CCNC(C)=N2)cc1 ; "Tf2OEt3N" ; CCc1nc2ccc(Cl)cn2c1C(=O)NCc1ccc(N2CCN(S(=O)(=O)C(F)(F)F)C(C)=N2)cc1]

Preparation of Intermediate H6

A mixture of intermediate A5 (200 mg, 0.435 mmol) and trimethyl orthoacetate (166 μL, 1.31 mmol) in acetic acid (3.6 mL) was stirred for 3 h at 100° C. The reaction mixture was evaporated in vacuo. The residue was diluted with DCM and K₂CO₃ (10%, aq.) was added. The layers were separated and the aqueous layer was extracted with DCM and MeOH (95/5) (twice). The combined organic layers were dried over MgSO₄, filtered and evaporated in vacuo. The residue was purified by preparative LC (irregular SiOH 15-40 μm, 12 g, dry loading, mobile phase: DCM/MeOH, gradient from 100:0 to 95:5) to give 132 mg of intermediate H6 as a yellow foam (77% purity, 57%).

Preparation of Compound 21

To a mixture of intermediate H6 (133 mg, 0.249 mmol) in anhydrous DCM (2.7 mL) and anhydrous Me-THF (2.5 mL) was added Et₃N (0.17 mL, 1.3 mmol). The mixture was cooled to 0° C. and trifluoromethanesulfonic anhydride (0.75 mL, 0.75 mmol) was added dropwise. The reaction mixture was stirred at 0° C. for 15 min and quenched with a small amount of MeOH and K₂CO₃ (10%, aq.). The layers were separated and the aqueous phase was extracted with DCM (twice). The combined organic extracts were washed with brine, dried over MgSO₄, filtered and evaporated in vacuo. The residue was purified by preparative LC (irregular SiOH 15-40 μm, 12 g, dry loading (Celite®), mobile phase: heptane/EtAOc, gradient from 80:20 to 0:100). A second purification was performed via reverse phase (stationary phase: YMC-actus Triart C18 10 μm 30*150 mm, mobile phase: NH₄HCO₃ (0.2% in water)/MeCN, gradient from 40:60 to 10:90) to give 52 mg of compound 21 as an off-white solid (38%).

¹H NMR (500 MHz, DMSO-d₆) δ ppm 9.06 (d, J=1.6 Hz, 1H) 8.44 (s, 1H) 7.66 (d, J=9.5 Hz, 1H) 7.45 (dd, J=9.6, 2.1 Hz, 1H) 7.30 (d, J=8.8 Hz, 2H) 7.16 (d, J=8.8 Hz, 2H) 4.46 (d, J=6.0 Hz, 2H) 4.00 (t, J=5.4 Hz, 2H) 3.82 (t, J=5.4 Hz, 2H) 2.97 (q, J=5.6 Hz, 2H) 2.26 (s, 3H) 1.25 (t, J=7.6 Hz, 3H).

Synthesis of Compound 22

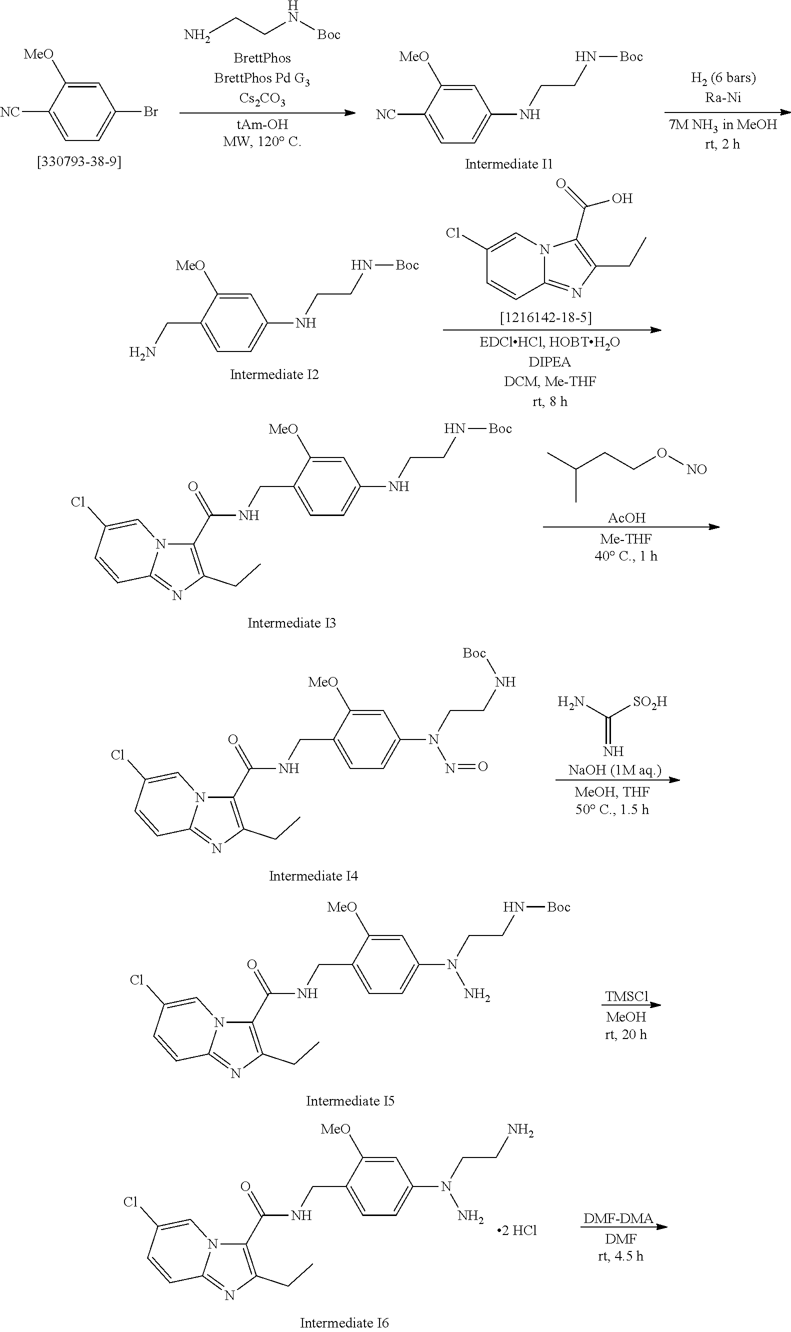

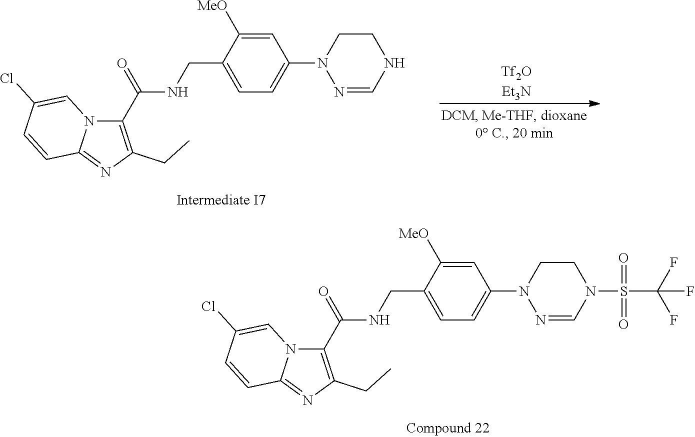 [SMILES: CCc1nc2ccc(Cl)cn2c1C(=O)NCc1ccc(N2CCNC=N2)cc1OC ; "Tf2OEt3N" ; CCc1nc2ccc(Cl)cn2c1C(=O)NCc1ccc(N2CCN(S(=O)(=O)C(F)(F)F)C=N2)cc1OC]

Preparation of Intermediate I1

A mixture of 4-bromo-2-methoxybenzonitrile [330793-38-9] (1.55 g, 7.31 mmol), N-boc-ethylenediamine (1.76 g, 11.0 mmol) and Cs₂CO₃ (4.76 g, 14.6 mmol) in anhydrous tert-amyl alcohol (46 mL) was purged with N₂. Brettphos Pd G3 (331 mg, 0.365 mmol) and Brettphos (392 mg, 0.731 mmol) were added and the reaction mixture was heated at 120° C. using a single mode microwave (Biotage Initiator60) for 1 h, and then for another 45 min. The two batches were filtered on a pad of Celite® and the filtrate was evaporated in vacuo. The residue was purified by preparative LC (irregular SiOH 15-40 μm, 120 g, dry loading (Celite®), mobile phase: heptane/EtAOc, gradient from 90:10 to 0:100) to give 1.64 g of intermediate I1 (74%).

Preparation of Intermediate 12

Intermediate 12 was prepared following the procedure reported for the synthesis of intermediate F2 starting from intermediate I1 and affording 1.55 g of a grey oil (94%).

Preparation of Intermediate 13

Intermediate 13 was prepared following the procedure reported for the synthesis of intermediate F3 starting from intermediate 12 and affording 765 mg of a beige solid (62%).

Preparation of Intermediate 14

Intermediate 14 was prepared following the procedure reported for the synthesis of intermediate F4 starting from intermediate 13 and affording 724 mg of a yellow solid (90%).

Preparation of Intermediate 15

Intermediate 15 was prepared following the procedure reported for the synthesis of intermediate F5 starting from intermediate 14 and affording 692 mg of a beige foam (99%).

Preparation of Intermediate 16

Intermediate E6 was prepared following the procedure reported for the synthesis of intermediate F6 starting from intermediate 15 and affording 710 mg of a beige solid (Quant.).

Preparation of Intermediate 17

A solution of intermediate 16 (270 mg, 0.551 mmol) and N,N-dimethylformamide dimethyl acetal (73.8 μL, 0.551 mmol) in anhydrous DMF (3.4 mL) was stirred at room temperature for 4.5 h. The reaction mixture was diluted with DCM and quenched K₂CO₃ (10%, aq.). The layers were separated and the aqueous phase was extracted with DCM and MeOH (95/5) (twice). The combined organic layers were dried over MgSO₄, filtered and evaporated in vacuo. The residue was purified by preparative LC (irregular SiOH 15-40 μm, 12 g, dry loading (Celite®), mobile phase: DCM/(DCM:MeOH, 80/20), gradient from 95:5 to 85:15) to give 100 mg of intermediate 17 as a white solid (42%).

Preparation of Compound 22

Under N₂ atmosphere and at 0° C., to a mixture of intermediate 17 (92.0 mg, 0.216 mmol) and Et₃N (150 μL, 1.08 mmol) in anhydrous DCM (3.1 mL), anhydrous Me-THE (3.1 mL) and anhydrous 1,4-dioxane (3.1 mL) was added dropwise trifluoromethanesulfonic anhydride (0.323 mL, 0.323 mmol). The reaction mixture was stirred at 0° C. for 10 min, and diluted with DCM and K₂CO₃ (10%, aq.). The layers were separated and the aqueous phase was extracted with DCM and MeOH (95/5) (twice). The combined organic extracts were dried over MgSO₄, filtered and evaporated in vacuo. The residue was purified by preparative LC (irregular SiOH 15-40 μm, 12 g, dry loading (Celite®), mobile phase: DCM/(DCM/MeOH, 95/5), gradient from 100:0 to 80/20). The solid was triturated in EtOAc. The supernatant was removed and the white solid was dried under vacuum for 1 h at 60° C. to give 28 mg of compound 22 (23%).

¹H NMR (400 MHz, DMSO-d₆) δ ppm 9.04 (d, J=1.5 Hz, 1H) 8.23 (t, J=5.7 Hz, 1H) 7.66 (d, J=9.7 Hz, 1H) 7.45 (dd, J=9.5, 2.1 Hz, 1H) 7.31 (s, 1H) 7.19 (d, J=8.3 Hz, 1H) 6.93 (d, J=2.0 Hz, 1H) 6.70 (dd, J=8.3, 2.0 Hz, 1H) 4.43 (d, J=5.7 Hz, 2H) 4.07 (br d, J=4.6 Hz, 2H) 3.86 (br d, J=5.3 Hz, 2H) 3.84 (s, 3H) 2.96 (d, J=7.5 Hz, 2H) 1.25 (t, J=7.5 Hz, 3H).

Synthesis of Compound 23

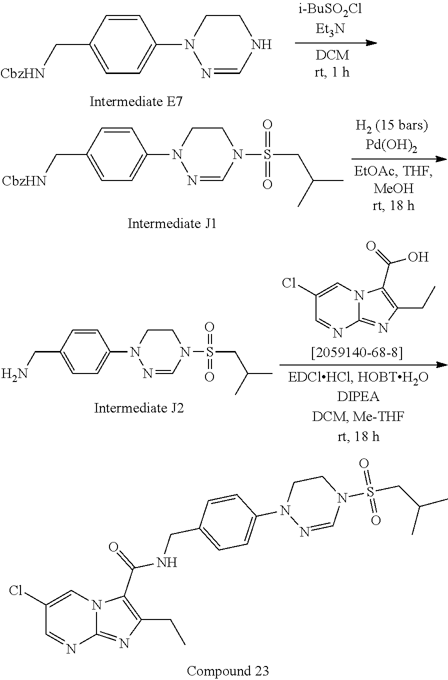 [SMILES: O=C(NCc1ccc(N2CCNC=N2)cc1)OCc1ccccc1 ; CC(C)CS(=O)(=O)N1C=NN(c2ccc(CNC(=O)OCc3ccccc3)cc2)CC1 ; CC(C)CS(=O)(=O)N1C=NN(c2ccc(CN)cc2)CC1 ; CCc1nc2ncc(Cl)cn2c1C(=O)O ; CCc1nc2ncc(Cl)cn2c1C(=O)NCc1ccc(N2CCN(S(=O)(=O)CC(C)C)C=N2)cc1]

Preparation of Intermediate J1

To a mixture of intermediate E7 (400 mg, 1.23 mmol) and Et₃N (0.857 mL, 6.17 mmol) in anhydrous DCM (18 mL) was added isobutanesulfonyl chloride (0.161 mL, 1.23 mmol) dropwise at 0° C. The reaction mixture was stirred at room temperature for 1 h. The reaction was quenched with NaHCO₃ (sat., aq.). The layers were separated and the aqueous phase was extracted with DCM and MeOH (95/5) (twice). The combined organic extracts were dried over MgSO₄, filtered and evaporated in vacuo. The residue was purified by preparative LC (irregular SiOH 15-40 μm, 24 g, dry loading (Celite®), mobile phase: heptane/EtOAc, gradient from 100:0 to 0:100, then mobile phase EtOAc/MeOH, gradient from 100:0 to 95:5) to give 406 mg of intermediate J1 as a green solid (74%).

Preparation of Intermediate J2

A mixture of intermediate J1 (406 mg, 0.913 mmol) and Pd(OH)₂ (264 mg, 0.941 mmol) in MeOH (20 mL), EtOAc (20 mL) and THF (5 mL) was stirred at room temperature under 15 bar of H₂ for 18 h. The reaction mixture was filtered off and rinsed with MeOH, EtOAc and THF. The filtrate was evaporated in vacuo to give 180 mg of intermediate J2 as a yellow solid (60%).

Preparation of Compound 23

A mixture of 6-chloro-2-ethyl-imidazo[1,2-a]pyrimidine-3carboxylic acid [2059140-68-8] (113 mg, 0.501 mmol), intermediate J2 (180 mg, 0.551 mmol), EDCI·HCl (96.0 mg, 0.501 mmol), HOBt·H₂O (76.7 mg, 0.501 mmol) and DIPEA (431 μL, 2.50 mmol) in DCM (10 mL) and Me-THF (6 mL) was stirred at room temperature for 18 h. The reaction mixture was diluted with DCM and washed with water (twice) and brine. The organic phase was dried over MgSO₄, filtered and evaporated in vacuo. The residue was purified by preparative LC (irregular SiOH 15-40 μm, 12 g, dry loading (Celite®), mobile phase: heptane/EtAOc, gradient from 90:10 to 0:100, then mobile phase: EtOAc/MeOH, gradient from 100:0 to 95:5) to give 101 mg of compound 23 as a slightly yellow solid (39%).

¹H NMR (500 MHz, DMSO-d₆) δ ppm 9.39 (d, J=2.8 Hz, 1H) 8.67 (d, J=2.6 Hz, 1H) 8.51 (t, J=6.0 Hz, 1H) 7.28 (d, J=8.7 Hz, 2H) 7.19 (s, 1H), 7.17 (d, J=8.8 Hz, 3H) 4.46 (d, J=6.0 Hz, 2H) 3.86 (t, J=4.8 Hz, 2H) 3.69 (t, J=4.9 Hz, 2H) 3.32 (d, J=6.6 Hz, 3H) 3.01 (q, J=7.5 Hz, 2H) 2.13 (m, 1H) 1.27 (t, J=7.6 Hz, 3H) 1.06 (s, 3H) 1.04 (s, 3H).

Synthesis of Compound 24

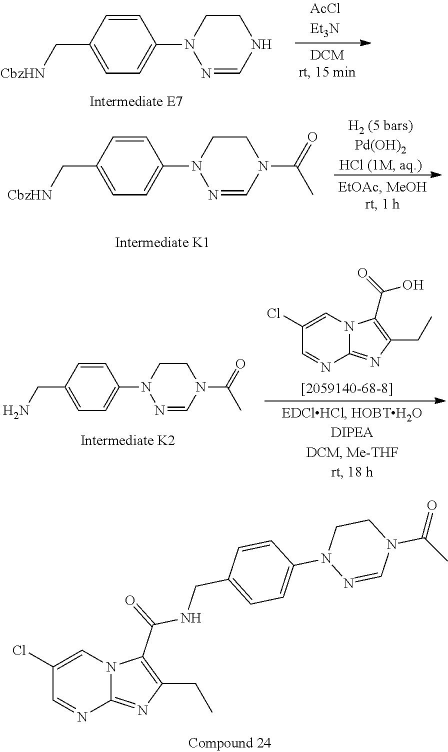 [SMILES: O=C(NCc1ccc(N2CCNC=N2)cc1)OCc1ccccc1 ; CC(=O)N1C=NN(c2ccc(CNC(=O)OCc3ccccc3)cc2)CC1 ; CC(=O)N1C=NN(c2ccc(CN)cc2)CC1 ; CCc1nc2ncc(Cl)cn2c1C(=O)O ; CCc1nc2ncc(Cl)cn2c1C(=O)NCc1ccc(N2CCN(C(C)=O)C=N2)cc1]

Preparation of Intermediate K1

To a mixture of intermediate E7 (550 mg, 1.70 mmol) and Et₃N (1.18 mL, 8.48 mmol) in anhydrous DCM (24 mL) at 0° C. was added acetyl chloride (0.145 mL, 2.04 mmol) dropwise. The reaction mixture was stirred at room temperature for 15 min, and the reaction was quenched with NaHCO₃ (sat., aq.). The layers were separated and the aqueous phase was extracted with DCM and MeOH (95/5) (twice). The combined organic extracts were dried over MgSO₄, filtered, and evaporated in vacuo. The residue was triturated in EtOAc and the solid was collected by filtration to afford 320 mg of intermediate K1 as a slightly yellow solid (52%).

Preparation of Intermediate K2

A mixture of intermediate K1 (256 mg, 0.698 mmol), Pd(OH)₂ (157 mg, 0.558 mmol) and HCl (1M in H₂O, 0.698 mL, 0.698 mmol) in MeOH (6.4 mL) and EtOAc (6.4 mL) was stirred at room temperature under 5 bars of H₂ for 1 h. The reaction mixture was filtered and rinsed with EtOAc and MeOH. The yellow solid was purified by preparative LC (irregular SiOH 15-40 μm, 12 g, dry loading (Celite®), mobile phase DCM/(DCM/MeOH/NH₃ aq., 80/20/0.5), gradient from 100:0 to 70:30) to give 130 mg of intermediate K2 (75%).

Preparation of Compound 24

To a mixture of 6-chloro-2-ethyl-imidazo[1,2-a]pyrimidine-3-carboxylic acid [2059140-68-8] (98.5 mg, 0.436 mmol), intermediate K2 (129 mg, 0.480 mmol) and DIPEA (752 μL, 4.36 mmol) in DCM (8.8 mL) and Me-THF (5.2 mL) were added EDCI·HCl (83.7 mg, 0.436 mmol) and HOBt·H₂O (66.8 mg, 0.436 mmol). The reaction mixture was stirred at room temperature for 16 h, filtered and the solid was washed with DCM to give 114 mg of compound 24 as a slightly yellow downy solid (59%).

¹H NMR (500 MHz, DMSO-d₆) δ ppm 9.38 (d, J=2.2 Hz, 1H) 8.61 (d, J=2.5 Hz, 1H) 8.26 (br t, J=6.0 Hz, 1H) 7.56 (br s, 1H) 7.28 (br d, J=8.5 Hz, 2H) 7.18 (d, J=8.5 Hz, 2H) 4.47 (d, J=5.7 Hz, 2H) 3.84 (br s, 2H) 3.64 (t, J=5.0 Hz, 2H) 3.01 (q, J=7.6 Hz, 3H) 2.23 (br s, 3H) 1.28 (t, J=7.4 Hz, 3H).

Synthesis of Compound 25

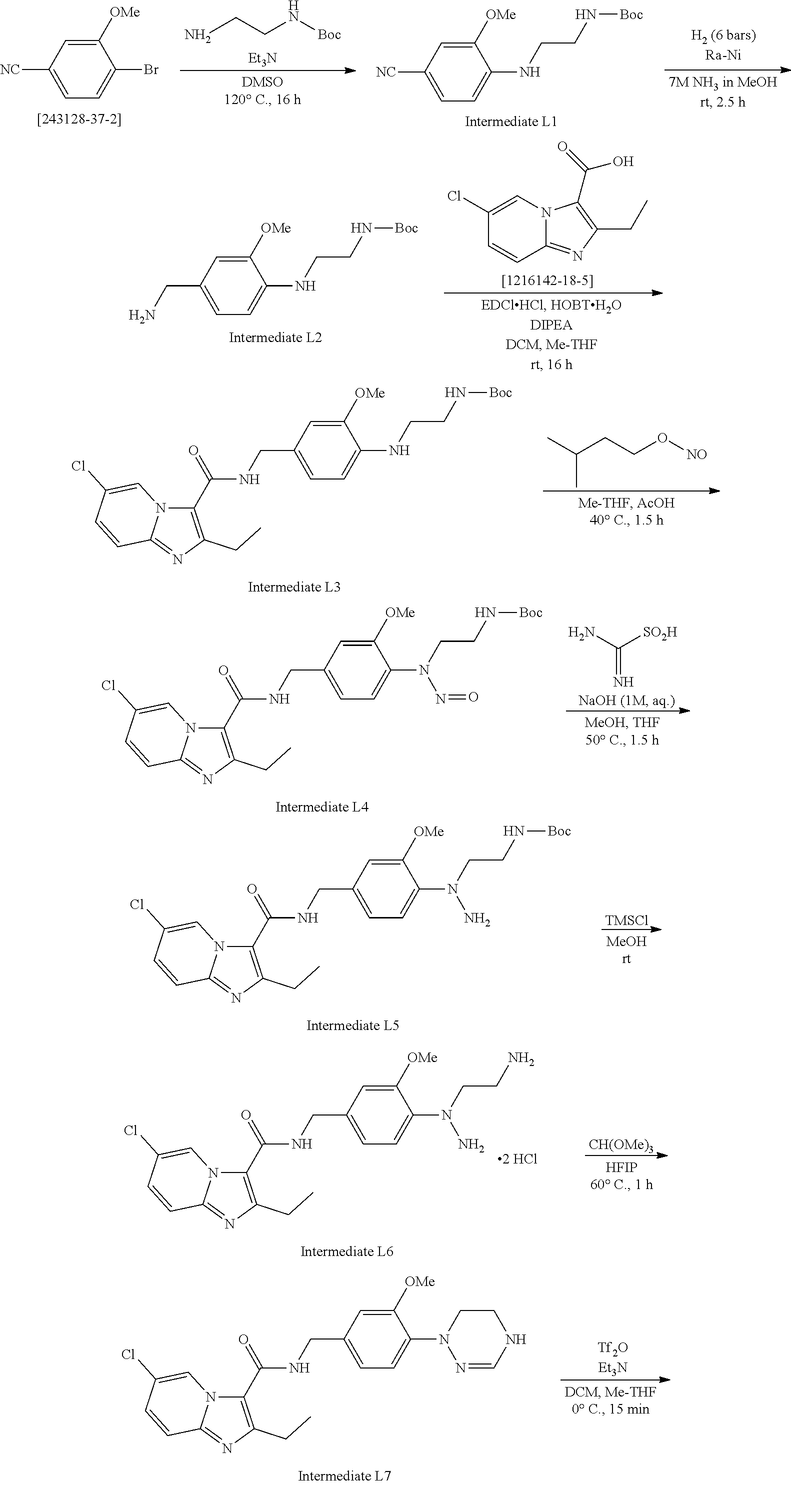

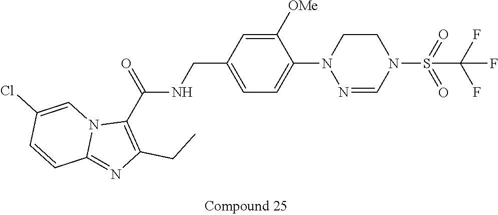 [SMILES: CCc1nc2ccc(Cl)cn2c1C(=O)NCc1ccc(N2CCN(S(=O)(=O)C(F)(F)F)C=N2)c(OC)c1]

Preparation of Intermediate L1

To a mixture of 4-fluoro-3-methoxy-benzonitrile [243128-37-2] (4.88 g, 32.3 mmol) and N-boc-ethylenediamine (18.0 mL, 0.129 mol) in DMSO (58 mL) was added Et₃N (6.65 mL, 42.0 mmol). The reaction mixture was stirred at 120° C. for 16 h. The reaction mixture was cooled down and poured in brine. EtOAc was added. The layers were separated and the aqueous phase was extracted with EtOAc (twice). The combined organic extracts were washed with a mixture of water and brine (1/1) (3 times), dried over MgSO₄, filtered and evaporated in vacuo. The residue was purified by preparative LC (irregular SiOH 15-40 μm, 330 g, dry loading (Celite®), mobile phase: heptane/EtOAc, gradient from 100:0 to 30:70) to give 5.23 g of intermediate L1 as a white solid (56%).

Preparation of Intermediate L2

Intermediate L2 was synthesized according to the procedure reported for the synthesis of intermediate F2 starting from intermediate L1 and affording 1.09 g of a green oil (Quant.).

Preparation of Intermediate L3

To a mixture of 6-chloro-2-ethylimidazo[1,2-a]pyridine-3-carboxylic [1216142-18-5](701 mg, 3.12 mmol), intermediate L2 (1.01 g, 3.43 mmol) and DIPEA (2.69 mL, 15.6 mmol) in DCM (60 mL) and Me-THF (40 mL) were added EDCI·HCl (598 mg, 3.12 mmol) and HOBt·H₂O (478 mg, 3.12 mmol). The reaction mixture was stirred at room temperature for 16 h and diluted with DCM and water. The layers were separated and the aqueous phase was extracted with DCM (twice). The combined organic extracts were washed with brine (twice), dried over MgSO₄, filtered and evaporated in vacuo. The residue was purified by preparative LC (irregular SiOH 15-40 μm, 80 g, dry loading (Celite®), mobile phase: heptane/EtOAc, gradient from 60:40 0:100) to give 1.078 g of intermediate L3 as a yellow solid (69%).

Preparation of Intermediate L4

Intermediate L3 (1.08 g, 2.15 mmol) was solubilized in Me-THF (21 mL) and acetic acid (1.23 mL, 21.5 mmol). Isopentyl nitrite (1.44 mL, 10.7 mmol) was added dropwise and the reaction mixture was stirred at 40° C. for 1.5 h. The reaction mixture was diluted with EtOAc and NaHCO₃ (sat., aq.). The layers were separated. The organic phase was washed with NaHCO₃ (sat., aq.) (twice) and brine, dried over MgSO₄, filtered and evaporated in vacuo. The residue was triturated in pentane and the supernatant was removed to give a yellow solid which was dried under vacuum to afford 1.127 g of intermediate L4 (99%).

Preparation of Intermediate L5

Intermediate L5 was prepared following the procedure reported for the synthesis of intermediate F5 starting from intermediate L4 and affording 1.07 g of an orange foam (97%).

Preparation of Intermediate L6

Intermediate L6 was prepared following the procedure reported for the synthesis of intermediate F6 starting from intermediate L5 and affording 1.10 g of a yellow powder (Quant.).

Preparation of Intermediate L7

A mixture of intermediate L6 (600 mg, 1.14 mmol) and trimethyl orthoformate (374 μL, 3.42 mmol) in HFIP (10.8 mL) was stirred at 60° C. for 1 h. The reaction mixture was diluted with EtOAc and quenched with K₂CO₃ (10%, aq.). The layers were separated and the organic phase was washed with H₂O and brine, dried over MgSO₄, filtered and evaporated in vacuo. The residue was purified by preparative LC (irregular SiOH 15-40 μm, 25 g, dry loading (Celite®), mobile phase: DCM/(DCM/MeOH, 80/20), gradient from 100:0 to 50:50) to give 290 mg of intermediate L7 as a slightly orange solid (60%).

Preparation of Compound 25

To a mixture of intermediate L7 (290 mg, 0.679 mmol) and Et₃N (0.472 mL, 3.40 mmol) in anhydrous DCM (10 mL) and anhydrous Me-THF (10 mL) was added dropwise trifluoromethanesulfonic anhydride (0.815 mL, 0.815 mmol) at 0° C. The reaction mixture was stirred at 0° C. for 15 min and diluted with DCM. A small amount of MeOH and K₂CO₃ (10%, aq.) were successively added. The layers were separated and the aqueous phase was extracted with DCM and MeOH (95/5) (twice). The combined organic extracts were washed with water and brine, dried over MgSO₄, filtered and evaporated. The residue was purified by preparative LC (irregular SiOH 15-40 μm, 25 g, dry loading (Celite®), mobile phase: heptane/EtOAc, gradient from 70:30 to 0:100). The yellow solid was triturated in Et₂O, sonicated and collected by filtration to give 135 mg of compound 25 as a beige solid (36%).

¹H NMR (500 MHz, DMSO-d₆) δ ppm 9.06 (d, J=1.6 Hz, 1H) 8.47 (br t, J=6.0 Hz, 1H) 7.66 (d, J=9.5 Hz, 1H) 7.46 (dd, J=9.5, 2.2 Hz, 1H) 7.29 (s, 1H) 7.21 (d, J=7.9 Hz, 1H) 7.08 (s, 1H) 6.96 (d, J=7.9 Hz, 1H) 4.52 (d, J=6.0 Hz, 2H) 4.06 (br t, J=4.4 Hz, 2H) 3.82 (s, 3H) 3.55 (br t, J=4.7 Hz, 2H) 3.01 (d, J=7.6 Hz, 2H) 1.27 (t, J=7.6 Hz, 3H)

Synthesis of Compound 26

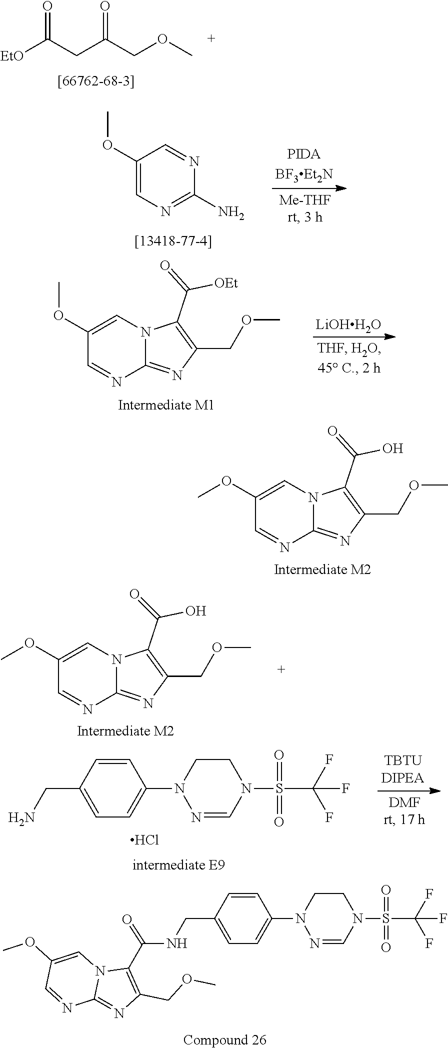

Preparation of Intermediate M1

To a mixture of 2-amino-5-methoxypyrimidine [13418-77-4] (4.75 g, 38.0 mmol), ethyl-3-oxovaleraethyl-3-oxovalerate [4949-44-4] (9.48 mL, 66.4 mmol) and (diacetoxyiodo)benzene (iodobenzenediacteate) (12.2 g, 38.0 mmol) in anhydrous Me-THF (150 mL) was added boron trifluoride etherate (0.993 mL, 3.80 mmol) dropwise. The reaction mixture was stirred at room temperature for 3 h. The two batches were combined and the mixture was diluted with EtOAc. NaHCO₃ (sat., aq.) was added. The layers were separated and the organic phase was washed with brine, dried over MgSO₄, filtered and concentrated in vacuo. The residue was purified by preparative LC (irregular SiOH 15-40 μm, 330 g, liquid injection (DCM), mobile phase: heptane/EtOAc, gradient from 85:15 to 50:50) to give 4.94 g of intermediate M1 as a yellow solid (26%).

Preparation of Intermediate M2

To a solution of intermediate M1 (500 mg, 2.01 mmol) in THE (10 mL) was added a solution of LiOH·H₂O (253 mg, 6.02 mmol) in water (5 mL). The reaction mixture was stirred for 2 h at 45° C., cooled to room temperature and HCl (1M, aq., 6 mL) was added followed by EtOAc. The layers were separated and the aqueous phase was extracted with DCM, then with a mixture of DCM and MeOH (95/5). The combined organic extracts were dried over MgSO₄, filtered and evaporated in vacuo to afford 80 mg of intermediate M2 (18%).

Preparation of Compound 26

To a mixture of intermediate M2 (80 mg, 0.362 mmol) and intermediate E9 (117 mg, 0.362 mmol) in DMF (2.44 mL) were successively added DIPEA (0.156 mL, 0.904 mmol) and TBTU (128 mg, 0.398 mmol). The reaction mixture was stirred at room temperature for 17 h. The reaction mixture was poured in EtOAc. The organic phase was washed with brine (twice), dried over MgSO₄, filtered and evaporated in vacuo. The residue was purified by preparative LC (irregular SiOH 15-40 μm, 24 g, liquid injection (DCM), mobile phase: heptane/EtOAc, gradient from 50:50 to 0:100) to give 78 mg of compound 26 as a white solid (41%).

¹H NMR (400 MHz, DMSO-d₆) δ ppm 9.40 (d, J=2.57 Hz, 1H) 8.68 (d, J=2.69 Hz, 1H) 8.53 (t, J=5.87 Hz, 1H) 7.30 (d, J=8.68 Hz, 2H) 7.15 (d, J=8.68 Hz, 2H) 4.46 (d, J=5.87 Hz, 2H) 4.06-4.18 (m, 2H) 3.85 (s, 3H) 3.69-3.78 (m, 2H) 3.01 (q, J=7.54 Hz, 2H) 1.27 (t, J=7.52 Hz, 3H).

Synthesis of Compound 27

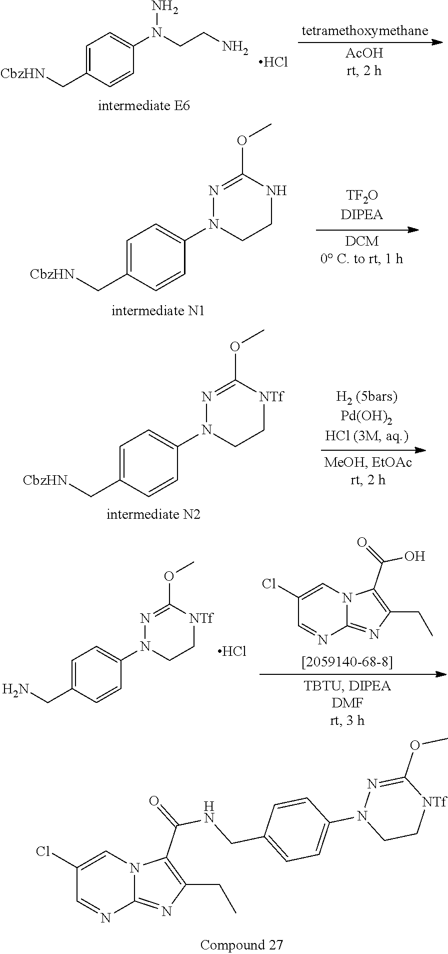

Preparation of Intermediate N1

A solution of intermediate E6 (3.00 g, 7.75 mmol) in acetic acid (30 mL) was treated with tetramethoxymethane (2.58 mL, 19.4 mmol) and stirred at room temperature for 2 h. The reaction mixture was poured in DCM and quenched with K₂CO₃ (10%, aq.). The layers were separated and the aqueous phase was extracted with DCM and MeOH (98/2). The combined organic extracts were dried over MgSO₄, filtered and evaporated in vacuo. The crude mixture was purified by preparative LC (irregular SiOH 15-40 μm, 80 g, liquid injection (DCM), mobile phase: heptane/EtOAc, gradient from 70:30 to 0:100) to give 1.09 g of intermediate N1 as an oil (40%).

Preparation of Intermediate N2

To a mixture of intermediate N1 (1.00 g, 2.82 mmol) and DIPEA (0.972 mL, 5.64 mmol) in DCM (15 mL) was added a solution of Tf₂O in DCM (1M in DCM, 2.96 mL, 2.96 mmol) dropwise over 10 min. The reaction mixture was stirred at room temperature for 30 min and diluted with DCM. The mixture was washed with NaHCO₃ (sat., aq.), dried over MgSO₄, filtered and evaporated in vacuo. The residue was purified by preparative LC (irregular SiOH 15-40 μm, 40 g, liquid injection (DCM), mobile phase: heptane/EtOAc, gradient from 80:20 to 40:60) to give 680 mg of intermediate N2 as a white solid (50%).

Preparation of Intermediate N3

In a steal bomb, a mixture of intermediate N2 (630 mg, 1.30 mmol), Pd(OH)₂ (132 mg, 0.470 mmol) and HCl (3M in H₂O, 0.432 mL, 1.30 mmol) in MeOH (5 mL) and EtOAc (5 mL) was hydrogenated under 5 bars of H₂ at room temperature for 2 h. The mixture was filtered on a pad of Celite® to give 503 mg of intermediate N3 as white solid (Quant.).

Preparation of Compound 27

A mixture of intermediate N3 (150 mg, 0.665 mmol), 6-chloro-2-ethyl-imidazo[1,2-a]pyrimidine-3-carboxylic acid [2059140-68-8] (284 mg, 0.731 mmol) and DIPEA (0.344 mL, 1.99 mmol) in DMF (4.5 mL) was treated with TBTU (235 mg, 0.731 mmol) and the reaction mixture was stirred at room temperature for 3 h. The reaction mixture was diluted with EtOAc, washed with water and brine, dried over MgSO₄, filtered and concentrated in vacuo. The residue was purified by preparative LC (irregular SiOH 40 μm, 24 g, liquid injection (DCM), mobile phase: heptane/EtOAc, gradient from 80:20 to 20:80). The white solid solubilized in warm EtOAc and the solution was cooled to room temperature, then to 0° C. The suspension was filtered off, washed with Et₂O, and dried under vacuum to give a solid (71 mg). The filtrate was evaporated in vacuo and combined with the solid. The residue was solubilized in warm i-PrOH, and cooled to room temperature. The suspension was slowly concentrated under vacuum (120 mbar) to obtain a thick solution. After filtration, the solid was washed with Et₂O, and dried under vacuum to afford 135 mg of compound 27 as a white solid (36%).

¹H NMR (400 MHz, DMSO-d₆) δ ppm 8.94 (d, J=3.06 Hz, 1H) 8.51 (d, J=3.06 Hz, 1H) 8.40 (t, J=5.87 Hz, 1H) 7.32 (d, J=8.68 Hz, 2H) 7.28 (s, 1H) 7.19 (d, J=8.68 Hz, 2H) 4.48 (d, J=5.87 Hz, 2H) 4.08 (t, J=4.65 Hz, 2H) 3.86 (s, 3H) 3.79-3.84 (m, 2H) 2.99 (q, J=7.50 Hz, 2H) 1.25 (t, J=7.52 Hz, 3H).

Synthesis of Compound 28

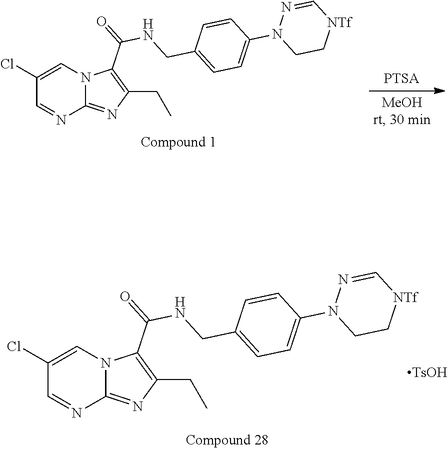

PTSA (108 mg, 567 μmol) was added to a suspension of compound 1 (300 mg, 567 mmol) in MeOH (7.8 mL). After sonication, the solution was stirred at room temperature for 1 h and the solvent was removed under reduced pressure. The residue was triturated in Et₂O and the solvent was removed under reduced pressure (operation repeated twice) to give 406 mg of compound 28 as an off-white solid (Quant.).

¹H NMR (400 MHz, DMSO-d₆) δ ppm 9.14 (s, 1H) 8.80 (t, J=5.7 Hz, 1H) 7.74-7.89 (m, 2H) 7.47 (d, J=8.1 Hz, 2H) 7.27-7.37 (m, 3H) 7.19 (d, J=8.7 Hz, 2H) 7.11 (d, J=7.8 Hz, 2H) 4.49 (d, J=5.9 Hz, 3H) 4.08 (t, J=4.4 Hz, 2H) 3.83 (t, J=4.8 Hz, 2H) 3.02 (q, J=7.5 Hz, 2H) 2.29 (s, 3H) 1.27 (t, J=7.5 Hz, 3H).

Synthesis of Compound 29

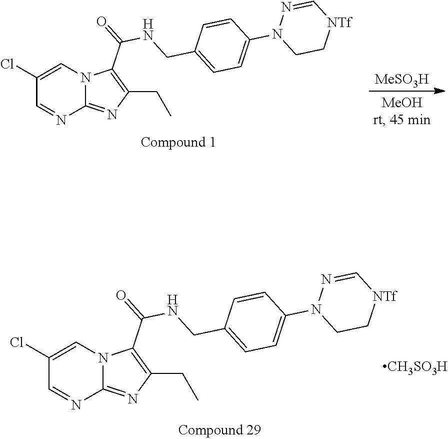

A solution of MeSO₃H in MeOH (9.1% v/v, 368 μL, 516 μmol) was added to a mixture of compound 1 (300 mg, 567 μmol) in MeOH (15 mL). The reaction mixture was stirred at room temperature for 45 min and evaporated to dryness. The residue was triturated in Et₂O and the solvent was removed under reduced pressure. The solid was dried under reduced pressure to give 355 mg of compound 29 as an off-white solid (Quant.).

¹H NMR (400 MHz, DMSO-d₆) δ ppm 9.13 (s, 1H) 8.74 (t, J=5.3 Hz, 1H) 7.82 (d, J=9.4 Hz, 1H) 7.73 (d, J=9.4 Hz, 1H) 7.33 (m, J=8.7 Hz, 2H) 7.29 (s, 1H) 7.19 (m, J=8.7 Hz, 2H) 4.49 (d, J=5.9 Hz, 2H) 4.08 (t, J=4.6 Hz, 2H) 3.83 (t, J=4.8 Hz, 2H) 3.02 (q, J=7.5 Hz, 2H) 2.32 (s, 3H) 1.27 (t, J=7.5 Hz, 3H).

Synthesis of Compound 30

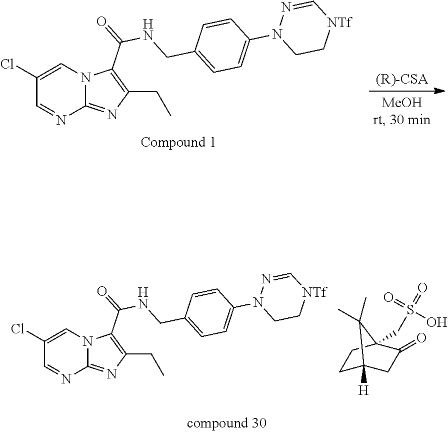

(1R)-(−)-Camphor-10-Sulfonic acid (110 mg, 473 μmol) was added to a solution of compound 1 (250 mg, 473 μmol) in anhydrous MeOH (5 mL). The reaction mixture was stirred at room temperature for 30 min and the solvent was removed under reduced pressure. The residue was triturated in Et₂O and the solvent was removed under reduced pressure to give 359 mg of compound 30 as a white solid (Quant.).

¹H NMR (400 MHz, DMSO-d₆) δ ppm 9.12 (d, J=1.3 Hz, 1H) 8.69 (t, J=5.3 Hz, 1H) 7.80 (m, 1H) 7.69 (m, 1H) 7.33 (d, J=8.6 Hz, 2H) 7.28 (s, 1H) 7.19 (d, J=8.7 Hz, 2H) 4.48 (d, J=5.7 Hz, 3H) 4.08 (t, J=4.6 Hz, 2H) 3.83 (t, J=4.8 Hz, 2H) 3.01 (q, J=7.6 Hz, 2H) 2.86 (d, J=14.7 Hz, 1H) 2.65-2.75 (m, 1H) 2.37 (d, J=14.7 Hz, 1H) 2.23 (dt, J=18.1, 3.9 Hz, 1H) 1.93 (t, J=4.5 Hz, 1H) 1.83-1.91 (m, 1H) 1.82 (s, 1H) 1.77 (s, 1H) 1.21-1.32 (m, 5H) 1.05 (s, 3H) 0.74 (s, 3H).

Synthesis of Compound 31

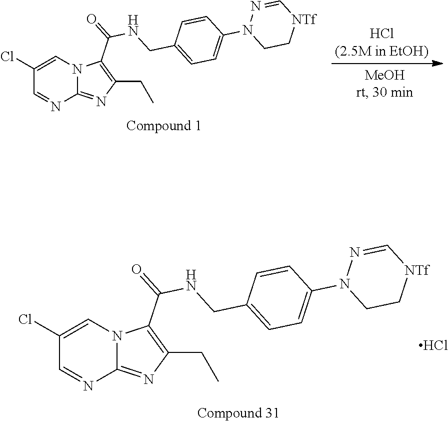

A solution of HCl in EtOH (2.5M, 89 μL, 473 μmol) was added to a mixture of compound 1 (250 mg, 473 μmol) in MeOH (2.7 mL). The reaction mixture was stirred at room temperature for 30 min, then evaporated in vacuo to dryness. The residue was triturated in Et₂O and the solvent was removed under reduced pressure to give 269 mg of compound 31 as a white solid (Quant.).

¹H NMR (400 MHz, DMSO-d₆) δ ppm 9.12 (s, 1H) 8.71 (m, 1H) 7.79 (d, J=9.4 Hz, 1H) 7.68 (d, J=8.8 Hz, 1H) 7.26-7.37 (m, 3H) 7.19 (d, J=8.7 Hz, 2H) 4.48 (d, J=5.9 Hz, 2H) 4.08 (t, J=4.5 Hz, 2H) 3.83 (t, J=4.8 Hz, 2H) 3.01 (q, J=7.6 Hz, 2H) 1.27 (t, J=7.5 Hz, 3H).

Synthesis of Compound 32

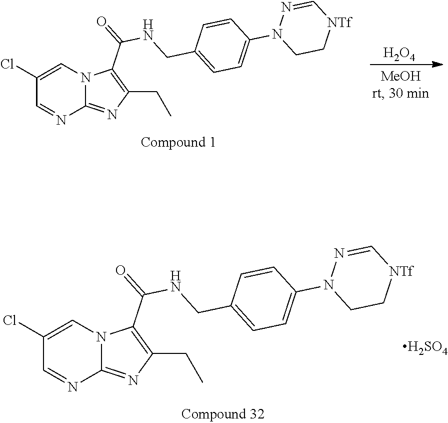

H₂SO₄ (13 μL, 238 μmol) was added to a solution of compound 1 (252 mg, 476 μmol) in MeOH (4.2 mL). The reaction mixture was stirred at room temperature for 30 min, then evaporated to dryness. The residue was triturated in Et₂O and the solvent was removed under reduced pressure. The white solid was dried at 60° C. for 6 h under vacuum to give 271 mg of compound 32 as a white solid (98%).

¹H NMR (400 MHz, DMSO-d₆) δ ppm 9.11 (s, 1H) 8.63 (t, J=5.5 Hz, 1H) 7.76 (d, J=9.5 Hz, 1H) 7.62 (d, J=9.8 Hz, 1H) 7.26-7.36 (m, 3H) 7.19 (d, J=8.7 Hz, 2H) 4.48 (d, J=5.9 Hz, 2H) 4.07 (t, J=4.7 Hz, 2H) 3.83 (t, J=4.7 Hz, 2H) 3.00 (q, J=7.5 Hz, 2H) 1.26 (t, J=7.5 Hz, 3H).

Synthesis of Compound 33

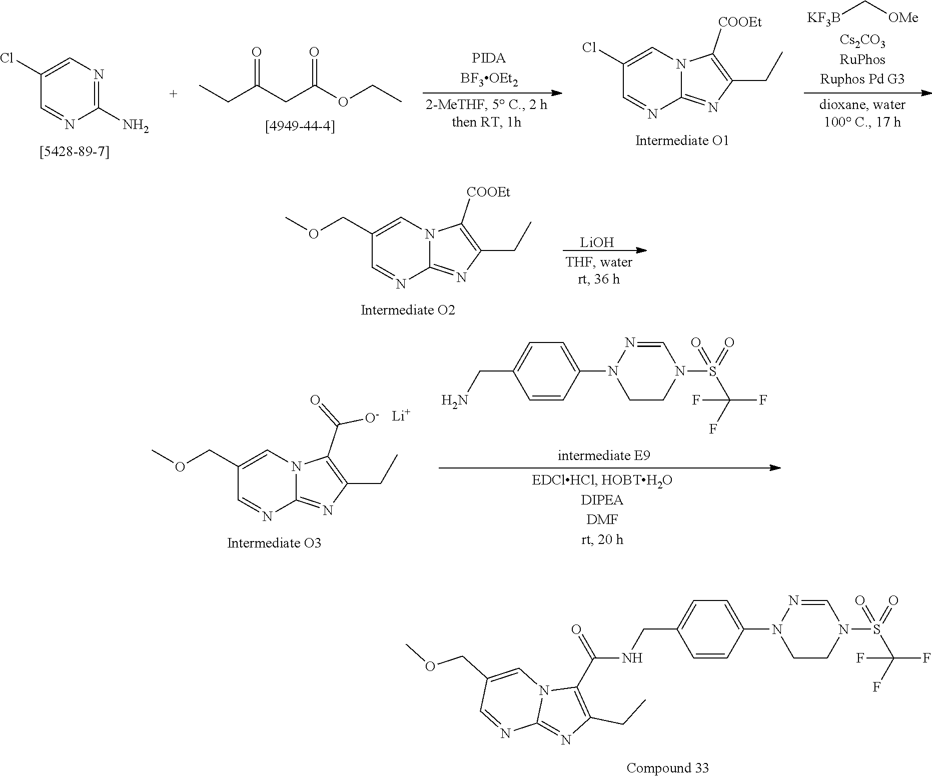

Preparation of Intermediate O1

A 2 L round bottom flask equipped with a dropping funnel was charged at 5° C. with a solution of 2-amino-5-chloropyrimidine [5428-89-7] (10 g, 77 mmol) in Me-THF (350 L). Ethyl-3-oxovalerate [4949-44-4] (20 mL, 140 mmol) and (diacetoxyiodo)benzene (iodobenzene diacetate) (25 g, 78 mmol) were added. Boron trifluoride diethyl etherate (1 mL, 3.8 mmol) was added dropwise over 30 min and the solution was stirred at 5° C. for 2 h. The mixture was warmed to room temperature and stirred for 1 h. The mixture was filtered. EtOAc and NaHCO₃ (sat., aq.) were added to the filtrate. The organic layer was dried over MgSO₄, filtered and concentrated in vacuo. The crude mixture was purified by preparative LC (irregular SiOH, 15-40 μm, 330 g, liquid injection (DCM), mobile phase: heptane/EtOAc, gradient from 85:15 to 50:50) to give intermediate O1 (2.98 g, 15%).

Preparation of Intermediate O2

A solution of intermediate O1 (1.00 g; 3.94 mmol), potassium (methoxymethyl) trifluoroborate [910251-11-5] (1.80 g, 11.8 mmol) and Cs₂CO₃ (3.85 g, 11.8 mmol) in 1,4-dioxane (10 mL) and water (1.4 mL) was purged with nitrogen. RuPhos (184 mg, 0.394 mmol) and RuPhos Pd G3 (330 mg, 0.394 mmol) were added. The reaction mixture was purged again with nitrogen and stirred at 100° C. for 17 h. The reaction mixture was concentrated in vacuo and purified by preparative LC (irregular SiOH 15-40 μm, 40 g, liquid injection (DCM), mobile phase: heptane/EtOAc, gradient from 75:25 to 0:100). The residue was purified by reverse phase (stationary phase: YMC-actus Triart C18 10 μm 30*150 mm, mobile phase: (aq. NH₄HCO₃ 0.2%)/MeCN, gradient from 70:30 to 30:70) to give intermediate O2 (212 mg, 20%) as a white solid.

Preparation of Intermediate O3

A mixture of intermediate O2 (130 mg, 0.494 mmol) and LiOH (14 mg, 0.585 mmol) in THE (2.3 mL) and water (2.3 mL) was stirred at room temperature for 36 h. The reaction mixture was evaporated in vacuo to afford 168 mg of intermediate O3 as a light-yellow gum. The crude product was used as such in next step.

Preparation of Compound 33

To a mixture of intermediate O3 (168 mg, 0.529 mmol) and DIPEA (0.275 mL, 1.59 mmol) in DMF (5 mL) were successively added HOBt·H₂O (83.0 mg, 0.542 mmol), EDCI·HCl (102 mg, 0.533 mmol) and intermediate E9 (223 mg, 0.536 mmol). The reaction mixture was stirred at room temperature for 20 h. DCM and water were added. The layers were separated and the organic layer was washed with NaHCO₃ (sat., aq.) and brine (3 times), dried over MgSO₄, filtered and evaporated. The crude mixture was purified by preparative LC (irregular SiOH 15-40 μm, 24 g, dry loading (Celite®), mobile phase: heptane/(EtOAc/MeOH, 9/1), gradient from 90:10 to 0:100). The residue (175 mg) was purified by reverse phase (stationary phase: YMC-actus Triart C18 10 μm 30*150 mm, 40 g, dry loading (Celite®), mobile phase: (aq. NH₄HCO₃ 0.2%)/MeCN, gradient from 90:10 to 30:70). MeCN was evaporated and the product was extracted with DCM (twice). The organic layer was dried over MgSO₄, filtered and evaporated in vacuo to afford 154 mg of a white solid. The product was purified by reverse phase (stationary phase: YMC-actus Triart C18 10 μm 30*150 mm, 40 g, dry loading (Celite®), mobile phase: (aq. NH₄HCO₃ 0.2%)/MeCN, gradient from 60:40 to 45:55). MeCN was evaporated and the product was extracted with DCM (twice). The organic layer was dried over MgSO₄, filtered and evaporated in vacuo. The product was triturated in MeCN and EtOAc, filtered and dried under high vacuum at 50° C. for 16 h to afford compound 33 (119 mg, 42%) as a white solid.

¹H NMR (400 MHz, DMSO-d₆) δ ppm 9.27 (d, J=2.3 Hz, 1H) 8.60 (d, J=2.4 Hz, 1H) 8.50 (t, J=6.0 Hz, 1H) 7.27-7.34 (m, 3H) 7.19 (d, J=8.7 Hz, 2H) 4.53 (s, 2H) 4.47 (d, J=5.9 Hz, 2H) 4.03-4.12 (m, 2H) 3.79-3.86 (m, 2H) 3.34 (s, 3H) 3.00 (q, J=7.5 Hz, 2H) 1.27 (t, J=7.5 Hz, 3H).

Synthesis of Compound 34

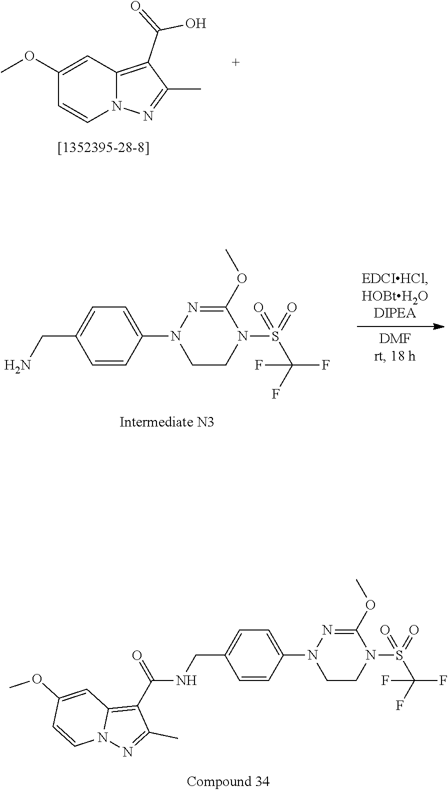

To a mixture of the 5-methoxy-2-methylpyrazolo[1,5-a]pyridine-3-carboxylic acid [1352395-28-8] (80 mg, 0.39 mmol), intermediate N3 (151 mg, 0.39 mmol) and DIPEA (201 μL, 1.17 mmol) in DMF (5 mL) were added EDCI·HCl (74 mg, 0.39 mmol) and HOBt·H₂O (59 mg, 0.39 mmol). The reaction mixture was stirred at room temperature for 18 h and concentrated in vacuo. The residue was diluted in EtOAc and water. The layers were separated and the aqueous phase was extracted with EtOAc. The combined organic layers were dried over MgSO₄, filtered and concentrated. The residue (229 mg) was purified by reverse phase (stationary phase: YMC-actus Triart C18 10 μm (30*150 mm), mobile phase: (aq. NH₄HCO₃ 0.2%)/MeCN, gradient from 50:50 to 25:75) affording 118 mg of compound 34.

¹H NMR (400 MHz, DMSO-d₆) δ ppm 8.49 (d, J=7.5 Hz, 1H) 7.85 (t, J=5.9 Hz, 1H) 7.22-7.29 (m, 3H) 7.14 (d, J=8.7 Hz, 2H) 6.62 (dd, J=7.5, 2.8 Hz, 1H) 4.41 (d, J=6.0 Hz, 2H) 4.07-4.12 (m, 2H) 3.84 (d, J=2.3 Hz, 6H) 3.69-3.75 (m, 2H) 2.52 (s, 3H).

Synthesis of Compound 35

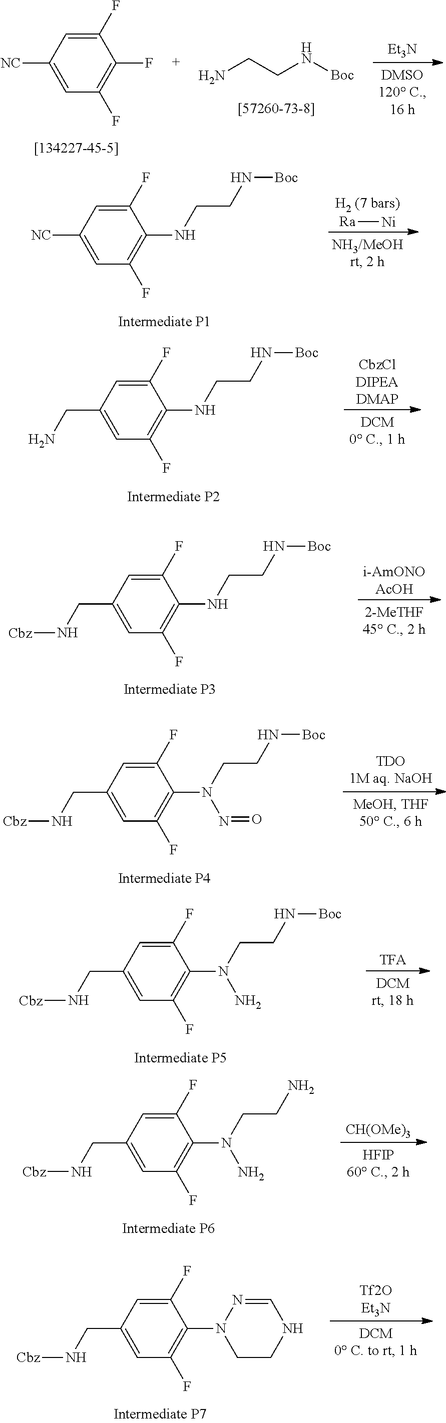

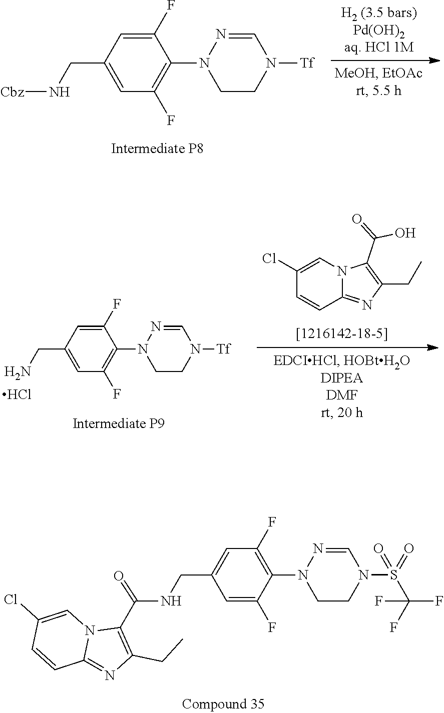

Preparation of Intermediate P1

In a round bottom flask, a solution of 3,4,5-trifluorobenzonitrile [134227-45-5] (5 g, 31.8 mmol), N-boc-1,2-diaminoethane [57260-73-8] (5.2 mL, 32.8 mmol) and Et₃N (17.7 mL, 127 mmol) in anhydrous DMSO (57 mL) was stirred at 120° C. for 16 h. The reaction mixture was cooled to room temperature and DMSO was evaporated with Genevac. EtOAc, water and NaCl were added. The layers were separated and the organic layer was washed with brine (3 times), dried over MgSO₄, filtered and evaporated in vacuo. The crude mixture was solubilized in EtOAc and SiOH was added. The dry loading was evaporated and washed with heptane (100 mL). The product was eluted with heptane/EtOAc (1:1, 3×100 mL). The filtrate was evaporated to afford 9.30 g of intermediate P1 as a colorless oil which crystallized on standing (98%).

Preparation of Intermediate P2

Intermediate P2 was prepared following the synthesis reported for intermediate E2, starting from intermediate P1 (31.3 mmol) and affording 9.3 g as a light blue gum (99%) which crystallized on standing.

Preparation of Intermediate P3

Intermediate P3 was prepared following the synthesis reported for intermediate E3, starting from intermediate P2 (6.64 mmol) and affording 1.63 g as a colorless oil (56%) which crystallized on standing.

Preparation of Intermediate P4

Intermediate P4 was prepared following the synthesis reported for intermediate E4, starting from intermediate P3 (3.74 mmol) and affording 1.91 g as a yellow oil (91%).

Preparation of Intermediate P5

Intermediate P5 was prepared following the synthesis reported for intermediate E5, starting from intermediate P4 (3.74 mmol) and affording 1.69 g as a yellow oil (100%) which crystallized on standing.

Preparation of Intermediate P6

A solution of intermediate P5 (1.69 g, 3.75 mmol) in anhydrous DCM (35 mL) was treated with TFA (3.5 mL, 45.7 mmol) and the reaction mixture was stirred at room temperature for 18 h. The reaction mixture was evaporated in vacuo to give 3.42 g of intermediate P6 as an orange gum.

Preparation of Intermediate P7

Trimethylorthoformate (1.24 mL, 11.3 mmol) was added to a solution of intermediate P6 (3.42 g, 3.78 mmol) in HFIP (35 mL) and the mixture was stirred at 60° C. for 2 h. The reaction mixture was cooled to room temperature, diluted with EtOAc and basified with NaHCO₃ (sat., aq.). The layers were separated and the aqueous layer was extracted with EtOAc (once). The combined organic layers were dried over MgSO₄, filtered and the solvent was removed under reduced pressure to give 2.0 g of intermediate P7 as a yellow gum.

Preparation of Intermediate P8

Triethylamine (1 mL, 7.19 mmol) was added to a solution of intermediate P7 (1.5 g, 2.83 mmol) in DCM (28 mL). The solution was then cooled to 0° C. (ice/water bath) and Tf₂O (1M in DCM, 3.4 mL, 3.4 mmol) was added dropwise over 5 min. The reaction mixture was stirred at 0° C. for 30 min. The mixture was slowly warmed to room temperature and stirred for 2 h. DCM, water and NaHCO₃ (10%, aq.) were added. The layers were separated, and the aqueous layer was extracted with DCM. The combined organic layers were dried over MgSO₄, filtered and evaporated. The residue (1.61 g) was purified by preparative LC (irregular SiOH, 30 μm, 80 g, liquid injection (DCM), mobile phase: heptane/EtOAc, gradient from 95:5 to 50:550) to afford 317 mg of intermediate P8 as an orange gum (23% over 3 steps).

Preparation of Intermediate P9

In a steal bomb, a mixture of intermediate P8 (317 mg, 0.644 mmol), palladium hydroxide, Pd 20% on carbon, nominally 50% water (120 mg, 0.171 mmol) and HCl (1M, aq., 0.64 mL, 0.64 mmol) in EtOAc (3.2 mL) and MeOH (3.2 mL) was hydrogenated under 5 bars of H₂ at room temperature for 4 h. The mixture was filtered. An extra amount of palladium hydroxide, Pd 20% on carbon, nominally 50% water (60 mg, 0.085 mmol) and HCl (1M, aq., 0.64 mL, 0.64 mmol) were added. The mixture was hydrogenated under 5 bars of H₂ at room temperature for 1.5 h. The reaction mixture was filtered and the filtrate was evaporated in vacuo to afford 269 mg of intermediate P9 as an orange gum. The crude product was used as such in next step.

Preparation of Compound 35

To a mixture of 6-chloro-2-ethylimidazo[1,2-a]pyridine-3-carboxylicacid [1216142-18-5] (80 mg, 0.356 mmol) and DIPEA (0.245 mL, 1.42 mmol) in DMF (3.5 mL) were successively added EDCI·HCl (72 mg, 0.376 mmol), HOBt·H₂O (60 mg, 0.392 mmol) and intermediate P9 (270 mg, 0.356 mmol). The reaction mixture was stirred at room temperature for 20 h. The crude mixture was taken-up in DCM and NaHCO₃ (sat., aq.) was added. The layers were separated and the organic layer was washed with brine (twice), dried over MgSO₄, filtered and evaporated in vacuo. The residue (409 mg) was purified by preparative LC (regular SiOH 30 μm, 24 g, mobile phase: heptane/(EtOAc/MeOH, 9/1), gradient from 80:20 to 20:80). A second purification was performed by reverse Phase (stationary phase: YMC-actus Triart C18 25 μm 30*150 mm, 40 g, dry loading (Celite®), mobile phase: (aq. NH₄HCO₃ 0.2%)/MeCN, gradient from 65:35 to 25:75). The desired fractions were combined and MeCN was evaporated. The product was extracted with DCM (3 times) and the organic layer was dried over MgSO₄, filtered and evaporated to give a colorless gum (81 mg). The product was triturated in pentane and Et₂O (1/1), evaporated and dried under high vacuum at 50° C. for 5 h to afford 66 mg of compound 35 as a light-yellow solid (24%).

¹H NMR (400 MHz, DMSO-d₆) δ ppm 9.11 (m, 1H) 8.45-8.53 (m, 1H) 7.69 (d, J=9.4 Hz, 1H) 7.48 (dd, J=9.7, 1.8 Hz, 1H) 7.29 (s, 1H) 7.18 (d, J=9.5 Hz, 2H) 4.54 (d, J=5.6 Hz, 2H) 4.05-4.13 (m, 2H) 3.61-3.70 (m, 2H) 3.03 (q, J=7.4 Hz, 2H) 1.23-1.35 (t, J=7.4 Hz, 3H).

Synthesis of Compound 36

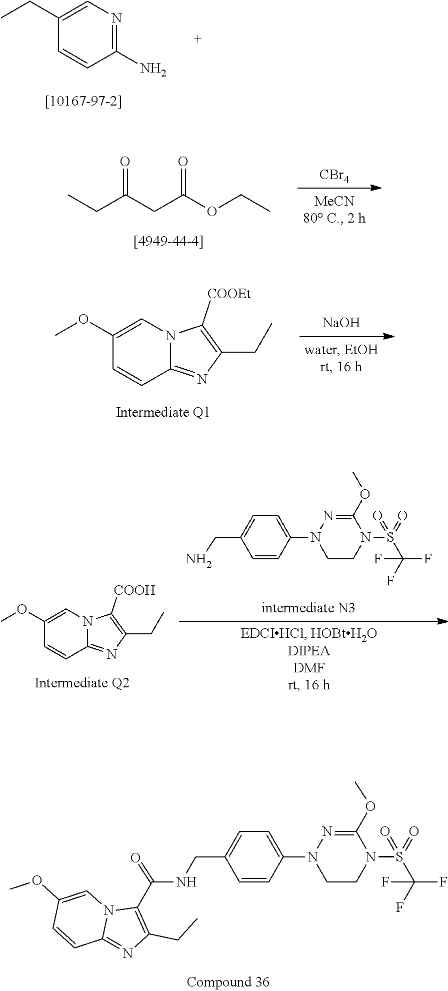

Preparation of Intermediate Q1

Carbone tetrabromide (16 g; 43.4 mmol) was added to a mixture of 2-amino-5-methoxypryridine [10167-97-2] (3 g, 24.2 mmol) and ethyl-3-oxovalerate[4949-44-4](5.2 mL, 36.6 mmol) in MeCN (50 mL). The reaction mixture was heated at 80° C. for 2 h. The reaction mixture was cooled to room temperature and concentrated to dryness. The residue (20 g) was purified by preparative LC (regular SiOH 30 μm, 330 g, dry loading (SiOH), mobile phase: heptane/EtOAc, gradient from 80:20 to 0:100) to give 1.89 g of intermediate Q1 as a greenish solid (32%).

Preparation of Intermediate Q2

To a solution of intermediate Q1 (1.89 g, 7.61 mmol) in water (20 mL) and EtOH (25 mL) was added NaOH (913 mg, 22.8 mmol). The reaction mixture was stirred at room temperature for 16 h. Additional quantity of NaOH (304 mg, 7.61 mmol) was added and the reaction mixture was stirred for 3 h. EtOH was concentrated. The mixture was acidified to pH 2-3 with HCl (1N). The white precipitate was filtered and washed with water and dried under high vacuum to give 750 mg of intermediate Q2 as a white solid (45%).

Preparation of Compound 36

To a mixture of intermediate Q2 (150 mg, 0.681 mmol) and DIPEA (0.48 mL, 2.79 mmol) in DMF (7 mL) were successively added EDCI·HCl (174 mg, 0.908 mmol), HOBt·H₂O (144 mg, 0.94 mmol) and intermediate N3 (265 mg, 0.681 mmol). The reaction mixture was stirred at room temperature for 16 h and evaporated. The residue was taken-up in DCM and NaHCO₃ (sat., aq.) was added. The layers were separated and the organic layer was washed with water and brine (twice), dried over MgSO₄, filtered and evaporated. The crude mixture was purified by preparative LC (regular SiOH 30 μm, 24 g, liquid injection (DCM), mobile phase: heptane/(EtOAc/MeOH, 9/1), gradient from 80:20 to 20:80). The fractions containing product were combined and evaporated to afford a white solid (304 mg). The product was recrystallized from MeCN, filtered and dried under high vacuum at 50° C. for 3 h to afford 200 mg of compound 36 as a white solid (53%).

¹H NMR (400 MHz, DMSO-d₆) δ ppm 8.65 (d, J=2.2 Hz, 1H) 8.23-8.32 (m, 1H) 7.53 (d, J=9.5 Hz, 1H) 7.29 (d, J=8.7 Hz, 2H) 7.13-7.21 (m, 3H) 4.46 (d, J=5.9 Hz, 2H) 4.06-4.17 (m, 2H) 3.85 (s, 3H) 3.72-3.82 (m, 5H) 2.95 (q, J=7.5 Hz, 2H) 1.24 (t, J=7.5 Hz, 3H).

Synthesis of Compound 37

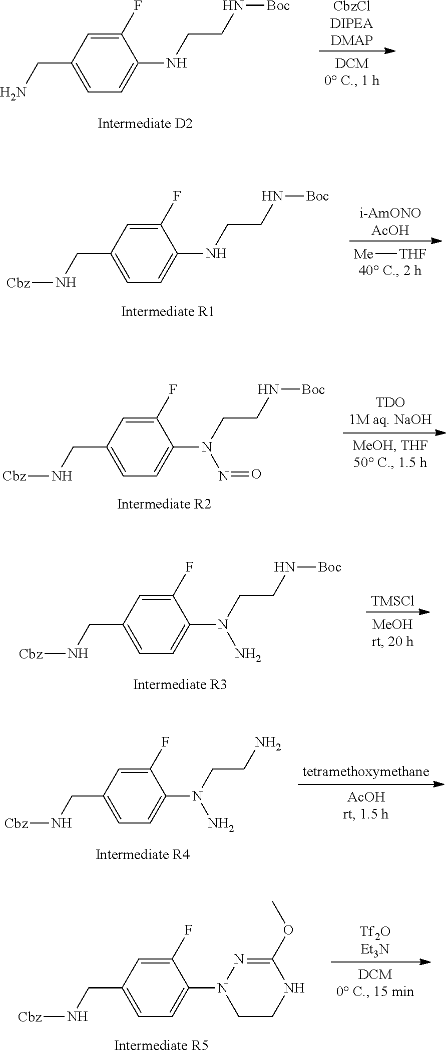

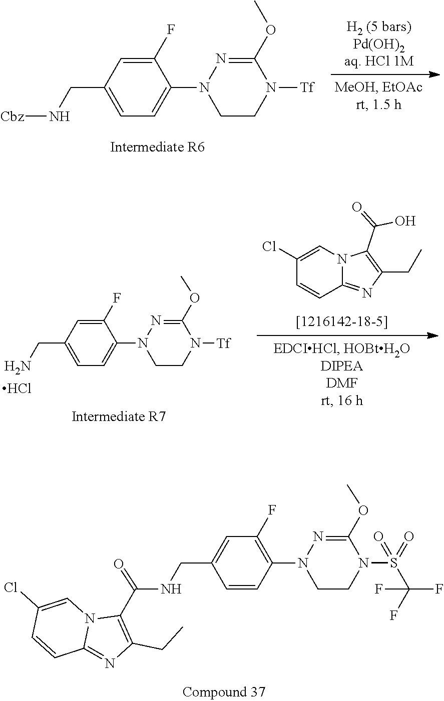

Preparation of Intermediate R1

Intermediate R₁ was prepared following the synthesis reported for intermediate E3, starting from intermediate D2 (7.06 mmol) and affording 2.53 g as an off-white solid (86%).

Preparation of Intermediate R2

Intermediate R₂ was prepared following the synthesis reported for intermediate E4, starting from intermediate R1 (6.06 mmol) and affording, 3.2 g as a yellow oil used as such for next step without purification.

Preparation of Intermediate R3

Intermediate R₃ was prepared following the synthesis reported for intermediate E5, starting from intermediate R2 (6.06 mmol theorical) and affording 2.22 g as a yellow oil (87% over 2 steps).

Preparation of Intermediate R4

To a solution of intermediate R3 (2.22 g, 5.13 mmol) in MeOH (52 mL) was added dropwise TMSCl (5.2 mL, 41 mmol). The reaction mixture was stirred at room temperature for 20 h and concentrated in vacuo. Et₂O was added to the residue and the gum was triturated. The solvent was removed under reduced pressure to give 2.06 g of intermediate R4 as a pale green solid (99%).

Preparation of Intermediate R5

A solution of intermediate R4 (1.00 g, 2.47 mmol) in acetic acid (25 mL) was treated with tetramethoxymethane (0.82 mL, 6.17 mmol) and stirred at room temperature for 1 h. Additional amount of tetramethoxymethane (0.82 mL, 6.17 mmol) was added and the mixture was stirred at room temperature for 30 min. The reaction mixture was poured in DCM and water. The mixture was basified with K₂CO₃ powder and the layers were separated. The aqueous layer was extracted with DCM (once) and the combined organic layers were dried over MgSO₄, filtered and evaporated in vacuo. The residue (685 mg) was purified by preparative LC (irregular SiOH 40 μm, 24 g, liquid injection (DCM), mobile phase: DCM/MeOH, gradient from 100:0 to 85:15) to give 445 mg of intermediate R5 as a colorless oil (48%).

Preparation of Intermediate R6

Intermediate R₆ was prepared following the synthesis reported for intermediate P8, starting from intermediate R5 (1.19 mmol) and affording 0.45 g as colorless oil (72%).

Preparation of Intermediate R7

Intermediate R₇ was prepared following the synthesis reported for intermediate P9, starting from intermediate R6 (0.61 mmol) and affording 0.24 g as colorless oil (96%).

Preparation of Compound 37

To a mixture of 6-chloro-2-ethylimidazo[1,2-a]pyridine-3-carboxylic acid [1216142-18-5] (87.3 mg, 0.388 mmol), intermediate R7 (158 mg, 0.388 mmol) and DIPEA (0.335 mL, 1.94 mmol) in DMF (5.3 mL) were successively added EDCI·HCl (74.5 mg, 0.388 mmol) and HOBt·H₂O (59.5 mg, 0.388 mmol). The reaction mixture was stirred at room temperature for 16 h and evaporated in vacuo. The crude mixture was purified by preparative LC (irregular SiOH 15-40 μm, 12 g, dry loading (Celite®), mobile phase: heptane/EtOAc, gradient from 80:20 to 30:70). The desired fractions were combined and evaporated under vacuum. The product (163 mg) was sonicated in Et₂O and filtered to give 118 mg of compound 37 as a white solid (53%).

¹H NMR (400 MHz, DMSO-d₆) δ ppm 9.09 (d, J=1.6 Hz, 1H) 8.47 (t, J=5.9 Hz, 1H) 7.68 (d, J=9.5 Hz, 1H) 7.42-7.50 (m, 2H) 7.16-7.25 (m, 2H) 4.49 (d, J=5.9 Hz, 2H) 4.07-4.15 (m, 2H) 3.83 (s, 3H) 3.53-3.61 (m, 2H) 3.00 (q, J=7.5 Hz, 2H) 1.27 (t, J=7.5 Hz, 3H).

Synthesis of Compound 38

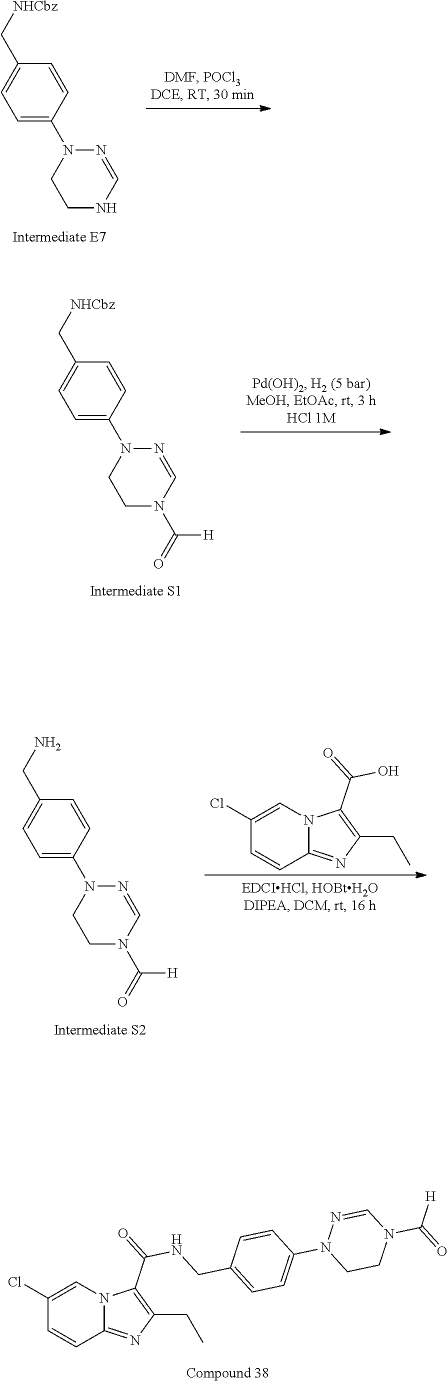 [SMILES: O=C(NCc1ccc(N2CCNC=N2)cc1)OCc1ccccc1 ; O=CN1C=NN(c2ccc(CNC(=O)OCc3ccccc3)cc2)CC1 ; NCc1ccc(N2CCN(C=O)C=N2)cc1 ; CCc1nc2ccc(Cl)cn2c1C(=O)O ; CCc1nc2ccc(Cl)cn2c1C(=O)NCc1ccc(N2CCN(C=O)C=N2)cc1]

Preparation of Intermediate S1

To a solution of DMF (103 μL, 1.33 mmol) in DCE (6.5 mL) at room temperature was added POCl₃ (123 μL, 1.33 mmol) and the mixture was stirred at room temperature for 30 min. Then the mixture was cooled down to 0° C. and intermediate E7 (430 mg, 1.33 mmol) in DCE (6.5 mL) was added dropwise and the mixture was stirred at 0° C. for 2 hours. Water and DCM were added. The aqueous layer was slowly basified with NaHCO₃ (s) to pH 8. The layers were separated, and the aqueous layer was extracted with DCM. The combined organic layers were washed with brine, dried over MgSO₄, filtered off and evaporated to afford 421 mg of intermediate S1 as a yellow solid. The crude was used as such in next step.

Preparation of Intermediate S2

In a steal vessel, a mixture of intermediate S1 (421 mg, 1.20 mmol), palladium hydroxide (100 mg, 0.14 mmol) and HCl 1M in H₂O (1.2 mL, 1.2 mmol) in MeOH (10.5 mL) and EtOAc (10.5 mL) was hydrogenated under 5 bar of H₂ at room temperature for 3 hours. The mixture was filtered on a pad of Celite® to give 413 mg of intermediate S2 as a yellow solid. The crude was used as such in next step.

Preparation of Compound 38

To a solution of 6-chloro-2-ethylimidazo[1,2-a]pyridine-3-carboxylicacid (CAS [1216142-18-5], 240 mg, 1.07 mmol) and diisopropylethylamine (0.75 mL, 4.35 mmol) in DCM (11 mL) were added EDCI·HCl (210 mg, 1.10 mmol) and HOBt·H₂O (170 mg, 1.11 mmol) then intermediate S2 (410 mg, 1.13 mmol) and the mixture was stirred at room temperature for 16 hours. DCM and water were added. The layers were separated, and the organic layer was washed with an aqueous saturated solution of NaHCO₃ and brine. The organic layer was dried over MgSO₄, filtered and evaporated. The crude was purified by Reverse Phase (Stationary phase: YMC-actus Triart C18 10 μm 30*150 mm, 40 g, dry loading (on Celite®), mobile phase: Gradient from 80% (aq. NH₄HCO₃ 0.2%), 20% MeCN to 40% (aq. NH₄HCO₃ 0.2%), 60% MeCN). MeCN was evaporated and the product was extracted with DCM/MeOH (9:1) (3 times). The organic layer was dried over MgSO₄, filtered and evaporated to afford 176 mg of a light-yellow solid. It was purified by Reverse phase (Stationary phase: YMC-actus Triart C18 10 μm 30*150 mm, 40 g, dry loading (on Celite®), mobile phase: Gradient from 60% (aq. NH₄HCO₃ 0.2%), 40% MeCN to 45% (aq. NH₄HCO₃ 0.2%), 55% MeCN over 16 CV). All fractions were combined to obtain 139 mg as a yellow solid. It was purified by Reverse phase (Stationary phase: YMC-actus Triart C18 10 μm 30*150 mm, liquid loading (DMSO), Mobile phase: Gradient from 70% (aq. NH₄HCO₃ 0.2%), 30% ACN to 50% (aq. NH₄HCO₃ 0.2%), 50% ACN) to afford 39 mg as a white solid. It was solubilized in DCM/MeOH then combined with a previous fraction, evaporated and dried under high vacuum (50° C., 2 h) to afford 68 mg as an off-white solid. It was co-evaporated in MeOH (5 times), then dried under high vacuum (50° C., 6 h) to give 65 mg of compound 38 as an off-white solid (12%) Major rotamer (84%) ¹H NMR (500 MHz, DMSO-d6, 350K) δ ppm 9.07 (s, 1H), 8.57 (s, 1H), 8.15 (br t, J=5.2 Hz, 1H), 7.61 (d, J=9.5 Hz, 1H), 7.53 (s, 1H), 7.39 (dd, J 5=9.6, 2.0 Hz, 1H), 7.28 (d, J=8.5 Hz, 2H), 7.19 (d, J=8.5 Hz, 2H), 4.47 (d, J=6.0 Hz, 2H), 3.78 (br t, J=4.7 Hz, 2H) 3.64 (br t, J=4.8 Hz, 2H), 2.97 (q, J=7.6 Hz, 2H), 1.26 (t, J=7.6 Hz, 3H). Minor rotamer (16%)¹H NMR (500 MHz, DMSO-d6, 350K) δ ppm 9.07 (s, 1H), 8.57 (s, 1H), 8.15 (br t, J=5.2 Hz, 1H), 7.61 (d, J=9.5 Hz, 1H), 7.53 (s, 1H), 7.39 (dd, J=9.6, 2.0 Hz, 1H), 7.28 (d, J=8.5 Hz, 2H), 7.19 (d, J=8.5 Hz, 2H), 4.47 (d, J=6.0 Hz, 2H), 3.90 (m, 2H) 3.73 (m, 2H), 2.97 (q, J=7.6 Hz, 2H), 1.26 (t, J=7.6 Hz, 3H).

Synthesis of Compound 39

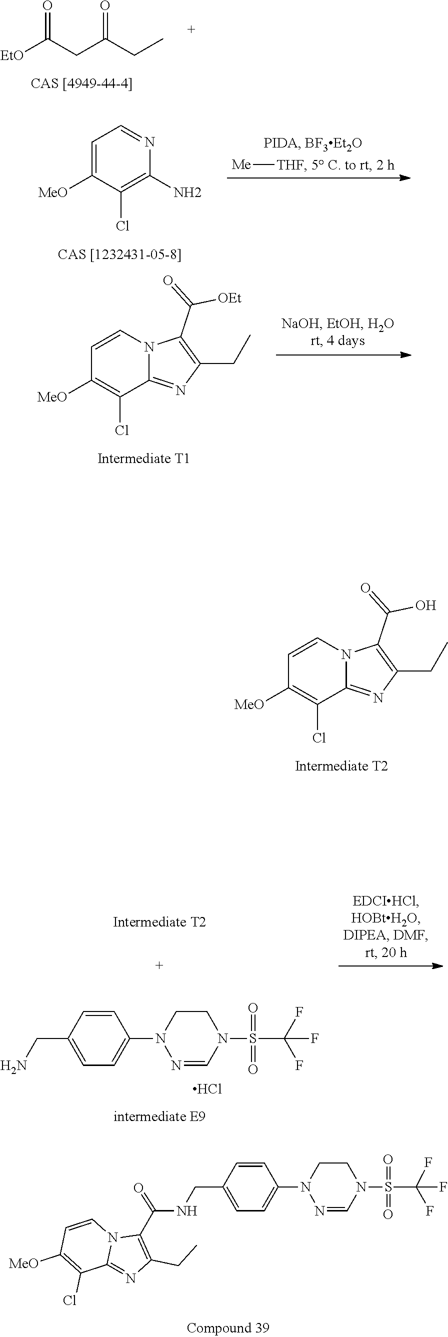

Preparation of Intermediate T1

To a solution of 3-chloro-4-methoxypyridine-2-amine (CAS [1232431-05-8], 0.2 g, 1.26 mmol) in 2-MeTHF (6 mL) at 5° C. under N₂ were added ethyl-3-oxovalerate (CAS [4949-44-4], 0.18 mL, 1.26 mmol) and iodobenzenediacetate ((diacetoxyiodo)benzene) (0.406 g, 1.26 mmol.), then borontrifluoride etherate (16.5 μL, 0.063 mmol) was added dropwise. The solution was stirred at the 5° C. for 30 min then warmed to room temperature and stirred for 2 hours. An extra amount of ethyl-3-oxovalerate (0.09 mL, 0.63 mmol), iodobenzenediacetate (0.203 g, 0.63 mmol) and borontrifluoride etherate (16.5 μL, 0.063 mmol) were added, the mixture was purged with N₂ and stirred at rt for 1 hour. EtOAc and water were added. The layers were separated, and the organic layer was dried over MgSO₄, filtered off and concentrated. The crude was purified by preparative LC (regular SiOH, 30 μm, 24 g liquid loading (DCM), mobile phase: Heptane 95%, EtOAc 5% isocratic for 3 CV then gradient to Heptane 60%, EtOAc 40% over 12 CV) to afford 295 mg of intermediate T1 as a white solid (83%).

Preparation of Intermediate T2

To a solution of intermediate T1 (270 mg, 0.96 mmol) in water (4.8 mL) and EtOH (4.8 mL) was added NaOH (115 mg, 2.88 mmol) and the mixture was stirred at room temperature for 4 days. The mixture was evaporated to afford 371 mg of intermediate T2 as a light-yellow solid (purity 71%). The crude was used as such in next step.

Preparation of Compound 39

To a solution of intermediate T2 (371 mg, 0.952 mmol) and diisopropyethylamine (0.50 mL, 2.90 mmol) in DMF (9.5 mL) were added HOBt·H₂O (160 mg, 1.05 mmol) and EDCI·HCl (195 mg, 1.02 mmol) then intermediate E9 (400 mg, 0.959 mmol). The mixture was stirred at rt for 20 hours. The mixture was evaporated then taken-up in DCM and an aqueous saturated solution of NaHCO₃ was added. The organic layer was separated and washed with brine, dried over MgSO₄, filtered and evaporated to give an orange gum. The crude was purified by preparative LC (irregular SiOH, 15-40 μm, 50 g, liquid loading (in DCM), mobile phase gradient: from Heptane 75%, EtOAc/MeOH (9:1) 25% to Heptane 25%, EtOAc/MeOH (9:1) 75% over 12 CV). Clean fractions were combined and evaporated to afford 312 mg as a light-yellow solid. It was purified by Reverse Phase (Stationary phase: YMC-actus Triart C18 10 μm 30*150 mm, 40 g, dry loading (on Celite®), mobile phase: Gradient from 55% (aq. NH₄HCO₃ 0.2%), 45% MeCN to 5% (aq. NH₄HCO₃ 0.2%), 95% MeCN over 12 CV) to afford 286 mg as an off-white solid. It was sonicated in MeCN (suspension) then filtered off. The solid was dried under high vacuum (50° C., 6 h) to afford 230 mg of compound 39 as a white solid (43%).

¹H NMR (400 MHz, DMSO-d6) δ ppm 8.94 (d, J=7.7 Hz, 1H), 8.35 (t, J=5.9 Hz, 1H), 7.26-7.35 (m, 3H), 7.12-7.23 (m, 3H), 4.45 (br d, J=5.9 Hz, 2H), 4.07 (br d, J=4.4 Hz, 2H), 3.99 (s, 3H), 3.82 (t, J=4.6 Hz, 2H), 2.95 (q, J=7.6 Hz, 2H), 1.24 (t, J=7.5 Hz, 3H).

Synthesis of Compound 40 and compound 41

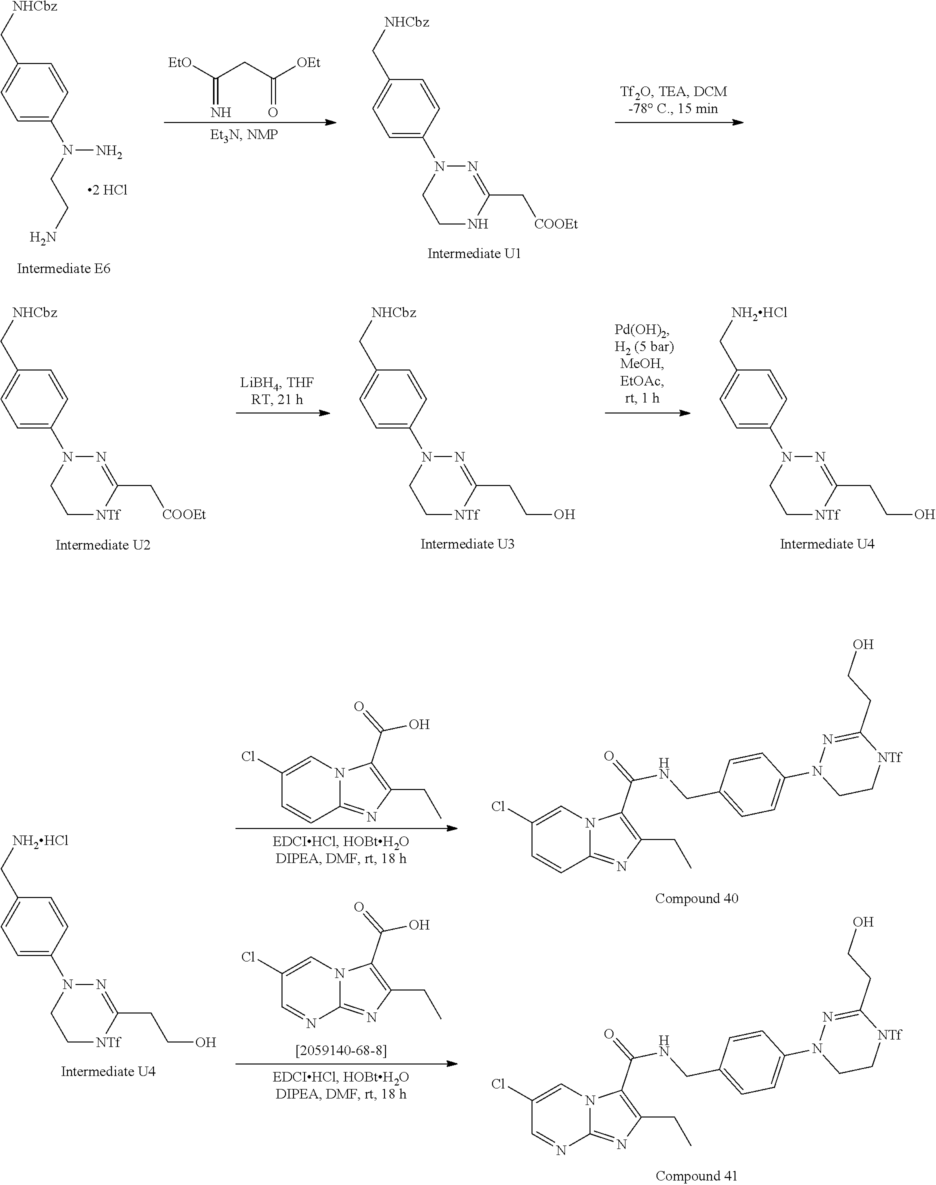

Preparation of Intermediate U1

A mixture of intermediate E6 (1.00 g, 2.58 mmol), ethyl-3-ethoxy-3-iminopropanoate hydrochloride (CAS [2318-25-4], 2.17 g, 7.75 mmol) and triethylamine (1.08 mL, 7.75 mmol) in NMP (14 mL) was stirred for 18 h at 150° C. in a sealed tube. The reaction mixture was diluted with EtOAc and water. The aqueous phase was extracted with EtOAc (×3). The combined organic phases were washed with NaCl sat., dried over MgSO₄ and concentrated to give 1.85 g as a brown oil. It was diluted in EtOAc and washed with a diluted solution of NaCl. The organic layer was dried over MgSO₄ and concentrated to give 1.03 g of intermediate U1. The crude product was used as such in the next step based on the theoretical quantity.

Preparation of Intermediate U2

At −78° C., to a solution of intermediate U1 (900 mg, 2.19 mmol) and triethylamine (914 μL, 6.58 mmol) in dry DCM (45 mL) was added dropwise Tf₂O 1M in DCM (3.1 mL, 3.1 mmol) and the reaction mixture was stirred for 15 min. The reaction mixture was diluted with DCM and water. The organic phase was dried over MgSO₄, filtered off and evaporated to give 1.0 g. The residue was purified by preparative LC (irregular SiOH 15-40 μm, 40 g, liquid loading (DCM), mobile phase gradient: (EtOAc/MeOH (90:10)) in heptane from 0 to 50% over 5 CV then isocratic for 5 CV) to give 456 mg of intermediate U2 as an orange-brown oil (38%).

Preparation of Intermediate U3

Lithium borohydride (276 μL; 0.553 mmol) was added to a solution of intermediate U2 (150 mg; 0.276 mmol) in THE (5 mL) and the solution was stirred at room temperature for 15 hours. Further lithium borohydride (276 μL, 0.553 mmol) was added and the reaction mixture was stirred for 6 hours. The reaction mixture was diluted with EtOAc and water. The aqueous layer was extracted once again with EtOAc and the combined organic layers were washed with brine (3 times) dried over MgSO₄, filtered and evaporated to dryness to give 132 mg of intermediate U3 (95%) as a yellow residue.

Preparation of Intermediate U4

Accordingly, intermediate U4 was prepared in the same way as intermediate S2 starting from intermediate U3 (0.132 g, 0.26 mmol) affording 0.11 g (quantitative).

Preparation of Compound 40

To a solution of 6-chloro-2-ethylimidazo[1,2-a]pyridine-3-carboxylicacid (CAS [1216142-18-5], 67 mg, 0.300 mmol), intermediate U4 (110 mg, 0.300 mmol), and diisopropylethylamine (155 μL, 0.901 mmol) in DMF (4 mL) were added EDCI·HCl (58 mg, 0.30 mmol) and HOBt·H₂O (46 mg, 0.30 mmol) and the reaction mixture was stirred at room temperature for 18 hours. The reaction mixture was concentrated. The residue was taken up in EtOAc and water. The organic layer was washed with NaCl sat, dried over MgSO₄, filtered off and concentrated to give 143 mg. The crude was purified by preparative LC (irregular SiOH 15-40 μm, 80 g, liquid loading (DCM), mobile phase gradient: (EtOAc/MeOH (90:10)) in heptane from 0 to 50% over 5 CV then isocratic for 5 CV) to give 100 mg as white solid. It was purified by reverse phase (spherical C18, 25 μm, 40 g YMC-ODS-25, dry loading (Celite®), mobile phase gradient: from 55% (aq. NH₄HCO₃ 0.2%), 45% MeCN to 75% (aq. NH₄HCO3 0.2%) MeCN) to give 19 mg and 59 mg of a residue which was co-evaporated with EtOH and MeCN affording 80 mg of compound 40 as a yellowish solid (combined yield: 57%).

¹H NMR (500 MHz, DMSO-d6) δ ppm 9.03-9.13 (m, 1H) 8.41 (br t, J=6.0 Hz, 1H) 7.66 (d, J=9.5 Hz, 1H) 7.45 (dd, J=9.5, 1.9 Hz, 1H) 7.32 (d, J=8.5 Hz, 2H) 7.16 (d, J=8.5 Hz, 2H) 4.66 (t, J=5.7 Hz, 1H) 4.47 (d, J=6.0 Hz, 2H) 3.96 (br t, J=5.0 Hz, 2H) 3.84 (t, J=4.9 Hz, 2H) 3.73 (q, J=6.6 Hz, 2H) 2.98 (q, J=7.6 Hz, 2H) 2.74 (t, J=6.9 Hz, 2H) 1.26 (t, J=7.6 Hz, 3H)

Preparation of Compound 41

Accordingly, compound 41 was prepared in the same way as compound 40, starting from 6-chloro-2-ethyl-imidazo[1,2-a]-pyrimidine-3-carboxylic acid (CAS [2059140-68-8], 0.32 mmol) and intermediate U4 (0.32 mmol) affording 0.067 g (37%) as green-light solid.

¹H NMR (500 MHz, DMSO-d6) δ ppm 9.39 (d, J=2.5 Hz, 1H) 8.68 (d, J=2.5 Hz, 1H) 8.55 (t, J=5.8 Hz, 1H) 7.31 (m, J=8.5 Hz, 2H) 7.15 (m, J=8.5 Hz, 2H) 4.70 (t, J=5.7 Hz, 1H) 4.47 (d, J=6.0 Hz, 2H) 3.95 (br t, J=4.9 Hz, 2H) 3.79-3.88 (m, 2H) 3.72 (q, J=6.6 Hz, 2H) 3.01 (q, J=7.4 Hz, 2H) 2.73 (t, J=6.8 Hz, 2H) 1.27 (t, J=7.6 Hz, 3H)

Synthesis of Compound 42

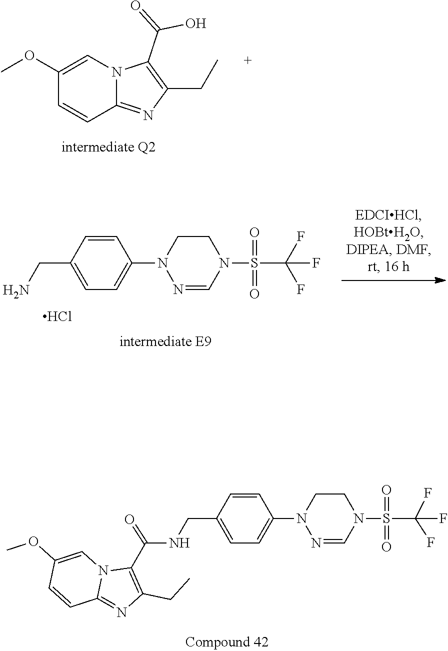 [SMILES: CCc1nc2ccc(OC)cn2c1C(=O)O ; NCc1ccc(N2CCN(S(=O)(=O)C(F)(F)F)C=N2)cc1 ; Cl ; CCc1nc2ccc(OC)cn2c1C(=O)NCc1ccc(N2CCN(S(=O)(=O)C(F)(F)F)C=N2)cc1]

To a solution of intermediate Q2 (125 mg, 0.568 mmol) in diisopropylethylamine (0.4 mL, 2.32 mmol) and DMF (6 mL) were added EDCI·HCl (145 mg, 0.756 mmol), HOBt·H₂O (120 mg, 0.784 mmol) then intermediate E9 (205 mg, 0.571 mmol). The mixture was stirred at room temperature for 16 hours. The reaction mixture was evaporated and taken-up in DCM and an aqueous saturated solution of NaHCO₃. The layers were separated, and the organic layer was washed with water, brine (twice), dried over MgSO₄, filtered and evaporated. The crude was purified by preparative LC (regular SiOH, 30 μm, 24 g, liquid loading (DCM), mobile phase gradient: from Heptane 80%, EtOAc/MeOH (9:1) 20% to Heptane 20%, EtOAc/MeOH (9:1) 80% over 12 CV) to afford 166 mg as a white solid. It was recrystallized form MeCN then filtered off and dried under high vacuum to afford 107 mg of compound 42 as a white solid (36%).

¹H NMR (400 MHz, DMSO-d6) δ ppm 8.64 (d, J=2.2 Hz, 1H), 8.30 (t, J=5.8 Hz, 1H), 7.53 (d, J=9.5 Hz, 1H), 7.27-7.36 (m, 3H), 7.14-7.22 (m, 3H), 4.47 (d, J=5.9 Hz, 2H), 4.08 (br t, J=4.5 Hz, 2H), 3.83 (br t, J=4.5 Hz, 2H) 3.76 (s, 3H), 2.95 (q, J=7.5 Hz, 2H), 1.24 (t, J=7.5 Hz, 3H).

Synthesis of Compound 43

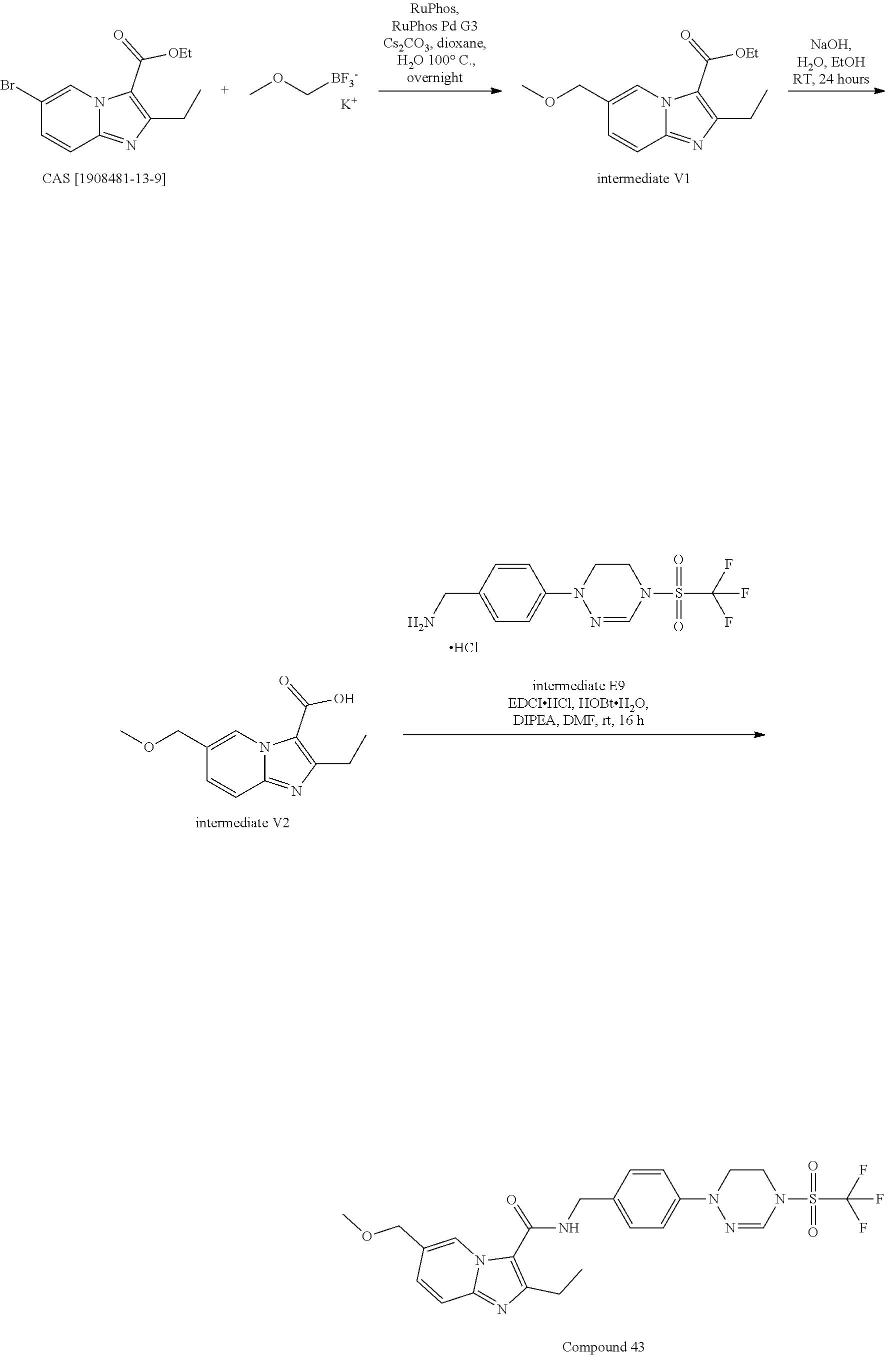

Preparation of Intermediate V1

In a sealed tube, a suspension of imidazo[1,2-a]-pyridine-3-carboxylic acid, 6-bromo-2-ethyl-ethyl ester (CAS [1908481-13-9], 400 mg, 1.35 mmol), potassium (methoxymethyl)trifluoroborate (614 mg, 4.04 mmol) and cesium carbonate (1.32 g, 4.04 mmol) in 1,4-dioxane (3.44 mL) and water (0.49 mL) was purged with N₂. RuPhos (62.8 mg, 0.135 mmol) and RuPhos Pd G3 (113 mg, 0.135 mmol) were added, the mixture was purged again with N₂ then stirred at 100° C. overnight. The mixture was filtered off then the filtrate was evaporated. The crude was purified by preparative LC (regular SiOH, 30 μm, 50 g, dry loading (on Celite®), mobile phase gradient: from heptane 90%, EtOAc/MeOH (9:1) 10% to Heptane 50%, EtOAc/MeOH (9:1) 50% over 12 CV) to obtain 317 mg of intermediate V1 as a colorless gum which crystallized on standing (66%).

Preparation of Intermediate V2

To a solution of intermediate V1 (317 mg, 0.894 mmol) in water (4 mL) and EtOH (4 mL) was added NaOH (107 mg, 2.68 mmol) and the mixture was stirred at room temperature for 24 hours. The mixture was evaporated to afford 518 mg of intermediate V2 as a yellow gum. The crude was used as such in next step.

Preparation of Compound 43

Accordingly, compound 43 was prepared in the same way as compound 42, starting from intermediate V2 (0.9 mmol) and intermediate E9 (0.84 mmol) affording 0.113 g (22%) as a white solid.

¹H NMR (500 MHz, DMSO-d6) δ ppm 8.93 (s, 1H), 8.38 (t, J=6.0 Hz, 1H), 7.58 (d, J=9.1 Hz, 1H), 7.26-7.36 (m, 4H), 7.19 (d, J=8.5 Hz, 2H), 4.43-4.51 (m, 4H), 4.08 (br t, J=4.6 Hz, 2H), 3.83 (t, J=4.7 Hz, 2H), 3.30 (s, 3H), 2.96 (q, J=7.4 Hz, 2H), 1.25 (t, J=7.6 Hz, 3H).

Synthesis of Compound 44

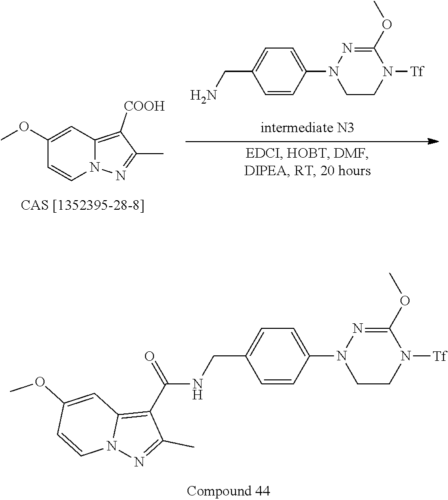

Accordingly, compound 44 was prepared in the same way as compound 42, starting from 5-methoxy-2-methylpyrrazolo[1,5-a]-pyridine-3-carboxylic acid (CAS [1352395-28-8], 0.37 mmol) and intermediate N3 (0.37 mmol) affording 0.19 g (42%) as a white solid.

¹H NMR (500 MHz, DMSO-d₆) δ ppm 8.51 (d, J=7.6 Hz, 1H) 7.91 (t, J=6.0 Hz, 1H) 7.43 (t, J=8.7 Hz, 1H) 7.26 (d, J=2.8 Hz, 1H) 7.12-7.23 (m, 2H) 6.64 (dd, J=7.6, 2.8 Hz, 1H) 4.44 (d, J=5.7 Hz, 2H) 4.07-4.15 (m, 2H) 3.86 (s, 3H) 3.82 (s, 3H) 3.53-3.60 (m, 2H) 2.53 (s, 3H)

Synthesis of Compound 45

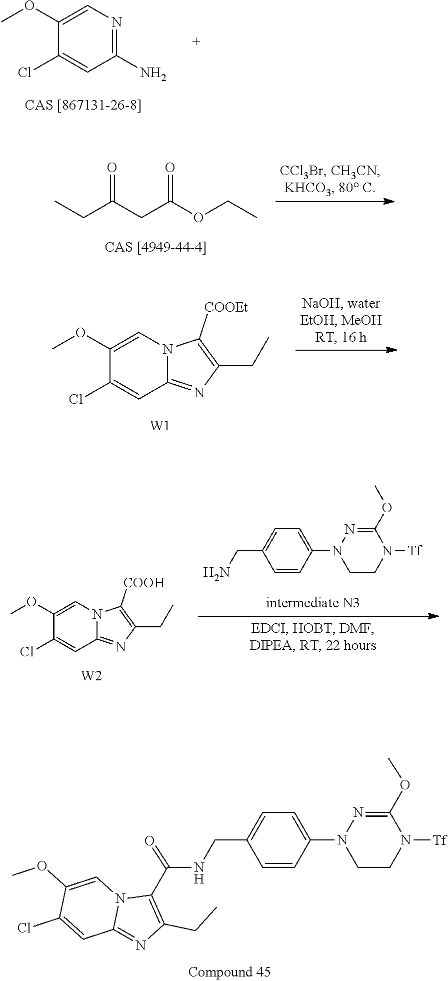

Preparation of Intermediate W1

To a solution of 4-chloro-5-methoxypyridin-2-amine (CAS [867131-26-8], 500 mg, 3.15 mmol) in dry acetonitrile (7.5 mL) were added ethyl 3-oxovalerateethyl 3-oxovalerate (0.90 mL, 6.3 mmol), bromotrichloromethane (1.1 mL, 11 mmol) and potassium bicarbonate (947 mg, 9.46 mmol). The mixture was stirred at 80° C. for 16 hours. The reaction mixture was diluted in EtOAc and water. The organic layer was then washed with brine, dried over MgSO₄, filtered off and evaporated. The residue was purified by preparative LC (irregular SiOH 15-40 μm, 40 g, dry loading on Celite®, mobile phase gradient: Heptane/EtOAc 95/5 to Heptane/EtOAc 40/60 in 15 CV) to give 458 mg of intermediate W1 as a yellow solid (51% yield).

Preparation of Intermediate W2

A mixture of intermediate W1 (456 mg, 1.61 mmol) and NaOH (194 mg, 4.86 mmol) in water (8.1 mL), EtOH (8.1 mL) and MeOH (9.8 mL) was stirred at room temperature for 16 hours. The reaction mixture was evaporated. The residue was solubilized with MeOH and acidified with a 3N aqueous solution of HCl. The solution was evaporated to give 726 mg of a yellow solid. DCM and MeOH were added to the yellow solid. The mixture was then filtered off and the filtrate was evaporated to give 443 mg of intermediate W2 as a beige solid (93% purity, quantitative).

Preparation of Compound 45

Accordingly, compound 45 was prepared in the same way as compound 42, starting from intermediate W2 (0.46 mmol) and intermediate N3 (0.46 mmol) affording 0.19 g (69%) as a beige solid.

¹H NMR (400 MHz, DMSO-d₆) δ ppm 8.77 (s, 1H) 8.32 (t, J=5.8 Hz, 1H) 7.86 (s, 1H) 7.29 (d, J=8.6 Hz, 2H) 7.15 (d, J=8.7 Hz, 2H) 4.46 (br d, J=5.7 Hz, 2H) 4.10 (br t, J=4.8 Hz, 2H) 3.87 (s, 3H) 3.85 (s, 3H) 3.74 (br t, J=4.8 Hz, 2H) 2.95 (q, J=7.5 Hz, 2H) 1.24 (t, J=7.5 Hz, 3H)

Synthesis of Compound 46

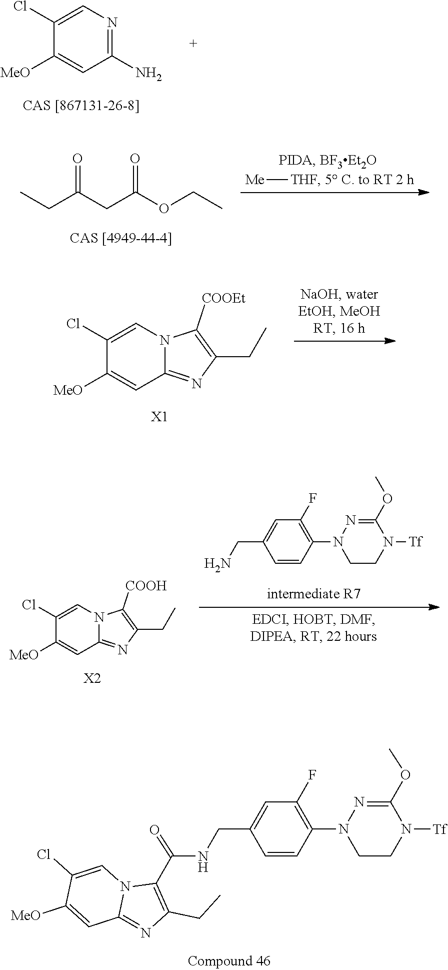

Preparation of Intermediate X1

Accordingly, intermediate X1 was prepared in the same way as intermediate T1 starting from 5-chloro-4-methoxypyridin-2-amine CAS [662117-63-7] (6.31 mmol) affording 1.23 g (69%) as a light-yellow solid.

Preparation of Intermediate X2

Accordingly, intermediate X2 was prepared in the same way as intermediate V2 starting from intermediate X1 (4.35 mmol) affording 0.83 g (75%) as a light-yellow solid.

Preparation of Compound 46

Accordingly, compound 46 was prepared in the same way as compound compound 42, starting from intermediate X2 (0.45 mmol) and intermediate R7 (0.43 mmol) affording 0.14 g (48%) as a white solid.

¹H NMR (500 MHz, DMSO-d6) δ ppm 9.11 (s, 1H), 8.27 (br t, J=5.8 Hz, 1H), 7.44 (t, J=8.5 Hz, 1H), 7.16-7.25 (m, 3H), 4.47 (br d, J=5.7 Hz, 2H), 4.08-4.13 (m, 2H), 3.95 (s, 3H), 3.83 (s, 3H), 3.54-3.59 (m, 2H), 2.96 (q, J=7.5 Hz, 2H), 1.27 (t, J=7.5 Hz, 3H)

Synthesis of Compound 47

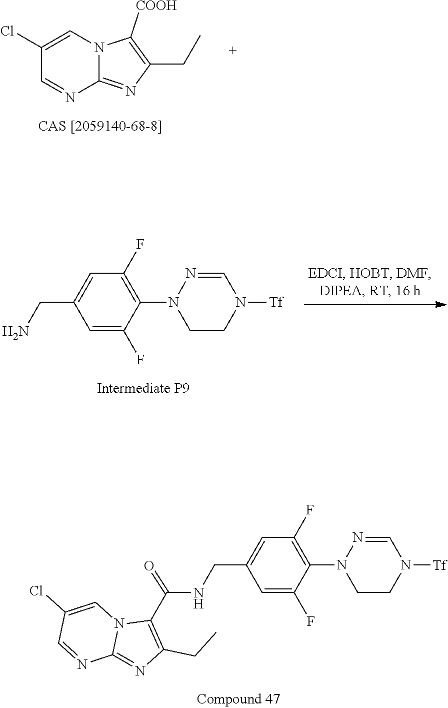

Accordingly, compound 47 was prepared in the same way as compound 42, starting from intermediate 6-Chloro-2-ethyl-imidazo[1,2-a]-pyrimidine-3-carboxylic acid CAS [2059140-68-8] (0.38 mmol) and intermediate P9 (0.31 mmol) affording 0.027 g (15%) as a white fluffy solid.

¹H NMR (400 MHz, DMSO-d₆) δ ppm 9.35 (d, J=2.7 Hz, 1H), 8.63 (d, J=2.7 Hz, 1H), 8.52 (t, J=5.9 Hz, 1H), 7.21 (s, 1H), 7.12 (d, J=9.4 Hz, 2H), 4.46 (br d, J=5.7 Hz, 2H), 4.01 (br s, 2H), 3.57 (br t, J=4.3 Hz, 2H), 2.98 (q, J=7.5 Hz, 2H), 1.23 (t, J=7.5 Hz, 3H)

Synthesis of Compound 48

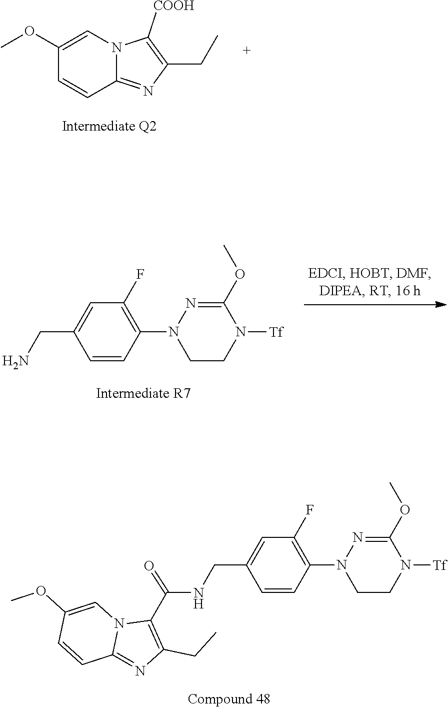

Accordingly, compound 48 was prepared in the same way as compound 42, starting from intermediate Q2 (0.52 mmol) and intermediate R7 (0.51 mmol) affording 0.15 g (52%) as a white solid.

¹H NMR (500 MHz, DMSO-d6) δ ppm 8.67 (d, J=2.2 Hz, 1H), 8.31 (t, J=5.8 Hz, 1H), 7.54 (d, J=9.8 Hz, 1H), 7.45 (t, J=8.7 Hz, 1H), 7.15-7.25 (m, 3H), 4.49 (d, J=5.7 Hz, 2H), 4.07-4.14 (m, 2H), 3.83 (s, 3H), 3.78 (s, 3H), 3.54-3.60 (m, 2H), 2.98 (q, J=7.6 Hz, 2H), 1.26 (t, J=7.6 Hz, 3H)

Synthesis of Compound 49

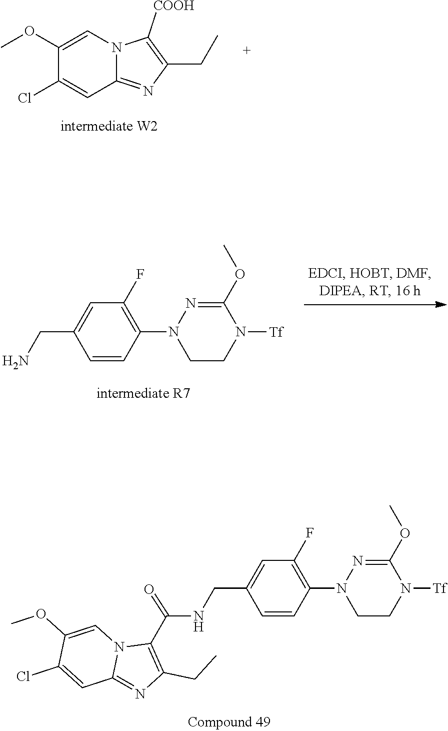

Accordingly, compound 49 was prepared in the same way as compound 42, starting from intermediate W2 (0.44 mmol) and intermediate R7 (0.44 mmol) affording 0.164 g (62%) as a white solid.

¹H NMR (500 MHz, DMSO-d₆) δ ppm 8.80 (s, 1H) 8.36 (br t, J=5.8 Hz, 1H) 7.87 (s, 1H) 7.45 (t, J=8.5 Hz, 1H) 7.15-7.26 (m, 2H) 4.50 (brd, J=5.7 Hz, 2H) 4.10 (brt, J=5.0 Hz, 2H) 3.87 (s, 3H) 3.82 (s, 3H) 3.56 (br t, J=5.0 Hz, 2H) 2.98 (q, J=7.6 Hz, 2H) 1.26 (t, J=7.6 Hz, 3H)

Synthesis of Compound 50

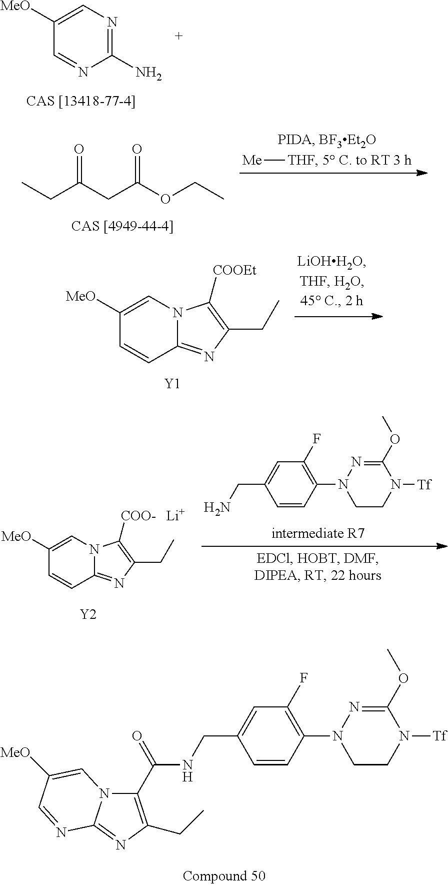

Preparation of Intermediate Y1

Accordingly, intermediate Y1 was prepared in the same way as intermediate X1 starting from 2-amino-5-methoxypyrimidine CAS [13418-77-4] (75.92 mmol) affording 4.94 g (26%) as a yellow solid.

Preparation of Intermediate Y2

To a solution of intermediate Y1 (150 mg, 0.602 mmol) in THF (3 mL) was added a solution of LiOH (75.8 mg, 1.81 mmol) in water (1.5 mL). The reaction mixture was stirred for 2 hours at 45° C. The mixture was evaporated to afford 218 mg of intermediate Y2 as a yellow solid. The crude was used as such in next step.

Preparation of Compound 50

Accordingly, compound 50 was prepared in the same way as compound 42, starting from intermediate Y2 (0.6 mmol) and intermediate R7 (0.55 mmol) affording 0.098 g (31%) as a white solid.

¹H NMR (400 MHz, DMSO-d6) δ ppm 8.96 (d, J=2.9 Hz, 1H), 8.52 (d, J=2.9 Hz, 1H), 8.41 (t, J=5.9 Hz, 1H), 7.45 (t, J=8.6 Hz, 1H), 7.15-7.26 (m, 2H), 4.50 (d, J=5.7 Hz, 2H), 4.08-4.14 (m, 2H), 3.86 (s, 3H), 3.83 (s, 3H), 3.53-3.59 (m, 2H), 3.02 (q, J=7.5 Hz, 2H), 1.28 (t, J=7.5 Hz, 3H)

Synthesis of Compound 51 and Compound 52

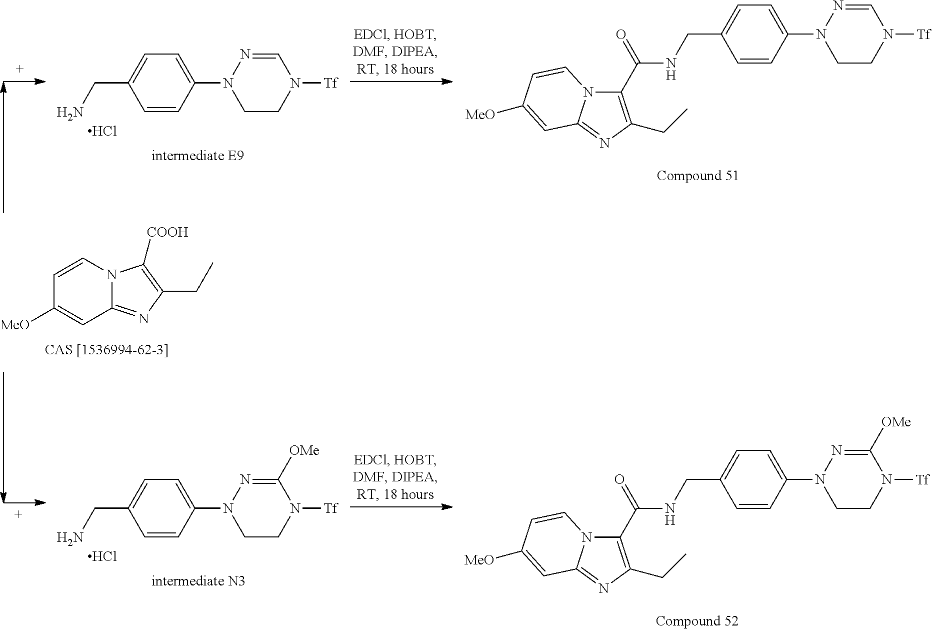

Preparation of Compound 51

Accordingly, compound 51 was prepared in the same way as compound 42, starting from 2-ethyl-7-methoxyimidazo[1,2-a]-pyridine-3-carboxylic acid (CAS [1536994-62-3], 0.46 mmol) and intermediate E9 (0.46 mmol) affording 0.195 g (72%) as a white solid.

¹H NMR (400 MHz, DMSO-d₆) δ ppm 8.83 (d, J=7.6 Hz, 1H) 8.19 (t, J=5.9 Hz, 1H) 7.25-7.34 (m, 3H) 7.18 (d, J=8.7 Hz, 2H) 7.00 (d, J=2.4 Hz, 1H) 6.70 (dd, J=7.6, 2.6 Hz, 1H) 4.44 (d, J=5.9 Hz, 2H) 4.07 (br t, J=4.4 Hz, 2H) 3.78-3.88 (m, 5H) 2.92 (q, J=7.5 Hz, 2H) 1.24 (t, J=7.5 Hz, 3H)

Preparation of Compound 52

Accordingly, compound 52 was prepared in the same way as compound 42, starting from 2-ethyl-7-methoxyimidazo[1,2-a]-pyridine-3-carboxylic acid (CAS [1536994-62-3], 0.46 mmol) and intermediate N3 (0.46 mmol) affording 0.178 g (69%) as a white solid.

¹H NMR (500 MHz, DMSO-d₆) δ ppm 8.84 (d, J=7.6 Hz, 1H) 8.16 (t, J=6.0 Hz, 1H) 7.28 (d, J=8.7 Hz, 2H) 7.14 (d, J=8.7 Hz, 2H) 6.99 (d, J=2.5 Hz, 1H) 6.70 (dd, J=7.7, 2.7 Hz, 1H) 4.43 (d, J=5.7 Hz, 2H) 4.10 (br t, J=5.0 Hz, 2H) 3.84 (m, 6H) 3.73 (br t, J=5.0 Hz, 2H) 2.91 (q, J=7.6 Hz, 2H) 1.25 (t, J=7.6 Hz, 3H)

Synthesis of Compound 53

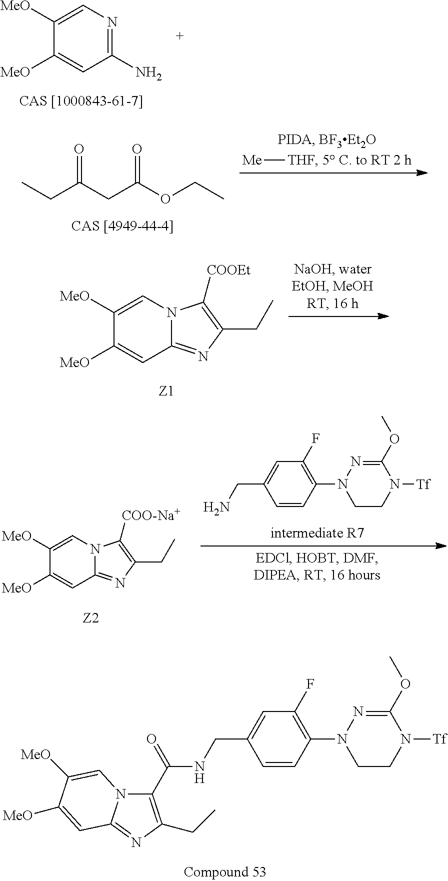

Preparation of Intermediate Z1

Accordingly, intermediate Z1 was prepared in the same way as intermediate X1 starting from 4,5-dimethoxy-pyridin-2-ylamine CAS [1000843-61-7] (1.3 mmol) affording 0.135 g (37%) as a light-yellow solid

Preparation of Intermediate Z2

Accordingly, intermediate Z2 was prepared in the same way as intermediate X2 starting from intermediate Z1 (0.49 mmol) affording 0.209 g (63%) as a light-yellow solid.

Preparation of Compound 53

Accordingly, compound 53 was prepared in the same way as compound 42, starting intermediate Z2 (0.48 mmol) and intermediate R7 (0.4 mmol) affording 0.149 g (39% over last 2 steps) as a white solid.

¹H NMR (400 MHz, DMSO-d6) δ ppm 8.67 (s, 1H), 8.11 (t, J=5.8 Hz, 1H), 7.44 (t, J=8.6 Hz, 1H), 7.15-7.23 (m, 2H), 7.05 (s, 1H), 4.47 (d, J=5.7 Hz, 2H), 4.07-4.14 (m, 2H), 3.87 (s, 3H), 3.83 (s, 3H), 3.76 (s, 3H), 3.53-3.59 (m, 2H), 2.95 (q, J=7.5 Hz, 2H), 1.25 (t, J=7.5 Hz, 3H)

Synthesis of Compound 54

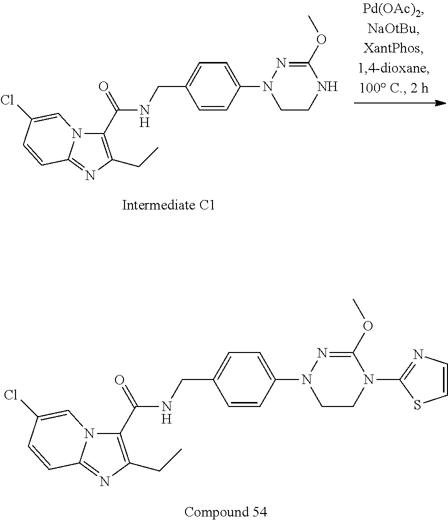 [SMILES: CCc1nc2ccc(Cl)cn2c1C(=O)NCc1ccc(N2CCNC(OC)=N2)cc1 ; CCc1nc2ccc(Cl)cn2c1C(=O)NCc1ccc(N2CCN(c3nccs3)C(OC)=N2)cc1]

A mixture of intermediate C1 (190 mg, 0.445 mmol), 2-bromothiazole (48.1 μL, 0.534 mmol) and sodium tert-butoxide (214 mg, 2.23 mmol) in dry 1,4-dioxane (5 mL) was purged with N₂ (3 times). XantPhos (51.5 mg, 89.0 μmol) and Pd(OAc)₂ (9.99 mg, 44.5 μmol) were added and the mixture was purged with N₂ (3 times). The reaction mixture was stirred at 100° C. for 2 hours. The reaction mixture was diluted with EtOAc/MeOH (95/5) and water. The aqueous layer was extracted with EtOAc (twice). The combined organic layers were washed with brine, dried over MgSO₄, filtered off and evaporated to give a yellow solid. The solid was purified by preparative LC (regular SiOH 30 μm, 25 g, dry loading (Celite®), mobile phase gradient: DCM 100% to DCM/(DCM: MeOH 80:20) 90/10 in 15 CV). The fractions containing product were combined and evaporated under vacuum to give a pale-yellow solid. The solid was triturated in Et₂O, filtered off, washed with Et₂O and then dried under vacuum to give 153 mg of compound 54 as a white solid (67% yield).

¹H NMR (500 MHz, DMSO-d₆) δ ppm 9.08 (d, J=1.5 Hz, 1H) 8.42 (t, J=5.9 Hz, 1H) 7.66 (d, J=9.6 Hz, 1H) 7.45 (dd, J=9.5, 2.1 Hz, 1H) 7.40 (d, J=3.7 Hz, 1H) 7.27 (d, J=8.7 Hz, 2H) 7.22 (d, J=8.7 Hz, 2H) 7.17 (d, J=3.7 Hz, 1H) 4.46 (d, J=5.8 Hz, 2H) 4.20 (t, J=5.1 Hz, 2H) 3.92 (s, 3H) 3.67 (t, J=5.1 Hz, 2H) 2.98 (q, J=7.6 Hz, 2H) 1.26 (t, J=7.6 Hz, 3H)

Synthesis of Compound 55

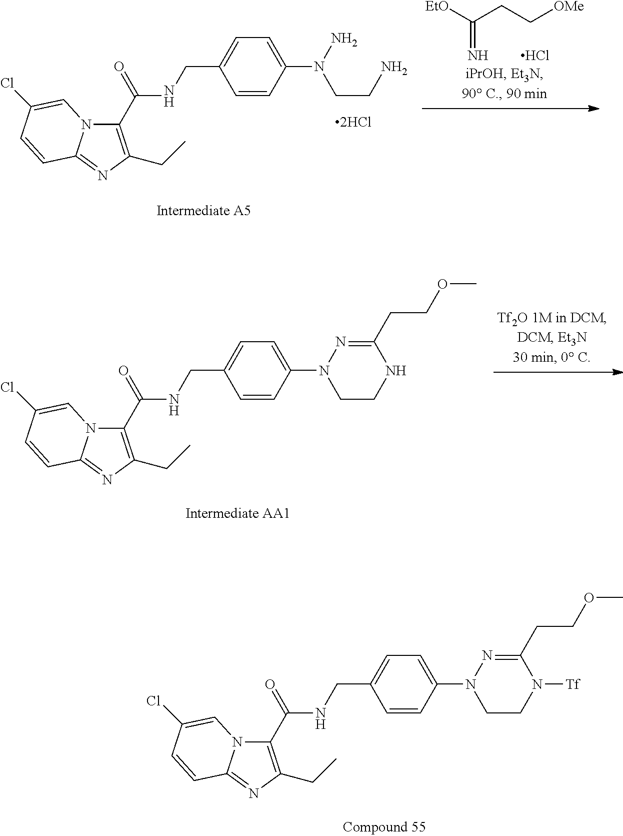

Preparation of Intermediate AA1

In a sealed tube, a mixture of intermediate A5 (300 mg, 0.652 mmol), 3-methoxypropionimidic acid ethyl ester hydrochloride (328 mg, 1.96 mmol) and triethylamine (272 μL, 1.96 mmol) in 2-propanol (6 mL) was stirred for 1.5 h at 90° C. After cooling to room temperature, the reaction mixture was concentrated. The residue was taken up in EtOAc and aqueous solution of NaHCO₃ (1%) was added. After separation, the aqueous phase was extracted with EtOAc (twice). The combined organic layers were dried over MgSO₄, filtered off and concentrated to give 280 mg of intermediate AA1 as a light-yellow oil which crystallized on standing (94%).

Preparation of Compound 55

Triethylamine (0.281 mL, 2.02 mmol) was added to a solution of intermediate AA1 (230 mg, 0.506 mmol) in dry DCM (4.6 mL). The solution was then cooled at 0° C. (ice/water bath). A 1M solution of Tf₂O (1.01 mL, 1.01 mmol) was added dropwise and the reaction mixture was stirred at 0° C. for 30 min. DCM and an aqueous solution of NaHCO₃ (10%) were added. The layers were separated, and the aqueous layer was extracted with DCM. The combined organic layers were dried over MgSO₄, filtered off and evaporated to obtain a brown gum which was purified by preparative LC (regular SiOH, 30 μm, 24 g, liquid loading (DCM), mobile phase gradient: from Heptane 90%, EtOAc/MeOH (9:1) 10% to Heptane 25%, EtOAc/MeOH (9:1) 75% over 12 CV). Fractions containing product were combined and evaporated to give 208 mg as a yellow solid. It was purified by Reverse phase (Stationary phase: YMC-actus Triart C18 25 μm 30*150 mm, 40 g, dry loading (Celite®) Mobile phase: Gradient from 60% (aq. NH₄HCO₃ 0.2%), 40% MeCN to 100% MeCN over 12 CV). Fractions containing product were combined and evaporated to afford 175 mg as a yellow solid. It was purified by preparative LC (regular SiOH, 30 μm, 24 g, liquid loading (DCM), mobile phase gradient: from Heptane 90%, EtOAc/MeOH (9:1) 10% to Heptane 25%, EtOAc/MeOH (9:1) 75% over 12 CV). Fractions containing product were combined and evaporated to give 146 mg as a white solid. This one was purified by Reverse phase (Stationary phase: YMC-actus Triart C18 25 μm 30*150 mm, 40 g, dry loading (Celite®) Mobile phase: Gradient from 60% (aq. NH₄HCO₃ 0.2%), 40% MeCN/MeOH (1:1) to 15% (aq. NH₄HCO₃ 0.2%), 85% MeCN/MeOH (1:1) over 14 CV). Fractions containing product were combined and evaporated to afford 129 mg as a white solid. It was purified by achiral SFC (Stationary phase: diethylaminopropyl 5 μm 150×21.2 mm, Mobile phase: 90% CO₂, 10% MeOH). Fractions containing product were combined and evaporated to afford 94 mg as a white solid. This one was sonicated in MeCN (10 mL) and evaporated (3 times) then MeCN (5 mL) was added, the product was filtered and dried under high vacuum (50° C., 2 h) to afford 84 mg of compound 55 as a white solid (28%)

¹H NMR (400 MHz, DMSO-d6) δ ppm 9.07 (d, J=1.5 Hz, 1H), 8.44 (br t, J=5.7 Hz, 1H), 7.67 (d, J=9.4 Hz, 1H), 7.45 (dd, J=9.4, 2.1 Hz, 1H), 7.32 (m, J=8.7 Hz, 2H), 7.16 (m, J=8.7 Hz, 2H), 4.47 (br d, J=5.9 Hz, 2H), 3.90-4.00 (m, 2H), 3.81-3.89 (m, 2H), 3.66 (t, J=6.7 Hz, 2H), 3.26-3.29 (m, 3H), 2.98 (q, J=7.5 Hz, 2H), 2.82 (t, J=6.7 Hz, 2H), 1.26 (t, J=7.5 Hz, 3H)

The following compounds were prepared in accordance with the procedures described herein:

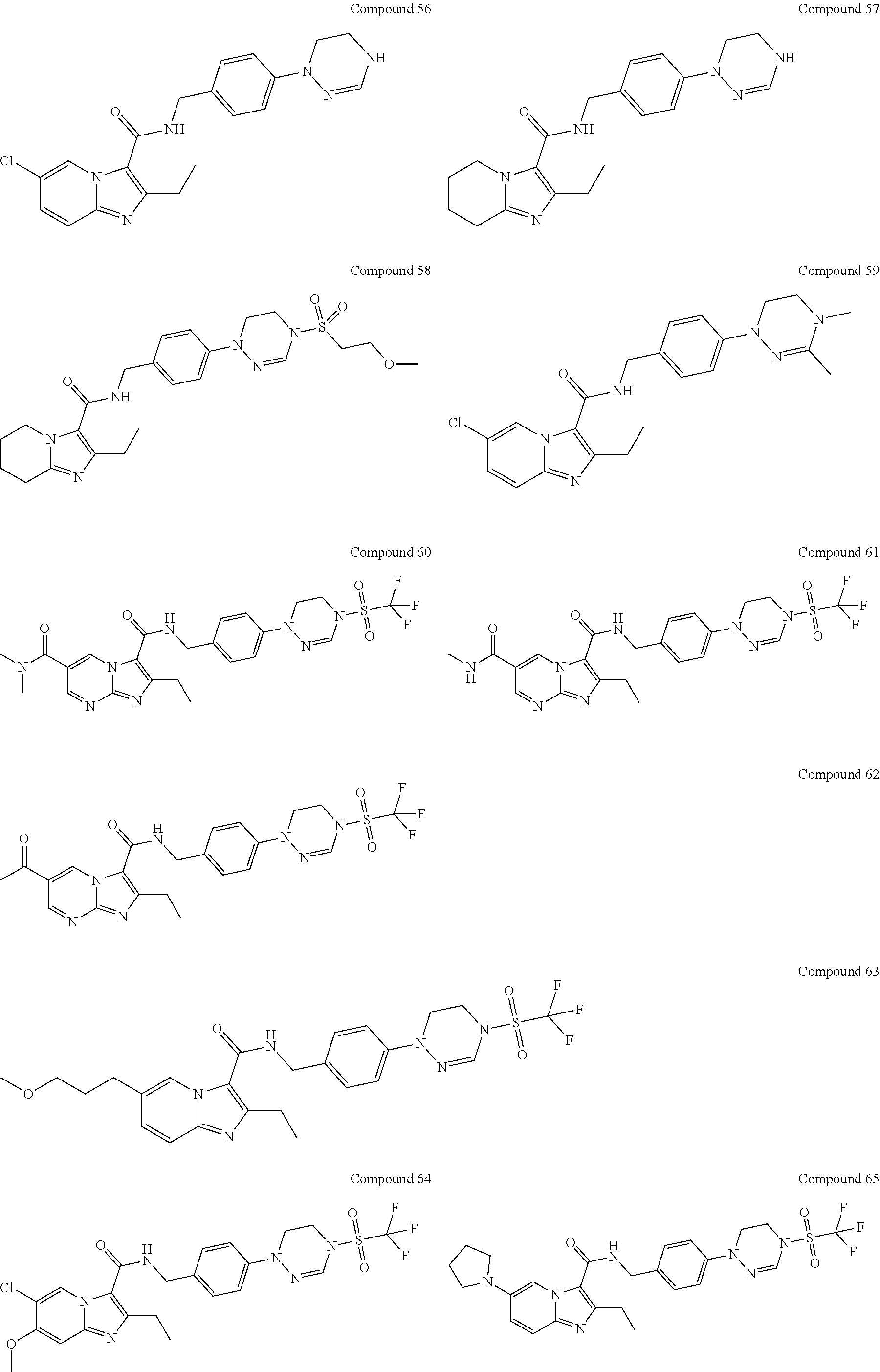

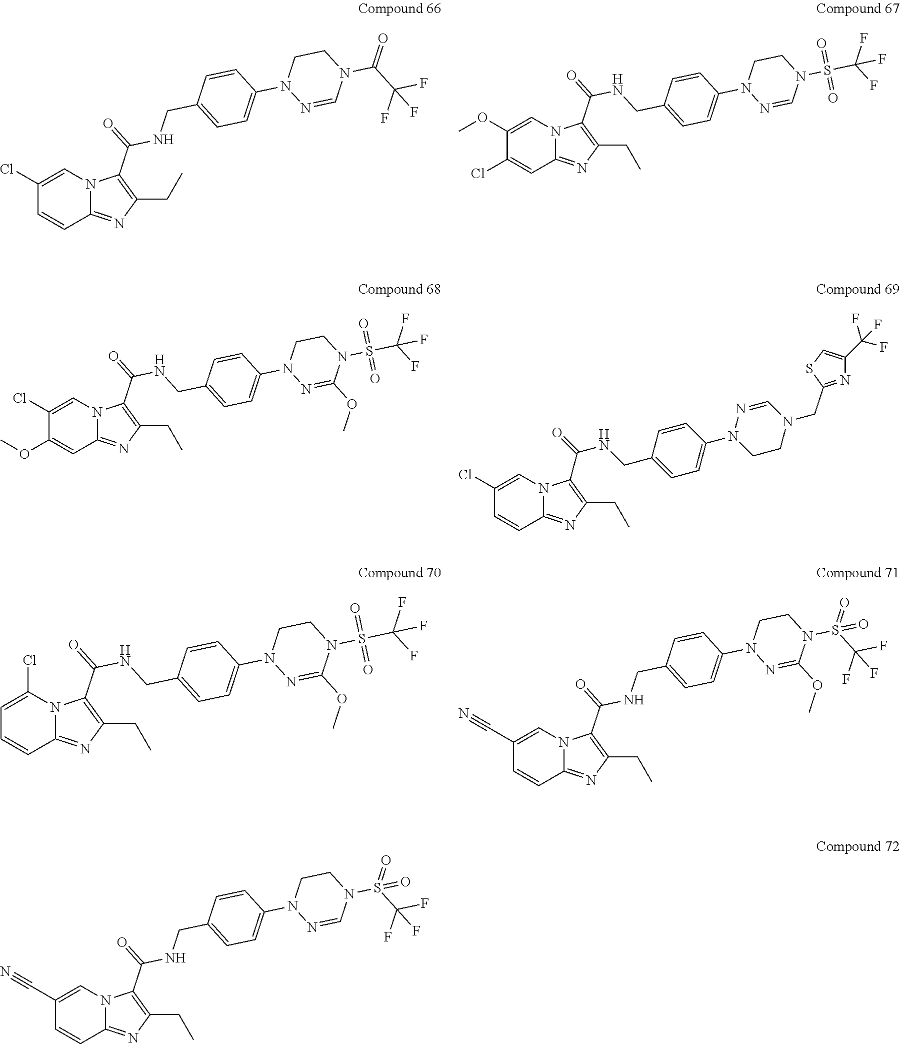

Synthesis of Compound 73

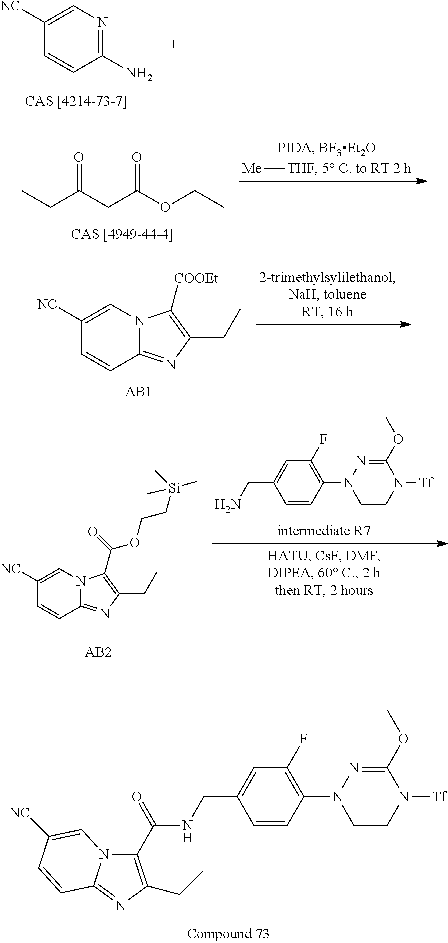

Preparation of Intermediate AB11

To a solution of 2-amino-5-cyanopyridine (CAS [4214-73-7]; 5 g, 42.0 mmol) in Me-THF (200 mL) at 5° C. were added iodobenzene diacetate (13.5 g, 41.9 mmol) and ethyl-3-oxovalerate (10 mL, 70.1 mmol). Then boron trifluoride etherate (550 μL, 2.10 mmol) was added dropwise. The solution was stirred at 5° C. for 1 h. The mixture was warmed to room temperature and stirred for 2 hours. EtOAc and a sat. solution of NaHCO₃ were added. The layers were separated, and the aqueous layer was extracted with EtOAc. The combined organic layers were washed with brine (twice), dried over MgSO₄, filtered off then evaporated to give 26 g of a brown liquid (which crystallized on standing). The crude product was purified by preparative LC (irregular SiOH, 15-40 μm, 330 g, Grace, dry loading (Celite®), mobile phase gradient: from heptane 85%, EtOAc 15% to heptane 30%, EtOAc 70%) to afford 3.14 g of the intermediate AB1 as a yellow solid (30%).

Preparation of Intermediate AB2

Under Nitrogen, NaH 60% (0.677 g; 16.9 mmol) was added to a solution of 2-(trimethylsilyl)ethanol (2.43 mL; 16.9 mmol) in dry toluene (50 mL) at 0° C. The reaction mixture was stirred at 0° C. for 15 min then intermediate AB1 (0.823 g; 3.38 mmol) was added and the reaction mixture was stirred for 16 h warming to room temperature. The reaction mixture was hydrolyzed with a aqueous saturated solution of NH4Cl and extracted with EtOAc. The aqueous layer was extracted with EtOAc (twice). The combined organic layers were dried over MgSO₄, filtered, evaporated to dryness and purified by preparative LC (Regular SiOH, 30-40 μm, 40 g, loading (DCM), mobile phase gradient: Heptane/EtOAc from 100:0 to 50:50). The fractions containing product were evaporated to give 559 mg of intermediate AB2 as a white solid (52%).

Preparation of Compound 73

Cesium fluoride (289 mg, 1.90 mmol) was added to a solution of intermediate AB2 (200 mg, 0.634 mmol) in F (8.4 mL) and the reaction mixture was stirred at 60° C. for 2 h. Then diisopropylethylamine (139 μL, 0.817 mmol) and HATU (267 mg, 0.701 mmol) were added and the reaction mixture was stirred at room temperature for 15 min (the reaction mixture turned to brown). Intermediate R₇ (266 mg, 0.634 mmol) was added and the reaction mixture was stirred at room temperature for 2 hours.

The reaction mixture was diluted with EtOAc, and the organic layer was washed with an aqueous solution of NaHCO3 1%, then with water and brine, dried over MgSO₄, filtered off and concentrated. DCM and MeOH were added to the residue. The mixture was filtered. The precipitate was dried under vacuum at 50° C. to give 160 mg of a crude product as a white solid.

The crude product was heated to reflux with EtOAc (15 mL) for 20 min then slowly cooled down to room temperature for 18 hours with slowly stirring.

The solid was filtered, rinced with cooled EtOAc and dried under vacuum at 60° C. to give 128 mg of compound 73 as white solid (36%).

1H NMR (400 MHz, DMSO-d6) δ ppm 9.50 (s, 1H) 8.63 (t, J=5.9 Hz, 1H) 7.78 (d, J=9.3 Hz, 1H) 7.66 (dd, J=9.3, 1.7 Hz, 1H) 7.45 (t, J=8.6 Hz, 1H) 7.13-7.31 (m, 2H) 4.51 (d, J=5.87 Hz, 2H) 4.06-4.19 (m, 2H) 3.53-3.62 (m, 2H) 3.02 (q, J=7.50 Hz, 2H) 1.28 (t, J=7.46 Hz, 3H).

Synthesis of Compound 74

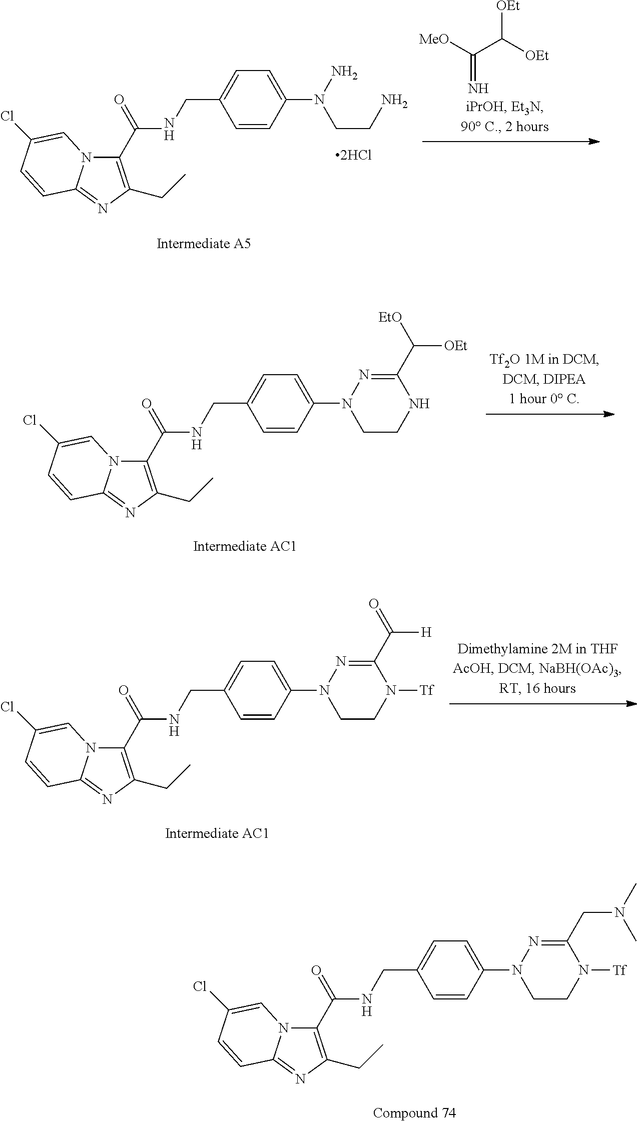

Preparation of Intermediate AC1

A mixture of intermediate A5 (500 mg, 1.09 mmol), methyl-2,2-diethoxyacetimidate (526 mg, 3.26 mmol) and triethylamine (453 μL, 3.26 mmol) in iPrOH (9.4 mL) was stirred for 2 h at 90° C. After cooling to room temperature, the reaction mixture was concentrated. The residue was taken up in EtOAc and water. After separation, the aqueous phase was extracted with EtOAc (once). The combined organic layers were washed with brine, dried over MgSO₄, filtered off and concentrated. The residue was purified by preparative LC (irregular SiOH 15-40 μm, 80 g, liquid loading (DCM), mobile phase gradient: EtOAc in heptane from 20 to 80% then isocratic). Fractions containing product were combined and evaporated to give 343 mg of intermediate AC1 as a white solid (63%).

Preparation of Intermediate AC2

Diisopropylethylamine (0.311 mL, 1.80 mmol) was added to a solution of intermediate AC1 (300 mg, 0.601 mmol) in DCM (5.5 mL). The solution was then cooled at 0° C. (ice/water bath). A 1M solution of Tf₂O in DCM (0.721 mL, 1.2 eq., 0.721 mmol) was added dropwise and the reaction mixture was stirred at 0° C. for 1 h. An extra amount of a 1M solution of Tf₂O in DCM (0.721 mL, 1.2 eq., 0.721 mmol) was added and the mixture was stirred at 0° C. for 1 hour. A saturated aqueous solution of NaHCO₃ and DCM were added. The layers were separated, and the aqueous layer was extracted with DCM. The combined organic layers were dried over MgSO₄, filtered off and evaporated to afford a brown gum. This crude product was purified by preparative LC (regular SiOH, 30 μm, 24 g, liquid loading (DCM), mobile phase gradient: from DCM 100% to DCM 85%, MeOH/AcOH (9:1) 15%) to afford 94 mg of intermediate AC2 as an orange gum.

Preparation of Compound 74

To a solution of intermediate AC2 (94 mg, 0.17 mmol) in AcOH (29 μL, 0.51 mmol) and DCM (1.5 mL) was added a 2M solution of dimethylamine in THE (0.25 mL, 0.51 mmol) and the mixture was stirred at room temperature for 6 hours. Then, sodium triacetoxyborohydride (71.5 mg, 0.34 mmol) was added and the mixture was stirred at room temperature for 16 hours. A saturated aqueous solution of NaHCO₃ was added carefully then the layers were separated. The aqueous layer was extracted with DCM (twice) then the combined organic layers were dried over MgSO₄, filtered off and evaporated. The crude product was purified by preparative LC (regular SiOH, 30 μm, 12 g, liquid loading (DCM), mobile phase gradient: from heptane 80%, EtOAc/MeOH (9:1) 20% to Heptane 15%, EtOAc/MeOH (9:1) 85%). Fractions containing product were combined and evaporated to give 68 mg as a light-yellow oil which was purified by Reverse phase (Stationary phase: YMC-actus Triart C18 25 μm 30*150 mm, 12 g, dry loading (Celite®) Mobile phase: Gradient from 55% (aq. NH₄HCO₃ 0.2%), 45% MeCN to 100% MeCN). Fractions containing product were combined and evaporated to afford a colorless oil which was triturated in Et₂O, dried under high vacuum (50° C., 1 h) to afford 40 mg of compound 74 as a white solid (40%).

¹H NMR (400 MHz, DMSO-d₆) δ ppm 9.06 (d, J=1.0 Hz, 1H) 8.44 (br t, J=5.8 Hz, 1H) 7.67 (d, J=9.7 Hz, 1H) 7.45 (dd, J=9.4, 1.8 Hz, 1H) 7.33 (br d, J=8.6 Hz, 2H) 7.19 (br d, J=8.6 Hz, 2H) 4.47 (br d, J=5.5 Hz, 2H) 3.90 (br dd, J=16.6, 4.2 Hz, 4H) 2.97 (q, J=7.5 Hz, 2H) 2.19 (s, 7H) 1.26 (t, J=7.5 Hz, 4H).

Synthesis of Compound 75

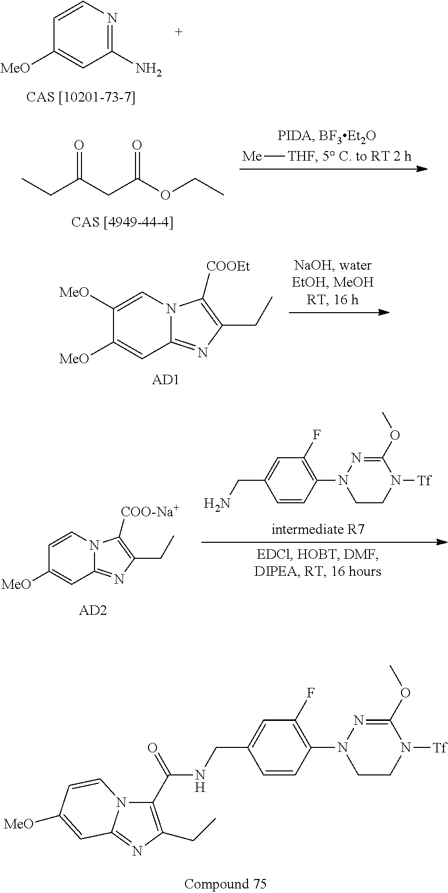

Preparation of Intermediate AD1

Carbon tetrabromide (26.9 g, 81.0 mmol) was added to a solution of 2-amino-4-methoxypyridine [CAS:10201-73-7] (5.02 g, 40.4 mmol) and ethyl-3-oxovalerate (8.69 mL, 60.8 mmol) in MeCN (85 mL) and the reaction mixture was stirred at 80° C. for 4 hours. The reaction mixture was evaporated until dryness then purified by preparative LC (regular SiOH, 30 μm, 330 g, dry loading (Celite®), mobile phase gradient: from Heptane/EtOAc 95/5 to EtOAc) to give 669 mg of intermediate AD1(16%).

Preparation of Intermediate AD2

To a mixture of intermediate AD1 (1.55 g, 6.24 mmol) in water (20 mL) and EtOH (20 mL) was added NaOH (752 mg, 18.8 mmol) and the mixture was stirred at room temperature for 2 days. The reaction mixture was evaporated to give 2.16 g of intermediate AD2 (Quant.)

Preparation of Compound 75

A mixture of intermediate AD2 (138 mg, 0.397 mmol), intermediate R7 (160 mg, 397 μmol), EDCI·HCl (99.1 mg, 0.517 mmol), HOBt (79.1 mg, 0.517 mmol) and diisopropylethylamine (205 μL, 1.19 mmol) in DMF (6 mL) was stirred at room temperature for 20 hours.

The residue was dissolved in EtOAc and water. The aqueous layer was extracted with EtOAc (twice). The combined organic layers were dried over MgSO₄, filtered off and evaporated to give an orange oil. The oil was purified by preparative LC (regular SiOH 30 μm, 12 g, dry loading (Celite®), mobile phase gradient: Heptane/EtOAc 70/30 to EtOAc 100%). The fractions containing product were combined and evaporated under vacuum to give a yellow solid which was triturated in Et₂O. The supernatant was removed by pipette and the solid was dried under vacuum to give 124 mg of a white solid which was co-evaporated in Et₂O (3 times) to give 120 mg of compound 75 as a white solid (46% yield).

¹H NMR (400 MHz, DMSO-d₆) δ ppm 8.86 (d, J=7.7 Hz, 1H) 8.21 (br t, J=5.8 Hz, 1H) 7.44 (t, J=8.5 Hz, 1H) 7.12-7.26 (m, 2H) 7.01 (d, J=2.3 Hz, 1H) 6.71 (dd, J=7.6, 2.5 Hz, 1H) 4.47 (br d, J=5.9 Hz, 2H) 4.07-4.15 (m, 2H) 3.84 (d, J=8.2 Hz, 6H) 3.52-3.61 (m, 2H) 2.94 (q, J=7.5 Hz, 2H) 1.26 (t, J=7.5 Hz, 3H).

Synthesis of Compound 76

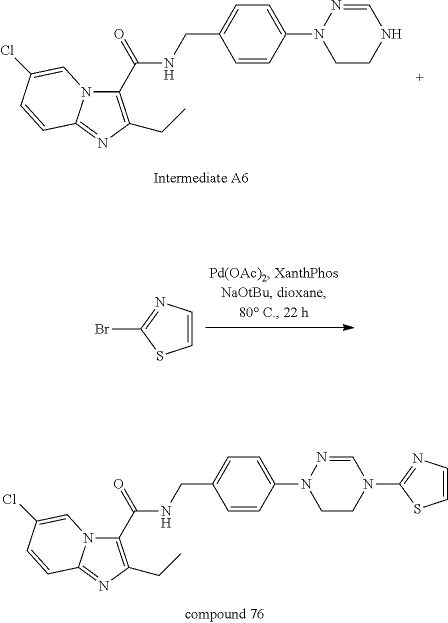 [SMILES: CCc1nc2ccc(Cl)cn2c1C(=O)NCc1ccc(N2CCNC=N2)cc1 ; Brc1nccs1 ; CCc1nc2ccc(Cl)cn2c1C(=O)NCc1ccc(N2CCN(c3nccs3)C=N2)cc1]

A mixture of intermediate A6 (30.0 mg, 75.6 μmol), 2-Bromothiazole (8.18 μL, 90.7 μmol) and NaOtBu (36.3 mg, 0.378 mmol) in dry 1,4-dioxane (1.3 mL) was purged with N₂ (3 times). XanthPhos (8.7 mg, 15 μmol) and Palladium II acetate (1.7 mg, 7.6 μmol) were then added and the mixture was purged with N₂ (3 times). The reaction mixture was stirred at 80° C. for 22 hours. The reaction mixture was diluted with EtOAc/MeOH and water. The aqueous layer was extracted with EtOAc (twice). The combined organic layer was washed with brine, dried over MgSO₄, filtered off and evaporated to give a brown solid. The solid was purified by preparative LC (regular SiOH 30 μm, 12 g, dry loading (Celite®), mobile phase gradient: DCM 100% to DCM/(DCM: MeOH 80:20) 30/70). The fractions containing product were combined and evaporated under vacuum to give 17 mg of compound 76 as yellow solid (47% yield).

¹H NMR (500 MHz, DMSO-d₆) δ ppm 9.07 (d, J=1.4 Hz, 1H) 8.45 (t, J=5.9 Hz, 1H) 7.63-7.69 (m, 2H) 7.45 (dd, J=9.5, 2.0 Hz, 1H) 7.39 (d, J=3.5 Hz, 1H) 7.26 (dd, J=36.7, 8.7 Hz, 2H) 7.16 (d, J=3.5 Hz, 1H) 4.46 (d, J=5.6 Hz, 2H) 4.00 (t, J=5.0 Hz, 2H) 3.78 (t, J=5.0 Hz, 2H) 2.98 (q, J=7.5 Hz, 2H) 1.26 (t, J=7.5 Hz, 4H).

The following compound was also prepared in accordance with the procedures described herein:

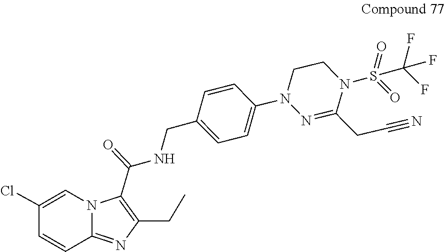 [SMILES: CCc1nc2ccc(Cl)cn2c1C(=O)NCc1ccc(N2CCN(S(=O)(=O)C(F)(F)F)C(CC#N)=N2)cc1]

B. Further Procedures

Synthesis of Compound 127

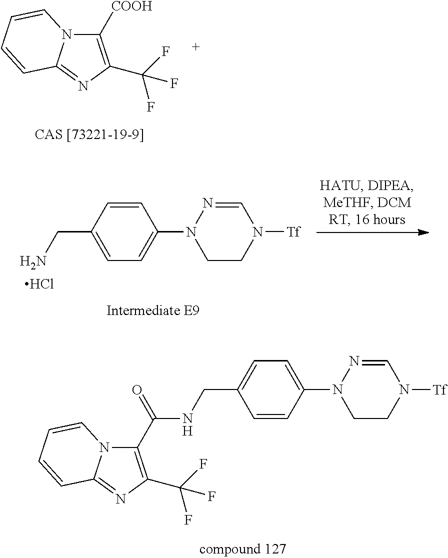

HATU (0.099 g, 0.26 mmol) was added to a solution of 2-(Trifluoromethyl)-imidazo[1,2-A]pyridine-3-carboxylic acid (CAS [73221-19-9], 0.052 g, 0.23 mmol) and DIPEA (0.097 mL, 0.56 mmol) in dry Me-THF (1.52 mL) and DCM (0.51 mL) under N₂. The solution was stirred at room temperature for 15 min. Then intermediate E9 (0.08 g, 0.25 mmol) was added and the reaction mixture was stirred at room temperature for 16 hours. The solvent was evaporated then the residue was diluted in ethyl acetate, washed with a saturated aqueous solution of NaHCO₃, water then brine. The organic layer was dried over MgSO₄, filtered and evaporated in vacuo to give a yellow oil, 0.167 g. Purification was carried out by flash chromatography over silica gel (12 g, irregular SiOH 25-40 μM, DCM/MeOH from 100/0 to 97/3). Pure fractions were collected and evaporated affording a colorless oil which crystallized on standing, 0.102 g. A purification was performed via Reverse phase (Stationary phase: YMC-actus Triart C18 10 μm 30*150 mm, Mobile phase: Gradient from 40% NH₄HCO₃ 0.2%, 60% ACN to 10% NH₄HCO₃ 0.2%, 90% ACN). Pure fractions were collected and evaporated affording 0.037 g as white foam. It was triturated with DIPE and a few Heptane, the precipitate was filtered off and dried under vacuum at 60° C. affording compound 127 as white powder, 0.032 g (26%).

¹H NMR (500 MHz, DMSO-d₆) δ ppm 9.23 (br s, 1H), 8.53 (br d, J=6.4 Hz, 1H), 7.79 (br d, J=8.9 Hz, 1H), 7.55 (br t, J=7.5 Hz, 1H), 7.25-7.37 (m, 3H), 7.20 (br d, J=8.1 Hz, 3H), 4.42-4.56 (m, 2H), 4.08 (br s, 2H), 3.84 (br s, 2H)

Synthesis of Compound 128

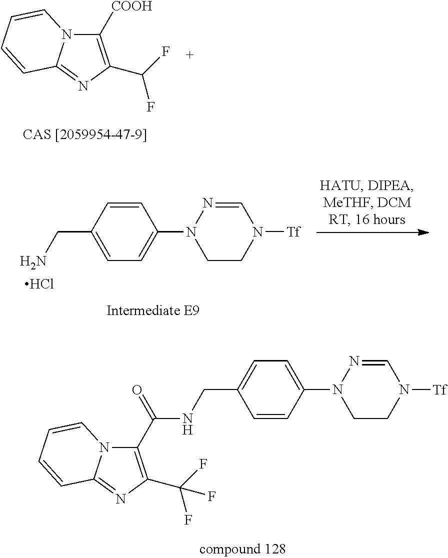

Accordingly, compound 128 was prepared in the same way as compound 127 starting from 2-(Difluoromethyl)-imidazo[1,2-A]pyridine-3-carboxylic acid (CAS [2059954-47-9], 0.23 mmol) and intermediate E9 affording a white powder, 0.045 g (39%).

¹H NMR (500 MHz, DMSO-d₆) δ ppm 8.96 (br t, J=5.6 Hz, 1H), 8.79 (d, J=7.0 Hz, 1H), 7.76 (d, J=9.0 Hz, 1H), 7.52 (t, J=7.8 Hz, 1H), 7.25-7.45 (m, 4H), 7.20 (d, J=8.7 Hz, 2H), 7.16 (td, J=6.9, 1.1 Hz, 1H), 4.48 (d, J=5.6 Hz, 2H), 4.08 (br t, J=4.5 Hz, 2H), 3.84 (t, J=4.8 Hz, 2H)

Synthesis of Compound 137

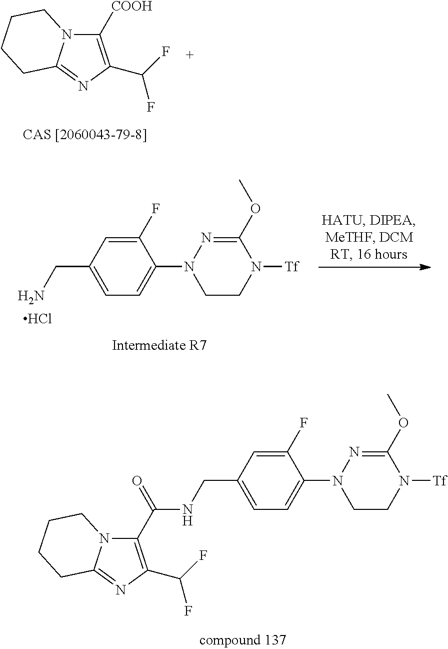

HATU (0.093 g, 0.24 mmol) was added to a solution of 2-(Difluoromethyl)-5H, 6H, 7H, 8H-imidazo[1,2-A]pyridine-3-carboxylic acid (0.046 g, 0.21 mmol) and DIPEA (0.091 mL, 0.53 mmol) in dry Me-THF (1.43 mL) and DCM (0.48 mL) under N₂. The solution was stirred at room temperature for 15 min. Then intermediate R7 (0.095 g, 0.23 mmol) was added and the reaction mixture was stirred at room temperature for 16 hours. The solvent was evaporated then the residue was diluted in ethyl acetate, washed with a saturated aqueous solution of NaHCO₃, water then brine. The organic layer was dried over MgSO₄, filtered and evaporated in vacuo to give a yellow oil, 0.271 g. Purification was carried out by flash chromatography over silica gel (12 g, irregular SiOH 25-40 μM, DCM/MeOH from 100/0 to 97/3). Pure fractions were collected and evaporated affording 0.112 g as colourless oil which crystalized on standing. It was triturated with DIPE and a few Heptane, the precipitate was filtered off and dried under vacuum at 60° C. affording compound 137 as white powder, 0.096 g (79%).

¹H NMR (500 MHz, DMSO-d₆) δ ppm 8.77 (br t, J=5.6 Hz, 1H), 7.44 (t, J=8.6 Hz, 1H), 7.10-7.19 (m, 2H), 6.95 (t, J=54.3 Hz, 1H), 4.40 (br d, J=5.8 Hz, 2H), 4.06-4.15 (m, 2H), 4.02 (br t, J=5.5 Hz, 2H), 3.83 (s, 3H), 3.54-3.60 (m, 2H), 2.78 (br t, J=6.3 Hz, 2H), 1.89 (br d, J=4.6 Hz, 2H), 1.83 (br d, J=5.5 Hz, 2H)

Synthesis of Compound 79

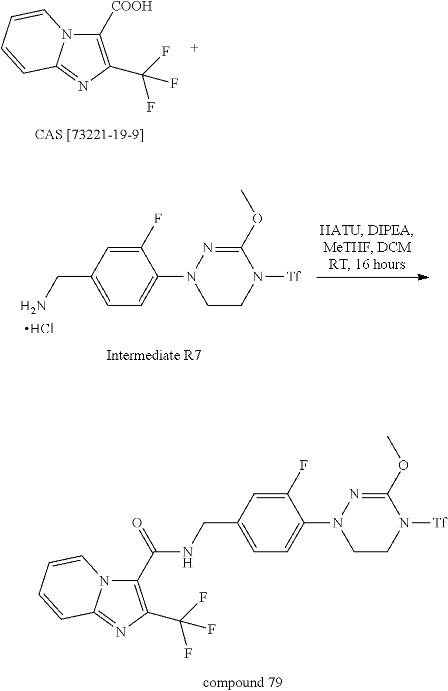

Accordingly, compound 79 was prepared in the same way as compound 137 starting from 2-(Trifluoromethyl)-imidazo[1,2-A]pyridine-3-carboxylic acid (CAS [73221-19-9], 0.21 mmol) and intermediate R-7 (0.23 mmol) affording a white powder, 0.09 g (70%).

¹H NMR (500 MHz, DMSO-d6) δ ppm 9.27 (t, J=5.8 Hz, 1H), 8.57 (d, J=6.9 Hz, 1H), 7.80 (d, J=9.2 Hz, 1H), 7.40-7.62 (m, 2H), 7.14-7.27 (m, 3H), 4.47-4.56 (m, 2H), 4.08-4.14 (m, 2H), 3.84 (s, 3H), 3.52-3.63 (m, 2H)

Synthesis of Compound 132

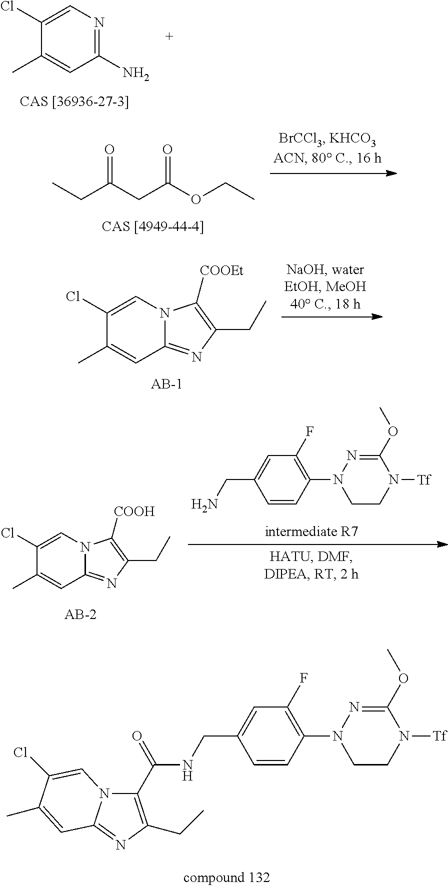

Preparation of Intermediate AB-1 In a sealed tube, to a solution of 2-amino-5-chloropicoline (CAS [36936-27-3], 1.00 g, 7.01 mmol) in ACN (12 mL) were added Ethyl-ethyl 3-oxovalerate (CAS [4949-44-4], 2.00 mL, 14.0 mmol), bromotrichloromethane (2.40 mL, 24.4 mmol) and potassium bicarbonate (2.12 g, 21.2 mmol). The mixture was stirred at 80° C. for 16 h. EtOAc and water were added. The organic layer was washed with brine, dried (MgSO₄), evaporated and purified by preparative LC (irregular SiOH, 15-40 μm, 80 g, mobile phase gradient: from heptane/EtOAc 90:10 to 10:90) The fractions containing product were combined and evaporated to afford 0.95 g of intermediate AB-1 as an orange solid (51%).

Preparation of Intermediate AB-2

To a mixture of intermediate AB-1 (180 mg, 0.675 mmol) in water (2.2 mL) and EtOH (2.2 ml) was added NaOH (81 mg, 2.03 mmol) and the mixture was stirred at 40° C. for 18 h.

The reaction mixture was evaporated to give 270 mg g of intermediate AB-2 (Quant. purity 65%).

Preparation of Compound 132

A mixture of intermediate AB-2 (150 mg, 0,374 mmol, purity 65%), intermediate R7 (151 mg, 0,374 mmol), HATU (157 mg, 0.414 mmol), DIPEA (82 μL, 0.48 mmol) and DMF (2.3 mL) was stirred at room temperature for 2 h. The reaction mixture was diluted with EtOAc, and the organic layer was washed with an aqueous solution of NaHCO₃ 1%, then with water and brine, dried over MgSO₄, filtered off, concentrated and purified by preparative LC (irregular SiOH, 15-40 μm, 40 g Grace, loading (DCM), mobile phase gradient: from Heptane/EtOAc: 50/50 to 0/100 in 7 CV then EtOAc 100% in 7 CV). The fractions containing product were combined and evaporated to give 116 mg as a white solid. It was purified by preparative LC (spherical C18 25 μm, 40 g YMC-ODS-25, (MeOH/MeCN), mobile phase gradient 0.2% aq. NH₄⁺HCO₃⁻/MeCN from 70:30 to 0:100). The fraction containing product were combined and evaporated to give 86 mg of compound 132 as white solid (39%).

1H NMR (400 MHz, DMSO-d6) δ ppm 9.12 (s, 1H), 8.35 (t, J=5.9 Hz, 1H), 7.64 (s, 1H), 7.45 (t, J=8.6 Hz, 1H), 7.11-7.27 (m, 2H), 4.48 (d, J=5.9 Hz, 2H), 4.11 (br t, J=5.2 Hz, 2H), 3.83 (s, 3H), 3.57 (br t, J=4.9 Hz, 2H), 2.99 (q, J=7.5 Hz, 2H), 2.40 (s, 3H), 1.26 (t, J=7.5 Hz, 3H)

Synthesis of Compound 141

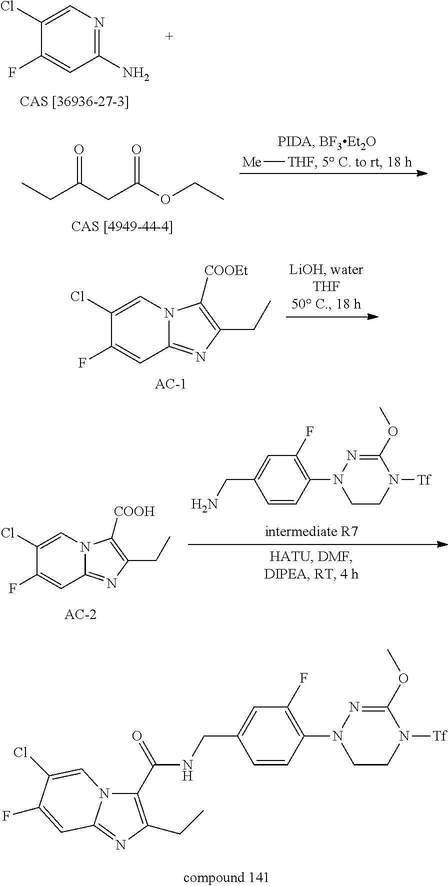

Preparation of Intermediate AC-1

To a solution of 5-Chloro-4-fluoro-2-pyridinamine (CAS [1393574-54-3], 250 mg, 1.71 mmol) in Me-THF (8 mL) at 5° C. were added iodobenzene diacetate (550 mg, 1.71 mmol) and ethyl-ethyl 3-oxovalerate (0.4 mL, 2.80 mmol). Then Boron trifluoride etherate (25 μL, 95.5 μmol) was added dropwise. The solution was stirred at 5° C. for 1 h. The mixture was warmed to room temperature and stirred for 18. EtOAc and water were added. The organic layer was washed with brine, dried (MgSO₄), evaporated and purified by preparative LC (irregular SiOH, 15-40 μm, 40 g, grace, loading (DCM) mobile phase gradient: from heptane/EtOAc 90:10 to 10:90 over 10 CV) to afford 119 mg of intermediate AC-1 as a pale brown solid (P1; 26%)

Preparation of Intermediate AC-2

A mixture of intermediate AC-1 (200 mg, 0.739 mmol), Lithium hydroxide (177 mg, 7.39 mmol), water (3.2 mL) and THE (4.4 mL) was stirred at 50° C. for 18h. EtOAc and aq. KHSO₄ 10% was added. The organic layer was dried (MgSO₄) and evaporated to give 179 mg of intermediate AC-2 as yellow solid (Quant.).

Preparation of Compound 141

Accordingly, compound 141 was prepared in the same way as compound 132 starting from intermediate AC-2 (0.78 mmol) and intermediate R7 affording 0.127 g (27%) as a white powder.

1H NMR (400 MHz, DMSO-d6) δ ppm 9.24 (d, J=7.3 Hz, 1H), 8.45 (br t, J=5.8 Hz, 1H), 7.79 (d, t, J=9.9 Hz, 1H), 7.45 (t, t, J=8.7 Hz, 1H), 7.12-7.27 (m, 2H), 4.49 (d, t, J=5.9 Hz, 2H), 4.11 (t, t, J=4.9 Hz, 2H), 3.83 (s, 3H), 3.57 (t, t, J=4.9 Hz, 2H), 2.99 (q, t, J=7.5 Hz, 2H), 1.27 (t, J=7.5 Hz, 3H)

Synthesis of Compound 158

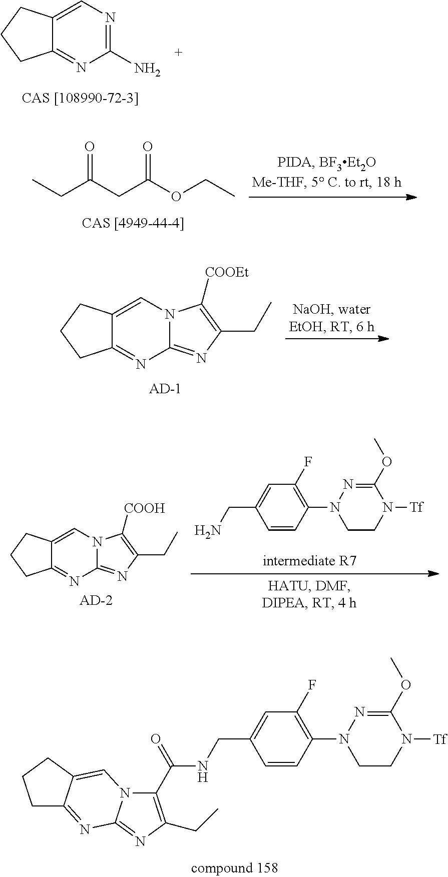

Preparation of Intermediate AD-1

Accordingly, compound AD-1 was prepared in the same way as compound AC-1 starting from 6,7-dihydro-5h-cyclopenta[d]pyrimidin-2-amine (CAS [108990-72-3], 7.4 mmol) affording 0.726 g (38%).

Preparation of Intermediate AD-2

Accordingly, compound AD-2 was prepared in the same way as compound AB-2 starting from AD-1 (0.77 mmol) affording 0.446 g (44%).

Preparation of Compound 158

Accordingly, compound 158 was prepared in the same way as compound 132 starting from intermediate AD-2 (0.77 mmol) and intermediate R7 affording 0.145 g (32%) as a white powder.

1H NMR (500 MHz, DMSO-d6) δ ppm 9.10 (s, 1H), 8.39 (t, J=6.0 Hz, 1H), 7.44 (t, J=8.5 Hz, 1H), 7.12-7.26 (m, 2H), 4.47 (d, J=5.9 Hz, 2H), 4.10 (t, J=4.8 Hz, 2H), 3.83 (s, 3H), 3.56 (t, J=4.8 Hz, 2H), 2.89-3.03 (m, 6H), 2.05-2.16 (m, 2H), 1.26 (t, J=7.6 Hz, 3H)

Preparation of Compound 193

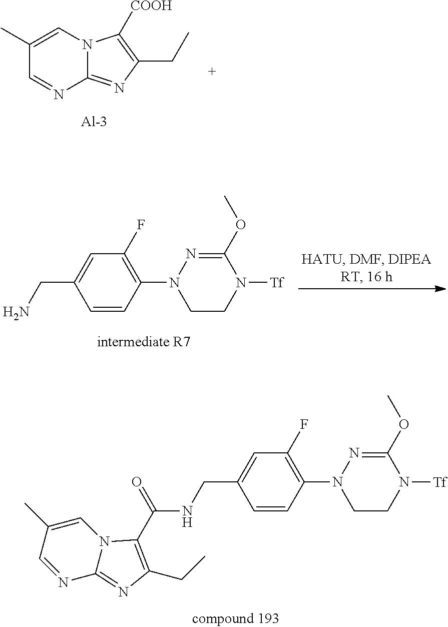

Accordingly, compound 193 was prepared in the same way as compound 158 starting from intermediate AI-3 (0.44 mmol) and intermediate R-7 (0.37 mmol) affording a white solid, 0.108 g (52%).

¹H NMR (400 MHz, DMSO) δ 9.19-9.10 (m, 1H), 8.51 (d, J=2.4 Hz, 1H), 8.44 (t, J=5.9 Hz, 1H), 7.44 (t, J=8.6 Hz, 1H), 7.26-7.14 (m, 2H), 4.49 (d, J=5.9 Hz, 2H), 4.14-4.03 (m, 2H), 3.83 (s, 3H), 3.59-3.53 (m, 2H), 3.01 (q, J=7.5 Hz, 2H), 2.34 (d, J=0.6 Hz, 3H), 1.28 (t, J=7.5 Hz, 3H).

Preparation of Compound 194

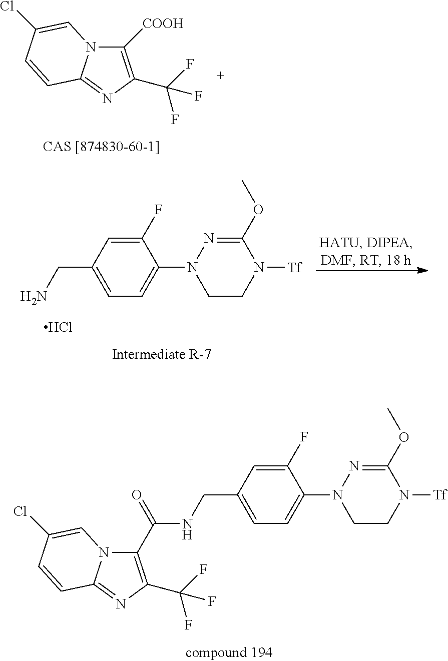

Accordingly, compound 194 was prepared in the same way as compound 158 starting from 6-Chloro-2-(trifluoromethyl)imidazo[1,2-a]pyridine-3-carboxylic acid (CAS [874830-60-1] (0.7 mmol) and intermediate R-7 (0.47 mmol) affording a white solid, 0.110 g (39%).

¹H NMR (400 MHz, DMSO) δ 9.23 (t, J=5.8 Hz, 1H), 8.35 (s, 1H), 7.70 (d, J=9.3 Hz, 1H), 7.52-7.37 (m, 2H), 7.19 (m, 2H), 4.51 (d, J=5.8 Hz, 2H), 4.17-4.07 (m, 2H), 3.84 (s, 3H), 3.63-3.55 (m, 2H), 2.34 (s, 3H).

Preparation of Compound 204

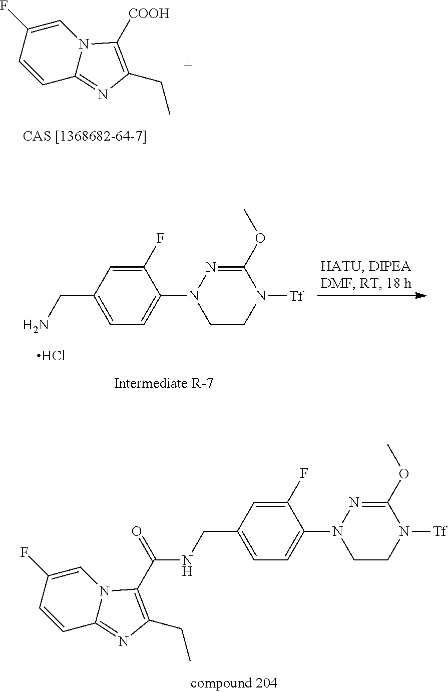

Accordingly, compound 204 was prepared in the same way as compound 158 starting from 2-ethyl-6-fluoroimidazo[1,2-a]pyridine-3-carboxylic acid (CAS [1368682-64-7], 0.84 mmol) and intermediate R-7 (0.7 mmol) affording a white solid, 0.132 g (34%).

¹H NMR (400 MHz, DMSO) δ 9.09-9.01 (m, 1H), 8.40 (t, J=5.9 Hz, 1H), 7.73-7.64 (m, 1H), 7.53-7.41 (m, 2H), 7.25-7.14 (m, 2H), 4.49 (d, J=5.9 Hz, 2H), 4.15-4.05 (m, 2H), 3.83 (s, 3H), 3.61-3.51 (m, 2H), 3.00 (q, J=7.5 Hz, 2H), 1.27 (t, J=7.5 Hz, 3H).

Preparation of Compound 206

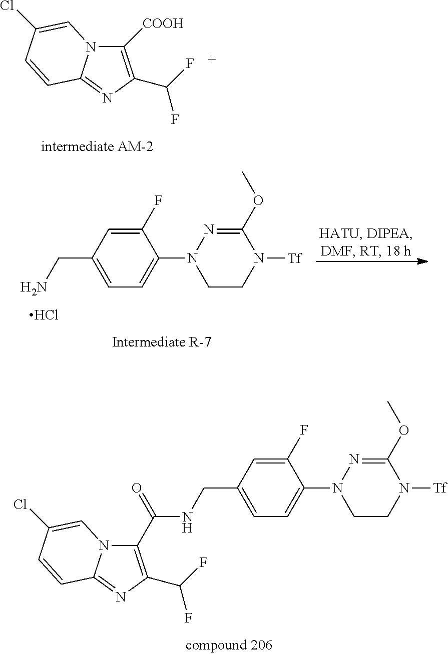

Accordingly, compound 206 was prepared in the same way as compound 158 starting from intermediate AM-2 (0.61 mmol) and intermediate R-7 (0.47 mmol) affording a beige powder, 0.07 g (24%).

¹H NMR (400 MHz, DMSO) δ 9.02 (t, J=5.7 Hz, 1H), 8.92 (d, J=1.7 Hz, 1H), 7.83 (d, J=9.6 Hz, 1H), 7.61 (dd, J=9.6, 2.0 Hz, 1H), 7.52-7.16 (m, 4H), 4.51 (d, J=5.7 Hz, 2H), 4.13-4.07 (m, 2H), 3.83 (s, 3H), 3.60-3.55 (m, 2H).

Preparation of Compound 209

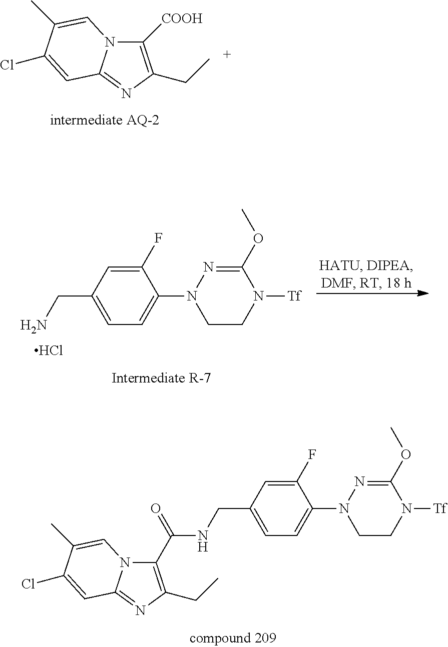

Accordingly, compound 209 was prepared in the same way as compound 158 starting from intermediate AQ-2 (0.56 mmol) and intermediate R-7 (0.4 mmol) affording a white powder, 0.142 g (59%).

¹H NMR (400 MHz, DMSO) δ 8.95 (s, 1H), 8.41 (t, J=5.9 Hz, 1H), 7.80 (s, 1H), 7.44 (t, J=8.6 Hz, 1H), 7.26-7.14 (m, 2H), 4.48 (d, J=5.9 Hz, 2H), 4.15-4.06 (m, 2H), 3.83 (s, 3H), 3.60-3.52 (m, 2H), 2.97 (q, J=7.5 Hz, 2H), 2.32 (s, 3H), 1.26 (t, J=7.5 Hz, 3H).

Preparation of Compound 210

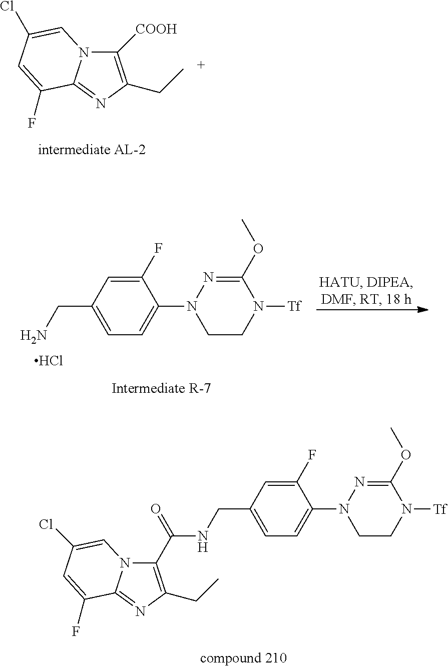

Accordingly, compound 210 was prepared in the same way as compound 158 starting from intermediate AL-2 (0.55 mmol) and intermediate R-7 (0.4 mmol) affording a white solid, 0.161 g (68%).

¹H NMR (400 MHz, DMSO) δ 8.92 (d, J=1.4 Hz, 1H), 8.60 (t, J=5.9 Hz, 1H), 7.62 (dd, J=10.6, 1.6 Hz, 1H), 7.45 (t, J=8.6 Hz, 1H), 7.26-7.15 (m, 2H), 4.50 (d, J=5.8 Hz, 2H), 4.15-4.06 (m, 2H), 3.83 (s, 3H), 3.61-3.52 (m, 2H), 3.01 (q, J=7.5 Hz, 2H), 1.27 (t, J=7.5 Hz, 3H).

Preparation of Intermediate AA-3

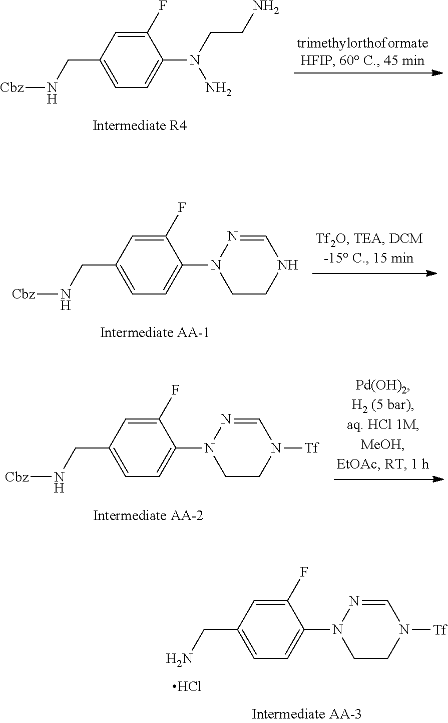

Preparation of Intermediate AA-1

A solution of intermediate R4 (19.6 g, 48.4 mmol) and Trimethylorthoformate (15.9 mL, 145 mmol) in HFIP (490 mL) was stirred at 60° C. for 45 min. The reaction mixture was evaporated. The residue was diluted in DCM and a 10% aq. solution of K₂CO₃ was added. The aqueous layer was extracted twice with DCM/MeOH (95/5). The combined organic layers were dried on MgSO₄, filtered off and evaporated. The crude (m=25.6 g) was purified by preparative LC (regular SiOH 30 μm, 330 g, dry loading (Celite®), mobile phase gradient: from Heptane 75%, EtOAc/MeOH (9:1) 25% to Heptane 25%, EtOAc/MeOH (9:1). Fractions containing product were combined and evaporated to give 14.61 g of intermediate AA-1 as a colorless oil which crystallized on standing (85%).

Preparation of Intermediate AA-2

To a solution of intermediate AA-1 (14.6 g, 42.7 mmol) and DIPE (22.1 mL, 128 mmol) in dry DCM (340 mL) at −5° C. (ice/NaCl solid) was added dropwise Tf₂O 1M in DCM (47 mL, 47 mmol) over 15 min using a dropping funnel and stirring was continued for 5 min. The reaction mixture was quenched with a saturated aqueous solution of NaHCO₃. The layers were separated, and the aqueous layer was extracted with DCM (twice). The combined organic layer was dried over MgSO₄, filtered off and concentrated. The crude (m=36.4 g) was purified by preparative LC (regular SiOH, 30 μm, 120 g, dry loading (Celite®), mobile phase gradient: Heptane/EtOAc 90/10 to 70/30). The fractions containing product were combined and evaporated under vacuum to give 10.18 g of intermediate AA-2 as a white solid (50%).

Preparation of Intermediate AA-3

In a steal bomb, a mixture of intermediate AA-2 (10.2 g, 21.5 mmol), Palladium hydroxide 20% on carbon nominally 50% water (3.01 g, 2.15 mmol) and aqueous HCl 3M (7.15 mL, 7.15 mmol) in MeOH (150 mL) and EtOAc (150 mL) was hydrogenated under 5 bar of H₂ at room temperature for 1 h. The mixture was filtered on a pad of Celite® and washed with MeOH. The filtrate was evaporated then co-evaporated with MeOH (twice) to give 7.86 g of intermediate AA-3.

Synthesis of Compound 163

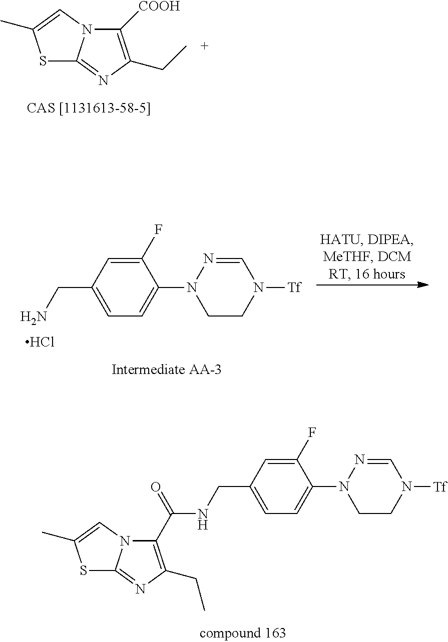

HATU (0.083 g, 0.22 mmol) was added to a solution of 6-ethyl-2-methylimidazo[2,1-b][1,3]thiazole-5-carboxylic acid (CAS [1131613-58-5], 0.04 g, 0.19 mmol) and DIPEA (0.082 mL, 0.48 mmol) in dry Me-THF (1.28 mL) and DCM (0.43 mL) under N₂. The solution was stirred at room temperature for 15 min. Then intermediate AA-3 (0.083 g, 0.22 mmol) was added and the reaction mixture was stirred at room temperature for 16 hours. The solvent was evaporated then the residue was diluted in ethyl acetate, washed with a saturated aqueous solution of NaHCO₃, water then brine. The organic layer was dried over MgSO₄, filtered and evaporated in vacuo to give a colorless oil. Purification was carried out by flash chromatography over silica gel (12 g, irregular SiOH 25-40 μM, DCM/MeOH from 100/0 to 97/3). Pure fractions were collected and evaporated affording a white foam, 0.096 g. It was triturated with DIPE and a few Heptane, the precipitate was filtered off and dried under vacuum at 60° C. affording compound 163 as white powder, 0.088 g, 86%.

¹H NMR (500 MHz, DMSO-d₆) δ ppm 8.14 (br t, J=5.8 Hz, 1H), 7.90 (s, 1H), 7.38 (s, 1H), 7.32 (t, J=8.5 Hz, 1H), 7.20 (br d, J=13.1 Hz, 1H), 7.16 (br d, J=8.2 Hz, 1H), 4.44 (br d, J=6.0 Hz, 2H), 4.10 (br s, 2H), 3.59-3.68 (m, 2H), 2.88 (q, J=7.5 Hz, 2H), 2.42 (s, 3H), 1.22 (t, J=7.5 Hz, 3H)

Synthesis of Compound 147

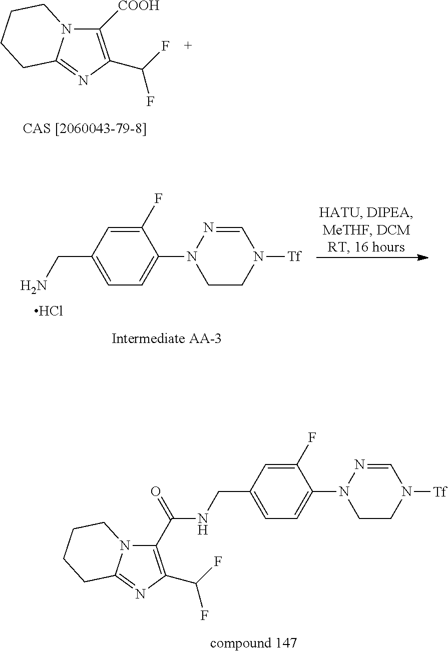

Accordingly, compound 147 was prepared in the same way as compound 163 starting from 2-(Difluoromethyl)-5H, 6H, 7H, 8H-imidazo[1,2-A]pyridine-3-carboxylic acid (CAS [2060043-79-8], 0.19 mmol) and intermediate AA-3 affording a white powder, 0.08 g (77%).

¹H NMR (400 MHz, DMSO-d₆) δ ppm 8.79 (br t, J=5.6 Hz, 1H), 7.38 (s, 1H), 7.33 (t, J=8.6 Hz, 1H), 7.07-7.23 (m, 2H), 6.95 (t, J=54.2 Hz, 1H), 4.41 (br d, J=5.9 Hz, 2H), 4.10 (br s, 2H), 4.02 (br t, J=5.5 Hz, 2H), 3.65 (br t, J=4.6 Hz, 2H), 2.68-2.91 (m, 2H), 1.89 (br d, J=4.3 Hz, 2H), 1.83 (br d, J=5.3 Hz, 2H)

Synthesis of Compound 159

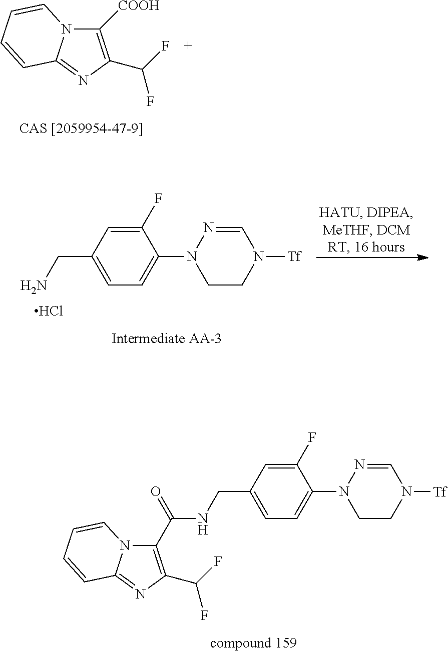

Accordingly, compound 159 was prepared in the same way as compound 163 starting from 2-(Difluoromethyl)-imidazo[1,2-A]pyridine-3-carboxylic acid (CAS [2059954-47-9], 0.19 mmol) and intermediate AA-3 affording a white powder, 0.084 g (82%).

¹H NMR (400 MHz, DMSO-d₆) δ ppm 9.00 (br s, 1H), 8.81 (br d, J=7.0 Hz, 1H), 7.77 (d, J=9.0 Hz, 1H), 7.08-7.59 (m, 7H), 4.52 (br s, 2H), 4.10 (br s, 2H), 3.66 (br t, J=4.5 Hz, 2H)

Synthesis of Compound 135

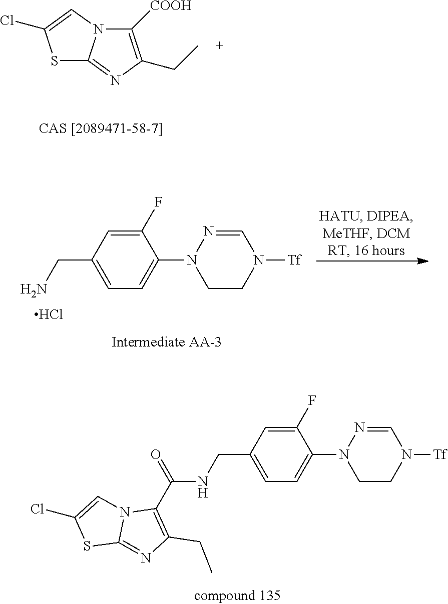

Accordingly, compound 135 was prepared in the same way as compound 163 starting from 2-Chloro-6-ethyl-2-methylimidazo[2,1-b][1,3]thiazole-5-carboxylic acid (CAS [2089471-58-7], 0.21 mmol) and intermediate AA-3 affording a white powder, 0.056 g (49%).

¹H NMR (500 MHz, DMSO-d₆) δ ppm 8.31 (m, 1H), 8.28 (br t, J=5.8 Hz, 1H), 7.38 (m, 1H), 7.33 (br t, J=8.5 Hz, 1H), 7.21 (br d, J=13.4 Hz, 1H), 7.16 (br d, J=8.2 Hz, 1H), 4.45 (br d, J=5.8 Hz, 2H), 4.10 (br s, 2H), 3.64 (br t, J=4.4 Hz, 2H), 2.89 (q, J=7.4 Hz, 2H), 1.22 (br t, J=7.5 Hz, 3H)

Synthesis of Compound 152

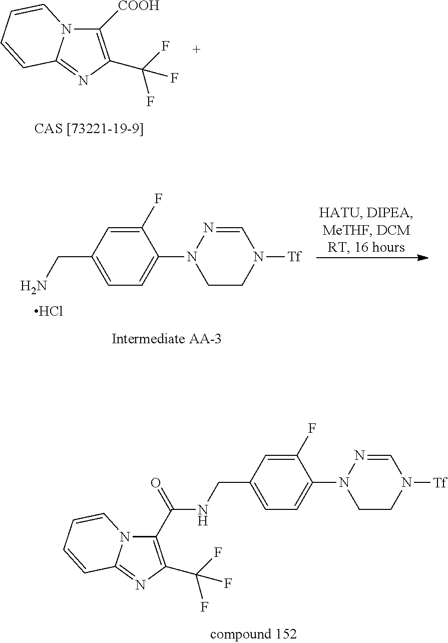

Accordingly, compound 152 was prepared in the same way as compound 163 starting from 2-(Trifluoromethyl)-imidazo[1,2-A]pyridine-3-carboxylic acid (CAS [73221-19-9], 0.92 mmol) and intermediate AA-3 affording a white powder, 0.418 g (82%).

¹H NMR (500 MHz, DMSO-d₆) δ ppm 9.29 (t, J=5.8 Hz, 1H), 8.57 (d, J=6.9 Hz, 1H), 7.80 (d, J=9.2 Hz, 1H), 7.56 (ddd, J=9.1, 6.9, 1.1 Hz, 1H), 7.39 (s, 1H), 7.36 (t, J=8.5 Hz, 1H), 7.22-7.26 (m, 1H), 7.18-7.22 (m, 2H), 4.53 (d, J=5.8 Hz, 2H), 4.11 (br t, J=4.3 Hz, 2H), 3.67 (t, J=4.7 Hz, 2H)

Synthesis of Compound 124

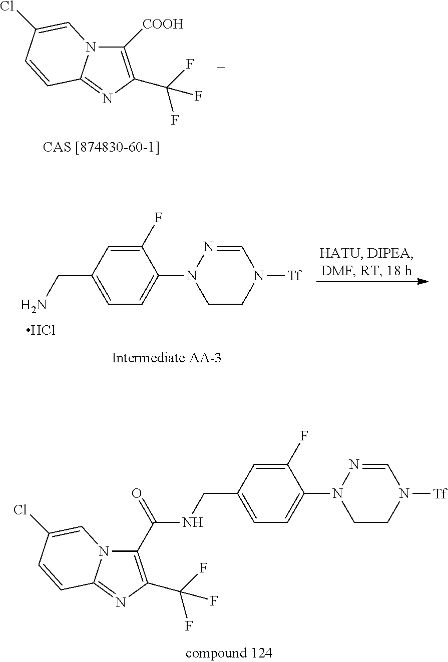

To a solution of 6-Chloro-2-(trifluoromethyl)imidazo[1,2-a]pyridine-3-carboxylic acid (CAS [874830-60-1], 100 mg, 0.378 mmol) and DIPEA (0.306 mL, 1.80 mmol) in DMF (1.7 mL) was added HATU (164 mg, 0.432 mmol). After 10 min of stirring, intermediate AA-3 (137 mg, 0.360 mmol) was added and the reaction mixture was stirred at room temperature for 18 h. The brown paste was purified by preparative LC (regular SiOH 30 μm, 25 g, dry loading (Celite®), mobile phase gradient: Heptane/EtOAc 90/10 to 30/70). The fractions containing product were combined and evaporated to give 216 mg as a yellow solid. It was triturated in Et₂O. The mixture was filtered off. The solid was rinsed with Et₂O, collected and dried under vacuum to give 172 mg as a white solid. It was dissolved in EtOAc and evaporated (3 times) to give 158 mg as a white solid. It was coevaporated with MeCN (3 times) and dried under vacuum to give 143 mg of compound 124 as a white solid (50%).

1H NMR (400 MHz, DMSO-d6) δ ppm 9.28 (br s, 1H), 8.75 (m, 1H), 7.87 (d, J=9.4 Hz, 1H), 7.65 (dd, J=9.4, 1.8 Hz, 1H), 7.31-7.41 (m, 2H), 7.15-7.30 (m, 2H), 4.54 (br d, J=4.1 Hz, 2H), 4.10 (br t, J=4.0 Hz, 2H), 3.67 (br t, J=4.6 Hz, 2H)

Synthesis of Compound 129

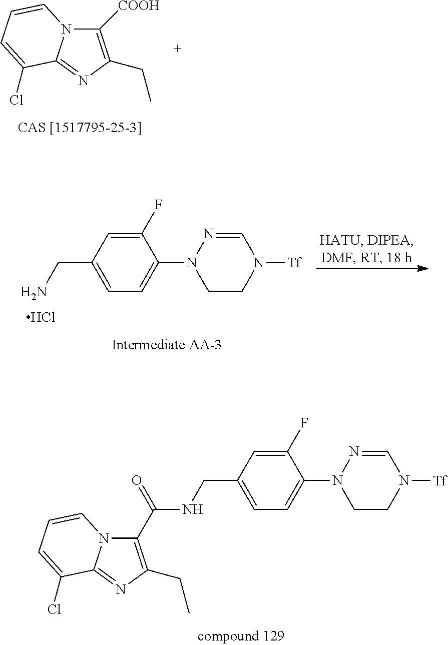

Accordingly, compound 129 was prepared in the same way as compound 124 starting from 8-chloro-2-ethylimidazo[1,2-a]pyridine-3-carboxylic acid (CAS [1517795-25-3], 0.6 mmol) and intermediate AA-3 affording 0.136 g (41%) as white powder.

¹H NMR (400 MHz, DMSO-d6) δ ppm 8.90 (br d, J=6.9 Hz, 1H), 8.59 (br t, J=5.6 Hz, 1H), 7.59 (br d, J=7.5 Hz, 1H), 7.30-7.46 (m, 2H), 7.15-7.29 (m, 2H), 7.01 (br t, J=7.1 Hz, 1H), 4.50 (d, J=5.9 Hz, 2H), 4.10 (br t, J=4.4 Hz, 2H), 3.65 (br t, J=4.9 Hz, 2H), 3.01 (q, J=7.5 Hz, 2H), 1.27 (br t, J=7.6 Hz, 3H)

Synthesis of Compound 133

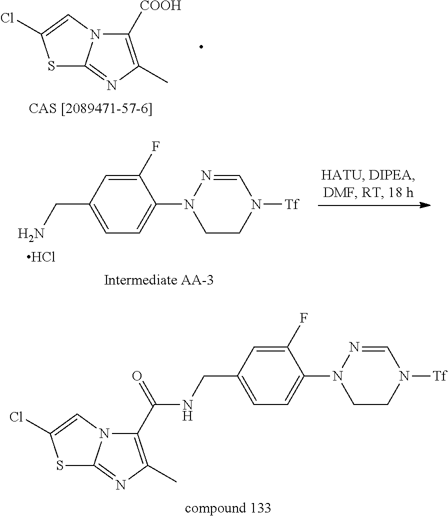

Accordingly, compound 133 was prepared in the same way as compound 124 starting from 2-chloro-6-methyl-imidazo[2,1-b]thiazole-5-carboxylic acid (CAS [2089471-57-6], 0.52 mmol) and intermediate AA-3 affording 0.142 g (51%) as white solid.

¹H NMR (400 MHz, DMSO-d6) δ ppm 8.31 (s, 1H), 8.25 (br t, J=5.9 Hz, 1H), 7.38 (br s, 1H), 7.33 (t, J=8.5 Hz, 1H), 7.14-7.25 (m, 2H), 4.45 (br d, J=5.9 Hz, 2H), 4.10 (br t, J=4.5 Hz, 2H), 3.64 (br t, J=4.8 Hz, 2H), 2.52 (s, 1H)

Synthesis of Compound 136

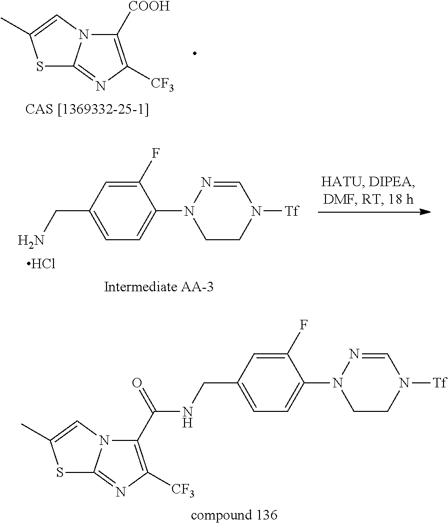

Accordingly, compound 136 was prepared in the same way as compound 124 starting from 2-Methyl-6-(trifluoromethyl)imidazo[2,1-b]thiazole-5-carboxylic acid (CAS [1369332-25-1], 0.58 mmol) and intermediate AA-3 affording 0.173 g (56%) as white powder.

¹H NMR (500 MHz, DMSO-d6) δ ppm 8.99 (br t, J=4.3 Hz, 1H), 7.86 (br s, 1H), 7.39, (m, 1H), 7.35 (br t, J=8.5 Hz, 1H), 7.14-7.24 (m, 2H), 4.47 (br d, J=5.5 Hz, 2H), 4.11 (m, 2H), 3.67 (br t, J=4.3 Hz, 2H), 2.48 (br s, 3H)

Synthesis of Compound 164

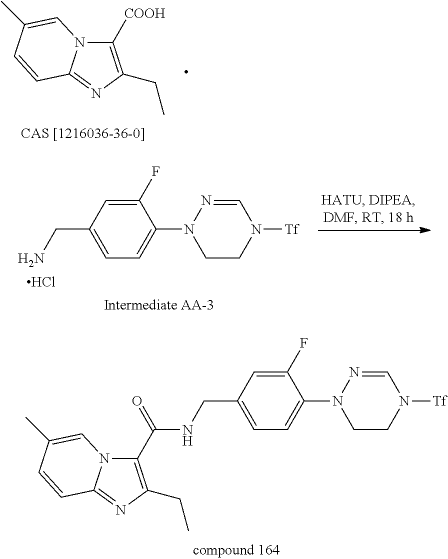

Accordingly, compound 164 was prepared in the same way as compound 124 starting from 2-ethyl-6-methylimidazo[1,2-a]pyridine-3-carboxylic acid (CAS [1216036-36-0], 0.64 mmol) and intermediate AA-3 affording 0.11 g (33%) as a white solid.

¹H NMR (400 MHz, DMSO-d6) δ ppm 8.75-8.84 (br s, 1H), 8.37 (t, J=6.0 Hz, 1H), 7.52 (d, J=8.9 Hz, 1H), 7.32-7.41 (m, 2H), 7.17-7.28 (m, 3H), 4.50 (br d, J=5.9 Hz, 2H), 4.11 (br t, J=4.2 Hz, 2H), 3.66 (t, J=4.7 Hz, 2H), 2.98 (q, J=7.5 Hz, 2H), 2.31 (s, 3H), 1.37 (t, J=7.5 Hz, 3H)

Synthesis of Compound 157

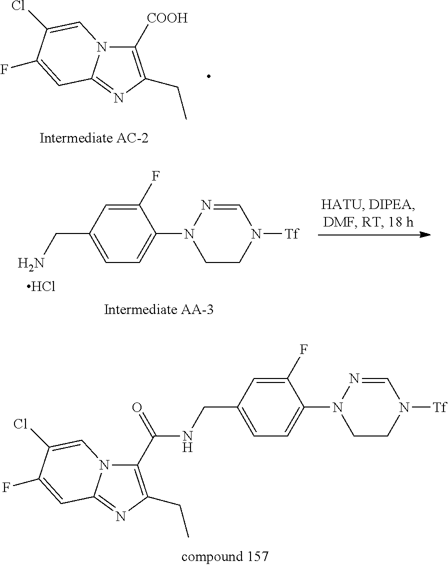

Accordingly, compound 157 was prepared in the same way as compound 124 starting from intermediate AC-2 (0.78 mmol) and intermediate AA-3 affording 0.106 g (24%) as white powder.

¹H NMR (400 MHz, DMSO-d6) δ ppm 9.23 (d, J=7.3 Hz, 1H), 8.42-8.53 (m, 1H), 7.80 (d, J=9.7 Hz, 1H), 7.29-7.40 (m, 2H), 7.17-7.28 (m, 2H), 4.50 (d, J=5.9 Hz, 2H), 4.07-4.13 (m, 2H), 3.65 (br t, J=4.6 Hz, 2H), 2.99 (q, J=7.5 Hz, 2H), 1.27 (t, J=7.5 Hz, 3H)

Synthesis of Compound 154

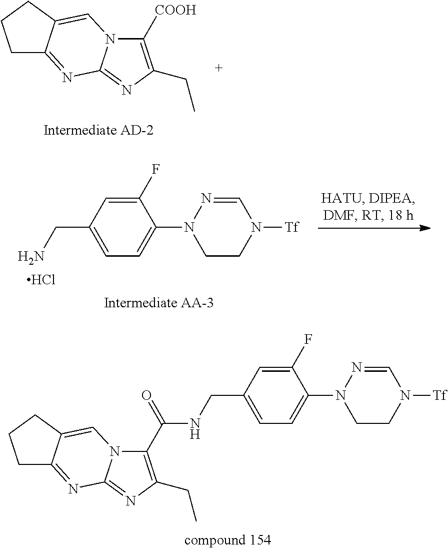

Accordingly, compound 154 was prepared in the same way as compound 124 starting from intermediate AD-2 (0.78 mmol) and intermediate AA-3 affording 0.092 g (21%) as white solid.

¹H NMR (400 MHz, DMSO-d6) δ ppm 9.23 (d, J=7.3 Hz, 1H), 8.42-8.54 (br t, J=5.9 Hz, 1H), 7.80 (d, J=9.8 Hz, 1H), 7.30-7.41 (m, 2H), 7.16-7.28 (m, 2H), 4.50 (br d, J=5.9 Hz, 2H), 4.10 (br t, J=4.9 Hz, 2H), 3.65 (br t, J=4.7 Hz, 2H), 2.99 (br q, J=7.4 Hz, 2H), 1.27 (br t, J=7.5 Hz, 3H)

Synthesis of Compound 156

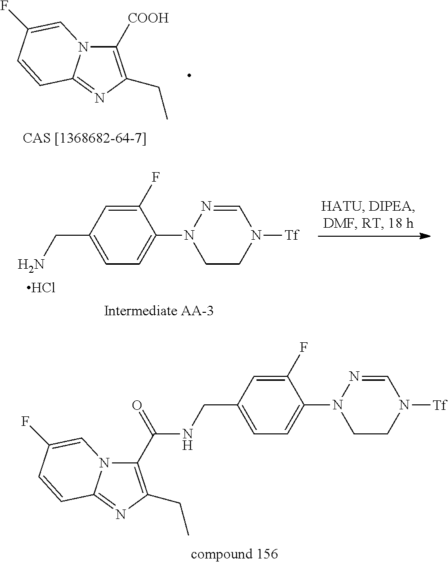

Accordingly, compound 156 was prepared in the same way as compound 124 starting from 2-ethyl-6-fluoroimidazo[1,2-a]pyridine-3-carboxylic acid (CAS [1368682-64-7], 0.27 mmol) and intermediate AA-3 affording a white solid, 0.096 g (68%).

¹H NMR (400 MHz, DMSO-d6) δ ppm 8.99-9.12 (m, 1H), 8.41 (br t, J=7.5 Hz, 1H), 7.65-7.77 (m, 1H), 7.44-7.57 (m, 1H), 7.32-7.40 (m, 2H), 7.18-7.28 (m, 2H), 4.51 (br t, J=5.9 Hz, 2H), 4.11 (br t, J=4.5 Hz, 2H), 3.66 (t, J=4.6 Hz, 2H), 3.01 (q, J=7.5 Hz, 2H), 1.28 (br t, J=7.5 Hz, 3H)

Synthesis of Compound 153

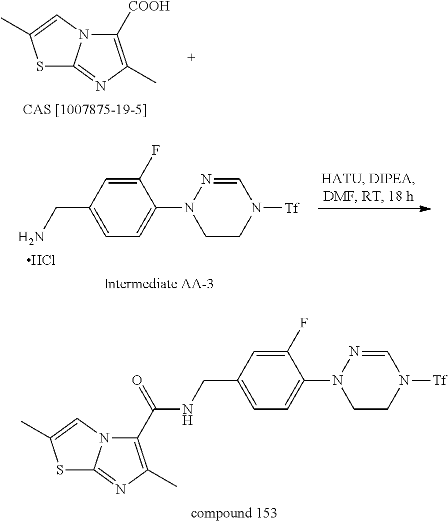

Accordingly, compound 153 was prepared in the same way as compound 124 starting from 2,6-dimethylimidazo[2,1-b][1,3]thiazole-5-carboxylic acid (CAS [1007875-19-5], 0.67 mmol) and intermediate AA-3 affording a white solid, 0.138 g (42%).

¹H NMR (500 MHz, DMSO-d6) δ ppm 8.11 (t, J=6.0 Hz, 1H), 7.84-7.95 (m, 1H), 7.38 (br s, 1H), 7.32 (br t, J=8.7 Hz, 1H), 7.14-7.23 (m, 2H), 4.45 (d, J=6.0 Hz, 2H), 4.10 (br t, J=4.4 Hz, 2H), 3.64 (br t, J=4.9 Hz, 2H), 2.51 (s, 3H), 2.41 (d, J=1.2 Hz, 3H)

Synthesis of Compound 146

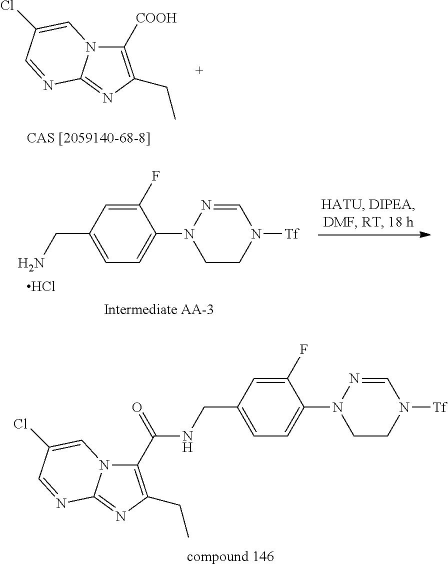

Accordingly, compound 146 was prepared in the same way as compound 124 starting from 6-chloro-2-ethyl-imidazo[1,2-a]pyrimidine-3-carboxylic acid (CAS [2059140-68-8], 0.26 mmol) and intermediate AA-3 affording a white solid, 0.154 g (74%).

¹H NMR (400 MHz, DMSO-d6) δ ppm 9.41 (d, J=2.7 Hz, 1H), 8.69 (d, J=2.7 Hz, 1H), 8.58 (m, 1H), 7.31-7.40 (m, 2H), 7.18-7.28 (m, 2H), 4.51 (m, 2H), 4.10 (br t, J=4.5 Hz, 2H), 3.65 (br t, J=4.8 Hz, 2H), 3.04 (br q, J=7.5 Hz, 2H), 1.29 (br t, J=7.5 Hz, 3H)

Synthesis of Compound 175

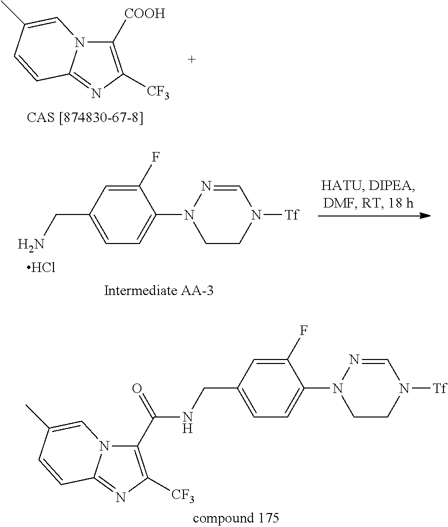

Accordingly, compound 175 was prepared in the same way as compound 124 starting from 6-methyl-2-(trifluoromethyl)imidazo[1,2-a]pyridine-3-carboxylic acid (CAS [874830-67-8], 0.53 mmol) and intermediate AA-3 affording 0.117 g (53%) as white powder.

¹H NMR (400 MHz, DMSO-d6) δ ppm 9.08 (s, 1H), 7.66 (d, J=9.2 Hz, 1H), 7.44 (t, J=8.4 Hz, 1H), 7.32 (dd, J=9.2, 1.6 Hz, 1H), 7.19 (s, 1H), 7.17-7.08 (m, 2H), 6.63 (br s, 1H), 4.64 (d, J=5.7 Hz, 2H), 4.13-4.04 (m, 2H), 3.74-3.65 (m, 2H), 2.41 (s, 3H).

Synthesis of Compound 125

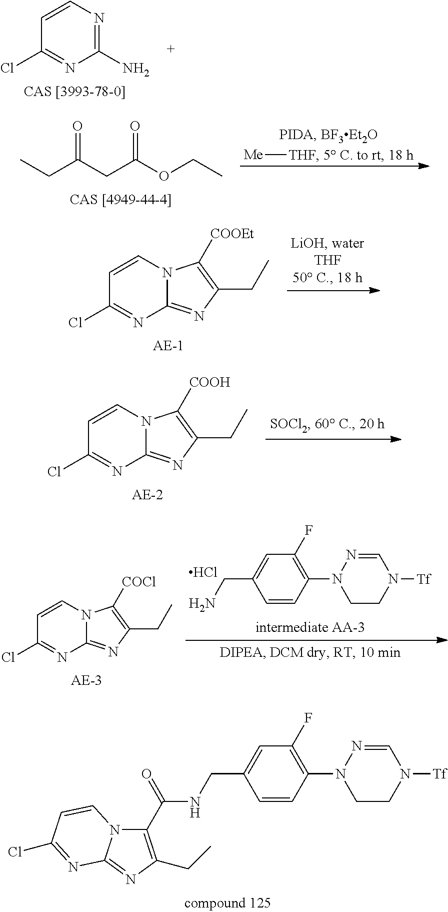

Preparation of Intermediate AE-1

Accordingly, intermediate AE-1 was prepared in the same way as intermediate AC-1 starting from 2-amino-4-chloropyrimidine (CAS [3993-78-0], 15.4 mmol) affording 0.94 g (26%).

Preparation of Intermediate AE-2

Accordingly, intermediate AE-2 was prepared in the same way as intermediate AC-2 starting from intermediate AE-1 (1.25 mmol) affording 0.26 g (92%).

Preparation of Intermediate AE-3

A mixture of intermediate AE-2 (175 mg, 0.776 mmol) in thionyl chloride (4.4 mL) was stirred at 60° C. for 20 h. The reaction mixture was evaporated to give 0.288 g as a brown paste. (The purity was calculated to give a quantitative yield).

Preparation of Compound 125

A mixture of intermediate AE-3 (288 mg, 0.779 mmol) and intermediate AA-3 (295 mg, 0.779 mmol) and DIPEA (0.331 mL, 1.95 mmol) in dry DCM (4.8 mL) was stirred at room temperature for 10 min. Water was added. The aqueous layer was extracted with DCM (once). The combined organic layers were washed with brine, dried over MgSO₄, filtered off and evaporated to give 0.4 g as a brown foam. It was purified by preparative LC (regular SiOH 30 μm, 25 g, dry loading (Celite®), mobile phase gradient: Heptane/EtOAc 90/10 to 50/50). The fraction containing products were combined and evaporated to give 0.229 g of a yellow foam. The yellow foam was sonicated in Et₂O. The precipitate was filtered off to give 146 mg of compound 125 as a white solid (33%).

¹H NMR (500 MHz, DMSO-d6) δ ppm 9.29 (d, J=7.2 Hz, 1H), 8.53-8.61 (m, 1H), 7.38 (br s, 1H), 7.34 (br t, J=8.7 Hz, 1H), 7.17-7.28 (m, 3H), 4.49 (br d, J=5.9 Hz, 2H), 4.08-4.12 (m, 2H), 3.65 (br t, J=4.9 Hz, 2H), 3.01 (br q, J=7.4 Hz, 2H), 1.27 (br t, J=7.4 Hz, 3H)

Synthesis of Compound 130

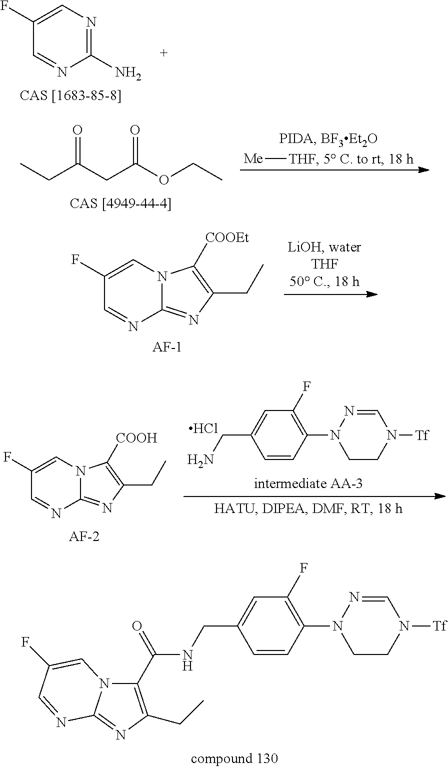

Preparation of Intermediate AF-1

Accordingly, intermediate AF-1 was prepared in the same way as intermediate AC-1 starting from 2-amino-5-fluoropyrimidine (CAS [1683-85-8], 17.68 mmol) affording 1.18 g (27%).

Preparation of Intermediate AF-2

To a solution of intermediate AF-1 (1.1 g, 4.64 mmol) in EtOH (24 mL) and water (24 mL) was added potassium carbonate (3.2 g, 23.2 mmol) and the mixture was heated at 65° C. and stirred for 3 h. (Alternative conditions re depicted in the scheme above.) The mixture was acidified to pH=1 with HCl 3M (no precipitation occurred) then evaporated in vacuo. The residue was taken up with EtOH/water (1:1), sonicated then filtered off (precipitate only contained K₂CO₃) and the filtrate was concentrated and then coevaporated twice with DCM to give 0.92 g of intermediate AF-2 as a brown solid (95%). The crude was used as such.

Preparation of Compound 130

Accordingly, compound 130 was prepared in the same way as compound 124 starting from intermediate AF-2 (0.96 mmol) and intermediate AA-3 affording a white solid, 0.194 g (39%).

¹H NMR (400 MHz, DMSO-d6) δ ppm 9.39-9.48 (m, 1H), 8.77-8.89 (m, 1H), 8.50-8.59 (m, 1H), 7.17-7.42 (m, 4H), 4.52 (br d, J=4.4 Hz, 2H), 4.07-4.13 (m, 2H), 3.62-3.68 (m, 2H), 3.05 (br q, J=7.2 Hz, 2H), 1.29 (br t, J=7.5 Hz, 3H)

Synthesis of Compound 131

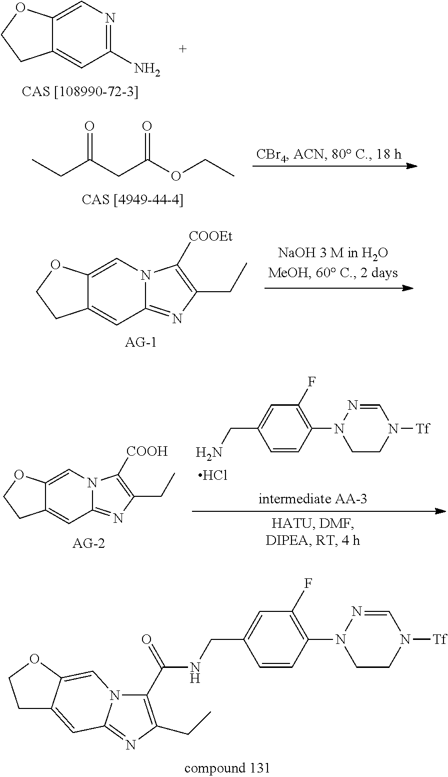

Preparation of Intermediate AG-1

To a solution of 2H, 3H-furo[2,3-c]pyridin-5-amine (CAS [1785357-12-1], 500 mg, 3.67 mmol) in ACN (8.4 mL) were added ethyloxovalerate (1.05 mL, 7.35 mmol) and boron tetrabromide (2.44 g, 7.35 mmol) and the reaction mixture was stirred at 80° C. for 18 h. The reaction mixture was diluted with EtOAc and the organic layer was washed with water and brine, dried over MgSO₄, filtered off, concentrated and purified by preparative LC (irregular SiOH, 15-40 μm, 40 g, liquid loading (DCM), mobile phase gradient: from Heptane/EtOAc: 100/0 to 0/100 in 10 CV then EtOAc 100% for 5 CV). The fractions containing product were combined and evaporated to give 0.21 g of intermediate AG-1 (22%).

Preparation of Intermediate AG-2

A mixture of intermediate AG-1 (186 mg, 0.715 mmol), aqueous NaOH 3M (1.19 mL, 3.57 mmol) and MeOH (2 mL) was stirred 60° C. for 2 days. The mixture was evaporated to give 0.33 g of intermediate AG-2 (purity was estimated to give a quantitative yield).

Preparation of Compound 131

Accordingly, compound 131 was prepared in the same way as compound 124 starting from intermediate AG-2 (0.71 mmol) and intermediate AA-3 affording a white solid, 0.09 g (23%).

¹H NMR (400 MHz, DMSO-d6) δ ppm 8.50 (s, 1H), 8.19-8.32 (m, 1H), 7.47 (s, 1H), 7.38 (br s, 1H), 7.29-7.36 (m, 1H), 7.14-7.25 (m, 2H), 4.61 (t, J=8.2 Hz, 2H), 4.47 (br d, J=5.7 Hz, 2H), 4.09 (br t, J=4.3 Hz, 2H), 3.65 (t, J=4.7 Hz, 2H), 3.25-3.32 (m, 2H), 2.94 (q, J=7.5 Hz, 2H), 1.24 (t, J=7.5 Hz, 3H)

Synthesis of Compound 134

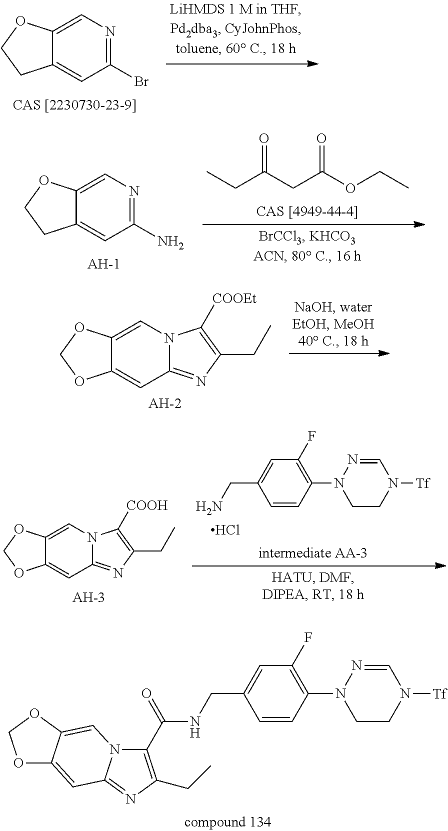

Preparation of Intermediate AH-1

A solution of 6-bromo-1,3-Dioxolo[4,5-c]-pyridine (CAS [2230730-23-9], 3.87 g, 19.2 mmol) in dry toluene (100 mL) was c with N₂ (3 times). Pd₂(dba)₃ (1.75 g, 1.92 mmol) and CyJohnPhos (2.80 g, 7.66 mmol) were added and the reaction mixture was degassed with N₂ (3 times). LiHMDS (1.0M in THF) (23 mL, 23 mmol) was then added dropwise at room temperature and the reaction mixture was stirred at 60° C. for 18 h. The reaction mixture was diluted in EtOAc, water and acidified with an aqueous solution of HCl (1N). The aqueous layer was extracted with EtOAc (twice). The aqueous layer was then basified with a solution of NaOH (3M) and extracted with EtOAc (3 times). The combined organic layers were dried over MgSO₄, filtered off and evaporated to give 1.84 g of intermediate AH-1 as a brown solid (70%).

Preparation of Intermediate AH-2

Accordingly, intermediate AH-2 was prepared in the same way as intermediate AB-1 starting from intermediate AH-1 (3.62 mmol) affording 0.165 g (17%).

Preparation of Intermediate AH-3

Accordingly, intermediate AH-3 was prepared in the same way as intermediate AB-2 starting from intermediate AH-2 (0.95 mmol) affording 0.421 g (purity was estimated to give a quantitative yield).

Preparation of Compound 134

Accordingly, compound 134 was prepared in the same way as compound 124 starting from intermediate AH-3 (0.45 mmol) and intermediate AA-3 affording a white solid, 0.194 g (84%).

¹H NMR (400 MHz, DMSO-d6) δ ppm 8.62 (br s, 1H), 8.24 (t, J=6.0 Hz, 1H), 7.38 (s, 1H), 7.34 (t, J=8.6 Hz, 1H), 7.14-7.24 (m, 2H), 7.08 (s, 1H), 6.16 (br s, 2H), 4.47 (br d, J=5.8 Hz, 2H), 4.07-4.12 (m, 2H), 3.65 (br t, J=4.6 Hz, 2H), 2.91 (q, J=7.5 Hz, 2H), 1.23 (t, J=7.5 Hz, 3H)

Synthesis of Compound 161

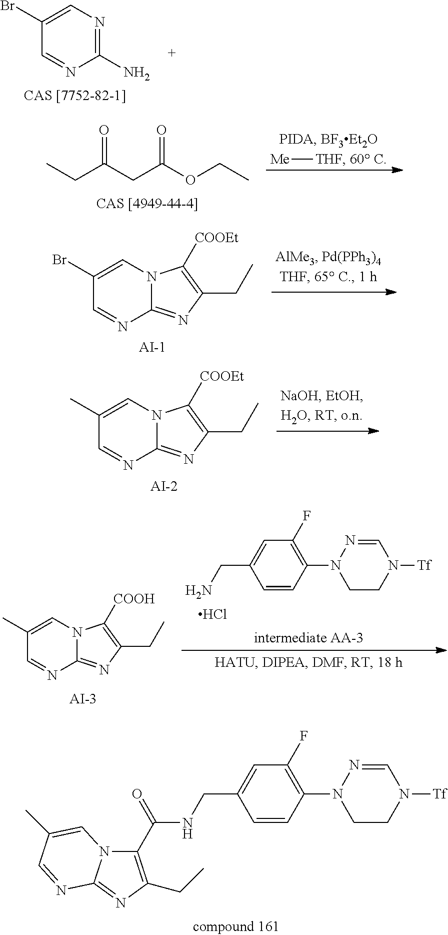

Preparation of Intermediate AI-1

2-amino-5-bromopyrimidine (10.0 g; 57.5 mmol) was suspended in dry 2-MeTHF (250 mL). ethyl 3-oxovalerate (8.2 mL, 57.5 mmol, 1 eq.) and iodobenzene diacetate (18.5 g, 57.5 mmol, 1 eq.) were added. boron trifluoride etherate (0.75 mL, 2.87 mmol, 0.05 eq.) was then added dropwise and the reaction mixture was stirred at 60° C. for 1.5 hours. An extra amount of ethyl ethyl 3-oxovalerate (4.10 mL, 28.7 mmol, 0.5 eq.), iodobenzene diacetate (9.25 g, 28.7 mmol, 0.5 eq.) and boron trifluoride etherate (0.75 mL, 2.87 mmol, 0.05 eq.) were added at room temperature and the mixture was stirred at 60° C. for 1h. The mixture was cooled down to room temperature then EtOAc and water were added. The organic layer was separated and washed with a saturated solution of NaHCO₃ (twice), then with brine (twice). The organic layer was dried over MgSO₄, filtered off and concentrated to give 19.7 g as a brown oil. The crude was purified by preparative LC (irregular SiOH, 15-40 μm, 330 g, dry loading (SiOH), mobile phase gradient: from DCM 100% to DCM 85%, EtOAc 15%) to give intermediate AI-1, 9.03 g as yellow crystals (53%).

Preparation of Intermediate AI-2

In a sealed tube under N₂, to a solution of intermediate AI-1 (500 mg, 1.68 mmol) and Pd(PPh₃)₄ (96.9 mg, 0.084 mmol) in THE (12 mL) degassed under N2 was added trimethylaluminum 2m in Hexanes (2 eq., 1.68 mL, 3.35 mmol). The mixture was purged again with N2 and was heated at 65° C. for 1 h. An extra amount of trimethylaluminum 2m in Hexanes (1 eq., 0.839 mL, 1.68 mmol) was added and the mixture was stirred at 65° C. for 1 h. The mixture was diluted with DCM, cooled down to 0° C. and 1 mL of water was added carefully. The mixture was stirred at room temperature overnight then MgSO₄ was added. After 30 min under stirring, the mixture was filtered over a plug of Celite® and evaporated to give 412 mg of as an orange gum. The crude was purified by preparative LC (regular SiOH, 30 μm, 40 g, dry loading (Celite®), mobile phase eluent: Heptane 95%, EtOAc 5% to Heptane 50%, EtOAc 50%). Fractions containing product were combined and concentrated to obtain intermediate AI-2, 354 mg of as a yellow gum (90%).

Preparation of Intermediate AI-3

To a solution of intermediate AI-2 (120 mg, 0.514 mmol) in water (1 mL) and EtOH (4 mL) was added NaOH (62 mg, 1.55 mmol) and the mixture was stirred at room temperature overnight. The mixture was evaporated then co-evaporated with EtOH to give intermediate AI-3, 190 mg as a yellow solid. The crude was used as such in next step.

Preparation of Compound 161

A mixture of intermediate AI-3 (190 mg, 0.518 mmol), HATU (280 mg, 0.736 mmol), DIPEA (0.163 mL, 0.958 mmol) and DMF (2.5 mL) was stirred at room temperature for 15 min then intermediate AA-3 (180 mg, 0.473 mmol) was added and stirring was continued over 3 days. DMF was evaporated. The residue was taken-up in DCM and water then washed with a saturated aqueous solution of NaHCO₃ (twice), brine (twice), dried over MgSO₄, filtered off and concentrated. The crude (m=378 mg) was purified by preparative LC (regular SiOH, 30 μm, 24 g, mobile phase gradient: from Heptane 85%, EtOAc/MeOH (9:1) 15% to Heptane 25%, EtOAc/MeOH (9:1) 75). Fractions containing product were combined and concentrated to afford 277 mg as a white solid. The solid was recrystallized from EtOAc, filtered off and dried under high vacuum to afford 162 mg of compound 161 as a white solid (54%).

¹H NMR (400 MHz, DMSO-d6) δ ppm 9.15 (d, J=1.2 Hz, 1H), 8.52 (br d, J=2.3 Hz, 1H), 8.44-8.49 (m, 1H), 7.38 (br s, 1H), 7.34 (m, J=8.6 Hz, 1H), 7.17-7.27 (m, 2H), 4.50 (br d, J=5.9 Hz, 2H), 4.07-4.13 (m, 2H), 3.65 (br t, J=4.6 Hz, 2H), 3.01 (q, J=7.5 Hz, 2H), 2.34 (br s, 3H), 1.28 (t, J=7.5 Hz, 3H)

Synthesis of Compounds 162, 148 & 151

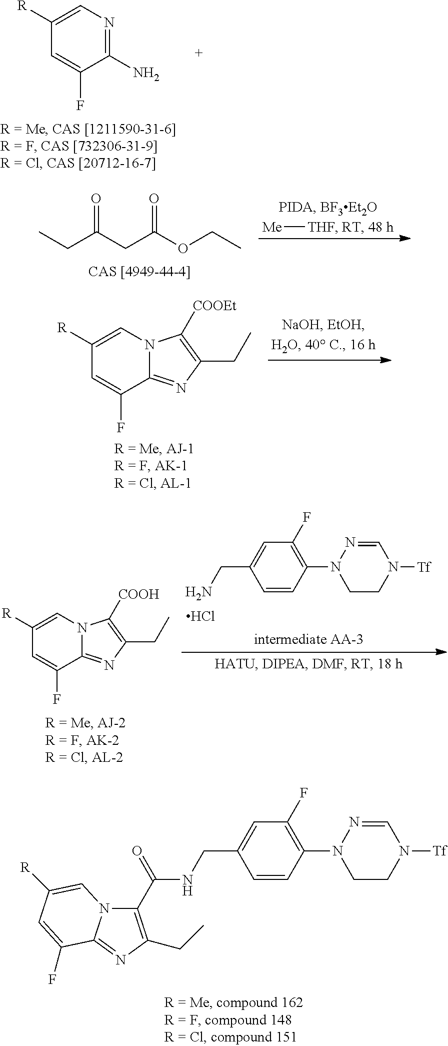

Preparation of Intermediate AJ-1

The reaction was performed in anhydrous conditions under nitrogen atmosphere.

To a solution of 3-Fluoro-5-methylpyridin-2-amine (2.00 g, 15.9 mmol) in 2-MeTHF (60 mL) at 5° C. under N₂ were added Ethyl propionylacetate (3.60 mL, 24.8 mmol), Iodobenze diacetate (7.80 g, 24.2 mmol) and Boron trifluoride diethyl etherate (200 μL, 1.62 mmol). The reaction was stirred 1 h at 5° C. then at room temperature for 48 h. EtOAc (200 mL) and water (200 mL) were added. The layers were separated, and the organic layer was washed with a saturated aqueous solution of NaHCO₃ (200 mL), brine (2×100 mL), dried over Na₂SO₄, filtered and evaporated to afford 4.92 g as a brown paste. The crude was purified via preparative LC (SiOH, 120 g, 50 μm, Eluent: Cyclohexane/EtOAc, from 95:05 to 50:5), fractions containing product were collected, evaporated and triturated with pentane (2×20 mL) to afford 1.68 g of intermediate AJ-1 as a white solid (42%).

Preparation of Intermediate AJ-2

To a solution of intermediate AJ-1 (500 mg, 2.00 mmol) in water (12.5 mL) and EtOH (12.5 mL) was added NaOH (275 mg, 6.880 mmol). The reaction mixture was stirred for 16 h at 40° C. The crude was washed with DCM (30 mL) and with EtOAc (30 mL), the aqueous phase was acidified with an aqueous solution of HCl (3N) until pH=2. The formed precipitate was recuperated using a sintered glass under vacuum, washed with water (2×2 mL) and dried in a vacuum chamber at 50° C. overnight to afford 415 mg of intermediate AJ-2 as an off-white solid (93%).

Preparation of Compound 162

Accordingly, compound 162 was prepared in the same way as compound 161 starting from intermediate AJ-2 (0.36 mmol) and intermediate AA-3 affording 0.113 g (48%) as white solid.

¹H NMR (400 MHz, DMSO-d6) δ ppm 8.61 (br s, 1H), 8.53 (br t, J=5.9 Hz, 1H), 7.31-7.40 (m, 2H), 7.17-7.27 (m, 3H), 4.50 (d, J=5.9 Hz, 2H), 4.10 (br t, J=4.5 Hz, 2H), 3.65 (br t, J=4.5 Hz, 2H), 2.98 (q, J=7.5 Hz, 2H), 2.31 (s, 3H), 1.26 (t, J=7.5 Hz, 3H)

Preparation of Intermediate AK-1

Accordingly, intermediate AK-1 was prepared in the same way as intermediate AJ-1 starting from 2-Amino-3,5-difluoropyridine (CAS [732306-31-9], 15.37 mmol) affording 0.89 g (23%) as white solid.

Preparation of Intermediate AK-2

Accordingly, intermediate AK-2 was prepared in the same way as intermediate AJ-2 starting from intermediate AK-1 (1.97 mmol) giving 0.345 g (78%).

Preparation of Compound 148

Accordingly, compound 148 was prepared in the same way as compound 161 starting from intermediate AK-2 (0.35 mmol) and intermediate AA-3 affording 0.189 g (82%) as white solid.

¹H NMR (500 MHz, DMSO-d6) δ ppm 8.92 (dd, J=4.7, 1.8 Hz, 1H), 8.58 (t, J=5.9 Hz, 1H), 7.64-7.74 (m, 1H), 7.38 (br s, 1H), 7.35 (t, J=8.5 Hz, 1H), 7.18-7.27 (m, 2H), 4.50 (d, J=5.9 Hz, 2H), 4.10 (br t, J=4.7 Hz, 2H), 3.65 (t, J=4.9 Hz, 2H), 3.01 (q, J=7.5 Hz, 2H), 1.27 (t, J=7.6 Hz, 3H)

Preparation of Intermediate AL-1

Accordingly, intermediate AL-1 was prepared in the same way as intermediate AJ-1 starting from 2-Amino-5-chloro-3-fluoropyridine (CAS [20712-16-7], 17.06 mmol) affording 0.52 g (11%) as white solid.

Preparation of Intermediate AL-2

Accordingly, intermediate AL-2 was prepared in the same way as intermediate AJ-2 starting from intermediate AL-1 (1.77 mmol) giving 0.26 g (60%).

Preparation of Compound 151

Accordingly, compound 151 was prepared in the same way as compound 161 starting from intermediate AL-2 (0.43 mmol) and intermediate AA-3 affording 0.104 g (38%) as white solid.

¹H NMR (400 MHz, DMSO-d6) δ ppm 8.92 (d, J=1.0 Hz, 1H), 8.58-8.67 (m, 1H), 7.63 (dd, J=10.6, 1.4 Hz, 1H), 7.31-7.40 (m, 2H), 7.17-7.28 (m, 2H), 4.51 (br d, J=5.6 Hz, 2H), 4.07-4.13 (m, 2H), 3.65 (t, J=4.6 Hz, 2H), 3.01 (q, J=7.4 Hz, 2H), 1.27 (t, J=7.4 Hz, 3H)

Synthesis of Compounds 145 & 144

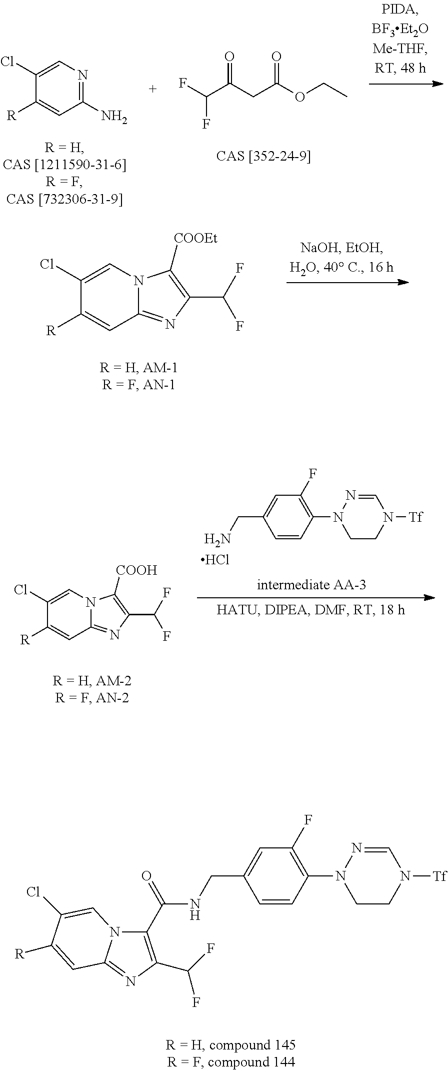

Preparation of Intermediate AM-1

Accordingly, intermediate AM-1 was prepared in the same way as AJ-1 starting from 2-amino-5-chloropyridine (CAS [1072-98-6], 3.89 mmol) and Ethyl 4,4-difluoro-3-oxobutyrate (CAS [352-24-9]) giving 0.248 g (23%) as white solid.

Preparation of Intermediate AM-2

Accordingly, intermediate AM-2 was prepared in the same way as intermediate AJ-2 starting from intermediate AM-1 (0.73 mmol) giving 0.175 g (96%).

Preparation of Compound 145

Accordingly, compound 145 was prepared in the same way as compound 161 starting from intermediate AM-2 (0.39 mmol) and intermediate AA-3 affording 0.164 g (64%) as white solid.

¹H NMR (500 MHz, DMSO-d6) δ ppm 9.04 (s, 1H), 8.88-8.96 (m, 1H), 7.83 (dd, J=9.6, 1 Hz, 1H), 7.61 (dd, J=9.6, 2.1 Hz, 1H), 7.46-7.47 (m, 1H), 7.33-7.40 (m, 2H), 7.19-7.30 (m, 2H), 4.51-4.54 (m, 2H), 4.08-4.12 (m, 2H), 3.66 (br t, J=4.9 Hz, 2H)

Preparation of Intermediate AN-1

Accordingly, intermediate AN-1 was prepared in the same way as AJ-1 starting from 5-Chloro-4-fluoropyridin-2-amine (CAS [1393574-54-3], 6.82 mmol) and Ethyl 4,4-difluoro-3-oxobutyrate (CAS [352-24-9]) giving 0.57 g (28%) as white solid.

Preparation of Intermediate AN-2

Accordingly, intermediate AN-2 was prepared in the same way as intermediate AJ-2 starting from intermediate AN-1 (0.85 mmol) giving 0.145 g (64%).

Preparation of Compound 144

Accordingly, compound 144 was prepared in the same way as compound 161 starting from intermediate AM-2 (0.41 mmol) and intermediate AA-3 affording 0.204 g (72%) as white solid.

¹H NMR (500 MHz, DMSO-d6) δ ppm 9.09 (d, J=7.2 Hz, 1H), 9.03-9.07 (m, 1H), 7.98 (d, J=9.6 Hz 1H), 7.20-7.40 (m, 4H), 4.52 (br d, J=4.6 Hz, 2H), 4.09-4.13 (m, 2H), 3.65-3.68 (m, 2H), 2.53 (br s, 1H)

Synthesis of Compound 138, 139 & 140 and compound 143

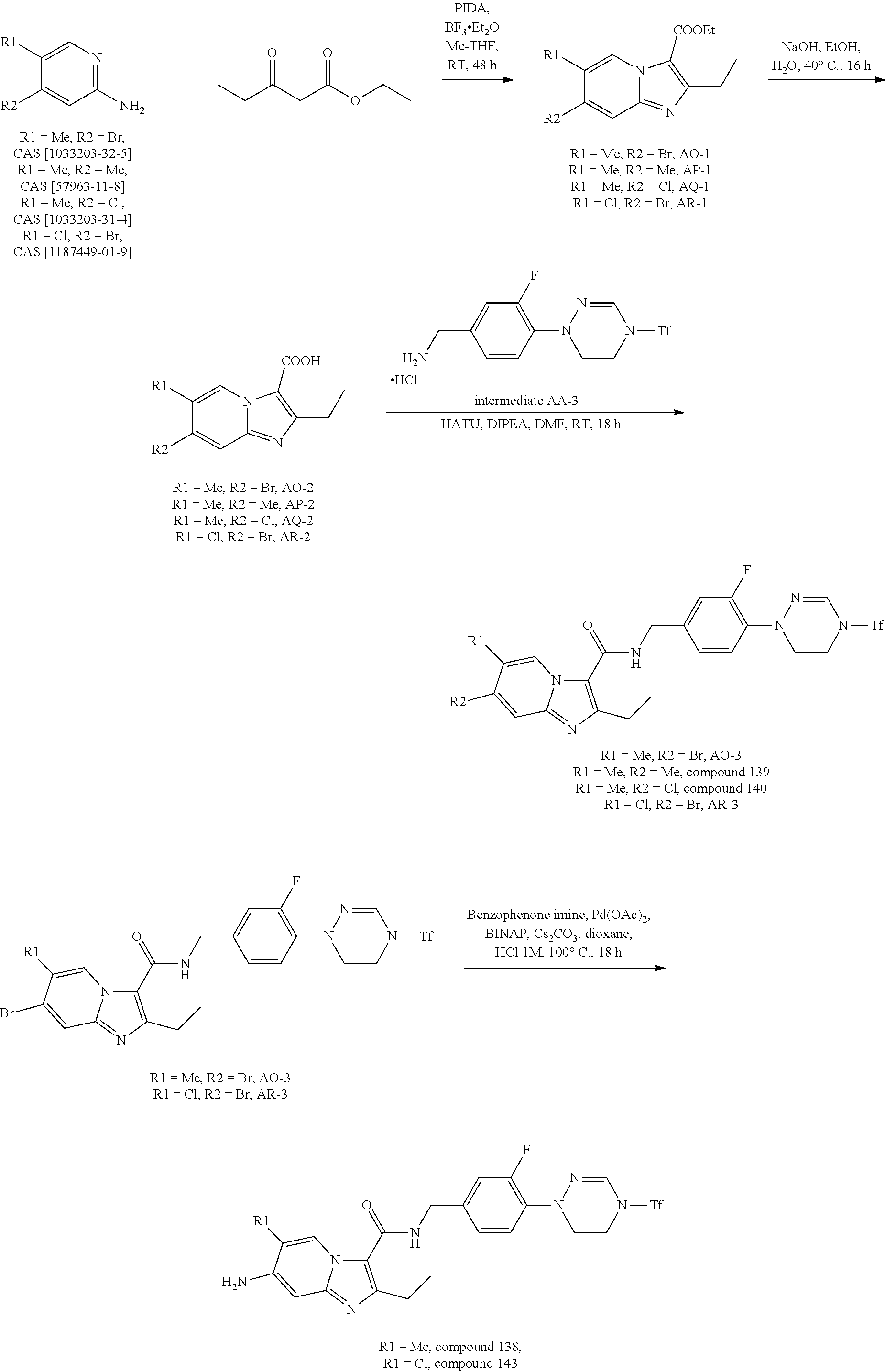

Preparation of Intermediate AO-1

Accordingly, intermediate AO-1 was prepared in the same way as AJ-1 starting from 4-bromo-5-methylpyridin-2-amine (CAS [1033203-32-5], 5.35 mmol) and ethyl 3-oxovalerate (CAS [4949-44-4]) giving 0.88 g (50%) as white solid.

Preparation of Intermediate AO-2

Accordingly, intermediate AO-2 was prepared in the same way as intermediate AJ-2 starting from intermediate AO-1 (0.48 mmol) giving 0.205 g (78%).

Preparation of Intermediate AO-3

Accordingly, intermediate AO-3 was prepared in the same way as compound 161 starting from intermediate AO-2 (0.49 mmol) and intermediate AA-3 affording 0.27 g (71%) as white solid.

Preparation of Compound 138

A mixture of intermediate AO-3 (210 mg, 0.347 mmol), benzophenone imine (116 μL, 0.694 mmol), cesium carbonate (226 mg, 0.694 mmol) and 1,4-dioxane (1.75 mL) was purged with N₂, Pd(OAc)₂ (3.9 mg, 0.017 mmol) and BINAP (21.6 mg, 0.0347 mmol) were added. The mixture was purged with N₂ and stirred at 100° C. for 18 h. The mixture was filtered over a pad of Celite® and the cake was washed with EtOAc. The organic layer was concentrated then the residue was stirred in 1,4-dioxane (2.5 ml) and aqueous HCl 1M (2.5 mL) at room temperature for 16 h. The mixture was diluted with EtOAc and slowly quenched with a saturated aqueous solution of NaHCO₃. The layers were separated, and the aqueous layer was extracted with EtOAc (twice). The organic layers were combined, dried over MgSO₄, filtered off and evaporated. The residue was purified by preparative LC (regular SiOH, 30 μm, 24 g, mobile phase eluent: from Heptane 90%, EtOAc/MeOH/aq. NH₃ (90:9.5:0.5) 10% to Heptane 20%, EtOAc/MeOH/aq. NH₃ (90:9.5:0.5) 80%). Fractions containing product were combined and concentrated to obtain 0.125 g as a white solid. This solid was recrystallized from EtOAc, filtered off and dried under high vacuum to obtain 97 mg of compound 138 as a white solid (52%).

¹H NMR (400 MHz, DMSO-d6) δ ppm 8.61-8.70 (m, 1H), 7.89 (t, J=6.0 Hz, 1H), 7.38 (s, 1H), 7.32 (t, J=8.5 Hz, 1H), 7.14-7.22 (m, 2H), 6.46-6.47 (m, 1H), 5.69-5.72 (m, 2H), 4.44 (br d, J=5.8 Hz, 2H), 4.10 (br t, J=4.3 Hz, 2H), 3.64 (t, J=4.6 Hz, 2H), 2.87 (q, J=7.5 Hz, 2H), 2.08 (s, 3H), 1.21 (t, J=7.5 Hz, 3H)

Preparation of Intermediate AP-1

Accordingly, intermediate AP-1 was prepared in the same way as AJ-1 starting from 4,5-dimethylpyridin-2-amine (CAS [57963-11-8], 4.09 mmol) and ethyl 3-oxovalerate (CAS [4949-44-4]) giving 0.73 g (72%) as white solid.

Preparation of Intermediate AP-2

Accordingly, intermediate AP-2 was prepared in the same way as intermediate AJ-2 starting from intermediate AP-1 (0.81 mmol) giving 0.3 g (quantitative).

Preparation of Compound 139

Accordingly, compound 139 was prepared in the same way as compound 161 starting from intermediate AP-2 (0.49 mmol) and intermediate AA-3 affording 0.142 g (58%) as white solid.

¹H NMR (500 MHz, DMSO-d6) δ ppm 8.78 (br s, 1H), 8.24 (t, J=5.9 Hz, 1H), 7.38 (s, 2H), 7.34 (t, J=8.5 Hz, 1H), 7.16-7.25 (m, 2H), 4.48 (d, J=5.9 Hz, 2H), 4.10 (br t, J=4.7 Hz, 2H), 3.65 (t, J=4.5 Hz, 2H), 2.95 (q, J=7.5 Hz, 2H), 2.30 (s, 3H), 2.22 (s, 3H), 1.25 (t, J=7.5 Hz, 3H)

Preparation of Intermediate AQ-1

Accordingly, intermediate AQ-1 was prepared in the same way as AJ-1 starting from 4-chloro-5-methylpyridin-2-amine (CAS [1033203-31-4], 7.01 mmol) and ethyl 3-oxovalerate (CAS [4949-44-4]) giving 0.39 g (20%) as white solid.

Preparation of Intermediate AQ-2

Accordingly, intermediate AQ-2 was prepared in the same way as intermediate AJ-2 starting from intermediate AQ-1 (0.45 mmol) giving 0.15 g (quantitative).

Preparation of Compound 140

Accordingly, compound 140 was prepared in the same way as compound 161 starting from intermediate AQ-2 (0.45 mmol) and intermediate AA-3 affording 0.23 g (68%) as white powder.

¹H NMR (500 MHz, DMSO-d6) δ ppm 8.95 (s, 1H), 8.45 (br t, J=5.9 Hz, 1H), 7.81 (br s, 1H), 7.38 (br s, 1H), 7.34 (t, J=8.5 Hz, 1H), 7.17-7.26 (m, 2H), 4.50 (d, J=5.9 Hz, 2H), 4.10 (br t, J=4.4 Hz, 2H), 3.65 (t, J=4.7 Hz, 2H), 2.97 (q, J=7.3 Hz, 2H), 2.32 (s, 3H), 1.26 (t, J=7.4 Hz, 3H)

Preparation of Intermediate AR-1

Accordingly, intermediate AR-1 was prepared in the same way as AJ-1 starting from 4-bromo-5-chloropyridin-2-amine (CAS [1187449-01-9], 9.64 mmol) and ethyl 3-oxovalerate (CAS [4949-44-4]) giving 0.655 g (21%).

Preparation of Intermediate AR-2

Accordingly, intermediate AR-2 was prepared in the same way as intermediate AJ-2 starting from intermediate AR-1 (2.05 mmol) giving 0.94 g (quantitative).

Preparation of Intermediate AR-3

Accordingly, intermediate AR-3 was prepared in the same way as compound 161 starting from intermediate AR-2 (2.06 mmol) and intermediate AA-3 affording 0.42 g (33%) as an off-white solid.

Preparation of Compound 143

Accordingly, compound 143 was prepared in the same way as compound 138 starting from intermediate AR-3 (0.4 mmol) giving 0.08 g (33%) as white solid.

¹H NMR (400 MHz, DMSO-d6) δ ppm 9.03 (s, 1H), 8.01 (t, J=5.7 Hz, 1H), 7.38 (s, 1H), 7.33 (t, J=8.6 Hz, 1H), 7.15-7.24 (m, 2H), 6.63 (br s, 1H), 6.12 (br s, 2H), 4.45 (d, J=5.9 Hz, 2H), 4.07-4.12 (m, 2H), 3.64 (t, J=4.5 Hz, 2H), 2.90 (q, J=7.5 Hz, 2H), 1.22 (t, J=7.5 Hz, 3H)

Synthesis of Compound 126

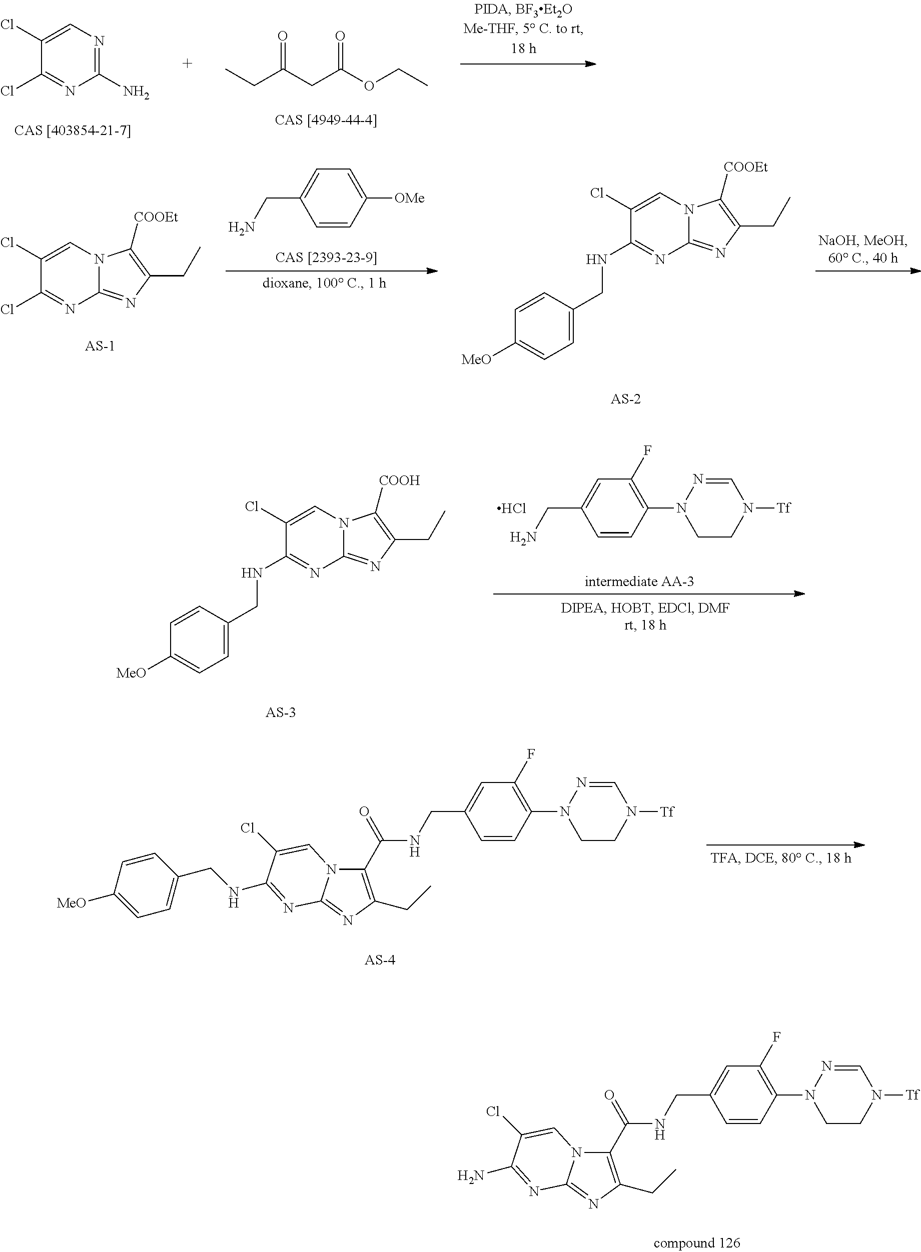

Preparation of Intermediate AS-1

To a solution of 4,5-dichloropyrimidin-2-amine (CAS [403854-21-7], 12.5 g, 76.2 mmol) in Me-THF (315 mL) at 0° C. were added iodobenzene diacetate (73.7 g, 229 mmol) and ethyl 3-oxovalerate (16.5 mL, 116 mmol). Then boron trifluoride etherate (1.92 mL, 15.2 mmol) was added dropwise. The mixture was stirred at 5° C. for 1 h and then at room temperature for 16 h. Extra boron trifluoride etherate (1.92 mL, 15.2 mmol) was added dropwise and the reaction mixture was stirred at room temperature for 28 h. EtOAc and water were added. The organic layer was washed with brine, dried over MgSO₄ and evaporated to give a brown oil. The oil was purified by preparative LC (irregular SiOH, 15-40 μm, 330 g, gradient: Heptane 100% to heptane/EtOAc 75/25). The fractions containing product were combined and evaporated to give a yellow mixture which was triturated in pentane. The supernatant was removed by pipette and the residue was dried under vacuum to give 1.16 g of intermediate AS-1 as a white solid (5%). The supernatant was evaporated to give a yellow mixture. The supernatant was removed by pipette to give 5.02 g of intermediate AS-1 as a yellow paste (32%).

Preparation of Intermediate AS-2

A mixture of intermediate AS-1 (5.02 g, 5.58 mmol, purity 32%), 4-methoxybenzylamine (CAS [2393-23-9], 2.19 mL, 16.7 mmol) and 1,4-dioxane (16 mL) was stirred at 100° C. for 1 h. The mixture was evaporated and purified by preparative LC (irregular SiOH, 15-40 μm, 120 g, dry loading (Celite®), mobile phase gradient: from Heptane/EtOAc: 70/30 to 30/70). The fractions containing product were combined and evaporated to give 1.6 g of intermediate AS-2 (74%).

Preparation of Intermediate AS-3

A mixture of intermediate AS-2 (0.900 g, 2.31 mmol), NaOH (278 mg, 6.94 mmol) and MeOH (9.2 mL) was stirred at 60° C. for 40 h. The mixture was evaporated to give 1.05 g of intermediate AS-3 (quantitative).

Preparation of Intermediate AS-4

A mixture of intermediate AS-3 (1.05 g, 2.30 mmol, purity 84%), EDCI·HCl (0.8783 g, 4.61 mmol), HOBT·H₂0 (0.706 mg, 4.61 mmol), DIPEA (1.19 ml, 6.91 mmol) and DMF (35 mL) was stirred at 50° C. for 30 min. Intermediate AA-3 (865 mg, 2.42 mmol) was added and the mixture was stirred at room temperature for 18 h. The reaction mixture was diluted with EtOAc and the organic layer was washed with water and brine, dried over MgSO₄, filtered off, concentrated and purified by preparative LC (irregular SiOH, 15-40 μm, 120 g, mobile phase gradient: from heptane/EtOAc 50/50 to 0/100). The fractions containing product were combined and evaporated to give 560 mg of intermediate AS-4 (36%).

Preparation of Compound 126

A mixture of intermediate AS-4 (560 mg, 0.820 mmol), TFA (4.5 mL) and DCE (4.5 mL) was stirred at 80° C. for 20 h. The mixture was evaporated and purified by preparative LC (spherical C18 25 μm, 120 g YMC-ODS-25, liquid loading (DMSO), mobile phase gradient 0.2% aq. NH₄⁺HCO₃⁻/MeCN from 75:25 to 20:80). The fractions containing product were evaporated to give 204 mg as white solid. and 350 mg of impure desired product. This second fraction was purified by preparative LC (spherical C18 25 μm, 120 g YMC-ODS-25, liquid loading (DMSO), mobile phase gradient 0.2% aq. NH₄⁺HCO₃⁻/MeCN from 75:25 to 20:80). The fractions containing product were evaporated to give 65 mg as white solid. Fractions of pure compounds were solubilized with EtOAc at reflux. The mixture was slowly cooled to room temperature with a slow stirring. The precipitate was filtered to give 0.355 g of compound 126 as a white solid (93%).

¹H NMR (500 MHz, DMSO-d6) δ ppm 9.06 (s, 1H), 8.12 (t, J=6.0 Hz, 1H), 6.99-7.64 (m, 6H), 4.45 (d, J=6.0 Hz, 2H), 4.09 (br d, J=5.2 Hz, 2H), 3.64 (t, J=4.7 Hz, 2H), 2.87 (q, J=7.4 Hz, 2H), 1.21 (t, J=7.5 Hz, 3H)

Synthesis of Compound 155

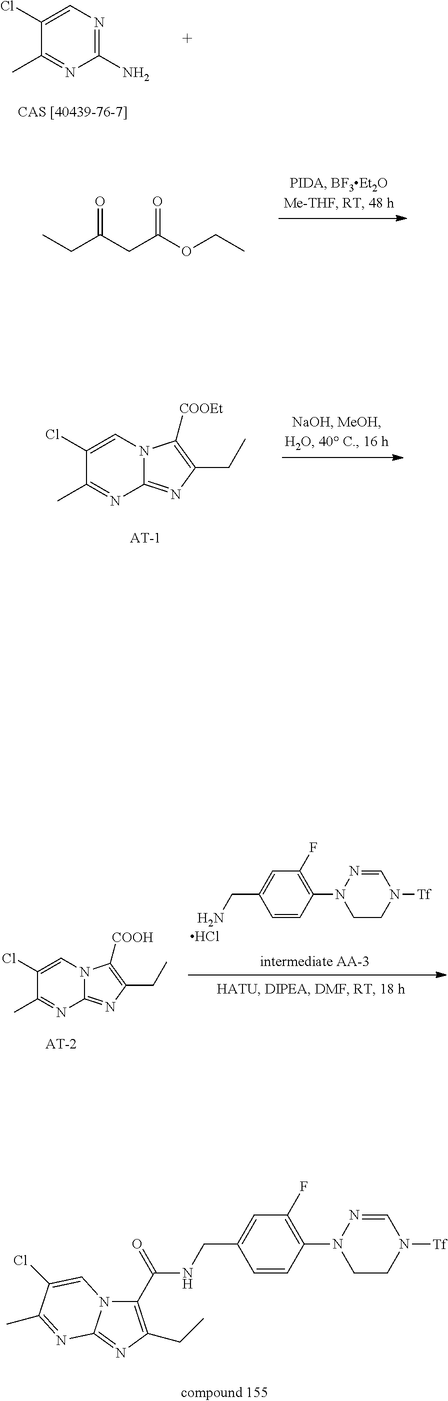

Preparation of Intermediate AT-1

Accordingly, intermediate AT-1 was prepared in the same way as AJ-1 starting from 5-chloro-4-methylpyrimidin-2-amine (CAS [40439-76-7], 6.96 mmol) and ethyl 3-oxovalerate (CAS [4949-44-4]) giving 0.37 g (20%) as white solid.

Preparation of Intermediate AT-2

Accordingly, intermediate AT-2 was prepared in the same way as intermediate AJ-2 starting from intermediate AT-1 (0.37 mmol) giving 0.165 g (quantitative).

Preparation of Compound 155

Accordingly, compound 155 was prepared in the same way as compound 161 starting from intermediate AT-2 (0.38 mmol) and intermediate AA-3 affording 0.055 g (26%) as white powder.

¹H NMR (500 MHz, DMSO-d₆) δ ppm 9.35 (br s, 1H), 8.48 (t, J=6.1 Hz, 1H), 7.30-7.40 (m, 2H), 7.16-7.28 (m, 2H), 4.50 (br d, J=5.6 Hz, 2H), 4.06-4.13 (m, 2H), 3.65 (br t, J=4.5 Hz, 2H), 3.01 (q, J=7.5 Hz, 2H), 2.62 (s, 3H), 1.27 (t, J=7.5 Hz, 3H)

Synthesis of Compound 150

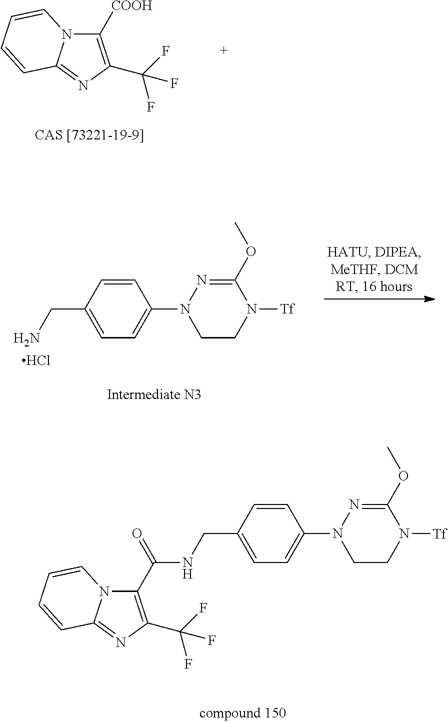

HATU (0.097 g, 0.26 mmol) was added to a solution of 2-(Trifluoromethyl)-imidazo[1,2-A]pyridine-3-carboxylic acid (CAS [73221-19-9], 0.051 g, 0.22 mmol) and DIPEA (0.096 mL, 0.56 mmol) in dry Me-THF (1.5 mL) and DCM (0.5 mL) under N₂. The solution was stirred at room temperature for 15 min. Then intermediate N3 (0.095 g, 0.24 mmol) was added and the reaction mixture was stirred at room temperature for 16 hours. The solvent was evaporated then the residue was diluted in ethyl acetate, washed with a saturated aqueous solution of NaHCO₃, water then brine. The organic layer was dried over MgSO₄, filtered and evaporated in vacuo to give a yellow oil, 0.314 g. Purification was carried out by flash chromatography over silica gel (12 g, irregular SiOH 25-40 μM, DCM/MeOH from 100/0 to 97/3). Pure fractions were collected and evaporated affording 0.119 g as white foam. It was triturated with DIPE and a few Heptane, the precipitate was filtered off and dried under vacuum at 60° C. affording compound 150 as white powder, 0.103 g (82%).

¹H NMR (500 MHz, DMSO-d₆) δ ppm 9.21 (br t, J=5.3 Hz, 1H), 8.53 (br d, J=6.7 Hz, 1H), 7.79 (br d, J=9.0 Hz, 1H), 7.55 (br t, J=7.8 Hz, 1H), 7.29 (br d, J=8.4 Hz, 2H), 7.13-7.22 (m, 3H), 4.47 (br d, J=5.5 Hz, 2H), 4.07-4.15 (m, 2H), 3.86 (s, 3H), 3.76 (br t, J=4.6 Hz, 2H)

Synthesis of Compound 88

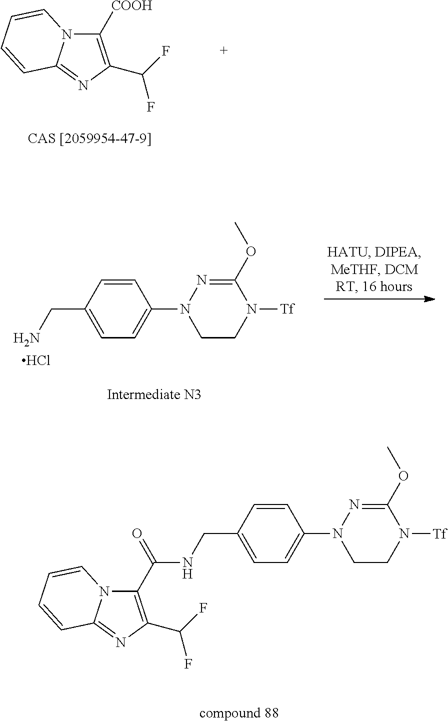

Accordingly, compound 88 was prepared in the same way as compound 150 starting from 2-(Difluoromethyl)-imidazo[1,2-A]pyridine-3-carboxylic acid (CAS [2059954-47-9], 0.23 mmol) and intermediate N3 affording a white powder, 0.104 g (86%).

¹H NMR (500 MHz, DMSO-d₆) δ ppm 8.94 (br t, J=5.1 Hz, 1H), 8.79 (d, J=7.0 Hz, 1H), 7.76 (d, J=9.0 Hz, 1H), 7.52 (t, J=7.9 Hz, 1H), 7.19-7.43 (m, 3H), 7.14-7.19 (m, 3H), 4.47 (br d, J=5.2 Hz, 2H), 4.07-4.14 (m, 2H), 3.85 (s, 3H), 3.71-3.79 (m, 2H)

Preparation of Compound 200

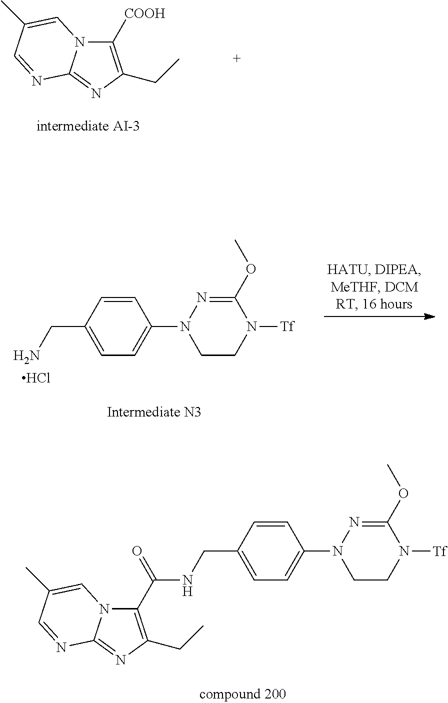

Accordingly, compound 200 was prepared in the same way as compound 150 starting from intermediate AI-3 (0.64 mmol) and intermediate N3 (0.51 mmol) affording a white powder, 0.085 g (31%).

¹H NMR (400 MHz, DMSO) δ 9.15-9.11 (m, 1H), 8.51 (d, J=2.3 Hz, 1H), 8.41 (t, J=5.9 Hz, 1H), 7.29 (d, J=8.7 Hz, 2H), 7.15 (d, J=8.7 Hz, 2H), 4.45 (d, J=5.8 Hz, 2H), 4.15-4.06 (m, 2H), 3.85 (s, 3H), 3.76-3.70 (m, 2H), 2.98 (q, J=7.5 Hz, 2H), 2.34 (s, 3H), 1.26 (t, J=7.5 Hz, 3H).

Synthesis of Compound 169 & compound 180

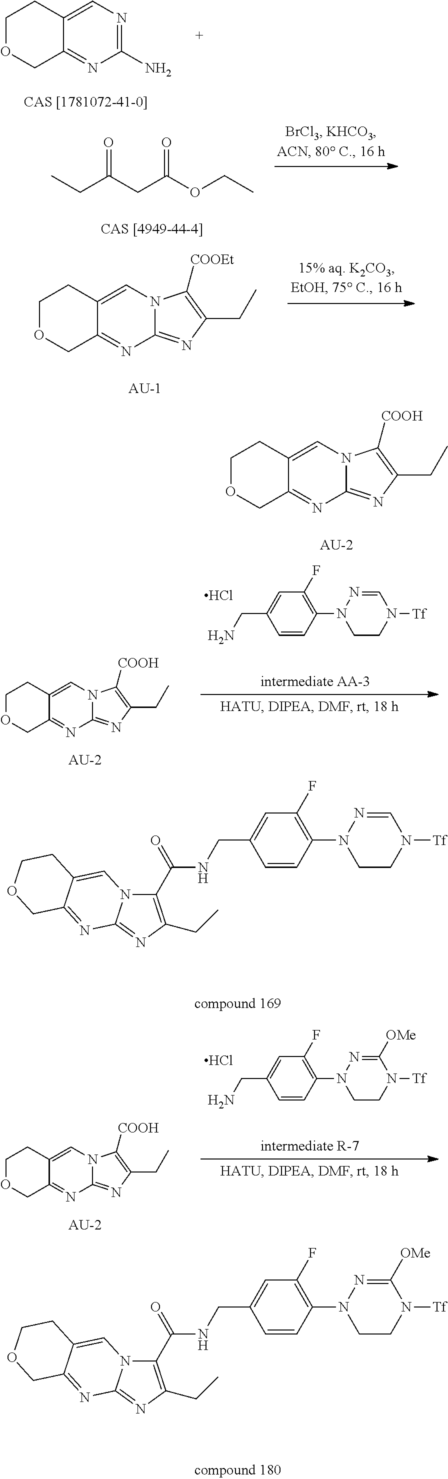

Preparation of Intermediate AU-1

In a screw top vial, a mixture of Ethyl propionylacetate (0.105 g, 0.73 mmol), 5H, 6H, 8H-pyrano[3,4-d]pyrimidin-2-amine (CAS [1781072-41-0], 0.11 g, 0.73 mmol), Potassium hydrogen carbonate (0.08 g, 0.8 mmol) and Bromotrichloromethane (0.143 mL, 1.45 mmol) in Acetonitrile (12 mL) at room temperature was stirred at 80° C. for 16 hours. Additional Ethyl propionylacetate (0.105 g, 0.73 mmol), Potassium hydrogen carbonate (0.08 g, 0.8 mmol) and Bromotrichloromethane (0.143 mL, 1.45 mmol) were added to the mixture and it was stirred at 80° C. for 24 hours. Then, the mixture was diluted with EtOAc and washed with sat. NaHCO₃ aq. solution (3×). The organic layer was dried over MgSO₄, filtered and concentrated in vacuo. The crude was purified by flash column chromatography over silica gel (12 g, EtOAc/Heptane from 0/100 to 100/0). The desired fractions were collected, and the solvent evaporated in vacuo to give intermediate AU-1 as a yellow sticky solid (0.084 g, 42%).

Preparation of Intermediate AU-2

In a screw top vial, Potassium carbonate 15% aqueous solution (0.8 mmol, 0.87 mmol) was added over a solution of intermediate AU-1 in EtOH (4 mL) at room temperature. The reaction mixture was heated at 75° C. and stirred for 36h. Then, HCl 2M aq. solution was added until pH 3, and the solvent was evaporated in vacuo to yield intermediate AU-2 as an orange solid, that was used in the next step without further purification (0.18 g, quantitative)/

Preparation of Compound 169

Accordingly, compound 169 was prepared in the same way as compound 161 starting from intermediate AU-2 (0.41 mmol) and intermediate AA-3 affording 0.051 g (28%) as white powder.

1H NMR (400 MHz, CDCl3) δ ppm 9.54 (s, 1H), 7.44 (t, J=8.5 Hz, 1H), 7.19 (s, 1H), 7.16-7.05 (m, 2H), 6.18 (br t, J=5.6 Hz, 1H), 4.84 (s, 2H), 4.64 (d, J=5.8 Hz, 2H), 4.13-4.05 (m, 2H), 4.02 (t, J=5.7 Hz, 2H), 3.71-3.63 (m, 2H), 3.05-2.89 (m, 4H), 1.45 (t, J=7.5 Hz, 3H).

Preparation of Compound 180

Accordingly, compound 180 was prepared in the same way as compound 161 starting from intermediate AU-2 (0.081 mmol) and intermediate R-7 affording 0.012 g (30%) as white powder.

¹H NMR (400 MHz, CDCl3) δ ppm 9.54 (s, 1H), 7.46 (t, J=8.6 Hz, 1H), 7.10 (m, 2H), 6.17 (br t, J=5.5 Hz, 1H), 4.84 (s, 2H), 4.63 (d, J=5.8 Hz, 2H), 4.15-4.05 (m, 2H), 4.02 (t, J=5.7 Hz, 2H), 3.89 (s, 3H), 3.65-3.55 (m, 2H), 3.07-2.92 (m, 4H), 1.45 (t, J=7.5 Hz, 3H).

Synthesis of Compound 177

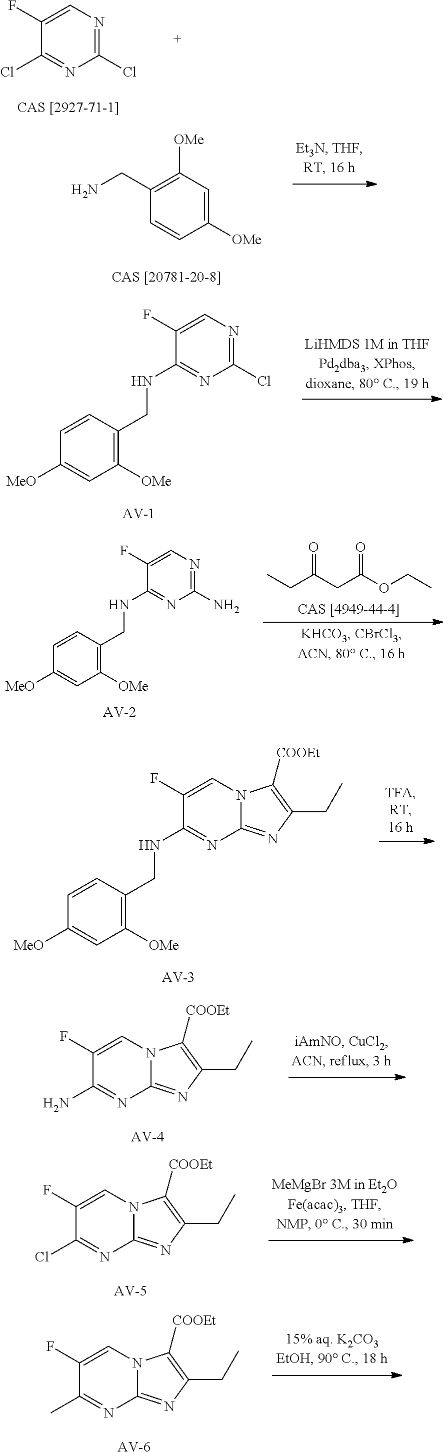

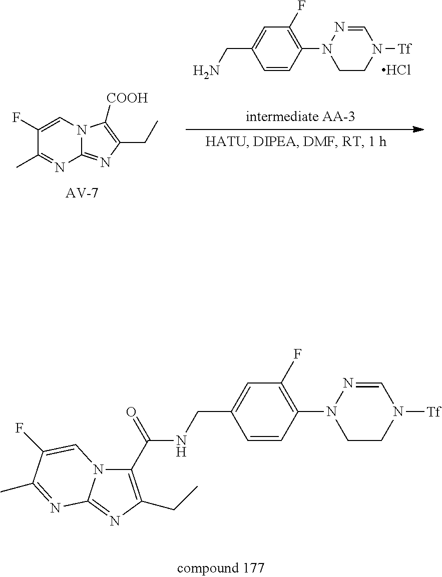

Preparation of Intermediate AV-1

The reaction was divided in two batches of 1.5 g each one. 2,4-Dimethoxybenzylamine (CAS [20781-20-8], 2.97 mL, 19.76 mmol) was added dropwise to a solution of 2,4-Dichloro-5-fluoropyrimidine (CAS [2927-71-1], 3 g, 17.97 mmol) and triethylamine (3 mL, 21.5 mmol) in THF dry in a round bottom flask under nitrogen at 0° C. The reaction mixture was allowed to warm to room temperature for 16 h. The mixture was diluted with saturated aqueous NaHCO₃ solution and extracted with EtOAc. The organic layer was separated, dried with MgSO₄, filtered and the solvents were evaporated in vacuo. The crude product was purified by flash column chromatography over silica gel (80 g, ethyl acetate in heptane from 100/0 to 20/80). The desired fractions were collected and concentrated in vacuo to yield intermediate AV-1 as a beige solid, 4.8 g (85%).

Preparation of Intermediate AV-2

The reaction was divided in two batches of 2.4 g each one.

Tris(dibenzylideneacetone)dipalladium (0) (0.7 g, 0.77 mmol) and XPhos (0.73 g, 1.53 mmol) were added to a solution of AV-1 (4.32 g, 15.32 mmol) in dry dioxane (31 mL) while nitrogen was bubbling in a glass pressure bottle. Then lithium bis(trimethylsilyl)amide solution, 1M in THE (33.7 mL, 33.7 mmol) was added dropwise and the resulting solution was heated at 80° C. for 3 h. Tris(dibenzylideneacetone)dipalladium(O) (0.7 g, 0.77 mmol), XPhos (0.73 g, 1.53 mmol) and lithium bis(trimethylsilyl)amide solution, 1M in THE (33.7 mL, 33.7 mmol) were added while nitrogen was bubbling and the reaction mixture was heated at 80° C. for 16 h. The reaction was acidified with HCl 1N solution and stirred for 30 min. Then the result was extracted with EtOAc. The aqueous layer was neutralized with 1N NaOH solution and extracted with DCM. The organic layer was separated, dried (MgSO₄), filtered and the solvents were evaporated in vacuo to yield intermediate AV-2 as a brown solid, 3.4 g (76%).

Preparation of Intermediate AV-3

The reaction was set up in 2 batches with the same quantity of reactive AV-2.

Potassium bicarbonate (0.6 g, 6.04 mmol) and Ethyl propionylacetate (0.89 mL, 6.04 mmol) were added to a solution of AV-2 (1.12 g, 4.02 mmol) in ACN (8.1 mL) in a screw top vial at rt. Then, Bromotricloromethane (1.19 mL, 12.07 mmol) was added at room temperature and the mixture was stirred at 80° C. for 16h. The batches were mixed to be worked out together. The mixture was diluted with water and extracted with EtOAc. The organic layer was dried (MgSO₄), filtered and concentrated in vacuo. The crude was purified by flash chromatography column over silica gel (25 g; EtOAc in Heptane 0/100 to 35/65). The desired fractions were collected and concentrated in vacuo to yield intermediate AV-3 as a yellow foam solid, 0.42 g (22%).

Preparation of Intermediate AV-4

TFA (9.64 mL, 128.43 mmol) was added to AV-3 (1.06 g, 2.37 mmol) in a round bottom flask at 0° C. The mixture was stirred at room temperature for 16 h. The mixture was neutralized with sat. aqueous NaHCO₃ solution and extracted with DCM. The organic layer was washed with water and concentrated in vacuo. The result was triturated with DIPE and the solid was filtered to yield intermediate AV-4 as a beige solid, 0.6 g (95%).

Preparation of Intermediate AV-5

Isoamylnitrite (CAS [110-46-3], 0.46 mL, 3.38 mmol) and copper (II) chloride (0.318 g, 2.36 mmol) were added to a suspension of AV-4 (0.6 g, 2.25 mmol) in dry ACN (36 mL) in a round bottom flask at room temperature. The mixture was stirred at reflux for 3 h. Water was added and the mixture was extracted with EtOAc. The organic layer was separated, dried (MgSO₄), filtered and the solvents were evaporated in vacuo. The crude was purified by flash chromatography column over silica gel (12 g; EtOAc in Heptane 0/100 to 10/90). The desired fractions were collected and concentrated in vacuo to yield intermediate AV-5 as a white solid, 0.315 g (51%).

Preparation of Intermediate AV-6

Iron (III) acetylacetonate (0.051 g, 0.14 mmol) was added to a solution of AV-5 (0.39 g, 1.41 mmol) in dry THE (8 mL) and NMP (0.7 mL) in a round bottom flask under nitrogen at 0° C. Then methylmagnesium bromide solution 3.0 M in diethyl ether (0.71 mL, 2.12 mmol) was added dropwise, and the reaction mixture was stirred at 0° C. for 30 min. TLC showed complete conversion. The reaction was quenched with saturated aqueous NH₄Cl solution. The mixture was extracted with ethyl acetate. The organic layer was separated, dried over MgSO₄, filtered and the solvents were evaporated in vacuo. The crude product was purified by flash column chromatography over silica gel (12 g; EtOAc in heptane 0/100 to 15/75). The desired fractions were collected and concentrated in vacuo to yield a white solid, intermediate AV-6, 0.325 g (91%).

Preparation of Intermediate AV-7

15% aqueous potassium carbonate (0.88 mL, 0.96 mmol) was added to a solution of AV-6 (0.152 g, 0.6 mmol) in EtOH (2 mL) in a screw top vial at room temperature. The mixture was stirred at 90° C. for 18 h. 15% aqueous potassium carbonate (0.88 mL, 0.96 mmol) was added to a reaction mixture. The mixture was stirred at 90° C. for 2 h. Then, 1M aqueous HCl solution was added until pH 7. The mixture was concentrated in vacuo to yield intermediate AV-7 as a white solid (0.188 g, quantitative).

Preparation of Compound 177

Intermediate AA-3 (0.158 g, 0.4 mmol) was added to a solution of AV-7 (0.187 g, 0.6 mmol), HATU (0.198 g, 0.52 mmol), and DIPEA (0.42 mL, 2.4 mmol) in dry DMF (5 mL) in a round bottom flask at room temperature. The mixture was stirred at room temperature for 1 h. Saturated aqueous NaHCO₃ solution was added and the mixture was extracted with EtOAc (×3). The combined organic layers were dried over MgSO₄, filtered and concentrated in vacuo. The crude product was purified by flash column chromatography over silica gel (12 g; (DCM/MeOH 9:1) in DCM 0/100 to 10/90). The desired fractions were collected and concentrated in vacuo. The result was triturated with DIPE and the solid was filtered to yield compound 177 as a beige solid, 0.092 g (41%).

¹H NMR (400 MHz, DMSO-d6) δ ppm 9.32 (d, J=5.5 Hz, 1H), 8.44 (br t, J=5.9 Hz, 1H), 7.38 (s, 1H), 7.34 (t, J=8.6 Hz, 1H), 7.25 (br d, J=13.2 Hz, 1H), 7.20 (br d, J=8.3 Hz, 1H), 4.50 (d, J=5.8 Hz, 2H), 4.17-4.02 (m, 2H), 3.72-3.58 (m, 2H), 3.02 (q, J=7.5 Hz, 2H), 2.56 (d, J=2.7 Hz, 3H), 1.28 (t, J=7.5 Hz, 3H).

Synthesis of Compound 142 and Compound 181

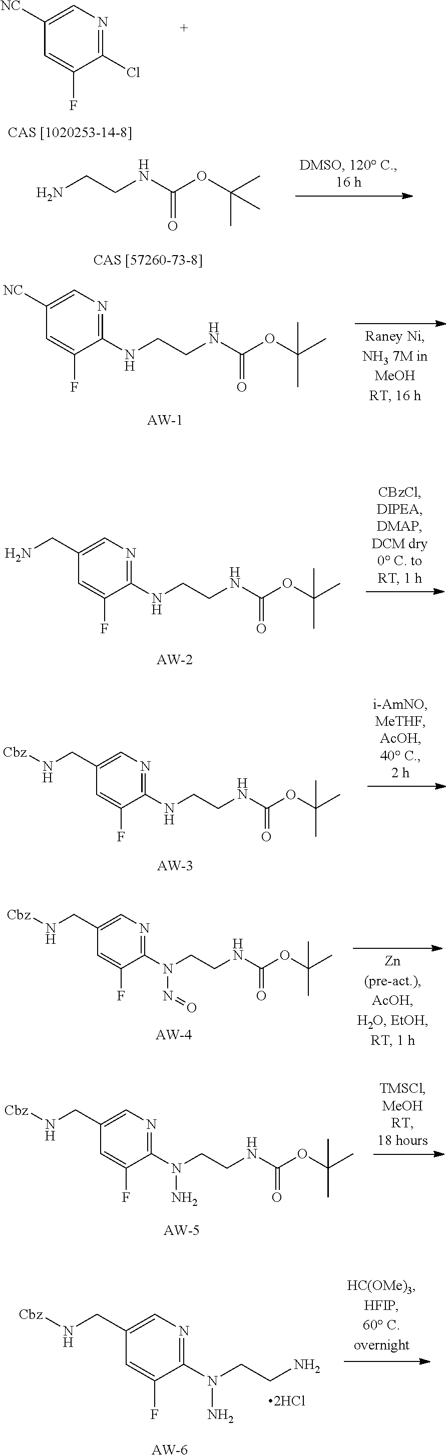

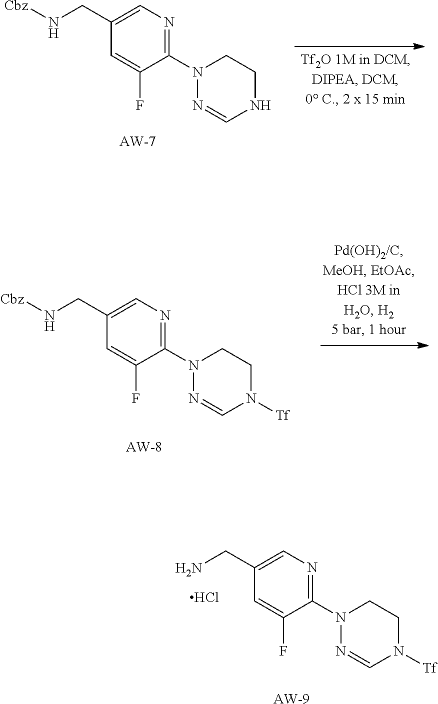

Preparation of Intermediate AW-1

A solution of 6-chloro-5-fluoronicotinonitrile (CAS [1020253-14-8], 13.57 g, 86.68 mmol), n-boc-1,2-diaminoethane (CAS [57260-73-8], 17.8 mL, 113 mmol) and Et₃N (48.2 mL, 347 mmol) in dry DMSO (155 mL) was stirred at 120° C. for 16 h. EtOAc and water were added to the reaction mixture. The layers were separated, and the organic layer was washed with brine (5 times), dried over MgSO₄, filtered off and evaporated to give an orange solid. The solid was purified by preparative LC (regular SiOH 30 μm, 330 g, liquid loading (DCM), mobile phase gradient: Heptane/EtOAc 95/5 to Heptane/EtOAc 40/60). The fractions containing product were combined and evaporated to give 22.55 g of intermediate AW-1 as a yellow solid (93% yield).

Preparation of Intermediate AW-2

To a solution of AW-1 (3.2 g, 11.42 mmol) in NH₃ (7M in MeOH) (179 mL), purged with nitrogen, was added Raney Nickel (5.3 g, 91.3 mmol) then the reaction mixture was hydrogenated under atmospheric pressure at room temperature for 16 hours. The mixture was filtered through a pad of Celite® and the Celite® was rinsed with MeOH and the filtrate was concentrated in vacuo. The residue was diluted in DCM, MgSO₄ was added. The mixture was filtered through a pad of Celite®, the Celite® was washed with DCM and the filtrate was evaporated in vacuo to give of mmotte_8598_1, 3.18 g, as colourless oil (96%).

Preparation of Intermediate AW-3

A round-bottom flask was charged with a solution of AW-2 (3.18 g, 10.96 mmol), DIPEA (2.17 mL, 12.6 mmol) and DMAP (0.04 g, 0.33 mmol) in dry DCM (68.2 mL). The reaction mixture was connected to a nitrogen flow then cooled down to 0° C. Benzylchloroformate (1.72 mL, 12.06 mmol) was added dropwise. The reaction mixture was then stirred at 0° C. for 1h. The reaction mixture was quenched by addition of water and stirred for 10 minutes at room temperature. The aqueous layer was extracted with DCM (twice). The combined organic layer was dried over MgSO₄, filtered off and evaporated to give 5.38 g as crude. Purification was carried out by flash chromatography over silica gel (120 g, irregular SiOH 25-40 μM, DCM/MeOH from 100/0 to 97/3). Pure fractions were collected and evaporated affording intermediate AW-3 as pale beige solid, 3.54 g (77%).

Preparation of Intermediate AW-4

AW-3 (3.54 g, 8.46 mmol) was solubilized at 40° C. in Me-THF (65 mL) and AcOH (4.84 mL, 84.59 mmol). Then isoamylnitrite (5.68 mL, 42.3 mmol) was added dropwise and the mixture was stirred at 40° C. for 2 hours. The solution was diluted in EtOAc (60 mL) and water (30 mL), washed with a saturated solution of NaHCO₃ (twice), brine, dried on MgSO₄ and evaporated to give 4.67 g as pale-yellow oil. Purification was carried out by flash chromatography over silica gel (80 g, irregular SiOH 25-40 μM, DCM/MeOH from 100/0 to 97/3). Pure fractions were collected and evaporated affording intermediate AW-4 as a yellow oil, 3.99 g (97% with 92% purity, used as such for next step).

Preparation of Intermediate AW-5

Zinc, dust (4.29 g, 65.63 mmol) was added to a solution of AW-4 (3.99 g, 8.2 mmol) and AcOH (7 mL, 123.05 mmol) in EtOH (170.9 mL) and water (42.7 mL) at room temperature. The mixture was stirred at room temperature for 1.5 hour. Water was added, the aqueous layer was extracted 3 times with DCM, the combined organic layers were dried over MgSO₄ and concentrated under reduced pressure giving a colourless oil, 4.12 g. Purification was carried out by flash chromatography over silica gel (80 g, irregular SiOH 25-40 μM, DCM/MeOH from 100/0 to 97/3). Pure fractions were collected and evaporated affording intermediate AW-5, 1.88 g as colourless oil (50%).

Preparation of Intermediate AW-6

To a solution of AW-5 (1.88 g, 4.08 mmol) in MeOH (40.2 mL) was added dropwise TMSCl (4.14 mL, 32.61 mmol). The reaction mixture was stirred at room temperature for 18 hours. The reaction mixture was concentrated in vacuo to give intermediate AW-6, 1.45 g (80%), used as such for next step.

Preparation of Intermediate AW-7

A solution of AW-6 (1.45 g, 3.21 mmol) and B (1.41 mL, 12.85 mmol) in C (32.4 mL) was stirred at 70° C. overnight. The reaction mixture was evaporated. The residue was diluted in DCM and a 10% aq. solution of K₂CO₃. The aqueous layer was extracted twice with DCM/MeOH (95/5). The combined organic layers were dried on MgSO₄, filtered off and evaporated to give a yellow solid. Purification was carried out by flash chromatography over silica gel (12 g, irregular SiOH 25-40 μM, DCM/MeOH from 100/0 to 90/10). Pure fractions were collected and evaporated affording intermediate AW-7 as colorless oil, 0.58 g, used as such for next step.

Preparation of Intermediate AW-8

To a solution of AW-7 (0.58 g, 1.69 mmol) and DIPEA (0.87 mL, 5.07 mmol) in dry DCM (14.6 mL), cooled at 5° C. in an ice bath, was added dropwise Tf₂O 1M in DCM (1.69 mL, 1.69 mmol). The reaction mixture was stirred at 5° C. for 15 min. The reaction mixture was immediately quenched with a saturated solution of NaHCO₃. The aqueous layer was extracted with DCM (twice). The combined organic layer was washed with brine (once), dried over MgSO₄, filtered off and evaporated. Purification was carried out by flash chromatography over silica gel (24 g, irregular SiOH 25-40 μM, DCM/MeOH from 100/0 to 97/3). Pure fractions were collected and evaporated affording intermediate AW-8, as pale-yellow oil which crystalized on standing, 0.59 g (73%).

Preparation of Intermediate AW-9

In a steal bomb, a mixture of AW-8 (0.59 g, 1.24 mmol), palladium hydroxide 20% on carbon nominally 50% water (0.17 g, 0.12 mmol) and aqueous HCl 3M (0.41 mL, 1.24 mmol) in MeOH (8.7 mL) and EtOAc (8.7 mL) was hydrogenated under 3 bar of H₂ at room temperature for 3 hours. The mixture was filtered on a pad of Celite® and washed with MeOH. The filtrate was evaporated then co-evaporated with MeOH (twice) to give intermediate AW-9, 0.484 g (90%) as pale beige powder.

Preparation of Compound 142

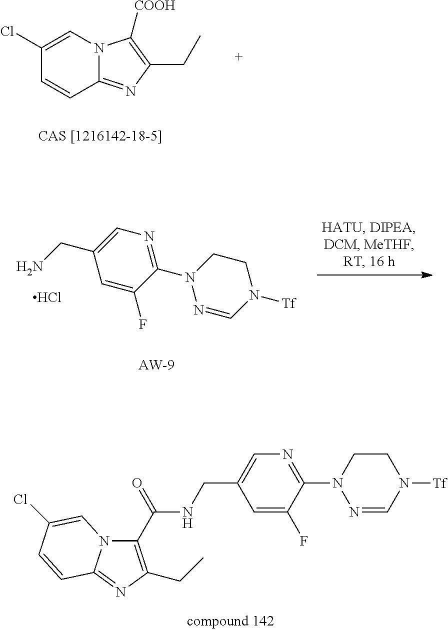

HATU (0.15 g, 0.4 mmol) was added to a solution of 6-Chloro-2-ethylimidazo[1,2-a]pyridine-3-carboxylic acid (CAS [1216142-18-5], 0.078 g, 0.35 mmol) and DIPEA (0.21 mL, 1.21 mmol) in dry Me-TIF (2.8 mL) and dry DCM (2 mL) under N₂ flow. The solution was stirred at room temperature for 15 min. Then AW-9 (0.118 g, 0.35 mmol) was added and the reaction mixture was stirred at room temperature for 16 hours. The solvent was evaporated then the residue was diluted in ethyl acetate, washed with a saturated aqueous solution of NaHCO₃, water then brine. The organic layer was dried over MgSO₄, filtered and evaporated in vacuo to give a brown residue. Purification was carried out by flash chromatography over silica gel (40 g, irregular SiOH 25-40 μM, solid deposit on Celite®, DCM/MeOH from 100/0 to 97/3). Pure fractions were collected and evaporated affording a pale-yellow powder, 0.512 g. A purification was performed via achiral SFC (Stationary phase: Whelk-O1 (S,S) 5 μm 250*30 mm, Mobile phase: 60% CO₂, 40% mixture of MeOH/DCM 80/20 v/v+0.3% iPrNH2). Pure fractions were collected and evaporated affording a white solid, 0.31 g. This was triturated with DIPE and a few Heptane, the precipitate was filtered off and dried under vacuum at 60° C. giving compound 142 as white powder, 0.29 g (47%).

¹H NMR (500 MHz, DMSO-d6) δ ppm 9.09 (d, J=1.4 Hz, 1H), 8.46 (t, J=5.8 Hz, 1H), 8.13 (br s, 1H), 7.63-7.75 (m, 2H), 7.47 (dd, J=9.4, 2.1 Hz, 1H), 7.37 (s, 1H), 4.51 (br d, J=5.8 Hz 2H), 4.13 (br t, J=4.5 Hz, 2H), 3.92 (t, J=4.8 Hz, 2H), 2.99 (q, J=7.5 Hz, 2H), 1.26 (t, J=7.5 Hz, 3H)

Preparation of Compound 181

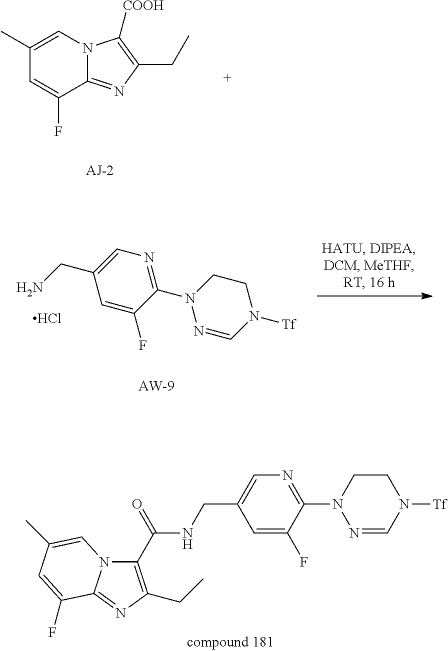

AW-9 (0.09 g, 0.24 mmol) was added to a solution of AJ-2 (0.099 g, 0.38 mmol), HATU (0.12 g, 0.31 mmol) and DIPE (0.25 mL, 1.43 mmol) in dry DMF (5 mL) in a round bottom flask at room temperature. The mixture was stirred at room temperature for 16 h. The mixture was diluted with an aqueous saturated NaHCO₃ solution and extracted with DCM. The organic layer was separated, dried (MgSO₄), filtered and the solvents concentrated in vacuo to yield a brown oil. The crude product was triturated with DCM and the solid was filtered and dried in vacuo to yield a white solid, compound 181, 0.059 g (45%).

¹H NMR (400 MHz, DMSO-d6) δ ppm 8.62 (s, 1H), 8.51 (br t, J=5.8 Hz, 1H), 8.13 (s, 1H), 7.69 (dd, J=12.7, 1.7 Hz, 1H), 7.37 (s, 1H), 7.22 (dd, J=11.7, 0.9 Hz, 1H), 4.51 (d, J=5.8 Hz, 2H), 4.17-4.10 (m, 2H), 3.96-3.89 (m, 2H), 2.97 (q, J=7.5 Hz, 2H), 2.31 (s, 3H), 1.26 (t, J=7.5 Hz, 3H).

Preparation of Compound 201

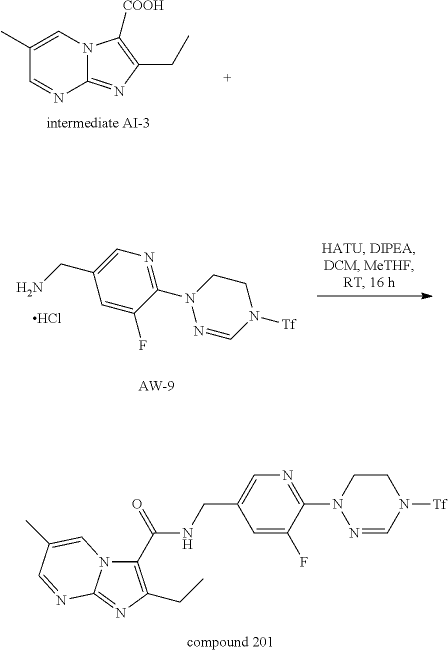

Accordingly compound 201 was prepared in the same way as compound 142 starting from intermediate AI-3 (0.64 mmol) and intermediate AW-9 (0.4 mmol) affording a white solid, 0.063 g (30%).

¹H NMR (400 MHz, DMSO) δ 9.19-9.12 (m, 1H), 8.51 (d, J=2.4 Hz, 1H), 8.44 (t, J=5.8 Hz, 1H), 8.13 (s, 1H), 7.69 (dd, J=12.7, 1.7 Hz, 1H), 7.36 (s, 1H), 4.51 (d, J=5.8 Hz, 2H), 4.13 (t, J=4.6 Hz, 2H), 3.96-3.87 (m, 2H), 3.00 (q, J=7.5 Hz, 2H), 2.34 (s, 3H), 1.27 (t, J=7.5 Hz, 3H).

Synthesis of Compound 213

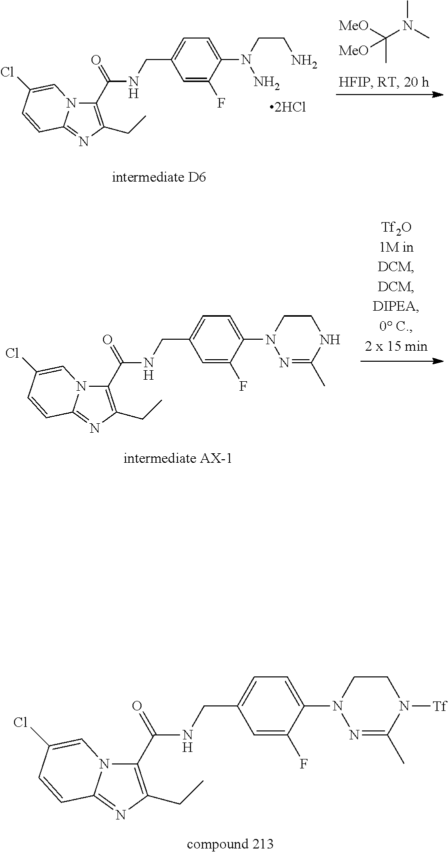

Preparation of Intermediate AX-1

N,N Dimethylacetamide dimethyl acetal (0.2 mL; 1.26 mmol) was added to a solution of intermediate D6 (0.3 g; 0.63 mmol) in HFIP (10.8 mL) and the mixture was stirred at room temperature for 20 h. The reaction mixture was diluted with EtOAc and treated with an aqueous saturated solution of NaHCO₃. The layers were separated, and the aqueous layer was extracted with EtOAc. The combined organic layers were dried over MgSO₄, filtered and the solvent was removed under reduced pressure to give a colorless oil. Purification was carried out by flash chromatography over silica gel (24 g, irregular SiOH 25-40 μM, DCM/MeOH from 95/5 to 90/10). Pure fractions were collected and evaporated affording intermediate AX-1 as colorless oil, 0.176 g (65%).

Preparation of Compound 213

To a solution of intermediate AX-1 (0.139 g, 0.32 mmol) and DIPEA (0.17 mL, 0.97 mmol) in dry DCM (2.8 mL), cooled at 5° C. in an ice bath, was added dropwise Tf₂O 1M in DCM (0.32 mL, 0.32 mmol). The reaction mixture was stirred at 5° C. for 15 min. The reaction mixture was immediately quenched with a saturated solution of NaHCO₃. The aqueous layer was extracted with DCM (twice). The combined organic layer was washed with brine (once), dried over MgSO₄ and filtered off to give a crude. Dry DCM (2.8 mL) was added to the crude, the solution was cooled down to 5° C. then DIPEA (0.056 mL, 0.32 mmol) was added, followed by Tf₂O 1M in DCM (0.13 mL, 0.13 mmol). The reaction mixture was stirred at 5° C. for 15 min. The reaction mixture was immediately quenched with a saturated solution of NaHCO₃. The aqueous layer was extracted with DCM (twice). The combined organic layer were washed with brine (once), dried over MgSO₄ and filtered off to give 0.217 g as an oil. Purification was carried out by flash chromatography over silica gel (12 g, irregular SiOH 25-40 μM, DCM/MeOH from 100/0 to 97/3). Pure fractions were collected and evaporated affording compound 213, as beige powder, 0.093 g (51%). Purification was carried out by flash chromatography over silica gel (12 g, irregular SiOH 25-40 μM, DCM/MeOH from 100/0 to 97/3). Pure fractions were collected and evaporated affording compound 213, as beige powder, 0.075 g (41%). This one was crystallized from DIPE/Heptane, triturated, filtered off and dried under vacuum at 60° C. affording compound 213 as white powder, 0.063 g (35%).

¹H NMR (500 MHz, DMSO-d₆) δ ppm 9.04-9.11 (m, 1H), 8.47 (t, J=5.9 Hz, 1H), 7.64-7.72 (m, 1H), 7.46 (dd, J=9.5, 2.1 Hz, 1H), 7.29-7.38 (m, 1H), 7.13-7.27 (m, 2H), 5.12-5.18 (m, 1H), 4.49 (d, J=6.0 Hz, 2H), 3.95-4.06 (m, 2H), 3.67-3.77 (m, 2H), 3.01 (q, J=7.5 Hz, 2H), 2.25 (s, 3H), 1.22-1.31 (t, J=7.5 Hz, 3H).

Synthesis of Intermediate AY-3

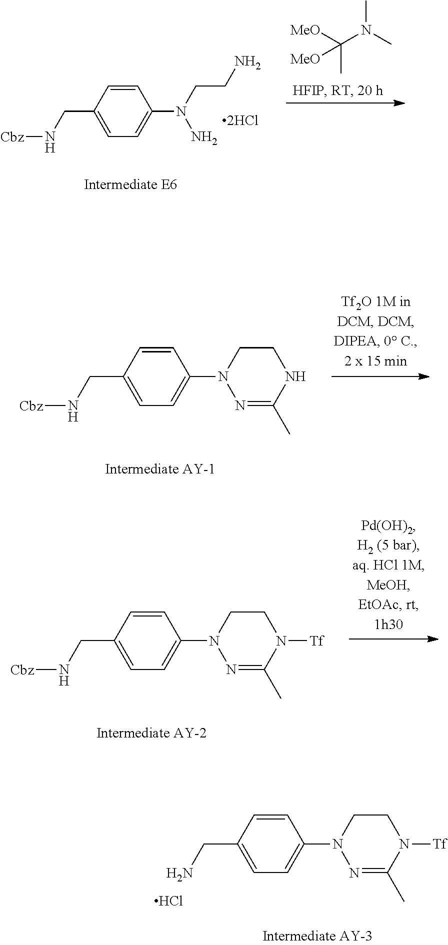

Preparation of Intermediate AY-1

N,N Dimethylacetamide dimethyl acetal (1.68 mL; 10.33 mmol) was added to a solution of intermediate E6 (2 g; 5.16 mmol) in HFIP (88 mL) and the mixture was stirred at room temperature for 20 h. The reaction mixture was diluted with EtOAc and treated with an aqueous saturated solution of NaHCO₃. The layers were separated, and the aqueous layer was extracted with EtOAc. The combined organic layers were dried over MgSO₄, filtered and the solvent was removed under reduced pressure. The residue was purified by preparative LC (irregular SiOH 40 μm, 40 g, from DCM/MeOH 95/5 to 90/10) to give 442 mg of intermediate AY-1 as a colorless residue which crystallized on standing (25%).

Preparation of Intermediate AY-2

Accordingly, intermediate AY-2 was prepared in the same way as compound 213 starting from AY-1 (1.31 mmol), yielding a beige powder, 0.388 g (63%).

Preparation of Intermediate AY-3

In a steal bomb, a mixture of AY-2 (0.39 g, 0.82 mmol), palladium hydroxide 20% on carbon nominally 50% water (0.12 g, 0.082 mmol) and aqueous HCl 1M (0.82 mL, 0.82 mmol) in MeOH (5.8 mL) and EtOAc (5.8 mL) was hydrogenated under 5 bar of H₂ at room temperature for 1.5 hour. The mixture was filtered on a pad of celite and washed with MeOH. The filtrate was evaporated to give intermediate AY-3, 0.32 g (96%, purity 92%), used as such for next step.

Preparation of Compound 214

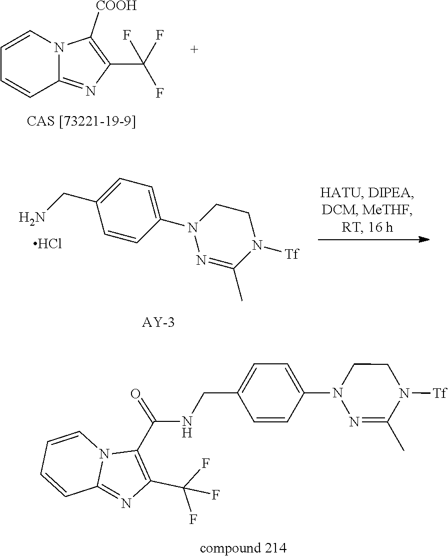

Accordingly, compound 214 was prepared in the same way as compound 181 starting from 2-(Trifluoromethyl)-imidazo[1,2-A]pyridine-3-carboxylic acid (CAS [73221-19-9], 0.34 mmol) and intermediate AY-3 (0.39 mmol) yielding a white powder, 0.098 g (52%).

¹H NMR (500 MHz, DMSO-d₆) δ ppm 9.17-9.29 (m, 1H), 8.48-8.58 (m, 1H), 7.73-7.83 (m, 1H), 7.49-7.60 (m, 1H), 7.30 (br d, J=8.2 Hz, 2H), 7.13-7.24 (m, 3H), 4.42-4.52 (m, 2H), 4.01 (br s, 2H), 3.84 (br d, J=4.3 Hz, 2H), 2.27 (s, 3H)

Preparation of Compound 215

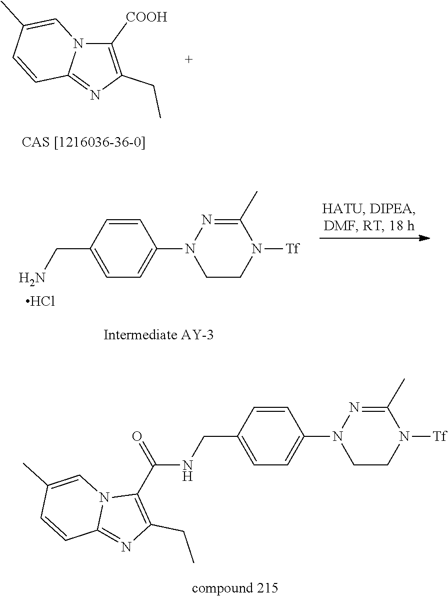

Accordingly, compound 215 was prepared in the same way as compound 181 starting from 2-ethyl-6-methylimidazo[1,2-a]pyridine-3-carboxylic acid (CAS [1216036-36-0], 0.34 mmol) and intermediate AY-3 (0.39 mmol) affording a white powder, 0.129 g (72%).

¹H NMR (500 MHz, DMSO-d6) δ ppm 8.77 (s, 1H), 8.29-8.36 (m, 1H), 7.47-7.54 (m, 1H), 7.27-7.33 (m, 2H), 7.21-7.25 (m, 1H), 7.14-7.19 (m, 2H), 4.41-4.49 (m, 2H), 4.06-4.09 (m, 1H), 3.96-4.05 (m, 2H), 3.79-3.84 (m, 2H), 2.90-3.02 (m, 2H), 2.31 (s, 3H) 2.26 (s, 3H), 1.20-1.30 (m, 3H)

Preparation of Compound 217

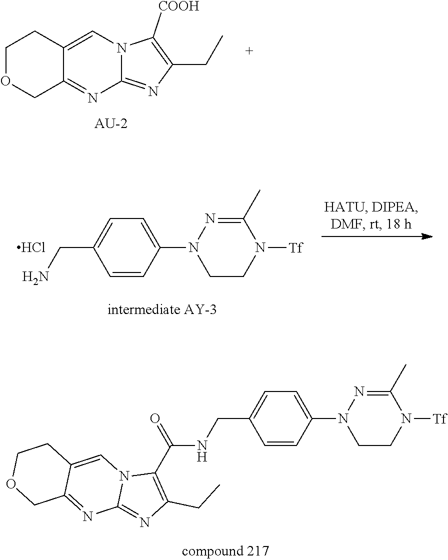

Accordingly, compound 217 was prepared in the same way as compound 181 starting from intermediate AU-2 (0.31 mmol) and intermediate AY-3 yielding a white foam, 0.018 g (10%).

¹H NMR (500 MHz, DMSO-d₆) δ ppm 9.17 (s, 1H), 8.40 (t, J=6.0 Hz, 1H), 7.27-7.35 (m, 2H), 7.12-7.21 (m, 2H), 4.69-4.77 (m, 2H), 4.41-4.49 (m, 2H), 3.98-4.04 (m, 2H), 3.91-3.97 (m, 2H), 3.79-3.84 (m, 2H), 2.95-3.01 (m, 2H), 2.89-2.94 (m, 2H), 2.25 (s, 3H), 1.22-1.29 (m, 4H)

Preparation of Compound 218

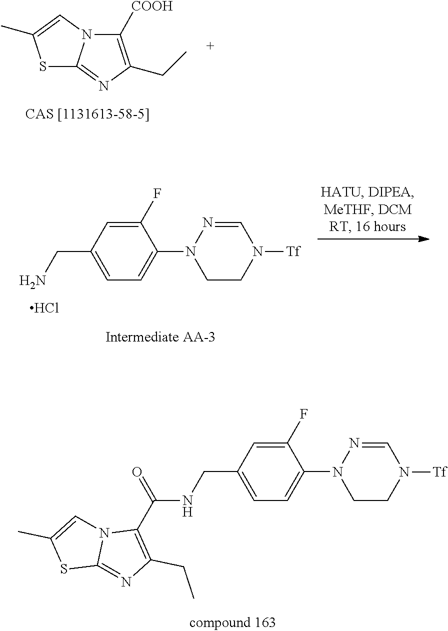

Accordingly compound 218 was prepared in the same way as compound 181 starting from 6-ethyl-2-methylimidazo[2,1-b][1,3]thiazole-5-carboxylic acid (CAS [1131613-58-5], 0.29 mmol) and intermediate AY-3 yielding a white foam, 0.059 g (38%).

¹H NMR (500 MHz, DMSO-d₆) δ ppm 8.09 (t, J=6.0 Hz, 1H), 7.80-7.91 (m, 1H), 7.21-7.32 (m, 2H), 7.08-7.19 (m, 2H), 4.40 (d, J=6.0 Hz, 2H), 4.00 (t, J=4.9 Hz, 2H), 3.81 (t, J=4.9 Hz, 2H), 2.85 (q, J=7.5 Hz, 2H), 2.40-2.46 (m, 3H), 2.22-2.28 (m, 3H), 1.20 (t, J=7.5 Hz, 3H)

Synthesis of Compound 216

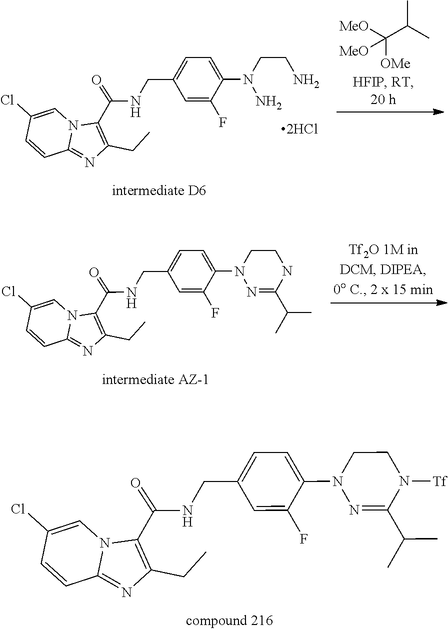

Preparation of Intermediate AZ-1

Trimethyl Orthoisobutyrate (0.2 mL; 1.26 mmol) was added to a solution of intermediate D (0.3 g; 0.63 mmol) in HFIP (10.8 mL) and the mixture was stirred at room temperature for 20 h. The reaction mixture was diluted with EtOAc and treated with an aqueous saturated solution of NaHCO₃. The layers were separated, and the aqueous layer was extracted with EtOAc. The combined organic layers were dried over MgSO₄, filtered and the solvent was removed under reduced pressure to give an oil. Purification was carried out by flash chromatography over silica gel (4 g, irregular SiOH, DCM/MeOH from 95/5 to 85/15). Pure fractions were collected and evaporated affording intermediate AZ-1 as colourless oil, 0.105 g (37%).

Preparation of Compound 216

To a solution of AZ-1 (0.11 g, 0.23 mmol) and DIPEA (0.12 mL, 0.69 mmol) in dry DCM (2 mL), cooled at 5° C. in a ice bath, was added dropwise Tf₂O 1M in DCM (0.23 mL, 0.23 mmol). The reaction mixture was stirred at 5° C. for 15 min. The reaction mixture was immediately quenched with a saturated solution of NaHCO₃. The aqueous layer was extracted with DCM (twice). The combined organic layer were washed with brine (once), dried over MgSO₄ and filtered off and evaporated. DCM (2 mL) was added to the residue, the solution was cooled down to 5° C. then DIPEA (0.04 mL, 0.23 mmol) was added, followed by Tf₂O 1M in DCM (0.092 mL, 0.092 mmol). The reaction mixture was stirred at 5° C. for 15 min. The reaction mixture was immediately quenched with a saturated solution of NaHCO₃. The aqueous layer was extracted with DCM (twice). The combined organic layer were washed with brine (once), dried over MgSO4 and filtered off to give 0.725 g. A purification was carried out by flash chromatography over silica gel (4 g, irregular SiOH 25-40 μM, Heptane/EtOAc from 90/10 to 70/30). Pure fractions were collected and evaporated affording a beige powder, 0.06 g. This one was triturated with DIPE and a few Heptane, the precipitate was filtered off and dried under vacuum at 60° C. affording compound 216 as white powder, 0.040 g.

¹H NMR (500 MHz, DMSO-d₆) δ ppm 9.03-9.18 (m, 1H), 8.47 (br t, J=5.5 Hz, 1H), 7.63-7.73 (m, 1H), 7.43-7.50 (m, 1H), 7.30-7.38 (m, 1H), 7.16-7.27 (m, 2H), 4.50 (br d, J=5.6 Hz, 2H), 3.87-3.94 (m, 2H), 3.80 (br s, 2H), 2.93-3.05 (m, 3H), 1.24-1.32 (m, 3H), 1.14-1.21 (m, 6H)

Synthesis of Intermediate BA-3

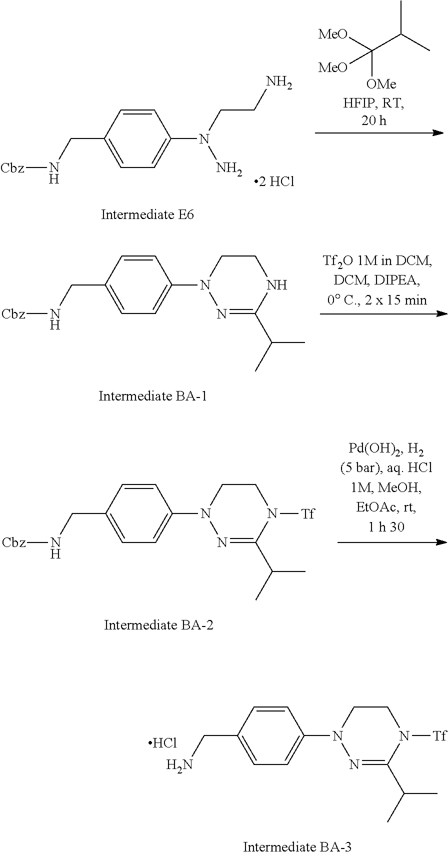

Preparation of Intermediate BA-1 According, intermediate BA-1 was prepared in the same way as AZ-1 starting from intermediate E6 (6.45 mol) yielding a colorless oil, 1.82 g (77%).

Preparation of Intermediate BA-2

Accordingly, intermediate BA-2 was prepared in the same way as compound 216 starting from BA-1 (4.97 mmol), yielding a beige powder, 1.58 g (58%).

Preparation of Intermediate BA-3

According, intermediate BA-3 was prepared in the same way as AY-3 starting from intermediate BA-2 (3.17 mol) yielding a beige solid, 1.39 g (91%, purity around 90%, used as such for next step).

The following compounds are/were also prepared in accordance with the methods described herein:

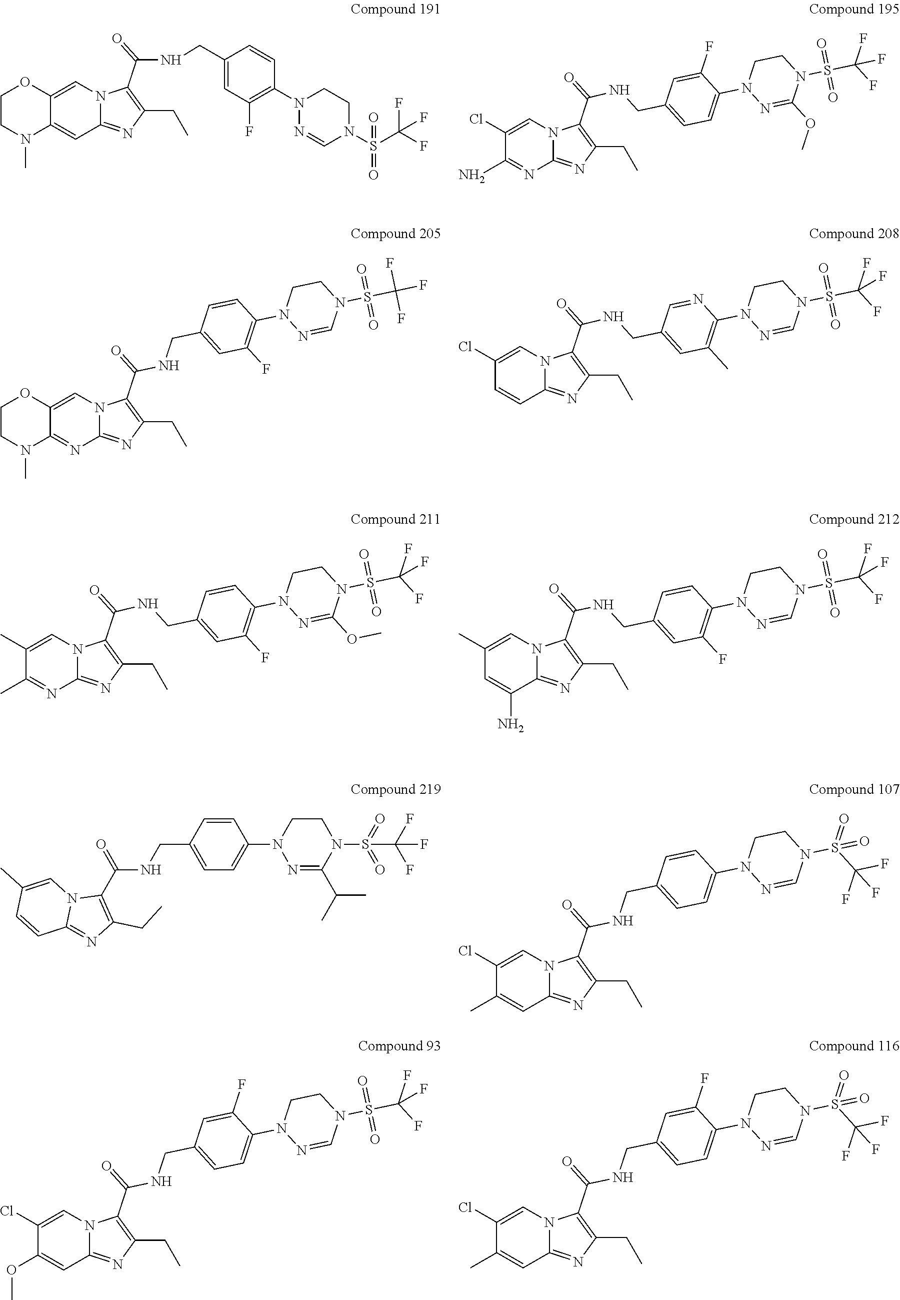

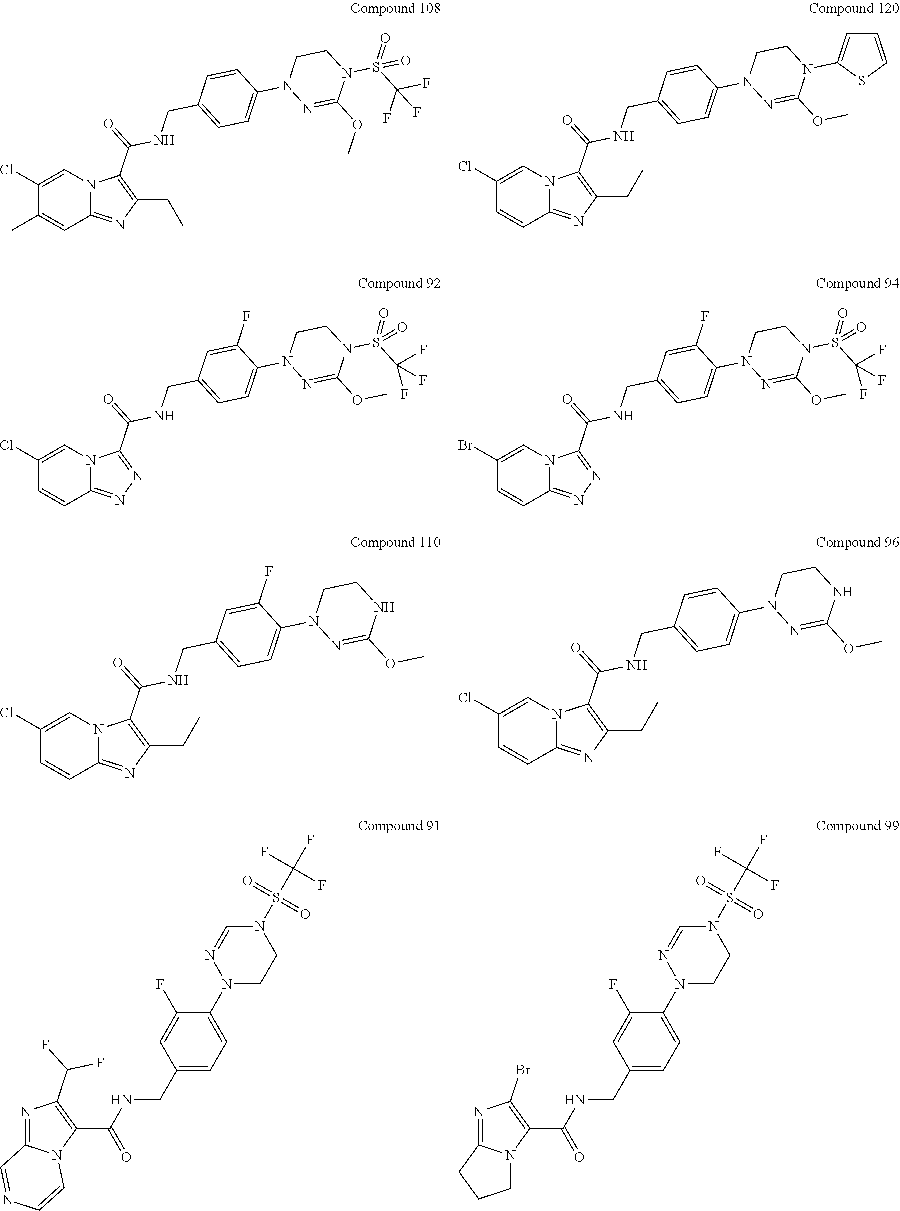

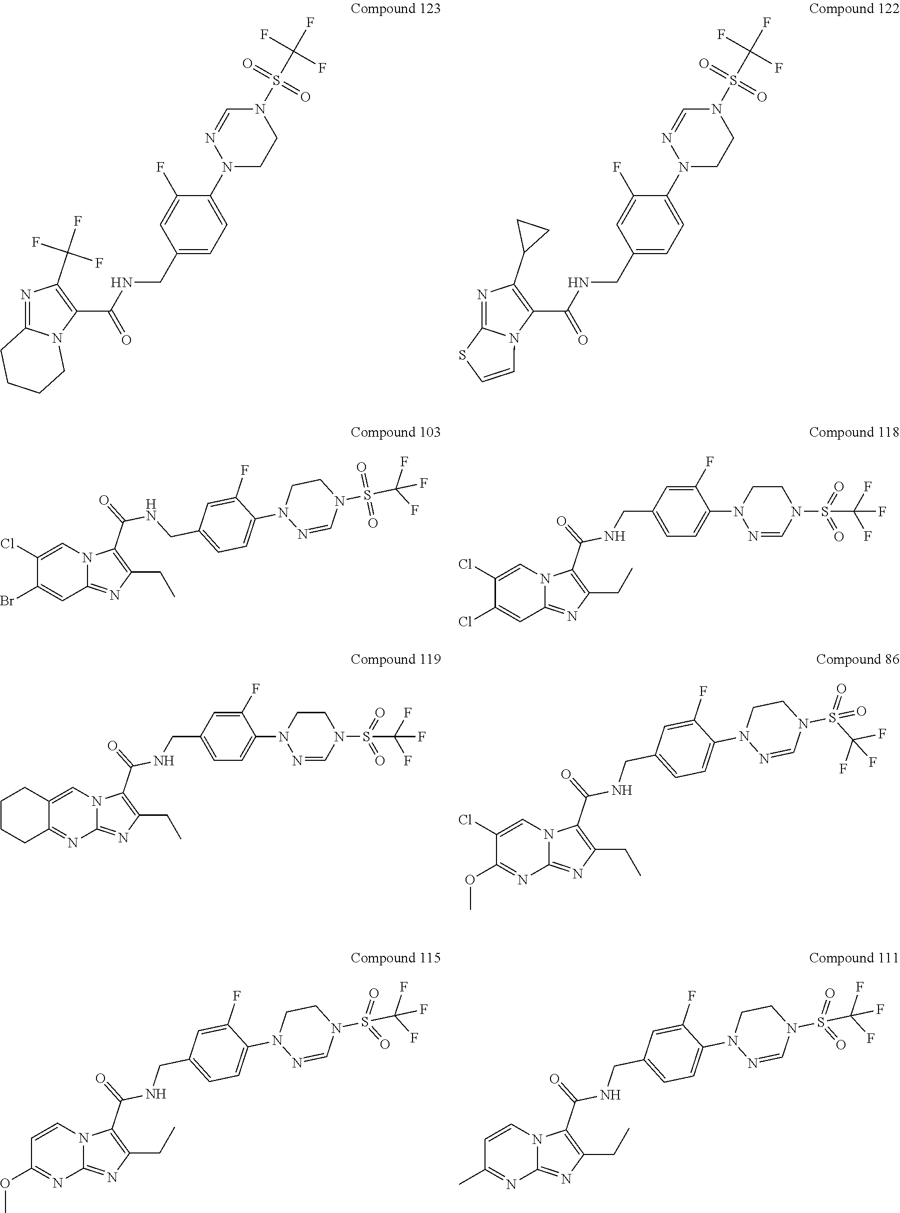

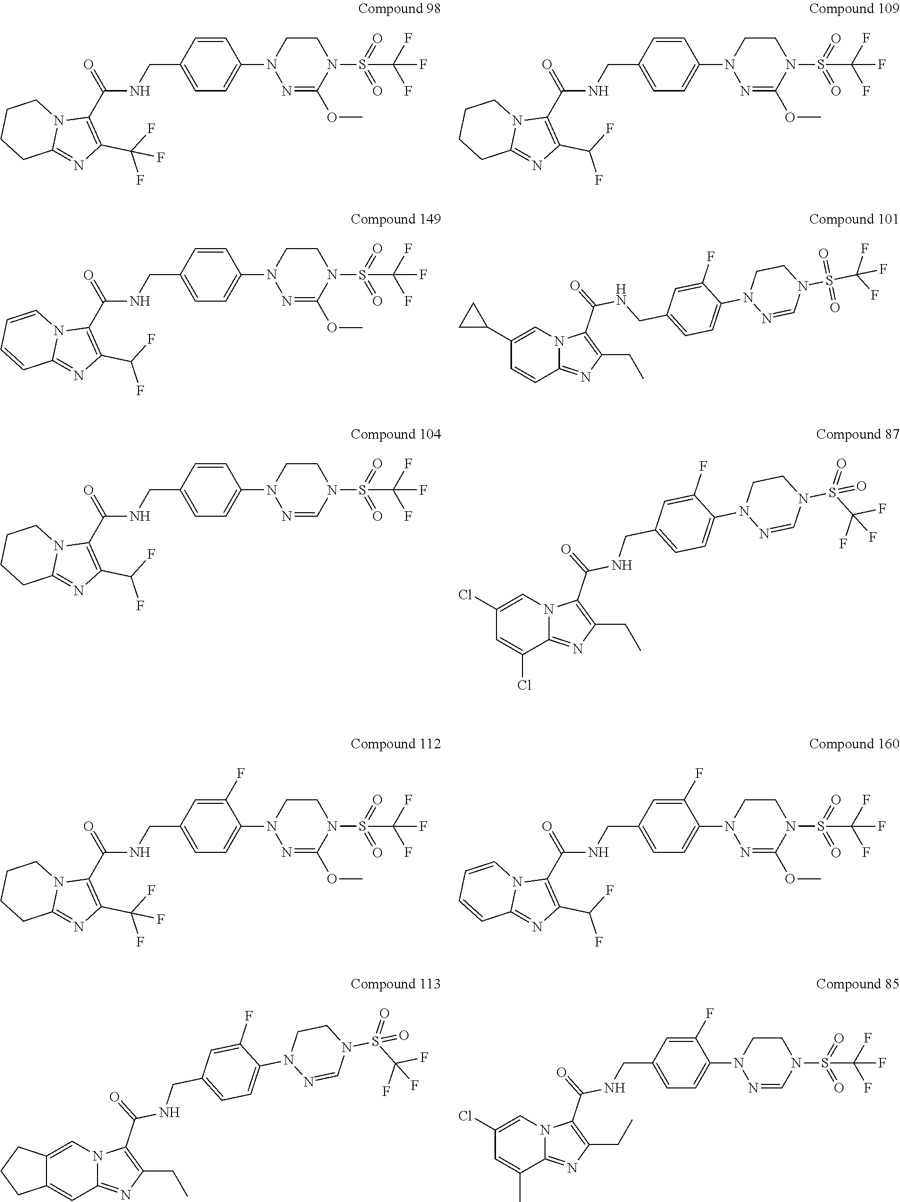

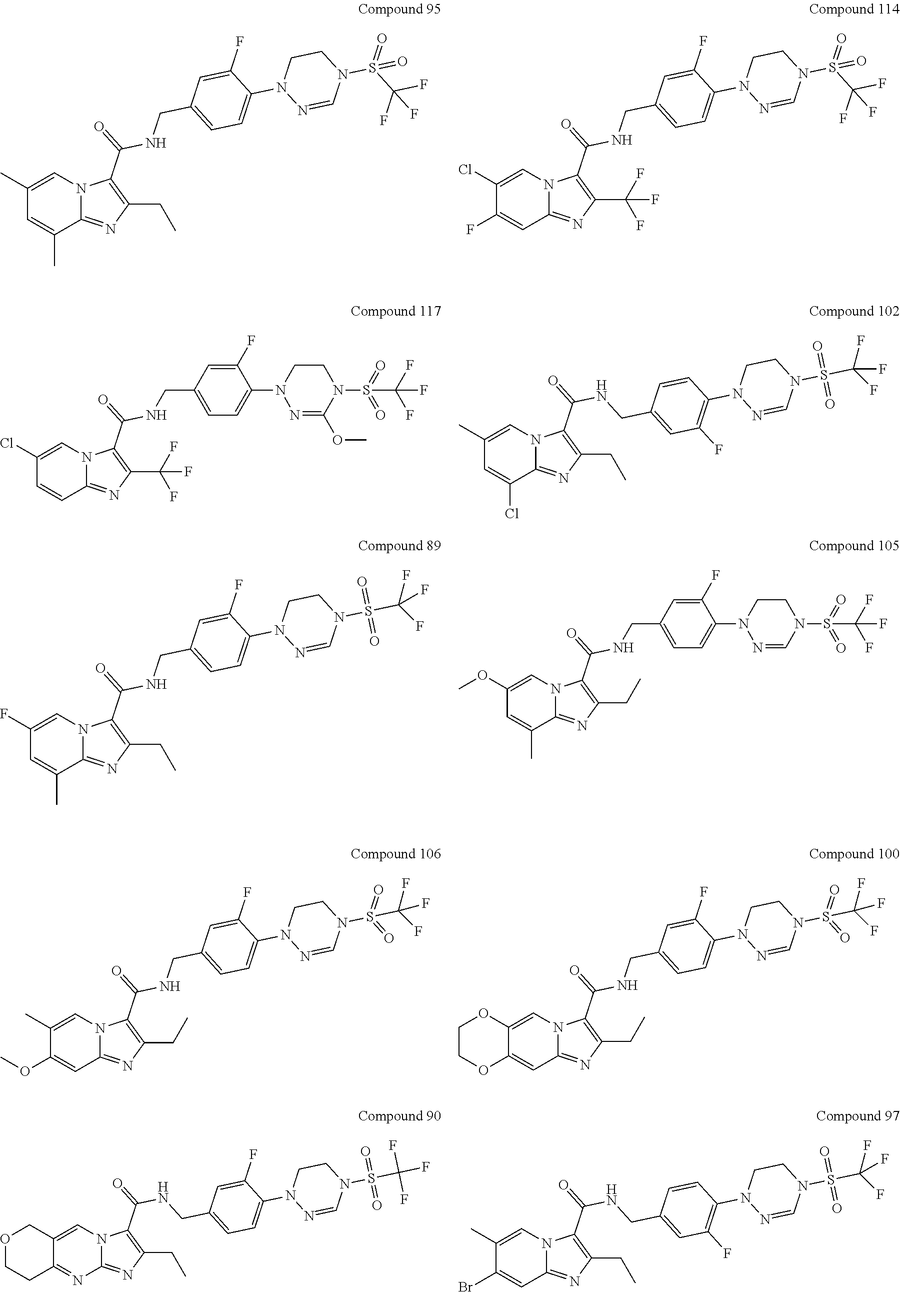

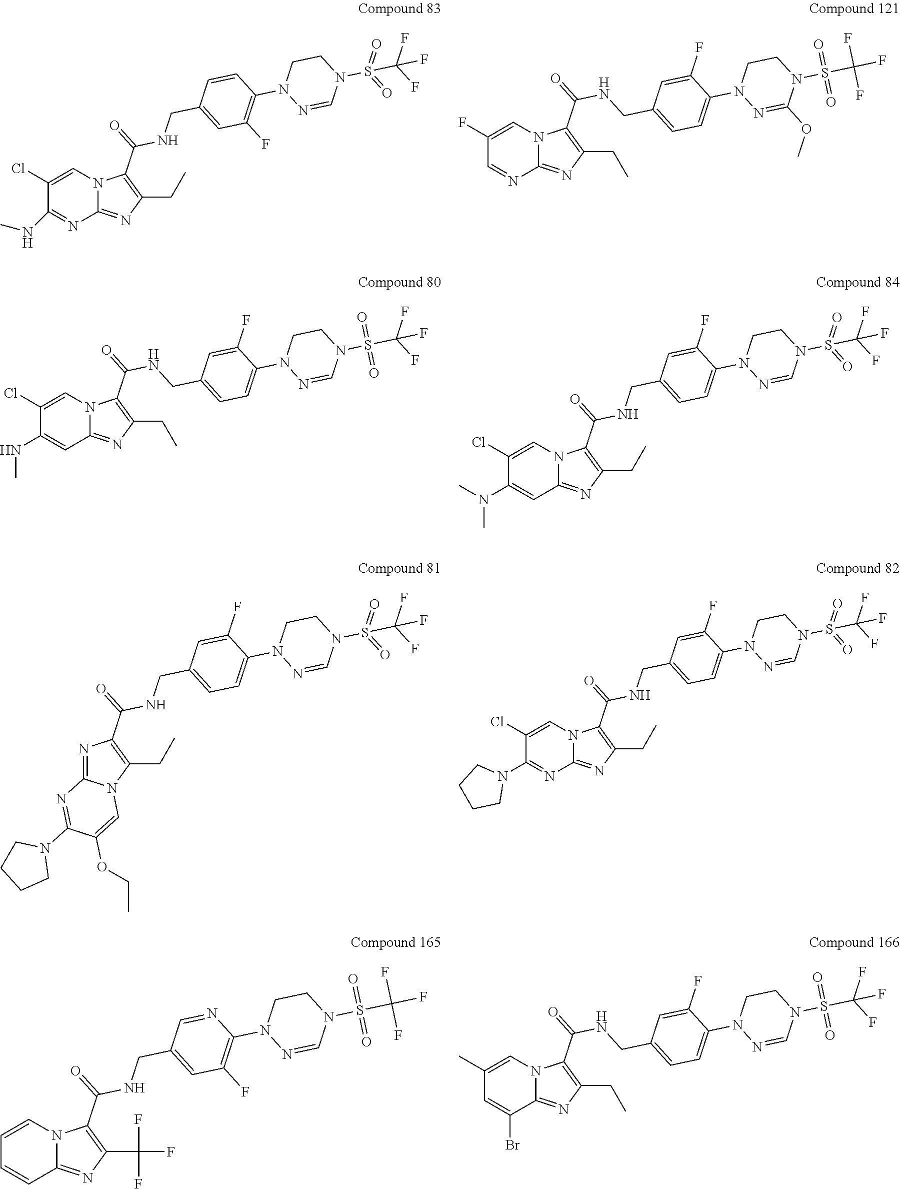

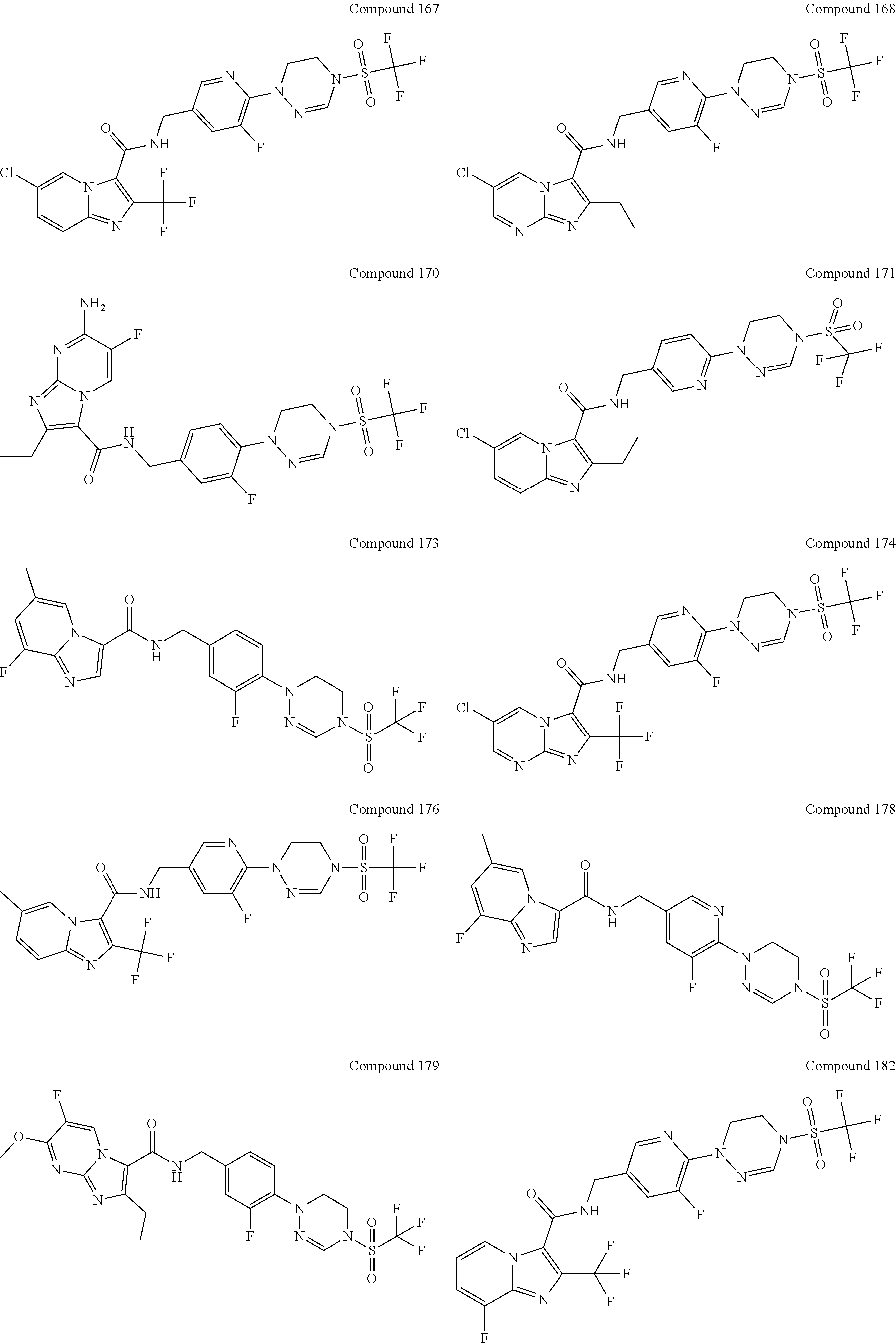

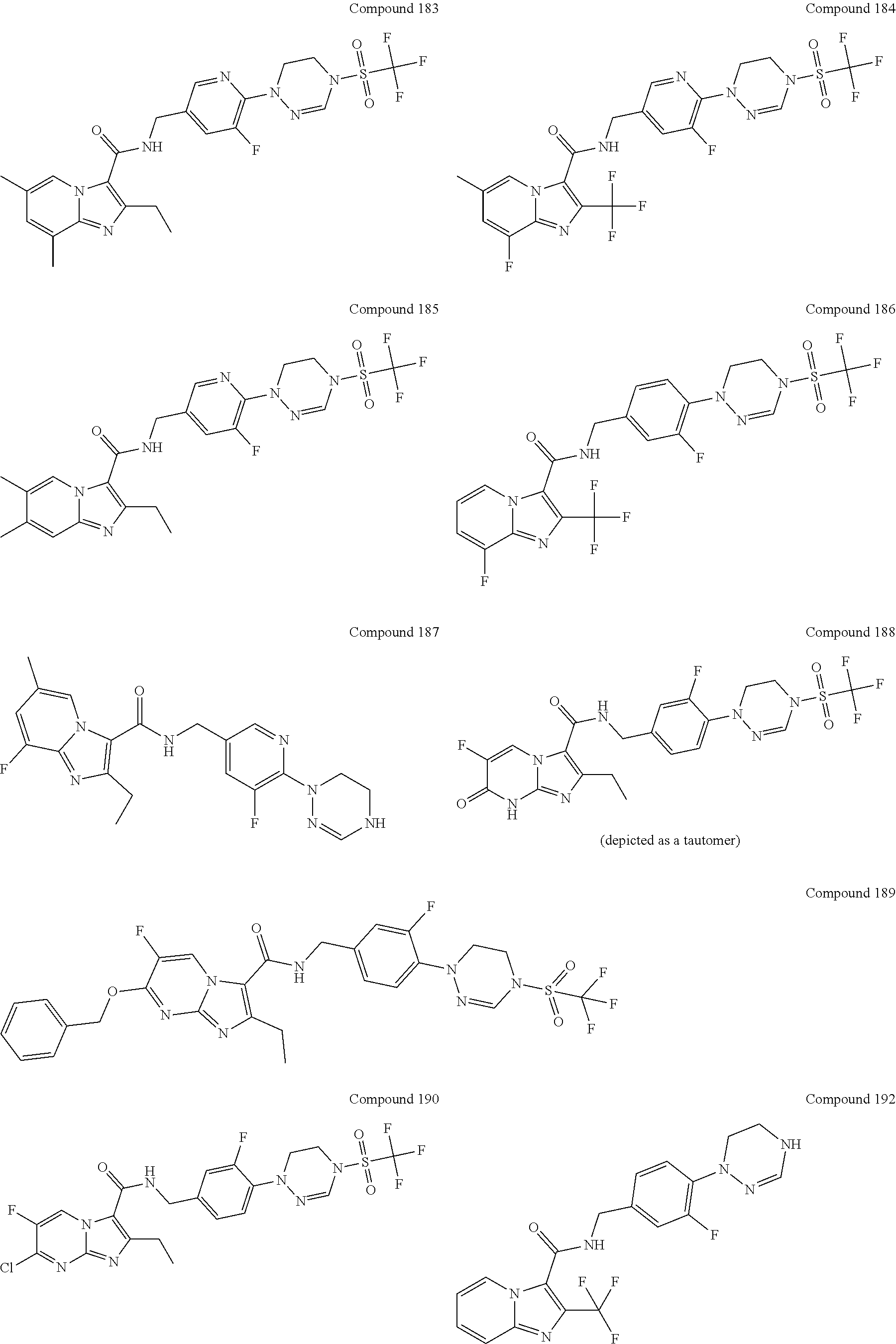

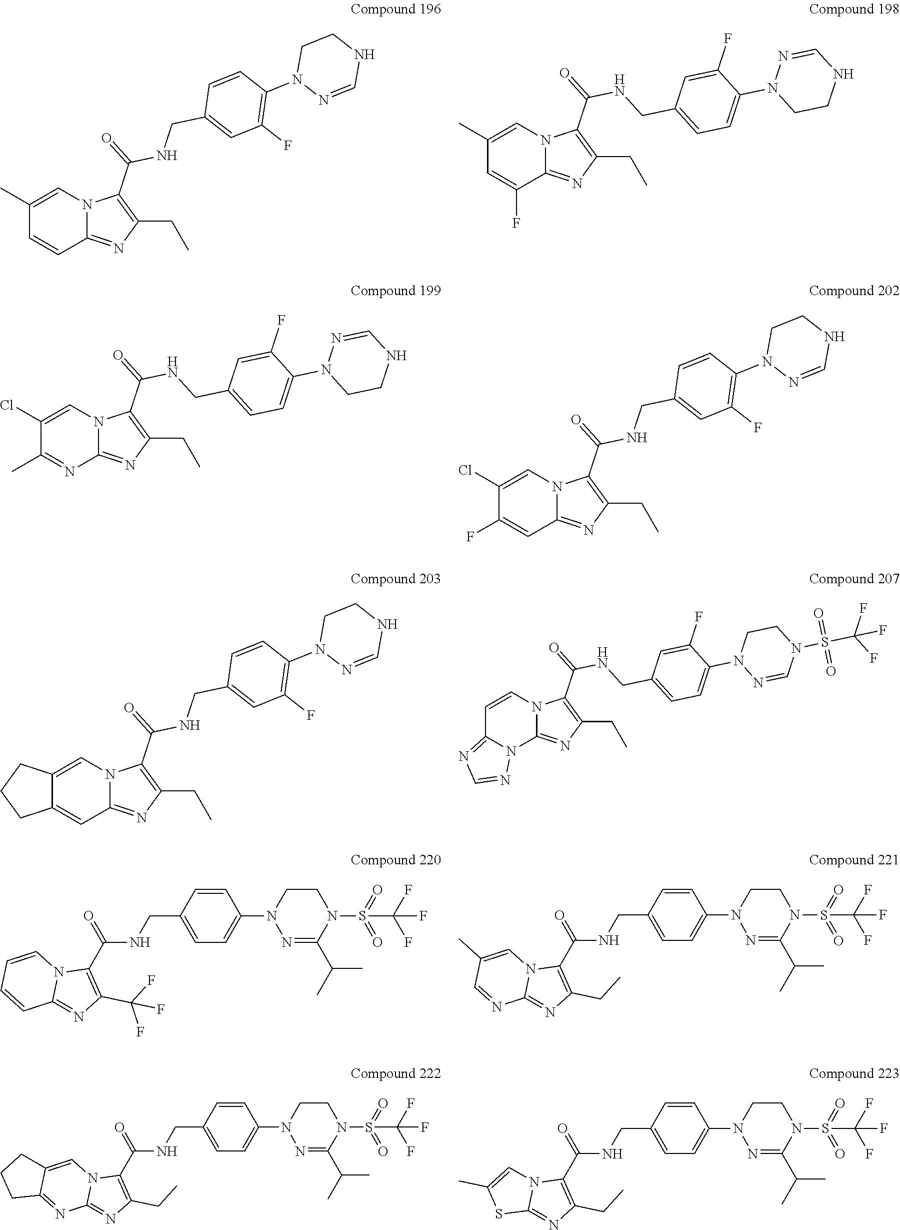

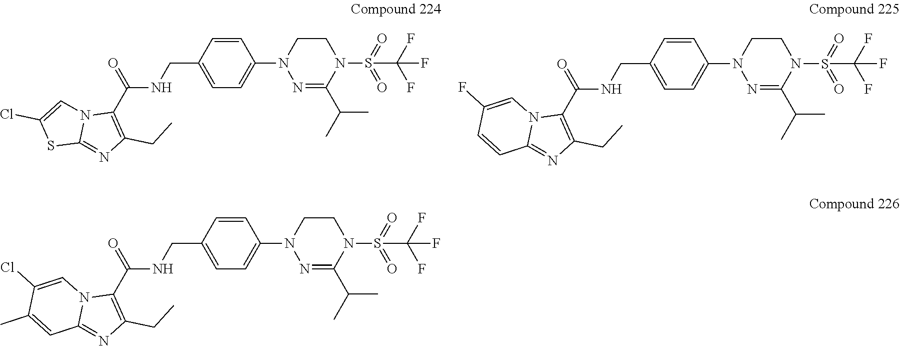 [SMILES: CCc1nc2sc(Cl)cn2c1C(=O)NCc1ccc(N2CCN(S(=O)(=O)C(F)(F)F)C(C(C)C)=N2)cc1 ; CCc1nc2ccc(F)cn2c1C(=O)NCc1ccc(N2CCN(S(=O)(=O)C(F)(F)F)C(C(C)C)=N2)cc1 ; CCc1nc2cc(C)c(Cl)cn2c1C(=O)NCc1ccc(N2CCN(S(=O)(=O)C(F)(F)F)C(C(C)C)=N2)cc1]

| | | LCMS | | | | | |
|---|---|---|---|---|---|---|---|
| Compound | Melting | Retention | | M | | | |
| number | point (° C.) | time | UV (%) | exact | [M + H]⁺ | [M − H]⁻ | Method |
| 1 | 183.46 | 3.2 | 98.9 | 528.1 | 529.1 | 527.2 | A |
| 31 | | 3.26 | 97.1 | 528.1 | 529.3 | 527.4 | A |
| 32 | | 3.25 | 96.86 | 528.1 | 529.3 | 527.4 | A |
| 29 | 195.09 | 3.25 | 96.17 | 528.1 | 529.3 | 527.4 | A |
| 28 | 199.08 | 3.26 | 98.84 | 528.1 | 529.3 | 527.4 | A |
| 30 | 213.87 | 3.26 | 96.73 | 528.1 | 529.3 | 527.4 | A |
| 21 | 157.49 | 3.36 | 100 | 542.1 | 543.2 | 541.3 | A |
| 19 | 196.34 | 2.73 | 96.2 | 518.2 | 519.2 | 517.3 | A |
| 2 | 178.31 | 3.52 | 99.8 | 556.1 | 557.2 | 555.3 | A |
| 56 | 236.36 | 2.32 | 99.8 | 396.1 | 397.1 | 395.1 | A |
| 20 | 209.36 | 2.65 | 98 | 474.1 | 475.1 | 473.2 | A |
| 3 | 189.22 | 2.82 | 100 | 498.2 | 499.3 | 557.4 [M + CH₃CO₂]⁻ | A |
| 57 | 211.47 | 1.85 | 95.7 | 366.2 | 367.2 | 425.4 [M + CH₃CO₂]⁻ | A |
| 13 | 215.87 | 3.06 | 97.1 | 529.1 | 530.3 | 528.3 | A |
| 58 | | 2.32 | 97.7 | 488.2 | 489.4 | 547.5 [M + CH₃CO₂]⁻ | A |
| 14 | 192.27 | 2.93 | 99.9 | 510.1 | 511.2 | 569.4 [M + CH₃CO₂]⁻ | A |
| 59 | 202.94 | 2.52 | 99.7 | 424.2 | 425.2 | 423.3 | A |
| 15 | 206.66 | 2.53 | 99 | 410.2 | 411.2 | 409.2 | A |
| 6 | | 3.28 | 99.4 | 558.1 | 559.3 | 557.4 | A |
| 16 | 158.10 | 3.21 | 100 | 546.1 | 547.5 | 545.5 | B |
| 4 | | 2.23 | 99.1 | 410.2 | 411.6 | 409.5 | B |
| 18 | 245.36 | 2.56 | 99.7 | 439.2 | 439.3 | 437.3 | A |
| 5 | | 3.29 | 100 | 542.1 | 543.3 | 541.4 | A |
| 17 | 196.82 | 3.08 | 97.6 | 516.2 | 517.4 | 515.5 | A |
| 22 | 175.12 | 3.29 | 92.2 | 558.1 | 559.4 | 557.4 | A |
| 23 | 207.21 | 2.94 | 97.82 | 517.2 | 518.4 | 516.5 | A |
| 10 | 171.42 | 2.97 | 99.59 | 513.1 | 514.3 | 512.4 | A |
| 24 | 258.01 | 2.38 | 97.6 | 439.2 | 440.3 | 438.4 | A |
| 26 | | 2.93 | 98.42 | 525.1 | 526.4 | 524.5 | A |
| 11 | 179.76 | 3.17 | 98.6 | 514.1 | 515.3 | 513.4 | A |
| 12 | 109.51, 164.64, 179.98 | 2.81 | 99.8 | 495.1 | 496.3 | 494.4 | A |
| 9 | 153.26, 177.06 | 3.3 | 99.6 | 546.1 | 547.3 | 545.4 | A |
| 8 | 167.12 | 3.15 | 98.8 | 544.1 | 545.5 | 543.5 | B |
| 25 | 125.56 | 3.24 | 97.97 | 558.1 | 559.3 | 557.5 | A |
| 7 | 208.41 | 3.19 | 98.34 | 541.1 | 542.3 | 540.5 | A |
| 27 | 174.88 | 3.16 | 100 | 559.1 | 560.3 | 558.4 | A |
| 60 | | 2.78 | 98.9 | 566.2 | 567.3 | 625.5 [M + AcO]⁻ | A |
| 61 | 193.44 | 2.74 | 97.6 | 552.2 | 553.3 | 611.4 [M + AcO]⁻ | A |
| 62 | | 2.94 | 97.2 | 537.1 | 538.2 | 596.5 [M + CH₃CO₂]⁻ | A |
| 33 | 225.92 | 2.85 | 98.52 | 539.2 | 540.3 | 538.3 | A |
| 38 | 255.93 | 2.52 | 98.91 | 424.1 | 425.1 | 423.2 | A |
| 39 | 211.19 | 3.08 | 98.89 | 558.1 | 559.2 | 557.3 | A |
| 40 | | 2.98 | 100 | 572.1 | 573.3 | 571.4 | A |
| 63 | 159.95 | 3.12 | 100 | 566.2 | 567.4 | 565.4 | A |
| 41 | | 2.82 | 98.72 | 573.1 | 574.3 | 572.7 | A |
| 34 | 86.24, 147.08 | 3.04 | 100 | 540.1 | 541.2 | 599.4 [M + AcO]⁻ | A |
| 35 | 152.93 | 3.25 | 99.56 | 564.1 | 565.3 | 563.4 | A |
| 64 | 193.79 | 3.26 | 98.82 | 558.1 | 559.3 | 557.3 | A |
| 65 | 228.15 | 3.3 | 98.84 | 563.2 | 564.4 | 562.5 | A |
| 42 | 147.57 | 3.06 | 98.5 | 524.1 | 525.3 | 523.4 | A |
| 43 | 169.05 | 3.02 | 100 | 538.2 | 539.3 | 537.4 | A |
| 66 | 211.30 | 3.08 | 98.23 | 492.1 | 493.2 | 491.2 | A |
| 67 | 206.99 | 3.22 | 99.6 | 558.1 | 559.3 | 557.3 | A |
| 36 | 142.03 | 3.11 | 98.33 | 554.2 | 555.3 | 553.3 | A |
| 37 | 193.36 | 3.37 | 99.74 | 576.1 | 577.2 | 575.3 | A |
| 44 | 173.29 | 3.1 | 99.83 | 558.1 | 559.3 | 617.5 [M + AcO]⁻ | A |
| 45 | 155.29 | 3.29 | 99.85 | 588.1 | 589.3 | 587.3 | A |
| 68 | | 3.29 | 99.62 | 588.1 | 589.3 | 587.5 | A |
| 46 | 176.82 | 3.34 | 100 | 606.1 | 607.3 | 605.4 | A |
| 47 | 149.44 | 3.09 | 100 | 565.1 | 566.3 | 564.4 | A |
| 69 | 163.46 | 2.99 | 98.43 | 561.1 | 562.3 | 560.2 | A |
| 70 | 138.71 | 3.01 | 99.79 | 558.1 | 559.3 | 617.6 [M + CH3COO]− | A |
| 48 | 74.60 | 3.19 | 99.7 | 572.1 | 573.3 | 571.4 | A |
| 49 | | 3.37 | 99.7 | 606.1 | 607.3 | 605.3 | A |
| 50 | 101.66/150.99 | 3.06 | 100 | 573.1 | 574.3 | 572.3 | A |
| 51 | 185.08 | 3.04 | 97.89 | 524.1 | 525.3 | 523.3 | A |
| 52 | 164.28 | 3.11 | 99.62 | 554.2 | 555.5 | 553.3 | A |
| 71 | | 3.14 | 98.26 | 549.1 | 550.3 | 548.4 | A |
| 72 | 216.25 | 3.07 | 98.33 | 519.1 | 520.2 | 518.2 | A |
| 53 | | 3.11 | 100 | 602.2 | 603.4 | 601.4 | A |
| 54 | 233.23 | 3.25 | 99.45 | 509.1 | 510.3 | 508.5 | A |
| 55 | 193.51 | 3.38 | 99.24 | 586.1 | 587.4 | 585.4 | A |
| 73 | 212.08 | 3.18 | 100 | 567.1 | 568.3 | 566.5 | A |
| 74 | | 3.42 | 99.2 | 585.2 | 586.4 | 584.5 | A |
| 75 | 135.16 | 3.16 | 97.19 | 572.1 | 573.3 | 571.4 | A |
| 76 | 232.40 | 2.95 | 99.82 | 479.1 | 480.3 | 478.4 | A |
| 77 | | 3.14 | 100 | 567.1 | 568.3 | 566.3 | A |

| | Melting | LCMS | | | | | |
|---|---|---|---|---|---|---|---|
| Compound | point (° C.) | Retention | | | | | |
| number | (DSC or MT) | time | UV (%) | M exact | [M + H]⁺ | [M − H]⁻ | Method |
| 132 | 179.05 | 3.52 | 99.47 | 590.1 | 591.5 | 589.4 | A |
| 107 | 207.55 | 3.35 | 98.4 | 542.1 | 543.4 | 541.3 | A |
| 93 | 187.84 | 3.24 | 99.46 | 576.1 | 577.5 | 575.5 | A |
| 116 | 175.40 | 3.39 | 99.8 | 560.1 | 561.4 | 559.4 | A |
| 108 | | 3.41 | 100 | 572.1 | 573.5 | 571.4 | A |
| 146 | 173.85 | 3.12 | 99.58 | 547.1 | 548.4 | 546.3 | A |
| 120 | 183.66 | 3.36 | 98.02 | 508.1 | 509.4 | 507.3 | A |
| 92 | | 3.22 | 99.71 | 549.1 | 550.4 | 548.3 | A |
| 94 | 192.26 | 3.26 | 99.24 | 593 | 594.3 | 592.3 | A |
| 141 | 198.76 | 3.46 | 98.37 | 594.1 | 594.4 | 593.4 | A |
| 110 | 175.59 | 2.75 | 98.27 | 444.1 | 445.3 | 443.3 | A |
| 96 | 133.99 | 2.7 | 98.5 | 426.2 | 427.3 | 425.3 | A |
| 156 | 151.06 | 3.13 | 99.16 | 530.1 | 531.4 | 529.4 | A |
| 164 | | 3.12 | 100 | 526.1 | 527.4 | 525.4 | A |
| 91 | | 2.74 | 100 | 513.1 | 514.2 | 512.2 | C |
| 99 | | 2.79 | 100 | 552 | 555 | 553.1 | C |
| 123 | 157.82 | 3.06 | 100 | 556.1 | 557.4 | 555.4 | A |
| 147 | 147.39 | 2.82 | 97 | 538.1 | 539.3 | 537.3 | B |
| 157 | 183.19 | 3.35 | 99.4 | 564.1 | 565.3 | 563.3 | A |
| 152 | 169.75 | 3.15 | 100 | 552.1 | 553.3 | 551.3 | A |
| 159 | 134.19 | 2.91 | 100 | 534.1 | 535.3 | 533.4 | B |
| 103 | 169.72 | 3.15 | 100 | 530.1 | 531.3 | 529.2 | A |
| 103 | 197.38 | 3.58 | 100 | 624 | 625.2 | 623.2 | A |
| 154 | 172.60 | 3.01 | 99.51 | 553.2 | 554.4 | 552.5 | A |
| 118 | 188.26 | 3.54 | 98.6 | 580 | 581.4 | 579.3 | A |
| 119 | 173.10 | 3.13 | 99.41 | 567.2 | 568.5 | 566.5 | A |
| 142 | 190.96 | 3.15 | 100 | 547.1 | 548.3 | 546.3 | A |
| 163 | 158.33 | 3.21 | 100 | 532.1 | 533.3 | 531.3 | A |
| 125 | 141.53 | 3.11 | 99.53 | 547.1 | 548.3 | 546.4 | A |
| 86 | 152.96 | 3.01 | 100 | 577.1 | 578.1 | 576.2 | A |
| 115 | 137.25 | 3.04 | 100 | 543.1 | 544.5 | 542.5 | A |
| 111 | 176.27 | 2.65 | 100 | 527.1 | 528.1 | 526.3 | C |
| 98 | 168.32 | 2.83 | 99.44 | 568.1 | 569.2 | 567.3 | C |
| 150 | 120.10 | 2.92 | 100 | 564.1 | 565.1 | 563.2 | C |
| 109 | 137.30 | 2.76 | 98.5 | 550.1 | 551.2 | 549.3 | C |
| 149 | 145.84 | 2.85 | 100 | 546.1 | 547.1 | 545.2 | C |
| 153 | 177.80 | 2.82 | 100 | 518.1 | 519 | 517.2 | C |
| 130 | | 2.75 | 100 | 51/1 | 532.1 | 530.2 | C |
| 133 | 165.25 | 3.27 | 99.55 | 538 | 539.3 | 537.3 | A |
| 126 | 239.37 | 2.88 | 100 | 562.1 | 563.4 | 561.3 | A |
| 129 | 135.91 | 3.19 | 99.77 | 546.1 | 547.4 | 545.4 | A |
| 101 | 171.00 | 3.26 | 100 | 552.2 | 553.5 | 551.5 | A |
| 161 | 167.34 | 2.93 | 100 | 527.1 | 528.4 | 526.4 | A |
| 88 | 194.58 | 3.02 | 100 | 538.1 | 539.3 | 537.4 | A |
| 127 | 179.93 | 3.12 | 99.4 | 534.1 | 535.3 | 533.3 | A |
| 104 | 154.40 | 2.93 | 100 | 520.1 | 521.4 | 519.4 | A |
| 128 | 170.22 | 3.04 | 100 | 516.1 | 517.3 | 515.3 | A |
| 158 | 163.01 | 3.09 | 100 | 583.2 | 584.5 | 582.5 | A |
| 87 | 171.19 | 3.51 | 99.75 | 580 | 581.3 | 579.4 | A |
| 155 | 181.61 | 3.2 | 100 | 561.1 | 562.4 | 560.4 | A |
| 151 | 183.62 | 3.39 | 100 | 564.1 | 565.3 | 563.4 | A |
| 112 | 146.66 | 3.12 | 100 | 586.1 | 587.4 | 585.4 | A |
| 137 | 136.03 | 3.04 | 100 | 568.1 | 569.4 | 567.3 | A |
| 160 | 127.38 | 3.14 | 99.38 | 564.1 | 565.4 | 563.4 | A |
| 113 | 194.44 | 3.27 | 99.1 | 552.2 | 553.5 | 551.5 | A |
| 85 | 158.44 | 3.48 | 100 | 560.1 | 561.4 | 559.3 | A |
| 145 | 149.39 | 3.29 | 100 | 568.1 | 569.3 | 567.3 | A |
| 95 | 154.17 & 147.66 | 3.29 | 100 | 540.2 | 541.4 | 539.4 | A |
| 124 | 161.38 | 3.35 | 100 | 586 | 587.3 | 585 | A |
| 144 | 146.89 | 3.35 | 100 | 586 | 587.3 | 585.3 | A |
| 114 | 169.31 | 3.4 | 97.63 | 604 | 605.3 | 603.3 | A |
| 117 | 164.15 | 3.42 | 100 | 616.1 | 617.3 | 615.3 | A |
| 102 | 174.33 | 3.32 | 100 | 560.1 | 561.4 | 559.4 | A |
| 148 | 157.00 | 3.24 | 99.21 | 548.1 | 549.3 | 547.3 | A |
| 89 | 153.88 | 3.29 | 98.1 | 544.1 | 545.4 | 543.4 | A |
| 162 | 160.65 | 3.22 | 99.3 | 544.1 | 545.4 | 543.4 | A |
| 105 | 172.82 | 3.21 | 98.93 | 556.2 | 557.4 | 555.4 | A |
| 143 | 226.52 | 301 | 100 | 561 | 562.3 | 560.3 | A |
| 136 | 147.95 | 3.37 | 100 | 572.1 | 573.3 | 571.3 | A |
| 135 | 167.06 | 3.14 | 99.4 | 552 | 553.1 | 551.1 | C |
| 131 | 199.18 | 3.04 | 98.71 | 554.1 | 555.4 | 553.4 | A |
| 134 | 185.60 | 3.02 | 100 | 556.1 | 557.3 | 555.3 | A |
| 106 | 198.37 | 3.2 | 99.02 | 556.2 | 557.4 | 555.3 | A |
| 100 | 174.31 | 3.02 | 100 | 570.1 | 571.3 | 569.3 | A |
| 139 | 176.81 | 3.21 | 100 | 540.2 | 541.4 | 539.4 | A |
| 140 | 183.14 | 3.41 | 99.01 | 560.1 | 561.3 | 559.3 | A |
| 90 | 233.3 (MT) | 3.2 | 97 | 569.1 | 570.1 | | D |
| 97 | | 3.45 | 1Â°Â° | 604.1 | 605.4 | 603.3 | A |
| 83 | | 3.02 | 99.46 | 576.1 | 577.3 | 575.3 | A |
| 121 | 149.7 (MT) | 3.712 | 98 | 561.1 | 562.1 | | D |
| 80 | | 3.13 | 97.74 | 575.1 | 576.4 | 574.3 | A |
| 138 | | 2.87 | 100 | 541.2 | 542.4 | 540.4 | A |
| 84 | 141.30 | 3.3 | 100 | 600.2 | 601.5 | 659.4 [M + CH3CO2]− | A |
| 81 | 173.01 | 3.46 | 100 | 626.2 | 627.5 | 685.5 [M + CH3COO]− | A |
| 82 | 187.66 | 3.45 | 100 | 616.1 | 617.4 | 615.4 | A |
| 165 | 189.8 (MT) | 3.553 | 99 | 553 | 554 | | D |
| 166 | | 3.12 | 100 | 604.1 | 605.1 | 603.2 | C |
| 167 | 153.0 (MT) | 3.89 | 98 | 587 | 588 | | D |
| 168 | 148.1 (MT) | 3.525 | 97 | 548.1 | 549.1 | | D |
| 169 | | 3.296 | 98 | 569.1 | 570.3 | | D |
| 170 | | 3.333 | 97 | 546.1 | 547.3 | | D |
| 171 | 203.43 | 3.22 | 99.48 | 529.1 | 530.3 | 538.2 | A |
| 173 | 183.2 (MT) | 3.82 | 99 | 516.1 | 516 | | D |
| 174 | 148.1 (MT) | 3.52 | 97 | 548 | 549 | | D |
| 175 | 146.4 (MT) | 4.048 | 98 | 566.1 | 567.1 | | D |
| 176 | 163.1 (MT) | 3.814 | 99 | 567.1 | 568.1 | | D |
| 177 | 166.4 (MT) | 3.693 | 95 | 545.1 | 546.1 | | D |
| 178 | 193.2 (MT) | 3.563 | 99 | 517.1 | 518.1 | | D |
| 179 | 169.8 (MT) | 3.721 | 99 | 561.1 | 562.1 | | D |
| 180 | | 3.457 | 99 | 599.1 | 600.1 | | D |
| 181 | 198.3 (MT) | 3.641 | 99 | 545.1 | 546.1 | | D |
| 182 | 184.7 | 3.762 | 97 | 571.1 | 572.1 | | D |
| 183 | 198.1 | 2.966 | 97 | 541.1 | 542.1 | | D |
| 184 | 193.2 (MT) | 3.931 | 98 | 585.1 | 586.1 | | D |
| 185 | 181.5 (MT) | 2.875 | 98 | 541.1 | 542.1 | | D |
| 186 | 158.1 (MT) | 3.987 | 99 | 570.1 | 571.2 | | D |
| 187 | 216.6 (MT) | 1.668 | 98 | 413.1 | 414.1 | | D |
| 188 | | 3.138 | 98 | 547.1 | 548.1 | | D |
| 189 | 169.8 (MT) | 4.286 | 99 | 637.1 | 638.2 | | D |
| 190 | | 4.015 | 98 | 567.1 | 566.1 | | D |
| 191 | 178.1 (MT) | 2.96 | 99 | 583.1 | 584.1 | | D |
| 192 | 189.9 (MT) | 1.816 | 98 | 420.1 | 421.1 | | D |
| 193 | 153.1 (MT) | 3.535 | 99 | 557.1 | 558.1 | | D |
| 194 | 137.9 (MT) | 4.136 | 98 | 596.1 | 597.1 | | D |
| 195 | 226.7 (MT) | 3.05 | 99 | 591.1 | 592.1 | | D |
| 196 | | 1.454 | 97 | 395.2 | 396.2 | | D |
| 198 | 173.1 (MT) | 1.807 | 99 | 412.2 | 413.2 | | D |
| 199 | 120.4 (MT) | 1.863 | 98 | 429.1 | 430.1 | | D |
| 200 | 166.4 (MT) | 3.431 | 99 | 539.1 | 540.1 | | D |
| 201 | 159.8 (MT) | 3.103 | 99 | 528.1 | 529.1 | | D |
| 202 | 214.9 (MT) | 2.03 | 97 | 432.1 | 433.1 | | D |
| 203 | 208.2 (MT) | 1.48 | 99 | 421.2 | 422.2 | | D |
| 204 | 186.4 (MT) | 3.655 | 99 | 560.1 | 561.1 | | D |
| 205 | 201.5 (MT) | 2.906 | 97 | 584.1 | 585.1 | | D |
| 206 | | 4.025 | 99 | 598.1 | 599.1 | | D |
| 207 | 157.42 | 2.88 | 100 | 553.1 | 554.4 | 552.4 | A |
| 208 | 178.11 | 3.29 | 100 | 543.1 | 544.3 | 542.3 | A |
| 209 | 159.7 (MT) | 3.881 | 99 | 590.1 | 591.1 | | D |
| 210 | 178.5 (MT) | 4.33 | 99 | 594.1 | 595.1 | | D |
| 211 | 153.35 | 3.07 | 100 | 571.2 | 572.4 | 570.5 | A |
| 212 | | 3.04 | 98 | 541.2 | 542.4 | 540.4 | A |
| 213 | 159.38 | 3.46 | 100 | 560.1 | 561.4 | 559.3 | A |
| 214 | 106.9 | 3.28 | 97 | 548.1 | 549.4 | 547.4 | A |
| 215 | 152.81 | 3.26 | 97 | 522.2 | 523.3 | 521.4 | A |
| 216 | | 3.74 | 96.2 | 558.14 | 589.4 | 587.6 | A |
| 217 | | 3.03 | 100 | 565.2 | 566.4 | 564.4 | A |
| 218 | | 3.33 | 100 | 528.1 | 529.3 | 527.5 | A |
| 219 | | 3.36 | 99 | 550.2 | 551.2 | 549.3 | C |
| 220 | | 3.36 | 98 | 576.1 | 577.1 | 575.2 | C |
| 221 | | 3.18 | 96 | 551.2 | 552.2 | 550.3 | C |
| 222 | | 3.25 | 98 | 577.2 | 578.2 | 576.4 | C |
| 223 | | 3.44 | 99 | 556.1 | 557.2 | 555.2 | C |
| 224 | | 3.7 | 99 | 576.1 | 577.2 | 575.2 | C |
| 225 | | 3.35 | 96 | 554.2 | 555.2 | 553.3 | C |
| 226 | | 3.65 | 96 | 584.2 | 585.2 | 583.3 | C |
| 79 | 113.95 | 3.22 | 99.6 | 582.1 | 583.4 | 581.4 | A |

### Examples

Synthesis of Intermediates BB-3, BC-3 and BD-3

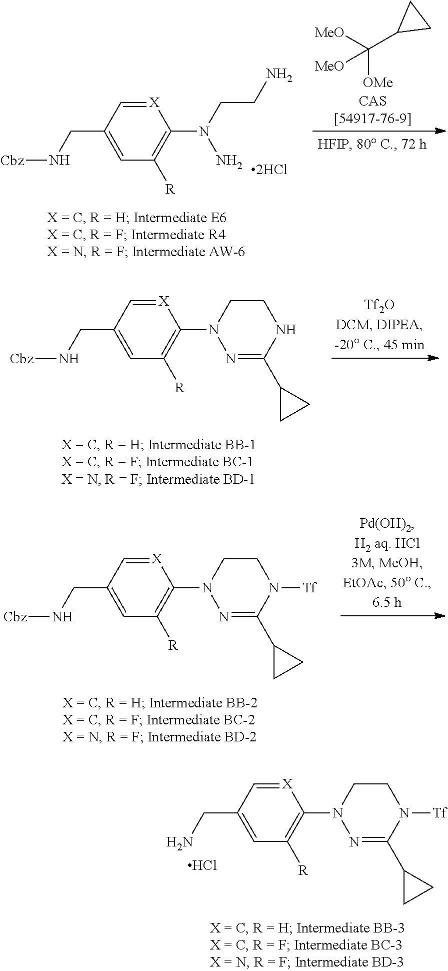

Preparation of Intermediate BB-1

(Trimethoxymethyl)cyclopropane (CAS [54917-76-9], 417 mg, 2.85 mmol) was added to a solution of intermediate E6 (1 g, 2.85 mmol) in HFIP (13 mL) in a glass pressure bottle while nitrogen was bubbling. The mixture was stirred at 80° C. for 72 h. The reaction mixture was cooled to room temperature and the solvent was removed in vacuo to give an orange oil. The crude product was purified by flash column chromatography over silica gel (25 g; (DCM/MeOH 9:1) in DCM from 0/100 to 90/10). The desired fractions were collected and concentrated in vacuo. The result was neutralized with NaHCO₃ (sat., aq.) and extracted with EtOAc. The combined organic layers were dried over MgSO₄, filtered and concentrated in vacuo to yield intermediate BB-1 (420 mg, 40%) as a beige foam solid.

Preparation of Intermediate BB-2

Tf₂O (194 μL, 1.15 mmol) in DCM (2 mL) was added dropwise to a stirred solution of intermediate BB-1 (420 mg, 1.15 mmol) and DIPEA (0.6 mL, 3.46 mmol) in DCM (8 mL) in a round bottom flask under N2 atmosphere at −20° C. The reaction mixture was stirred at −20° C. for 15 min. Then more Tf₂O (39 μL, 0.23 mmol) in DCM (1 mL) was added dropwise at −20° C. and the mixture was stirred at −20° C. for 30 min. The reaction was quenched at −20° C. with NaHCO₃ (sat., aq.) and extracted with DCM. The combined organic layers were dried over MgSO₄, filtered, and concentrated in vacuo. The crude product was purified by flash column chromatography (silica 25 g; EtOAc in Heptane from 0/100 to 20/80). The desired fractions were collected and concentrated in vacuo to yield intermediate BB-2 (438 mg, 75%) as a white solid.

Preparation of Intermediate BB-3

In a round bottom flask, palladium hydroxide, Pd 20% on carbon, nominally 50% water (62 mg, 0.088 mol) was added to a stirred solution of intermediate BB-2 (438 mg, 0.88 mmol) and HCl (3M, aq., 0.29 mL, 0.88 mmol) in EtOAc (12 mL) and MeOH (12 mL) at rt under nitrogen atmosphere. Then, nitrogen atmosphere was replaced by hydrogen and the reaction mixture was stirred at 50° C. for 5 h. An extra amount of palladium hydroxide, Pd 20% on carbon, nominally 50% water (31 mg, 0.044 mmol) was added to the reaction mixture at rt under nitrogen atmosphere. Then, nitrogen atmosphere was replaced by hydrogen and the reaction mixture was stirred at 50° C. for 1.5 h. The mixture was filtered through a pad of Celite® and the filtrate was concentrated in vacuo to intermediate BB-3 (351 mg, 99%) as a yellow solid. The crude product was used as such in the next step.

Preparation of Intermediate BC-1

Accordingly, intermediate BC-1 was prepared in the same way as intermediate BB-1, starting from intermediate R4 (1.23 mmol) affording 404 mg (77%) as a beige foam.

Preparation of Intermediate BC-2

Accordingly, intermediate BC-2 was prepared in the same way as intermediate BB-2, starting from intermediate BC-1 (1.06 mmol) affording 243 mg (40%) as a beige solid.

Preparation of Intermediate BC-3

Accordingly, intermediate BC-3 was prepared in the same way as intermediate BB-3, starting from intermediate BC-2 (0.47 mmol) affording 199 mg (quant.) as a yellow solid.

Preparation of Intermediate BD-1

Accordingly, intermediate BD-1 was prepared in the same way as intermediate BB-1, starting from intermediate AW-6 (2.4 mmol) affording 610 mg (60%) as a beige foam.

Preparation of Intermediate BD-2

Accordingly, intermediate BD-2 was prepared in the same way as intermediate BB-2, starting from intermediate BD-1 (0.7 mmol) affording 326 mg (85%) as a white solid.

Preparation of Intermediate BD-3

Accordingly, intermediate BD-3 was prepared in the same way as intermediate BB-3, starting from intermediate BD-2 (0.62 mmol) affording 260 mg (94%) as a yellow solid.

Synthesis of Compound 227

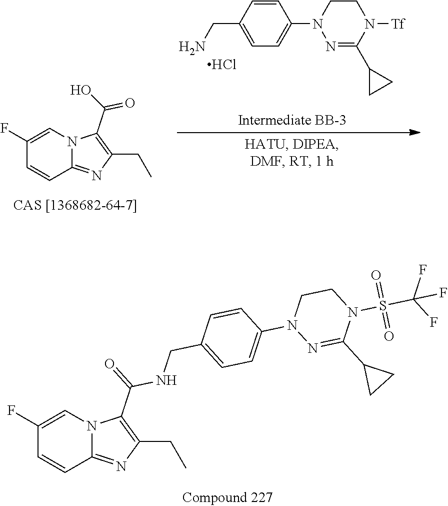

Intermediate BB-3 (115 mg, 0.29 mmol) was added to a solution of 2-ethyl-6-fluoroimidazo[1,2-a]pyridine-3-carboxylic acid (CAS [1368682-64-7], 87 mg, 0.35 mmol), HATU (165 mg, 0.43 mmol) and DIPEA (261 mg, 2.02 mmol) in DMF (3 mL) in a round bottom flask at rt. The mixture was stirred at rt for 1 h. NaHCO₃ (sat., aq.) was added and the mixture was extracted with EtOAc (×3). The combined organic layers were dried over MgSO₄, filtered and concentrated in vacuo. The crude product was purified by flash column chromatography (silica, 12 g; EtOAc in DCM from 0/100 to 25/75). The desired fractions were collected and concentrated in vacuo. The result was triturated with DIPE/DCM (9:1) and filtrated to yield compound 227 (100 mg, 62%) as a beige solid.

1H NMR (400 MHz, DMSO-d₆) δ 9.03 (dd, J=5.1, 2.4 Hz, 1H), 8.35 (t, J=5.8 Hz, 1H), 7.68 (dd, J=9.8, 5.4 Hz, 1H), 7.51-7.44 (m, 1H), 7.30 (d, J=8.6 Hz, 2H), 7.11 (d, J=8.6 Hz, 2H), 4.45 (d, J=5.9 Hz, 2H), 3.97 (t, J=4.9 Hz, 2H), 3.80 (t, J=5.0 Hz, 2H), 2.97 (q, J=7.5 Hz, 2H), 1.97-1.87 (m, 1H), 1.25 (t, J=7.5 Hz, 3H), 0.97-0.88 (m, 2H), 0.88-0.80 (m, 2H).

Synthesis of Compound 228

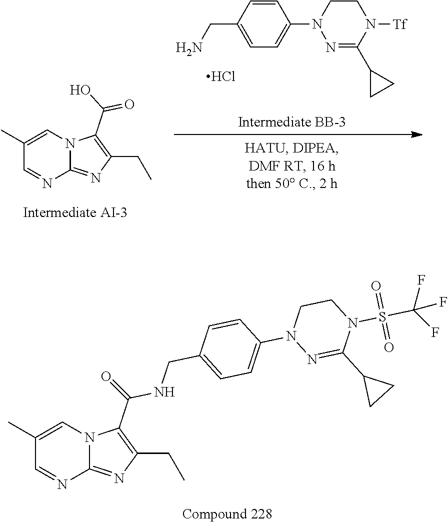

Accordingly, compound 228 was prepared in the same way as compound 227, starting from intermediate AI-3 (0.35 mmol) and intermediate BB-3 (0.39 mmol) stirring the mixture at rt for 16 h and at 50° C. for 2 h affording 63 mg (30%) as a white solid.

1H NMR (400 MHz, DMSO-d₆) δ 9.13 (s, 1H), 8.50 (d, J=2.3 Hz, 1H), 8.40 (t, J=5.9 Hz, 1H), 7.29 (d, J=8.6 Hz, 2H), 7.11 (d, J=8.6 Hz, 2H), 4.45 (d, J=5.9 Hz, 2H), 3.98 (t, J=4.8 Hz, 2H), 3.80 (t, J=4.9 Hz, 2H), 2.98 (q, J=7.5 Hz, 2H), 2.34 (s, 3H), 1.93-1.84 (m, 1H), 1.26 (t, J=7.5 Hz, 3H), 0.94-0.89 (m, 2H), 0.87-0.81 (m, 2H).

Synthesis of Compound 229

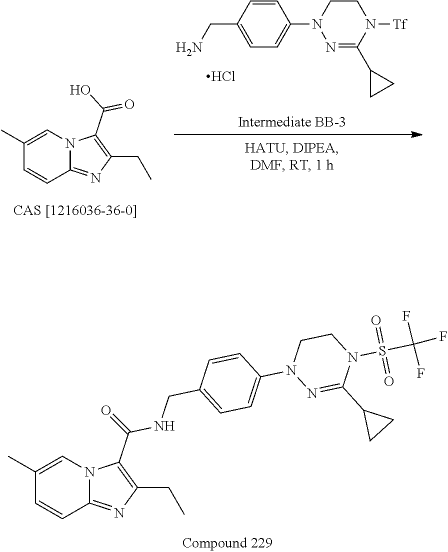

Accordingly, compound 229 was prepared in the same way as compound 227, starting from 2-ethyl-6-methylimidazo[1,2-a]pyridine-3-carboxylic acid (CAS [1216036-36-0], 0.35 mmol) and intermediate BB-3 (0.29 mmol) affording 53 mg (33%) as a white solid.

1H NMR (400 MHz, DMSO-d₆) δ 8.77 (s, 1H), 8.29 (t, J=5.9 Hz, 1H), 7.50 (d, J=9.1 Hz, 1H), 7.29 (d, J=8.6 Hz, 2H), 7.23 (dd, J=9.1, 1.6 Hz, 1H), 7.11 (d, J=8.7 Hz, 2H), 4.44 (d, J=5.9 Hz, 2H), 3.98 (t, J=4.8 Hz, 2H), 3.80 (t, J=5.0 Hz, 2H), 2.94 (q, J=7.5 Hz, 2H), 2.30 (s, 3H), 1.96-1.84 (m, 1H), 1.24 (t, J=7.5 Hz, 3H), 0.96-0.89 (m, 2H), 0.89-0.79 (m, 2H).

Synthesis of Compound 230

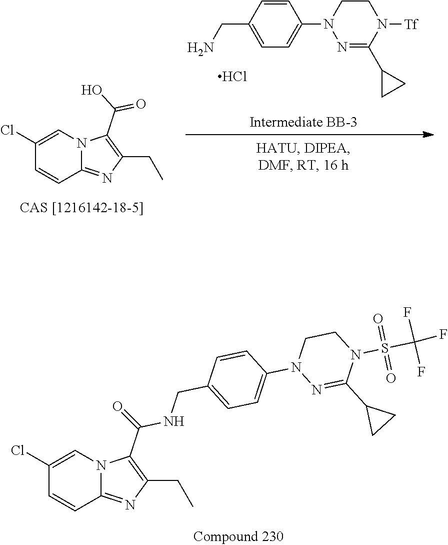

Accordingly, compound 230 was prepared in the same way as compound 227, starting from 6-chloro-2-ethylimidazo[1,2-a]pyridine-3-carboxylic acid (CAS [1216142-18-5], 0.6 mmol) and intermediate BB-3 (0.5 mmol) affording 99 mg (34%) as a white solid.

1H NMR (400 MHz, DMSO-d₆) δ 9.07 (d, J=1.3 Hz, 1H), 8.42 (t, J=5.8 Hz, 1H), 7.66 (d, J=9.5 Hz, 1H), 7.45 (dd, J=9.5, 2.0 Hz, 1H), 7.30 (d, J=8.5 Hz, 2H), 7.11 (d, J=8.6 Hz, 2H), 4.45 (d, J=5.8 Hz, 2H), 3.97 (t, J=4.8 Hz, 2H), 3.80 (t, J=4.9 Hz, 2H), 2.97 (q, J=7.5 Hz, 2H), 1.93-1.83 (m, 1H), 1.25 (t, J=7.5 Hz, 3H), 0.98-0.89 (m, 2H), 0.84 (dd, J=5.1, 2.8 Hz, 2H).

Synthesis of Compound 231

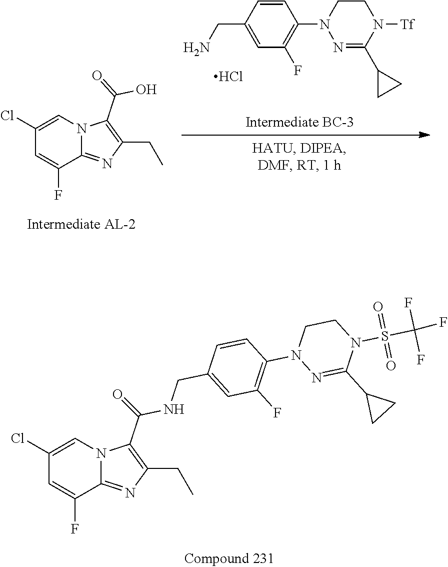

Accordingly, compound 231 was prepared in the same way as compound 227, starting from intermediate AL-2 (0.37 mmol) and intermediate BC-3 (0.31 mmol) affording 51 mg (27%) as a white solid.

1H NMR (400 MHz, DMSO-d₆) δ 8.91 (s, 1H), 8.59 (br s, 1H), 7.62 (d, J=10.5 Hz, 1H), 7.27-7.15 (m, 3H), 4.48 (d, J=4.5 Hz, 2H), 4.01-3.92 (m, 2H), 3.79-3.70 (m, 2H), 3.00 (q, J=7.5 Hz, 2H), 1.96-1.86 (m, 1H), 1.27 (t, J=7.5 Hz, 3H), 0.94-0.86 (m, 2H₁), 0.85-0.77 (m, 2H).

Synthesis of Compound 232

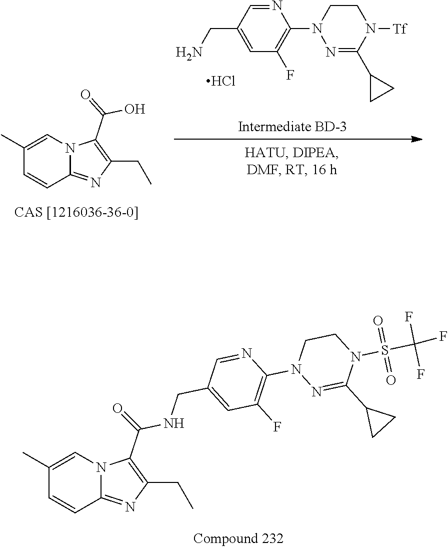

Accordingly, compound 232 was prepared in the same way as compound 227, starting from 2-ethyl-6-methylimidazo[1,2-a]pyridine-3-carboxylic acid (CAS [1216036-36-0], 0.43 mmol) and intermediate BD-3 (0.35 mmol) affording 97 mg (47%) as a white solid.

1H NMR (400 MHz, DMSC-d₆) δ 8.80 (s, 1), 8.31 (t, J=5.4 Hz, 1H), 8.08 (s, 1H), 7.63 (d, J=13.2 Hz, 1H), 7.51 (d, J=9.0 Hz, 1H), 7.24 (d, J=9.0 Hz, 1H), 4.47 (d, J=5.6 Hz, 2H), 4.05 (s, 2H), 3.97 (d, J=4.8 Hz, 2H), 2.95 (q, J=7.4 Hz, 2H), 2.30 (s, 3H), 2.01-1.86 (m, 1H), 1.25 (t, J=7.4 Hz, 3H), 0.98-0.74 (m, 4H)

Synthesis of Intermediates BE-3 and BF-3

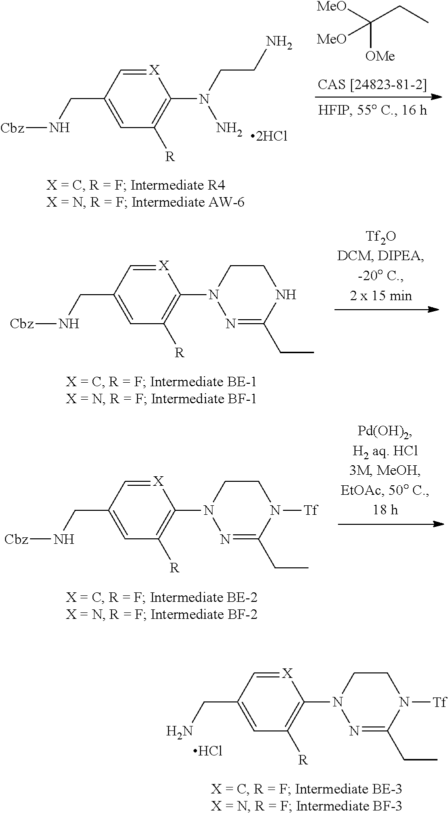

Preparation of Intermediate BE-1

Accordingly, intermediate BE-1 was prepared in the same way as intermediate BB-1, starting from intermediate R4 (7.35 mmol) and trimethyl orthopropionate (CAS [24823-81-2], 14.69 mmol) affording 1.82 g (64%) as a brown solid.

Preparation of Intermediate BE-2

Accordingly, intermediate BE-2 was prepared in the same way as intermediate BB-2, starting from intermediate BE-1 (1.48 mmol) affording 333 mg (34%) as a colorless oil.

Preparation of Intermediate BE-3

Accordingly, intermediate BE-3 was prepared in the same way as intermediate BB-3, starting from intermediate BE-2 (0.98 mmol) affording 469 mg (810%) as a yellow solid.

Preparation of Intermediate BF-1

Accordingly, intermediate BF-1 was prepared in the same way as intermediate BB-1, starting from intermediate AW-6 (5.18 mmol) and trimethyl orthopropionate (CAS [24823-81-2], 20.72 mmol) affording 1.35 g (70%) as a yellow oil.

Preparation of Intermediate BF-2

Accordingly, intermediate BF-2 was prepared in the same way as intermediate BB-2, starting from intermediate BF-1 (3.31 mmol) affording 1.22 g (68° %) as an orange solid.

Preparation of Intermediate BF-3

Accordingly, intermediate BF-3 was prepared in the same way as intermediate BB-3, starting from intermediate BF-2 (2.58 mmol) affording 997 mg (92%) as a colorless oil.

Synthesis of Compound 233

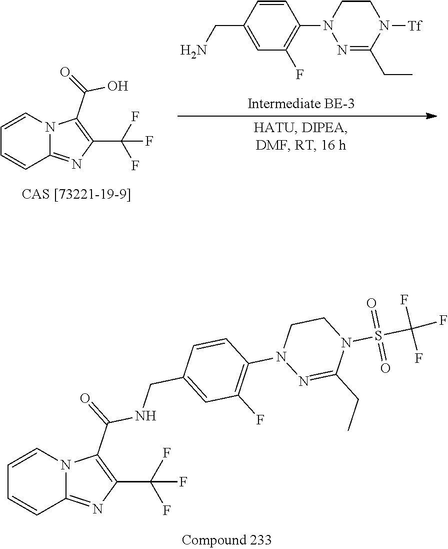

Accordingly, compound 233 was prepared in the same way as compound 227, starting from 2-(trifluoromethyl)imidazo[1,2-a]pyridine-3-carboxylic acid (CAS [73221-19-9], 0.64 mmol) and intermediate BE-3 (0.53 mmol) affording 52 mg (17%) as a white solid. 1H (400 MHz, DMSO-d₆) δ 9.18 (t, J=5.6 Hz, 1H), 8.48 (d, J=6.9 Hz, 1H), 7.71 (d, J=9.1 Hz, 1H), 7.53-7.40 (m, 1H), 7.28 (t, J=8.6 Hz, 1H), 7.12 (dd, J=16.6, 10.3 Hz, 3H), 4.43 (d, J=5.7 Hz, 2H), 3.93-3.83 (m, 2H), 3.70 (d, J=4.3 Hz, 2H), 2.52 (q, J=7.2 Hz, 2H), 1.07 (t, J=7.2 Hz, 3H).

Synthesis of Compound 234

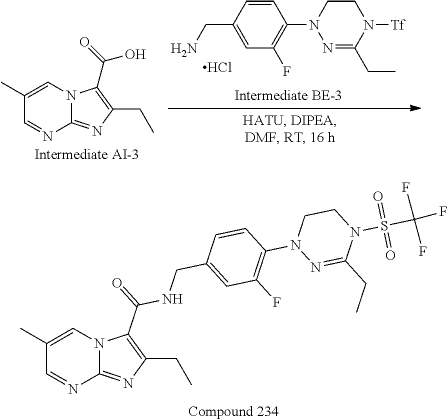

Accordingly, compound 234 was prepared in the same way as compound 227, starting from intermediate AI-3 (0.64 mmol) and intermediate BE-3 (0.53 mmol) affording 99 mg (32%) as a white solid.

1H NMR (400 MHz, DMSO-d₆) δ 9.15 (s, 1H), 8.51 (d, J=2.1 Hz, 1H), 8.44 (t, J=5.9 Hz, 1H), 7.34 (t, J=8.6 Hz, 1H), 7.20 (dd, J=16.8, 11.3 Hz, 2H), 4.49 (d, J=5.9 Hz, 2H), 3.99-3.92 (m, 2H), 3.81-3.70 (m, 2H), 3.01 (q, J=7.5 Hz, 2H), 2.60 (q, J=7.2 Hz, 2H), 2.34 (s, 3H), 1.28 (t, J=7.5 Hz, 3H), 1.14 (t, J=7.2 Hz, 3H).

Synthesis of Compound 235

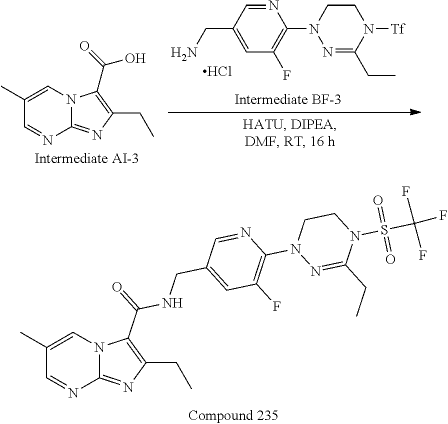

Accordingly, compound 235 was prepared in the same way as compound 227, starting from intermediate AI-3 (0.64 mol) and intermediate BF-3 (0.46 mmol) affording 72 mg (28%) as a white solid.

1H NMR (400 MHz, DMSO-d₆) δ 9.15 (dd, J=2.4, 1.1 Hz, 1H), 8.51 (d, J=2.4 Hz, 1H), 8.43 (t, J=5.8 Hz, 1H), 8.10 (s, 1H), 7.66 (dd, J=13.1, 1.8 Hz, 1H), 4.49 (d, J=5.8 Hz, 2H), 4.05 (dd, J=6.4, 3.6 Hz, 2H). 3.98 (dd, J=6.5, 3.6 Hz. 2H). 3.00 (q. J=7.5 Hz, 2H), 2.58 (q, J=7.2 Hz, 2H), 2.34 (d, J=0.7 Hz, 3H), 1.27 (t, J=7.5 Hz, 3H), 1.11 (t, J=7.3 Hz, 3H).

Synthesis of Compound 236

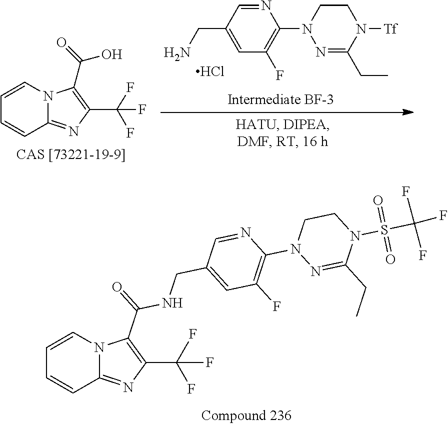

Accordingly, compound 236 was prepared in the same way as compound 227, starting from 2-(trifluoromethyl)imidazo[1,2-a]pyridine-3-carboxylic acid (CAS [73221-19-9], 0.58 mmol) and intermediate BF-3 (0.42 mmol) affording 69 mg (28%) as a white solid.

1H NMR (400 MHz, DMSO-d₆) δ 9.26 (t, J=5.7 Hz, 1H), 8.58 (d, J=7.0 Hz, 1H), 8.10 (s, 1H), 7.79 (d, J=9.1 Hz, 1H), 7.64 (dd, J=13.1, 1.7 Hz, 1H), 7.56 (ddd, J=9.1, 6.8, 1.2 Hz, 1H), 7.20 (td, J=6.9, 1.1 Hz, 1H), 4.52 (d, J=5.6 Hz, 2H), 4.05 (dd, J=6.3, 3.3 Hz, 2H), 4.00 (dd, J=6.1, 3.0 Hz, 2H), 2.59 (q, J=7.2 Hz, 2H), 1.12 (t, J=7.3 Hz, 3H).

Synthesis of Intermediate BG-3

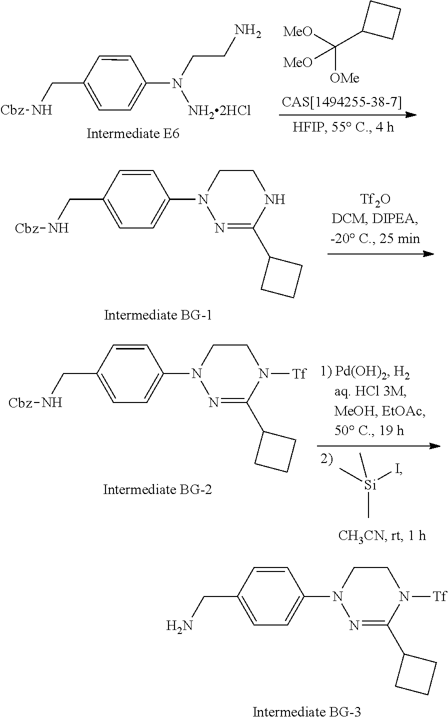

Preparation of Intermediate BG-1

Accordingly, intermediate BG-1 was prepared in the same way as intermediate BB-1, starting from intermediate E6 (1.29 mmol) and 1,1,1-trimethoxy-2-methylpropane (CAS [1494255-38-7], 2.58 mmol) affording 237 mg (41%) as a beige foam.

Preparation of Intermediate BG-2

Accordingly, intermediate BG-2 was prepared in the same way as intermediate BB-2, starting from intermediate BG-1 (0.65 mmol) affording 141 mg (21%) as a beige solid.

Preparation of Intermediate BG-3

In a round bottom flask, palladium hydroxide, Pd 20% on carbon, nominally 50% water (19 mg, 0.028 mmol) was added to a stirred solution of intermediate BG-2 (140 mg, 0.28 mmol) and HCl (3M, aq., 0.09 mL, 0.28 mmol) in EtOAc (3 mL) and MeOH (3 mL) at rt under nitrogen atmosphere. Then, nitrogen atmosphere was replaced by hydrogen and the reaction mixture was stirred at 50° C. for 16 h. An extra amount of palladium hydroxide, Pd 20% on carbon, nominally 50% water (39 mg, 0.055 mmol) was added to the reaction mixture at rt under nitrogen atmosphere. Then, nitrogen atmosphere was replaced by hydrogen and the reaction mixture was stirred at 50° C. for 3 h. The mixture was filtered through a pad of Celite® and the filtrate was concentrated in vacuo. The residue was taken into CH₃CN (4 mL) and iodotrimethylsilane (0.12 mL, 0.9 mmol) was added dropwise under nitrogen atmosphere. The reaction mixture was stirred at rt for 1 h. The mixture was diluted with DCM and washed with sat. aq. Na₂S₂O₂ solution. The combined organic layers were dried over anhydrous MgSO₄, filtered and concentrated in vacuo affording intermediate BG-3 (90 mg, 33%) as an orange solid. The crude product was used as such in the next step.

Synthesis of Compound 237

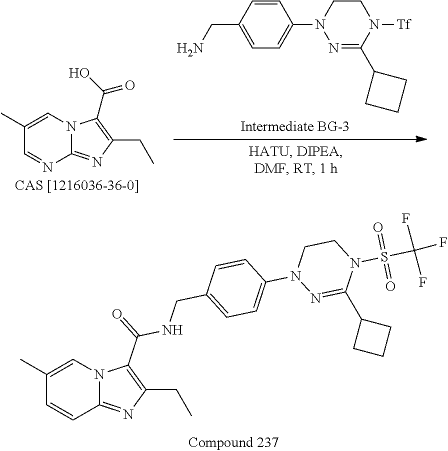

Accordingly, compound 237 was prepared in the same way as compound 227, starting from 2-ethyl-6-methylimidazo[1,2-a]pyridine-3-carboxylic acid (CAS [1216036-36-0], 0.36 mmol) and intermediate BG-3 (0.24 mmol) affording 20 mg (15%) as a beige foam.

1H NMR (400 MHz, DMSO-d₆) δ 8.78 (s, 1H), 8.30 (t, J=5.9 Hz, 1H), 7.50 (d, J=9.1 Hz, 1H), 7.33 (d, J=8.7 Hz, 2H), 7.25 (d, J=1.6 Hz, 3H), 4.46 (d, J=5.9 Hz, 2H), 3.96 (t, J=4.9 Hz, 2H), 3.83 (t, J=5.0 Hz, 2H), 3.48 (p, J=8.2 Hz, 1H), 2.95 (q, J=7.5 Hz, 2H), 2.38-2.26 (m, 5H), 2.25-2.15 (m, 2H), 1.90 (dd, J=18.5, 9.6 Hz, 1H), 1.81 (dt, J=19.4, 7.4 Hz, 1H), 1.25 (t, J=7.5 Hz, 3H).

Synthesis of intermediates BH-3 and BI-3

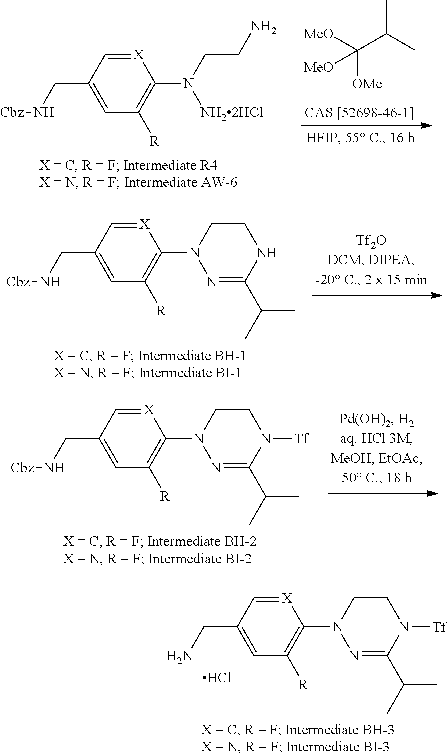

Preparation of Intermediate BH-1

Accordingly, intermediate BH-1 was prepared in the same way as intermediate BB-1, starting from intermediate R4 (0.62 mmol) and 1,1,1-trimethoxy-2-methylpropane (CAS [52698-46-1], 2.47 mmol) affording 156 mg (59%) as an orange solid.

Preparation of Intermediate BH-2

Accordingly, intermediate BH-2 was prepared in the same way as intermediate BB-2, starting from intermediate BH-1 (0.41 mmol) affording 151 mg (65%) as a beige solid.

Preparation of Intermediate BH-3

Accordingly, intermediate BH-3 was prepared in the same way as intermediate BB-3, starting from intermediate BH-2 (0.28 mmol) affording 116 mg (97%) as a yellow solid.

Preparation of Intermediate BI-1

Accordingly, intermediate BI-1 was prepared in the same way as intermediate BB-1, starting from intermediate AW-6 (4.92 mmol) and 1,1,1-trimethoxy-2-methylpropane (CAS [52698-46-1], 19.69 mmol) affording 1.06 g (53%) as a light yellow oil.

Preparation of Intermediate BI-2

Accordingly, intermediate BI-2 was prepared in the same way as intermediate BB-2, starting from intermediate BI-1 (2.74 mmol) affording 869 mg (55%) as a pale pink solid.

Preparation of Intermediate BI-3

Accordingly, intermediate BI-3 was prepared in the same way as intermediate BB-3, starting from intermediate BI-2 (1.67 mmol) affording 702 mg (90%) as a pale yellow solid.

Synthesis of Compound 238

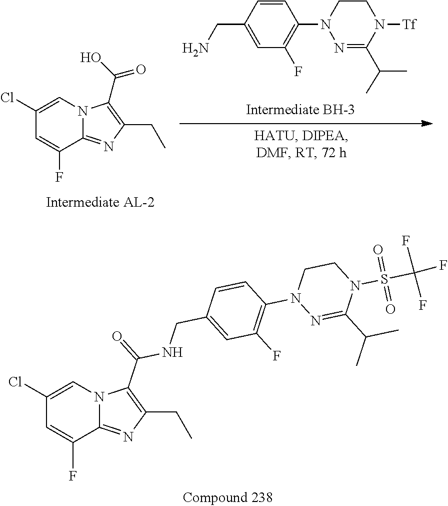

Accordingly, compound 238 was prepared in the same way as compound 227, starting from intermediate AL-2 (0.36 mmol) and intermediate BH-3 (0.27 mmol) affording 68 mg (40%) as a beige solid.

1H NMR (400 MHz, DMSO-d₆) δ 8.92 (d, J=1.4 Hz, 1H), 8.60 (t, J=5.9 Hz, 1H), 7.62 (dd, J=10.6, 1.7 Hz, 1H), 7.33 (t, J=8.6 Hz, 1H), 7.27-7.21 (m, 1H), 7.21-7.17 (m, 1H), 4.49 (d, J=5.9 Hz, 2H), 3.95-3.85 (m, 2H), 3.84-3.74 (m, 2H), 3.08-2.95 (m, 3H), 1.28 (t, J=7.5 Hz, 3H), 1.17 (d, J=6.7 Hz, 6H).

Synthesis of Compound 239

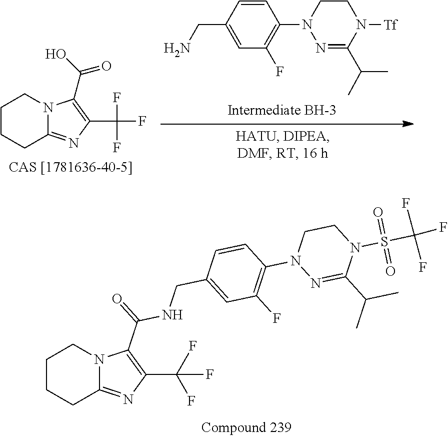

Accordingly, compound 239 was prepared in the same way as compound 227, starting from 2-(trifluoromethyl)-5H, 6H, 7H, 8H-imidazo[1,2-a]pyridine-3-carboxylic acid (CAS [1781636-40-5], 0.5 mmol) and intermediate BH-3 (0.36 mmol) affording 84 mg (39%) as a beige solid.

¹H NMR (400 MHz, DMSO-d₆) δ 9.10 (t, J=5.9 Hz, 1H), 7.32 (t, J=8.6 Hz, 1H), 7.13 (dd, J=13.0, 11.1 Hz, 2H), 4.40 (d, J=5.9 Hz, 2H), 3.94-3.86 (m, 4H), 3.81 (d, J=4.0 Hz, 2H), 2.98 (dt, J=13.3, 6.7 Hz, 1H), 2.78 (t, J=6.3 Hz, 2H), 1.91 (d, J=4.1 Hz, 2H), 1.84 (d, J=4.9 Hz, 2H), 1.18 (s, 3H), 1.17 (s, 3H).

Synthesis of Compound 240

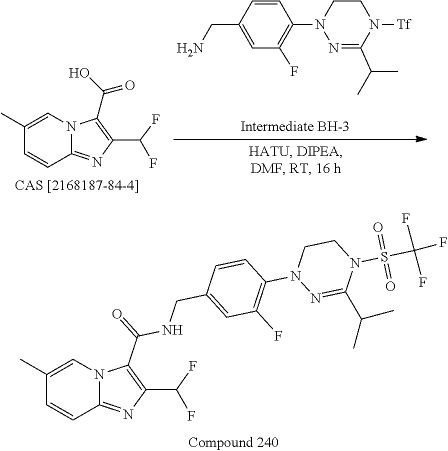

Accordingly, compound 240 was prepared in the same way as compound 227, starting from 2-(difluoromethyl)-6-methylimidazo[1,2-a]pyridine-3-carboxylic acid (CAS [2168187-84-4], 0.5 mmol) and intermediate BH-3 (0.36 mmol) affording 95 mg (44%) as a beige solid.

1H NMR (400 MHz, DMSO-d₆) δ 8.93 (t, J=5.8 Hz, 1H), 8.61 (s, 1H), 7.67 (d, J=9.2 Hz, 1H), 7.46-7.30 (m, 3H), 7.29-7.21 (m, 1H), 7.22-7.17 (m, 1H), 4.50 (d, J=5.8 Hz, 2H), 3.93-3.86 (m, 2H), 3.81 (t, J=4.4 Hz, 2H), 2.98 (hept, J=6.7 Hz, 1H), 2.34 (s, 3H), 1.18 (d, J=6.7 Hz, 6H).

Synthesis of Compound 241

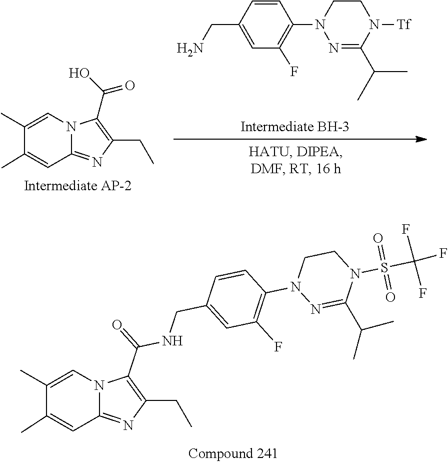

Accordingly, compound 241 was prepared in the same way as compound 227, starting from intermediate AP-2 (0.41 mmol) and intermediate BH-3 (0.3 mmol) affording 73 mg (42%) as a white solid.

1H NMR (400 MHz, DMSO-d₆) δ 8.78 (s, 1H), 8.24 (t, J=6.0 Hz, 1H), 7.38 (s, 1H), 7.32 (t, J=8.5 Hz, 1H), 7.25-7.15 (m, 2H), 4.47 (d, J=5.9 Hz, 2H), 3.93-3.85 (m, 2H), 3.83-3.76 (m, 2H), 3.02-2.91 (m, 3H), 2.30 (s, 3H), 2.22 (s, 3H), 1.25 (t, J=7.5 Hz, 3H), 1.17 (d, J=6.7 Hz, 6H).

Synthesis of Compound 242

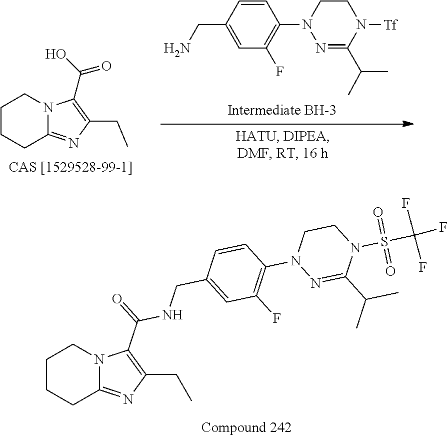

Accordingly, compound 242 was prepared in the same way as compound 227, starting from 2-ethyl-5H, 6H, 7H, 8H-imidazo[1,2-a]pyridine-3-carboxylic acid (CAS [1529528-99-1], 0.41 mmol) and intermediate BH-3 (0.3 mmol) affording 33 mg (19%) as a beige solid.

1H NMR (400 MHz, DMSO-d₆) δ 8.67 (s, 1H), 7.32 (t, J=8.3 Hz, 1H), 7.15 (t, J=12.6 Hz, 2H), 4.40 (d, J=4.9 Hz, 2H), 4.06 (s, 2H), 3.89 (s, 2H), 3.79 (s, 2H), 3.03-2.94 (m, 1H), 2.86 (s, 2H), 2.71 (d, J=7.2 Hz, 2H), 1.87 (d, J=25.4 Hz, 4H), 1.17 (d, J=6.0 Hz, 9H).

Synthesis of Compound 243

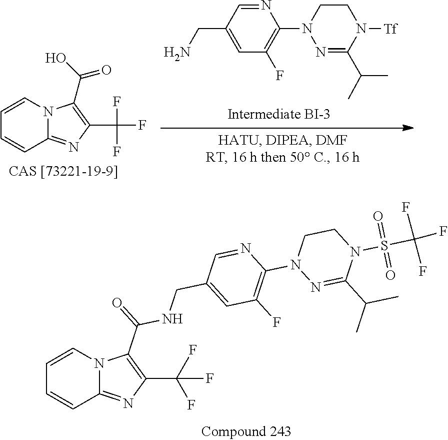

Accordingly, compound 243 was prepared in the same way as compound 227, starting from 2-(trifluoromethyl)imidazo[1,2-a]pyridine-3-carboxylic acid (CAS [73221-19-9], 0.51 mmol) and intermediate BI-3 (0.39 mmol) affording 24 mg (10%) as a beige foam.

1H NMR (400 MHz, CDCl₃) δ 9.29 (d, J=7.1 Hz, 1H), 8.05 (s, 1H), 7.77 (d, J=9.1 Hz, 1H), 7.51-7.46 (m, 1H), 7.44 (dd, J=12.4, 1.4 Hz, 1H), 7.10 (t, J=6.9 Hz, 1H), 6.65 (s, 1H), 4.63 (d, J=5.7 Hz, 2H), 4.17-4.09 (m, 2H), 3.93-3.86 (m, 2H), 3.19-3.02 (m, 1H), 1.22 (d, J=6.7 Hz, 6H).

Synthesis of Compound 244

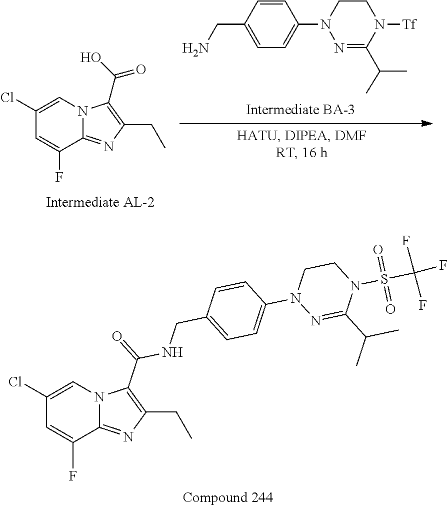

Accordingly, compound 244 was prepared in the same way as compound 227, starting from intermediate AL-2 (0.51 mmol) and intermediate BA-3 (0.4 mmol) affording 82 mg (35%) as a beige solid.

1H NMR (400 MHz, DMSO-d₆) δ 8.92-8.88 (m, I1H), 8.58 (t, J=5.9 Hz, 1H), 7.61 (dd, J=10.6, 1.7 Hz, 1H), 7.32 (d, J=8.7 Hz, 2H), 7.18 (d, J=8.7 Hz, 2H), 4.46 (d, J=5.9 Hz, 2H), 3.92 (t, J=5.0 Hz, 2H), 3.84 (t, J=5.0 Hz, 2H), 3.04-2.93 (m, 3H), 1.26 (t, J=7.5 Hz, 3H), 1.20 (d, J=6.7 Hz, 6H).

Synthesis of Compound 245

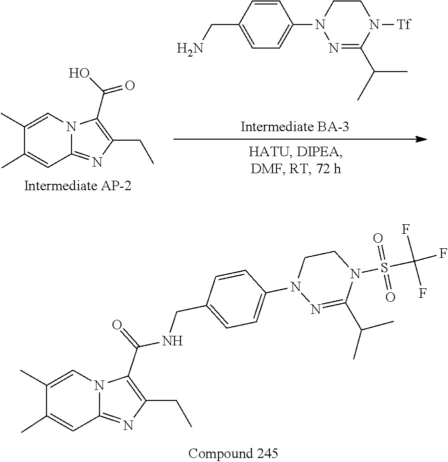

Accordingly, compound 245 was prepared in the same way as compound 227, starting from intermediate AP-2 (0.61 mmol) and intermediate BA-3 (0.47 mmol) affording 87 mg (32%) as a white solid.

1H NMR (400 MHz, DMSO-d₆) δ 8.77 (s, 1H), 8.19 (t, J=5.9 Hz, 1H), 7.37 (s, 1H), 7.31 (d, J=8.7 Hz, 2H), 7.17 (d, J=8.7 Hz, 2H), 4.44 (d, J=5.9 Hz, 2H), 3.92 (t, J=5.0 Hz, 2H), 3.83 (t, J=5.1 Hz, 2H), 3.01-2.88 (m, 3H), 2.30 (s, 3H), 2.22 (s, 3H), 1.26-1.17 (m, 9H).

Synthesis of Compound 246

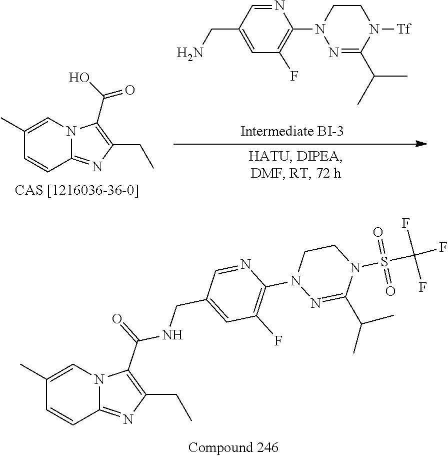

Accordingly, compound 246 was prepared in the same way as compound 227, starting from 2-ethyl-6-methylimidazo[1,2-a]pyridine-3-carboxylic acid (CAS: [1216036-36-0], 0.51 mmol) and intermediate BI-3 (0.39 mmol) affording 90 mg (40%) as a beige solid.

1H NMR (400 MHz, CDCl₃) δ 9.15 (s, 1H), 8.02 (s, 1H), 7.50 (d, J=9.1 Hz, 1H), 7.43 (dd, J=12.5, 1.6 Hz, 1H), 7.19 (dd, J=9.1, 1.3 Hz, 1H), 6.32 (s, 1H), 4.61 (d, J=5.9 Hz, 2H), 4.15-4.03 (m, 2H), 3.93-3.77 (m, 2H), 3.14-3.02 (m, 1H), 2.97 (q, J=7.6 Hz, 2H), 2.35 (s, 3H), 1.39 (t, J=7.6 Hz, 3H), 1.19 (d, J=6.7 Hz, 6H).

Synthesis of Compound 247

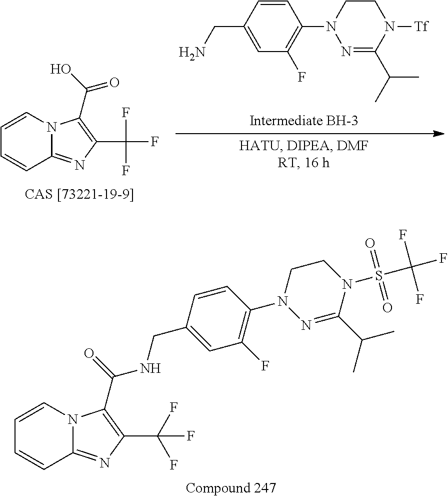

Accordingly, compound 247 was prepared in the same way as compound 227, starting from 2-(trifluoromethyl)imidazo[1,2-a]pyridine-3-carboxylic acid (CAS: [73221-19-9], 1.5 mmol) and intermediate BH-3 (1.07 mmol) affording 308 mg (48%) as a beige solid.

1H NMR (400 MHz, DMSO-d₆) δ 9.26 (t, J=5.7 Hz, 1H), 8.57 (d, J=6.9 Hz, 1H), 7.79 (d, J=9.1 Hz, 1H), 7.59-7.52 (m, 1H), 7.35 (t, J=8.5 Hz, 1H), 7.27-7.15 (m, 3H), 4.52 (d, J=5.7 Hz, 2H), 3.95-3.88 (m, 2H), 3.86-3.78 (m, 2H), 3.05-2.90 (m, 1H), 1.18 (d, J=6.7 Hz, 6H).

Synthesis of Compound 248

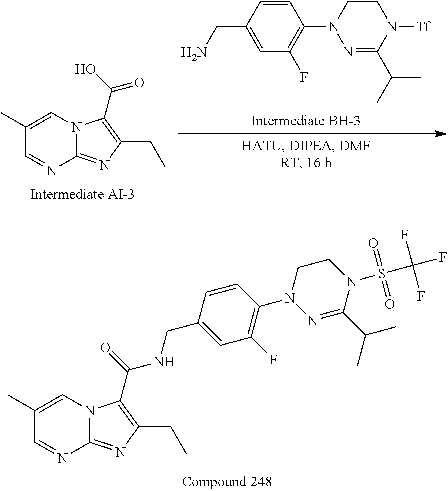

Accordingly, compound 248 was prepared in the same way as compound 227, starting from intermediate AI-3 (1.39 mmol) and intermediate BH-3 (1.07 mmol) affording 287 mg (47%) as a white solid.

1H NMR (400 MHz, DMSO-d₆) δ 9.17-9.12 (m, 1H), 8.51 (d, J=2.3 Hz, 1H), 8.44 (t, J=5.9 Hz, 1H), 7.33 (t, J=8.6 Hz, 1H), 7.26-7.21 (m, 1H), 7.20-7.17 (m, 1H), 4.48 (d, J=5.9 Hz, 2H), 3.94-3.86 (m, 2H), 3.79 (t, J=4.5 Hz, 2H), 3.08-2.91 (m, 31H), 2.34 (s, 3H), 1.28 (t, J=7.5 Hz, 3H), 1.17 (d, J=6.7 Hz, 6H).

Synthesis of Compound 249

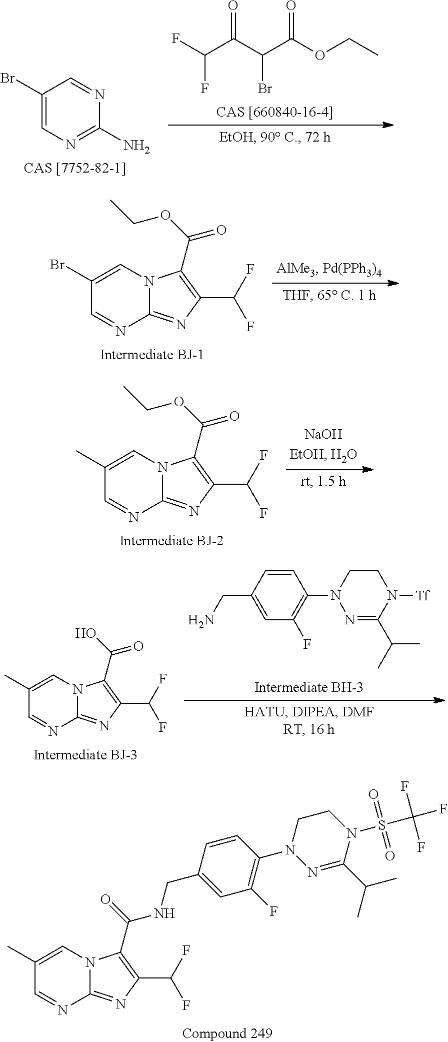

Preparation of Intermediate BJ-1

2-Amino-5-bromopyrimidine (CAS [7752-82-1], 677 mg, 3.89 mmol) was added to a stirred solution of ethyl 2-bromo-4,4-difluoro-3-oxobutanoate (CAS [660840-16-4], 1.43 g, 5.84 mmol) in EtOH (25 mL) in a glass pressure bottle. The mixture was stirred at 90° C. for 72 h. The mixture was evaporated in vacuo. The crude was diluted with EtOAc and neutralized with NaHCO₃ (sat., aq.). The organic layer was dried with MgSO₄ and concentrated in vacuo. The crude product was purified by flash column chromatography (silica, 25 g; EtOAc in heptane from 0/100 to 40/70). The desired fractions were collected and concentrated in vacuo to yield intermediate BJ-1 (247 mg, 20%) as a pale yellow solid.

Preparation of Intermediate BJ-2

Trimethylaluminum (2M in hexanes, 1.15 mL, 2.29 mmol) was added dropwise to a solution of intermediate BJ-1 (247 mg, 0.76 mmol) and Pd(PPh₄)₃ (CAS [14221-01-3], 44 mg, 0.038 mmol) in dry THF (6 mL) in a round bottom flask 2-neck charged with a condenser under nitrogen atmosphere at rt. Then the mixture was stirred at 65° C. for 1 h. The mixture was cooled to 0° C. and diluted with DCM. Then 5 ml of water was added dropwise and the mixture was stirred at rt for 1 h. Then MgSO₄ was added and the mixture was stirred at rt for 30 min. The mixture was filtered through a pad of Celite®, washed with ethyl acetate and concentrated in vacuo. The crude product was purified by flash column chromatography (silica, 12 g; EtOAc in heptane 0/100 to 35/65). The desired fractions were collected and concentrated in vacuo to yield intermediate BJ-2 (142 mg, 72%) as a yellow solid.

Preparation of Intermediate BJ-3

NaOH (52 mg, 1.29 mmol) was added to a solution of intermediate BJ-2 (122 mg, 0.43 mmol) in EtOH (3.36 mL) and H₂O (0.86 mL) in a round bottom flask at rt. The mixture was stirred at rt for 1.5 h. 1 M aqueous HCl solution was added until pH 7. The mixture was concentrated in vacuo to yield intermediate BJ-3 (105 mg, quant.) as an orange solid.

Preparation of Compound 249

Accordingly, compound 249 was prepared in the same way as compound 227, starting from intermediate BJ-3 (0.5 mmol) and intermediate BH-3 (0.5 mmol) affording 72 mg (24%) as a white solid.

1H NMR (400 MHz, DMSO-d₆) δ 9.00 (d, J=6.3 Hz, 2H), 8.69 (d, J=2.2 Hz, 1H), 7.52-7.16 (m, 4H), 4.51 (d, J=4.3 Hz, 2H), 3.93-3.76 (m, 4H), 2.98 (dt, J=133, 6.7 Hz, 1H), 2.38 (s, 3H), 1.17 (d, J=6.7 Hz, 6H).

Synthesis of Compound 250

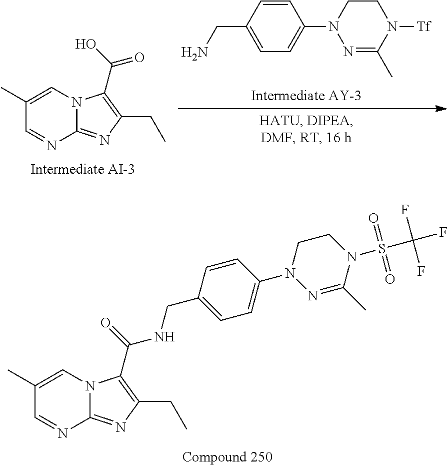

Accordingly, compound 250 was prepared in the same way as compound 227, starting from intermediate AI-3 (0.68 mmol) and intermediate AY-3 (0.78 mmol) affording 72 mg (20%) as a white solid.

¹H NMR (500 MHz, DMSO-d₆) δ ppm 9.12 (br s, 1H) 8.51 (d, J=1.83 Hz, 1H) 8.44 (br t, J=5.65 Hz, 1H) 7.30 (br d, J=8.39 Hz, 2H) 7.16 (br d, J=8.54 Hz, 2H) 4.46 (br d, J=5.65 Hz, 2H) 4.01 (br t, J=1.00 Hz, 2H) 3.82 (br t, J=4.65 Hz, 2H) 2.98 (q, J=7.48 Hz, 2H) 2.34 (s, 3H) 2.26 (s, 3H) 1.26 (br t, J=7.48 Hz, 3H).

Synthesis of Intermediates BK-3 and BL-3

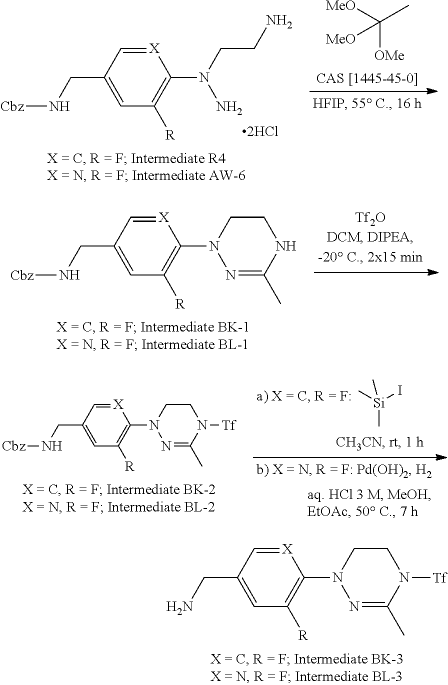

Preparation of Intermediate BK-1

Accordingly, intermediate BK-1 was prepared in the same way as intermediate BB-1, starting from intermediate R4 (6.53 mmol) and trimethyl orthoacetate (CAS [1445-45-0], 13.07 mmol) affording 1.72 g (710%) as a brown solid.

Preparation of Intermediate BK-2

Accordingly, intermediate BK-2 was prepared in the same way as intermediate BB-2, starting from intermediate BK-1 (1.4 mmol) affording 291 mg (42%) as a colourless oil.

Preparation of Intermediate BK-3

Iodotrimethylsilane (0.15 mL, 1.03 mmol) was added dropwise to a stirred solution of intermediate BK-2 (201 mg, 0.41 mmol) in CH₃CN (4 mL) under nitrogen atmosphere. The mixture was stirred for 1h. The mixture was diluted with water and extracted with DCM. Then aqueous saturated Na₂SO₃ solution was added to the aqueous layer until pH=8 and then extracted with dichloromethane (×5). The combined organic layers were dried over MgSO₄, filtered, and concentrated in vacuo to yield intermediate BK-3 (150 mg, quant.) as a colourless oil.

Preparation of Intermediate BL-1

Accordingly, intermediate BL-1 was prepared in the same way as intermediate BB-1, starting from intermediate AW-6 (5.18 mmol) and trimethyl orthoacetate (CAS [1445-45-0], 20.72 mmol) affording 970 mg (520%) as a yellow oil.

Preparation of Intermediate BL-2

Accordingly, intermediate BL-2 was prepared in the same way as intermediate BB-2, starting from intermediate BL-1 (2.46 mmol) affording 737 mg (60%) as an orange solid.

Preparation of Intermediate BL-3

Accordingly, intermediate BL-3 was prepared in the same way as intermediate BB-3, starting from intermediate BL-2 (1.88 mmol) affording 602 mg (89%) as a pale yellow solid.

Synthesis of Compound 251

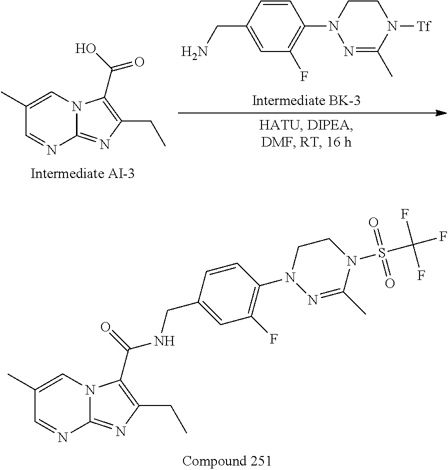

Accordingly, compound 251 was prepared in the same way as compound 227, starting from intermediate AI-3 (0.43 mmol) and intermediate BK-3 (0.43 mmol) affording 81 mg (35%) as a white solid.

1H NMR (400 MHz, DMSO-d₆) δ 9.06 (s, 1H), 8.43 (d, J=1.9 Hz, 1H), 8.37 (t, J=6.1 Hz, 1H), 7.25 (t, J=8.5 Hz, 1H), 7.11 (dd, J=17.7, 11.1 Hz, 2H), 4.40 (d, J=5.6 Hz, 2H), 3.92 (d, J=4.8 Hz, 2H), 3.64 (d, J=4.2 Hz, 2H), 2.92 (q, J=7.5 Hz, 2H), 2.25 (s, 3H), 2.16 (s, 3H), 1.19 (t, J=7.5 Hz, 3H).

Synthesis of Compound 252

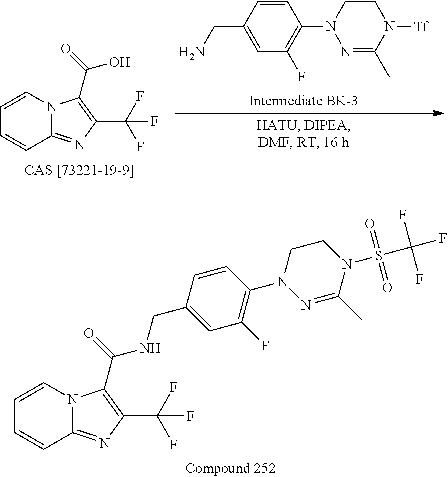

Accordingly, compound 252 was prepared in the same way as compound 227, starting from 2-(trifluoromethyl)imidazo[1,2-a]pyridine-3-carboxylic acid (CAS [73221-19-9], 0.19 mmol) and intermediate BK-3 (0.19 mmol) affording 33 mg (30%) as a white solid.

1H NMR (400 MHz, DMSO-d₆) δ 9.27 (t, J=5.6 Hz, 1H), 8.56 (d, J=6.8 Hz, 1H), 7.80 (d, J=9.0 Hz, 1H), 7.60-7.52 (m, 1H), 7.35 (t, J=8.6 Hz, 1H), 7.24-7.15 (m, 3H), 4.52 (d, J=5.7 Hz, 2H), 4.01 (d, J=4.8 Hz, 2H), 3.74 (s, 2H), 2.25 (s, 3H).

Synthesis of Compound 253

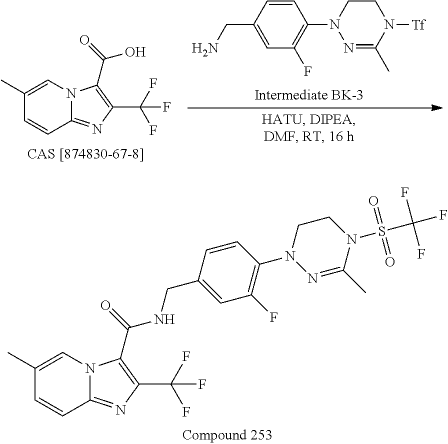

Accordingly, compound 253 was prepared in the same way as compound 227, starting from 6-methyl-2-(trifluoromethyl)imidazo[1,2-a]pyridine-3-carboxylic acid (CAS [874830-67-8], 0.58 mmol) and intermediate BK-3 (0.4 mmol) affording 19 mg (8%) as a white solid.

1H NMR (400 MHz, DMSO-d₆) δ 9.24 (t, J=5.7 Hz, 1H), 8.35 (s, 1H), 7.70 (d, J=9.3 Hz, 1H), 7.42 (dd, J=9.3, 0.8 Hz, 1H), 7.35 (t, J=8.6 Hz, 1H), 7.20 (dd, J=16.1, 11.0 Hz, 2H), 4.52 (d, J=5.7 Hz, 2H), 4.10-3.92 (m, 2H), 3.81-3.66 (m, 2H), 2.34 (s, 3H), 2.25 (s, 3H).

Synthesis of Compound 254

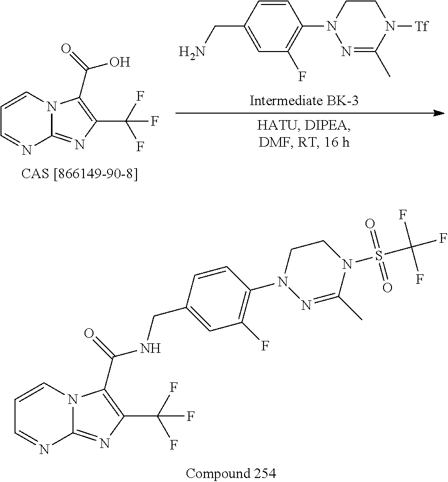

Accordingly, compound 254 was prepared in the same way as compound 227, starting from 2-(trifluoromethyl)imidazo[1,2-a]pyrimidine-3-carboxylic acid (CAS [866149-90-8], 0.85 mmol) and intermediate BK-3 (0.58 mmol) affording 77 mg (23%) as a white solid.

1H NMR (400 MHz, DMSO-d₆) δ 9.27 (s, 1H), 9.03 (dd, J=7.0, 1.9 Hz, 1H), 8.84 (dd, J=4.1, 1.9 Hz, 1H), 7.39-7.31 (m, 2H), 7.27-7.1 (m, 2H), 4.52 (d, J=4.0 Hz, 2H), 4.15-3.96 (m, 2H), 3.79-3.70 (m, 2H), 2.25 (s, 3H).

Synthesis of Compound 255

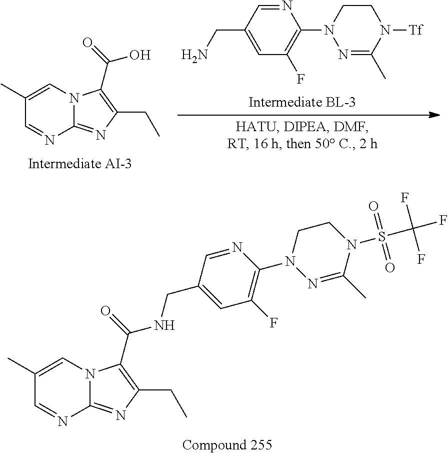

Accordingly, compound 255 was prepared in the same way as compound 227, starting from intermediate AI-3 (0.64 mmol) and intermediate BL-3 (0.46 mmol) affording 65 mg (26%) as a white solid.

1H NMR (400 MHz, DMSO-d₆) δ 9.15 (dd, J=2.4, 1.1 Hz, 1H), 8.51 (d, J=2.4 Hz, 1H), 8.43 (t, J=5.8 Hz, 1H), 8.10 (s, 1H), 7.66 (dd, J=13.0, 1.8 Hz, 1H), 4.49 (d, J=5.8 Hz, 2H), 4.12-4.05 (m, 2H), 3.94 (dd, J=6.0, 3.9 Hz, 2H), 2.99 (q, J=7.5 Hz, 2H), 2.34 (s, 3H), 2.22 (s, 3H), 1.27 (t, J=7.5 Hz, 3H).

Synthesis of Compound 256

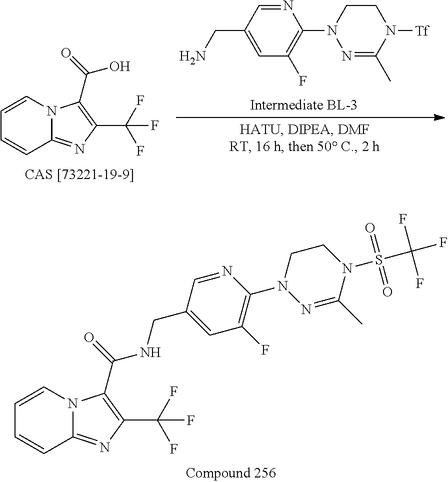

Accordingly, compound 256 was prepared in the same way as compound 227, starting from 2-(trifluoromethyl)imidazo[L1,2-a]pyridine-3-carboxylic acid (CAS [73221-19-9], 0.58 mmol) and intermediate BL-3 (0.42 mmol) affording 46 mg (20%) as a white solid.

1H NMR (400 MHz, DMSO-d₆) δ 9.26 (t, J=5.7 Hz, 1H), 8.58 (d, J=7.0 Hz, 1H), 8.10 (s, 1H), 7.79 (d, J=9.1 Hz, 1H), 7.64 (dd, J=13.0, 1.7 Hz, 1H), 7.56 (ddd, J=9.1, 6.8, 1.2 Hz, 1H), 7.20 (td, J=6.9, 1.1 Hz, 1H), 4.53 (d, J=5.7 Hz, 2H), 4.13-4.05 (m, 2H), 3.96 (dd, J=6.1, 3.9 Hz, 2H), 2.23 (s, 3H).

Synthesis of Compound 257

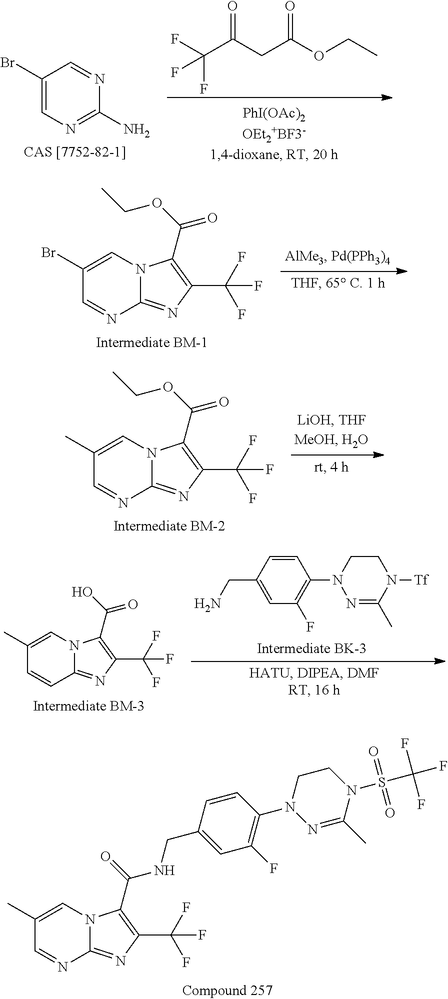

Preparation of Intermediate BM-1

In a round bottom flask, boron trifluoride diethyl etherate (71 μl, 0.57 mmol) was added dropwise to a stirred solution of 2-amino-5-bromopyrimidine (CAS [7752-82-1], 2 g, 11.49 mmol), ethyl 4,4,4-trifluoroacetoacetate (2.09 mL, 11.49 mmol) and (diacetoxyiodo)benzene (3.7 g, 11.49 mmol) in dry 1-4 dioxane (54 mL) at rt. The reaction mixture was stirred at rt for 2 h. Then, then additional amounts of ethyl 4,4,4-trifluoroacetoacetate (1.05 mL, 5.75 mmol), (diacetoxyiodo)benzene (1.85 g, 5.75 mmol) and boron trifluoride diethyl etherate (71 μl, 0.57 mmol) were added and the reaction mixture was stirred at rt for 16 h. Then, additional amounts of ethyl 4,4,4-trifluoroacetoacetate (1.05 mL, 5.75 mmol), (diacetoxyiodo)benzene (1.85 g, 5.75 mmol) and boron trifluoride diethyl etherate (71 μl, 0.57 mmol) were added and the reaction mixture was stirred at rt for 2 h. The mixture was diluted with EtOAc and washed with NaHCO₃ (sat., aq.). The organic layer was washed with brine, dried over anhydrous MgSO₄, filtered and concentrated in vacuo. The crude product was purified by flash column chromatography (silica 120 g; EtOAc in heptane from 0/100 to 20/80). The desired fractions were collected and concentrated in vacuo to yield intermediate BM-1 (1.48 g, 38%) as a yellow powder.

Preparation of Intermediate BM-2

Accordingly, intermediate BM-2 was prepared in the same way as intermediate BJ-2, starting from intermediate BM-1 (4.38 mmol) affording 889 mg (74%) as a beige solid.

Preparation of Intermediate BM-3

LiOH (42 mg, 0.99 mmol) was added to a solution of intermediate BM-2 (180 mg, 0.66 mmol) in THE (1.8 mL), MeOH (1.8 mL) and H₂O (1.8 mL) in a round bottom flask at rt. The mixture was stirred at rt for 4 h. 2 M aqueous HCl solution was added until pH 7. The mixture was concentrated in vacuo to yield intermediate BM-3 (189 mg, 91%) as a white solid. The crude product was used in the next step without further purification.

Preparation of Compound 257

Accordingly, compound 257 was prepared in the same way as compound 227, starting from intermediate BM-3 (0.89 mmol) and intermediate BK-3 (0.4 mmol) affording 33 mg (14%) as a white solid.

1H NMR (400 MHz, DMSO-d₆) δ 9.24 (t, J=5.7 Hz, 1H), 8.84 (s, 1H), 8.74 (d, J=2.3 Hz, 1H), 7.35 (t, J=8.6 Hz, 1H), 7.20 (dd, J=17.9, 10.9 Hz, 2H), 4.52 (d, J=5.8 Hz, 2H), 4.02 (dd, J=8.4, 4.7 Hz, 2H), 3.77-3.70 (m, 2H), 2.38 (s, 3H), 2.25 (s, 3H).

Synthesis of Compound 258

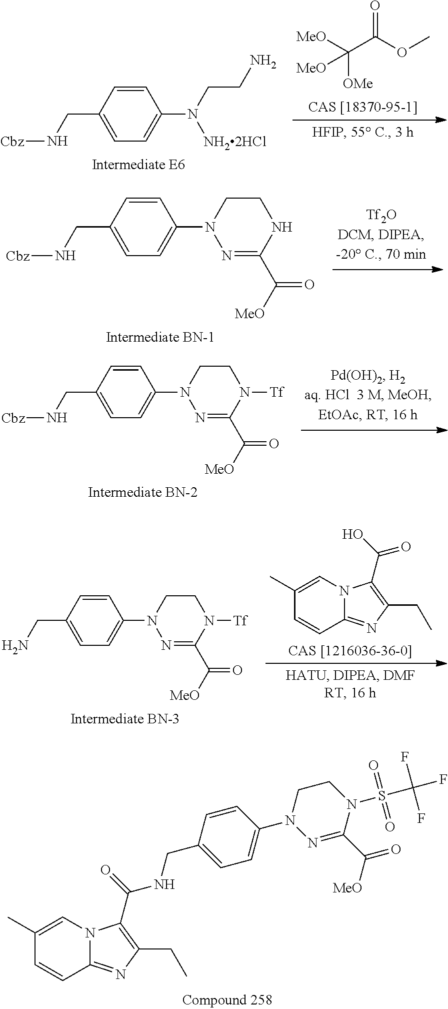

Preparation of Intermediate BN-1

Accordingly, intermediate BN-1 was prepared in the same way as intermediate BB-1, starting from intermediate E6 (4.68 mmol) and 2,2,2-trimethoxyacetic acid methyl ester (CAS [18370-95-1], 18.72 mmol) affording 1.42 g (79%) as a yellow solid.

Preparation of Intermediate BN-2

Accordingly, intermediate BN-2 was prepared in the same way as intermediate BB-2, starting from intermediate BN-1 (3.39 mmol) affording 1.21 g (68%) as a yellow solid.

Preparation of Intermediate BN-3

In a round bottom flask, palladium hydroxide, Pd 20% on carbon, nominally 50% water (165 mg, 0.24 mmol) was added to a stirred solution of intermediate BN-2 (1.21 g, 2.35 mmol) and HCl (3M, aq., 0.78 mL, 2.35 mmol) in EtOAc (20 mL) and MeOH (20 mL) at rt under nitrogen atmosphere. Then, nitrogen atmosphere was replaced by hydrogen and the reaction mixture was stirred at rt for 16 h. The mixture was filtered through a pad of Celite® and the filtrate was concentrated in vacuo affording intermediate BN-3 (987 mg, 96%) as a yellow solid. The crude product was used as such in the next step.

Preparation of Compound 258

Accordingly, compound 258 was prepared in the same way as compound 227, starting from 2-ethyl-6-methylimidazo[1,2-a]pyridine-3-carboxylic acid (CAS [1216036-36-0], 1.6 mmol) and intermediate BN-3 (1.06 mmol) affording compound 258 (635 mg, 95% pure, quant.) as a yellow oil.

A small amount of compound 258 (50 mg) was dissolved in DCM (10 mL). The solvent was distilled off under vacuo. The crude was purified by reverse phase (Phenomenex Gemini C18 30×100 mm 5 μm Column; from 59% [25 mM NH4HCO3]-41% [ACN: MeOH 1:1] to 17% [25 mM NH4HCO3]-83% [ACN: MeOH 1:1]. The desired fractions were collected and the solvents were concentrated in vacuo partially. The mixture was extracted with DCM. The combined organic layers were dried over anhydrous MgSO4, filtered and concentrated in vacuo. Diethylether and pentane were added and dried under vacuo to affording pure compound 258 (25 mg) as a yellowish solid.

1H NMR (400 MHz, DMSO-d₆) δ 8.78 (s, 1H), 8.36 (t, J=5.9 Hz, 1H), 7.51 (d, J=9.1 Hz, 1H), 7.41 (d, J=8.6 Hz, 2H), 7.36 (d, J=8.6 Hz, 2H), 7.24 (d, J=9.1 Hz, 1H), 4.50 (d, J=5.9 Hz, 2H), 4.10-3.97 (m, 2H), 3.87 (s, 2H), 3.80 (s, 3H), 2.96 (q, J=7.5 Hz, 2H), 2.3 1 (s, 3H), 1.25 (t, J=7.5 Hz, 3H).

Synthesis of Compound 259

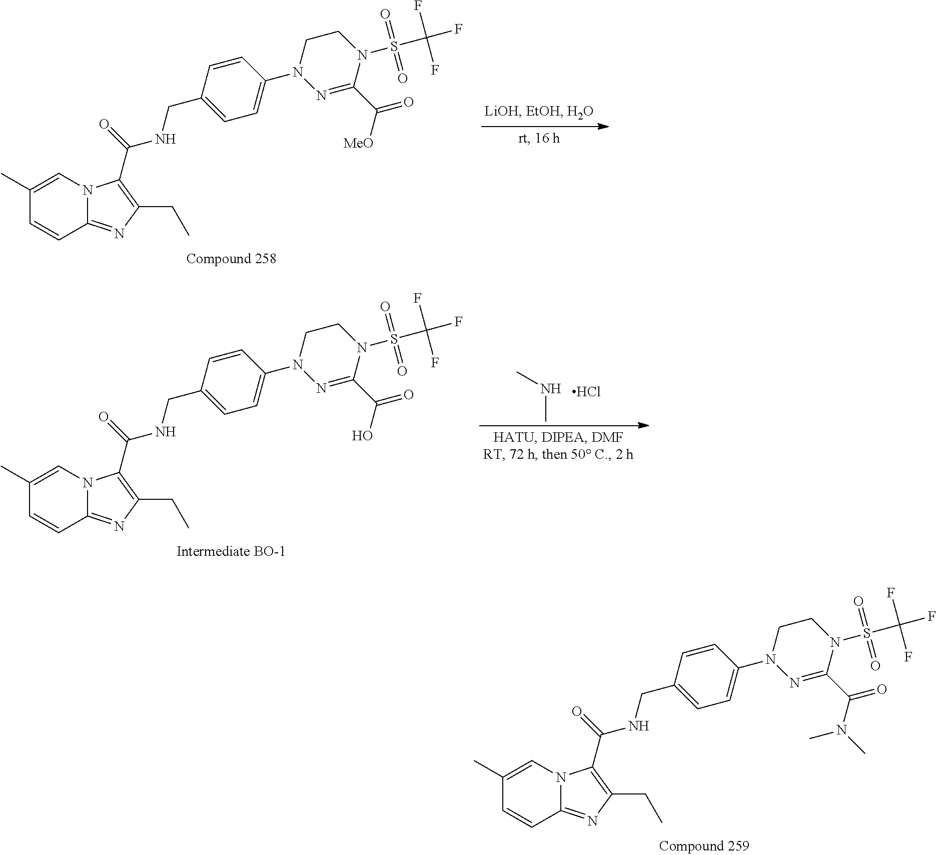

Preparation of Intermediate BO-1

In a round bottom flask, lithium hydroxide monohydrate (30 mg, 0.71 mmol) was added to a stirred solution of compound 258 (135 mg, 0.24 mmol) in EtOH (3 mL) and H₂O (1.5 mL) at rt. The reaction mixture was stirred at rt for 16 h. The solvents were removed in vacuo to yield intermediate BO-1 (135 mg, quant.) as a yellow oil. The crude product was used without further purification in the next step.

Preparation of Compound 259

Dimethylamine hydrochloride (29 mg, 0.36 mmol) was added to a solution of intermediate BO-1 (134 mg, 0.24 mmol), HATU (91 mg, 0.24 mmol) and DIPEA (167 μl, 0.96 mmol) in DMF (4 mL) in a round bottom flask at rt. The mixture was stirred at rt for 72 h. Additional amounts of HATU (46 mg, 0.12 mmol), DIPEA (83 μl, 0.48 mmol) and dimethylamine hydrochloride (20 mg, 0.24 mmol) were added to the solution mixture and it was stirred at 50° C. for 2 h. The reaction mixture was diluted with H₂O and brine and extracted with AcOEt (×3). The combined organic layers were dried over anhydrous MgSO₄, filtered and concentrated in vacuo. The crude product was purified by flash column chromatography (silica, 12 g; (DCM/MeOH (9:1) in DCM from 0/100 to 30/70). The desired fractions were collected and concentrated in vacuo. The crude was purified by reverse phase (Phenomenex Gemini C18 30×100 mm 5 μm Column; from 59% [25 mM NH₄HCO₃]-41% [ACN: MeOH 1:1] to 17% [25 mM NH₄HCO₃]-83% [ACN: MeOH 1:1]. The desired fractions were collected and extracted with DCM. The combined organic layers were dried over anhydrous MgSO₄, filtered and concentrated in vacuo. Diethylether and pentane were added and dried under vacuo to yield compound 259 (76 mg, 54%) as a pale yellow foam.

1H NMR (400 MHz, DMSO-d₆) δ 8.78 (s, 1H), 8.32 (t, J=5.7 Hz, 1H), 7.50 (d, J=9.1 Hz, 1H), 7.35 (d, J=8.6 Hz, 2H), 7.24 (d, J=8.7 Hz, 3H), 4.47 (d, J=5.9 Hz, 2H), 3.96 (s, 4H), 3.09 (s, 3H), 3.00-2.89 (m, 5H), 2.30 (s, 3H), 1.25 (t, J=7.5 Hz, 3H).

Synthesis of Compound 260

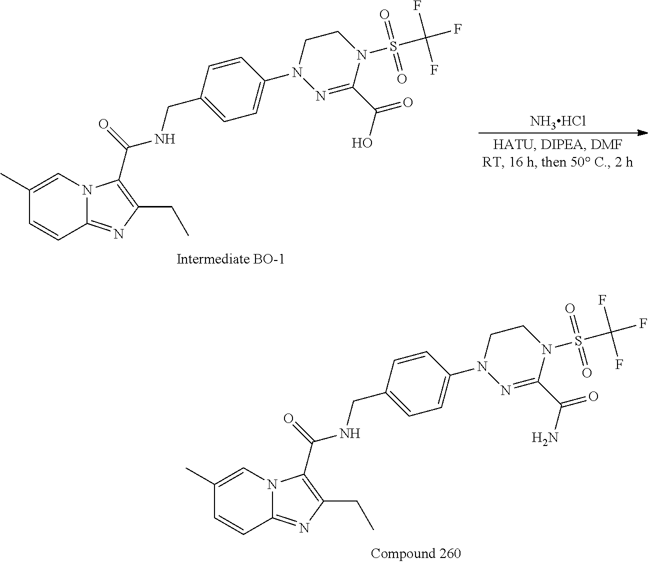 [SMILES: CCc1nc2ccc(C)cn2c1C(=O)NCc1ccc(N2CCN(S(=O)(=O)C(F)(F)F)C(C(=O)O)=N2)cc1 ; "NH3·HCl" ; CCc1nc2ccc(C)cn2c1C(=O)NCc1ccc(N2CCN(S(=O)(=O)C(F)(F)F)C(C(N)=O)=N2)cc1]

Accordingly, compound 260 was prepared in the same way as compound 259, starting from intermediate B-1 (0.27 mmol) and ammonium chloride affording compound 260 (28 mg, 19%) as a beige solid.

1H NMR (400 MHz, DMSO-d₆) δ 8.78 (d, J=0.8 Hz, 1H), 8.35 (t, J=6.0 Hz, 1H), 7.68 (s, 1H), 7.51 (d, J=9.5 Hz, 1H), 7.45 (d, J=8.8 Hz, 2H), 7.36 (d, J=8.8 Hz, 2H), 7.33 (s, 1H), 7.24 (dd, J=9.1, 1.7 Hz, 1H), 4.49 (d, J=5.9 Hz, 2H), 3.90 (d, J=52.6 Hz, 4H), 2.95 (q, J=7.5 Hz, 2H), 2.31 (s, 3H), 1.25 (t, J=7.5 Hz, 3H).

The following compounds are/were also prepared in accordance with the methods described herein:

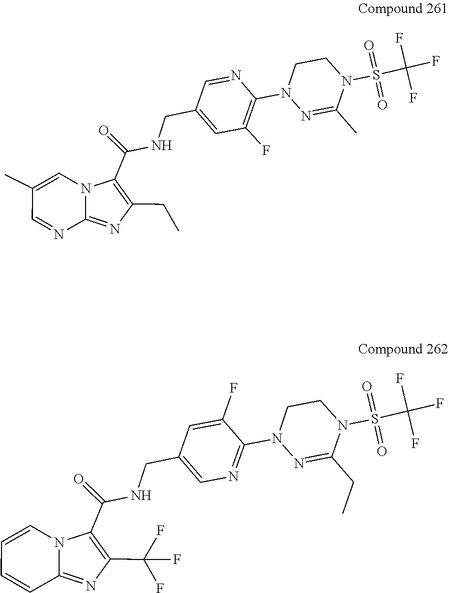

Characterizing Data Table

| | Melting | LCMS | | | | |
|---|---|---|---|---|---|---|
| Compound | point (° C.) | Retention | UV | M | [M + | |
| number | (MT) | time | (%) | exact | H]⁺ | Method |
| 227 | 159.8 | 3.826 | 99 | 552.2 | 553.3 | D |
| 228 | 136.3 | 3.742 | 98 | 549.2 | 550.2 | D |
| 229 | 131.2 | 3.229 | 99 | 584.2 | 549.2 | D |
| 230 | 181.5 | 4.166 | 99 | 568.1 | 569.1 | D |
| 231 | 131.3 | 4.564 | 99 | 604.1 | 605.1 | D |
| 232 | 153.1 | 3.104 | 96 | 567.2 | 568.1 | D |
| 233 | 101.3 | 4.205 | 98 | 580.1 | 581.1 | D |
| 234 | 133 | 3.812 | 96 | 555.2 | 556.2 | D |
| 235 | 141.3 | 3.608 | 99 | 556.2 | 557.1 | D |
| 236 | 173 | 4.051 | 99 | 581.1 | 582.1 | D |
| 237 | 134.6 | 3.453 | 99 | 562.2 | 563.2 | D |
| 238 | 141.4 | 4.721 | 99 | 606.1 | 607.1 | D |
| 239 | 131.2 | 4.226 | 99 | 598.2 | 599.1 | D |
| 240 | 62.7 | 4.447 | 99 | 590.2 | 591.1 | D |
| 241 | 131.3 | 3.428 | 99 | 582.2 | 583.2 | D |
| 242 | 137.9 | 3.155 | 98 | 558.2 | 559.2 | D |
| 243 | N/A (foam) | 4.263 | 99 | 595.1 | 596.1 | D |
| 244 | 153.1 | 4.666 | 99 | 588.1 | 589.1 | D |
| 245 | 151.3 | 3.354 | 99 | 564.2 | 565.2 | D |
| 246 | 72.8 | 3.323 | 99 | 569.2 | 570.2 | D |
| 247 | 107.8 | 4.387 | 99 | 594.1 | 595.1 | D |
| 248 | 136.3 | 4.018 | 99 | 569.2 | 570.2 | D |
| 249 | 168.1 | 4.177 | 99 | 591.1 | 592.1 | D |
| 250 | n.m. | 3.07 | 100 | 523.2 | 524.3 | E |
| 251 | 126.3 | 3.556 | 99 | 541.2 | 542.2 | D |
| 252 | 121.2 | 3.999 | 98 | 566.1 | 567.1 | D |
| 253 | 124.6 | 4.410 | 99 | 580.1 | 581.2 | D |
| 254 | 184.4 | 3.720 | 99 | 567.1 | 568.1 | D |
| 255 | 154.7 | 3.266 | 99 | 542.1 | 543.1 | D |
| 256 | 153 | 3.761 | 99 | 567.1 | 568.1 | D |
| 257 | 133 | 3.860 | 99 | 581.1 | 582.2 | D |
| 258 | 149.7 | 2.772 | 99 | 566.2 | 567.2 | D |
| 259 | 136.4 | 2.535 | 99 | 579.2 | 580.2 | D |
| 260 | 173 | 2.328 | 99 | 551.2 | 552.2 | D |
| 261 | 154.7 | 3.266 | 99 | 542.5 | 543.1 | D |
| 262 | 173 | 4.051 | 99 | 581.5 | 582.1 | D |

1. Biological Assays/Pharmacological Examples

MIC Determination for Testing Compounds Against M. tuberculosis.

Test 1 Test compounds and reference compounds were dissolved in DMSO and 1 μl of solution was spotted per well in 96 well plates at 200× the final concentration. Column 1 and column 12 were left compound-free, and from column 2 to 11 compound concentration was diluted 3-fold. Frozen stocks of Mycobacterium tuberculosis strain (EH4.0 in this case; other strains may be used e.g. H37Rv) expressing green-fluorescent protein (GFP) were previously prepared and titrated. To prepare the inoculum, 1 vial of frozen bacterial stock was thawed to room temperature and diluted to 5×10 exp5 colony forming units per ml in 7H9 broth. 200 μl of inoculum, which corresponds to 1×10 exp5 colony forming units, were transferred per well to the whole plate, except column 12. 200 μl 7H9 broth were transferred to wells of column 12. Plates were incubated at 37° C. in plastic bags to prevent evaporation. After 7 days, fluorescence was measured on a Gemini EM Microplate Reader with 485 excitation and 538 nm emission wavelengths and IC₅₀ and/or pIC₅₀ values (or the like, e.g. IC₅₀, IC₉₀, pIC₉₀, etc) were (or may be) calculated.

Test 2

Appropriate solutions of experimental/test and reference compounds were made in 96 well plates with 7H9 medium. Samples of Mycobacterium tuberculosis strain H37Rv were taken from cultures in logarithmic growth phase. These were first diluted to obtain an optical density of 0.3 at 600 nm wavelength and then diluted 1/100, resulting in an inoculum of approximately 5×10 exp5 colony forming units per ml. 100p of inoculum, which corresponds to 5×10 exp4 colony forming units, were transferred per well to the whole plate, except column 12. Plates were incubated at 37° C. in plastic bags to prevent evaporation. After 7 days, resazurin was added to all wells. Two days later, fluorescence was measured on a Gemini EM Microplate Reader with 543 excitation and 590 nm emission wavelengths and MIC₅₀ and/or pIC₅₀ values (or the like, e.g. IC₅₀, IC₉₀, pIC₉₀, etc) were (or may be) calculated.

Test 3: Time Kill Assays

Bactericidal or bacteriostatic activity of the compounds can be determined in a time kill kinetic assay using the broth dilution method. In this assay, the starting inoculum of M. tuberculosis (strain H37Rv and H37Ra) is 10⁶ CFU/ml in Middlebrook (1×) 7H9 broth. The test compounds are tested alone or in combination with another compound (e.g. a compound with a different mode of action, such as with a cytochrome bd inhibitor) at a concentration ranging from 10-30 μM to 0.9-0.3 μM respectively. Tubes receiving no antibacterial agent constitute the culture growth control. The tubes containing the microorganism and the test compounds are incubated at 37° C. After 0, 1, 4, 7, 14 and 21 days of incubation samples are removed for determination of viable counts by serial dilution (10⁰ to 10⁻⁶) in Middlebrook 7H9 medium and plating (100 μl) on Middlebrook 7H111 agar. The plates are incubated at 37° C. for 21 days and the number of colonies are determined. Killing curves can be constructed by plotting the log₁₀ CFU per ml versus time. A bactericidal effect of a test compound (either alone or in combination) is commonly defined as 2-log₁₀ decrease (decrease in CFU per ml) compared to Day 0. The potential carryover effect of the drugs is limited by using 0.40 charcoal in the agar plates, and by serial dilutions and counting the colonies at highest dilution possible used for plating.

Results

Compounds of the invention/examples, for example when tested in Test 1 described above, may typically have a pIC₅₀ from 3 to 10 (e.g. from 4.0 to 9.0, such as from 5.0 to 8.0)

2. Biological Results
Compounds of the examples were tested in Test 1 (and/or Test 2) described above (in section “Pharmacological Examples”) and the following results were obtained:

Biological Data Table

### Reference Examples

| Compound | | Compound | | Compound | |
|---|---|---|---|---|---|
| number | pIC₅₀ | number | pIC₅₀ | number | pIC₅₀ |
| 1 | 8.15 | 132 | 8.08 | 97 | 7.47 |
| 31 | 8.04 | 107 | 7.62 | 83 | 6.43 |
| 32 | 7.94 | 93 | 7.31 | 121 | 7.89 |
| 29 | 8 | 116 | 7.82 | 80 | 6.30 |
| 28 | 8.6 | 108 | 7.69 | 138 | 8.21 |
| 30 | 7.9 | 146 | 8.42 | 84 | 6.70 |
| 21 | 8.6 | 120 | 7.89 | 81 | 6.30 |
| 19 | 7.4 | 92 | 7.27 | 82 | 6.30 |
| 2 | 7.8 | 94 | 7.41 | 165 | |
| 56 | 3.8 | 141 | 8.31 | 166 | 6.61 |
| 20 | 7.5 | 110 | 7.73 | 167 | 7.06 |
| 3 | 7.5 | 96 | 7.44 | 168 | 7.41 |
| 57 | 6.3 | 156 | 8.86 | 169 | 8.00 |
| 13 | 8.4 | 164 | 9.61 | 170 | 6.50 |
| 58 | 6.6 | 91 | 7.19 | 171 | 7.60 |
| 14 | 8.2 | 99 | 7.49 | | |
| 59 | 6.7 | 123 | 7.99 | 173 | 7.30 |
| 15 | 7.6 | 147 | 8.42 | 174 | 6.30 |
| 6 | 8.2 | 157 | 8.86 | 175 | 8.80 |
| 16 | 8.2 | 152 | 8.70 | 176 | 7.80 |
| 4 | 7.4 | 159 | 8.92 | 177 | 8.10 |
| 18 | 7.8 | 122 | 7.98 | 178 | 6.70 |
| 5 | 8.1 | 103 | 7.55 | 179 | 6.90 |
| 17 | 7.7 | 154 | 8.77 | 180 | 8.40 |
| 22 | 6.4 | 118 | 7.86 | 181 | 8.20 |
| 23 | 7.9 | 119 | 7.87 | 182 | 7.50 |
| 10 | 7.2 | 142 | 8.33 | 183 | 6.70 |
| 24 | 7.8 | 163 | 9.50 | 184 | 7.50 |
| 26 | 8.6 | 125 | 8.01 | 185 | 7.33 |
| 11 | 7.1 | 86 | 7.05 | 186 | 8.44 |
| 12 | 8.5 | 115 | 7.80 | 187 | 6.30 |
| 9 | 9.2 | 111 | 7.74 | 188 | 6.30 |
| 8 | 6.6 | 98 | 7.48 | 189 | 6.30 |
| 25 | 8 | 150 | 8.53 | 190 | 6.76 |
| 7 | 7.2 | 109 | 7.70 | 191 | 7.42 |
| 27 | 8.7 | 149 | 8.45 | 192 | <6.301 |
| 60 | 6.7 | 153 | 8.75 | 193 | 9.05 |
| 61 | 6.3 | 130 | 8.06 | 194 | 8.62 |
| 62 | 6.3 | 133 | 8.12 | 195 | 7.98 |
| 33 | 7.6 | 126 | 8.01 | 196 | 6.46 |
| 38 | 7.2 | 129 | 8.05 | 198 | 7.03 |
| 39 | 7.1 | 101 | 7.53 | 199 | <6.301 |
| 40 | 7.2 | 161 | 9.11 | 200 | 8.26 |
| 63 | 6.3 | 88 | 7.08 | 201 | 7.92 |
| 41 | 7.3 | 127 | 8.02 | 202 | 6.41 |
| 34 | 8 | 104 | 7.55 | 203 | <6.301 |
| 35 | 8.3 | 128 | 8.04 | 204 | 8.35 |
| 64 | 6.5 | 158 | 8.86 | 205 | 7.18 |
| 65 | 6.3 | 87 | 7.05 | 206 | 8.61 |
| 42 | 8.2 | 155 | 8.79 | 207 | 6.73 |
| 43 | 8 | 151 | 8.58 | 208 | 7.16 |
| 66 | 6.6 | 112 | 7.76 | 209 | 7.95 |
| 67 | 6.7 | 137 | 8.21 | 210 | 8.52 |
| 36 | 8.4 | 160 | 8.92 | 211 | 8.64 |
| 37 | 8.4 | 113 | 7.79 | 212 | 7.59 |
| 44 | 8.6 | 85 | 7.02 | 213 | 8.46 |
| 45 | 7.4 | 145 | 8.39 | 214 | 7.87 |
| 68 | 6.3 | 95 | 7.42 | 215 | 9.04 |
| 46 | 7 | 124 | 8.00 | 216 | 8.67 |
| 47 | 8.5 | 144 | 8.38 | 217 | 7.85 |
| 69 | 6.9 | 114 | 7.79 | 218 | 8.24 |
| 70 | 6.3 | 117 | 7.83 | 79 | 8.43 |
| 48 | 8.8 | 102 | 7.53 | | |
| 49 | 7.63 | 148 | 8.42 | | |
| 50 | 8.97 | 89 | 7.08 | | |
| 51 | 7.34 | 162 | 9.41 | | |
| 52 | 7.38 | 105 | 7.56 | | |
| 71 | 6.78 | 143 | 8.35 | | |
| 72 | 6.8 | 136 | 8.20 | | |
| 53 | 7.09 | 135 | 8.14 | | |
| 54 | 7.96 | 131 | 8.06 | | |
| 55 | 7.59 | 134 | 8.12 | | |
| 73 | 7.29 | 106 | 7.59 | | |
| 74 | 8.17 | 100 | 7.50 | | |
| 75 | 8.2 | 139 | 8.24 | | |
| 76 | 7.87 | 140 | 8.26 | | |
| 77 | 8.1 | 90 | 7.13 | | |

| Compound number | pIC₅₀ |
|---|---|
| 227 | 7.94 |
| 228 | 8.84 |
| 229 | 8.97 |
| 230 | 8.18 |
| 231 | 9.05 |
| 232 | 8.82 |
| 233 | 8.97 |
| 234 | 9.49 |
| 235 | 8.2 |
| 236 | 8.21 |
| 237 | 8.24 |
| 238 | 8.8 |
| 239 | 8.05 |
| 240 | 9.22 |
| 241 | 8.46 |
| 242 | 8.62 |
| 243 | |
| 244 | 8.34 |
| 245 | 7.86 |
| 246 | 8.58 |
| 247 | 8.86 |
| 248 | 9.65 |
| 249 | 8.73 |
| 250 | 8.8 |
| 251 | 9.36 |
| 252 | 8.92 |
| 253 | 8.73 |
| 254 | 7.27 |
| 255 | 8.21 |
| 256 | 7.73 |
| 257 | 8.22 |
| 258 | 8.21 |
| 259 | 7.97 |
| 260 | 7.49 |

3. Further Data on Representative Compounds, Including Reference Compounds and/or Compounds of the Invention/Examples

The compounds of the invention/examples may have advantages associated with in vitro potency, kill kinetics (i.e. bactericidal effect) in vitro, PK properties, food effect, safety/toxicity (including liver toxicity, coagulation, 5-LO oxygenase), metabolic stability, Ames II negativity, MNT negativity, aqueous based solubility (and ability to formulate) and/or cardiovascular effect e.g. on animals (e.g. anesthetized guinea pig). The data below that was generated/calculated may be obtained using standard methods/assays, for instance that are available in the literature or which may be performed by a supplier (e.g. Microsomal Stability Assay—Cyprotex, Mitochondrial toxicity (Glu/Gal) assay—Cyprotex, as well as literature CYP cocktail inhibition assays). In some instances, GSH was measured (reactive metabolites, glucuronidation) to observe if a dihydrodiol is observed by LCMS (fragmentation ions), which would correspond to a dihydroxylation on the core heterocycle.

This following data was generated on Compound 1:
c Log P=4.3/TPSA=107.7
CVS (Na Ch, Ca Ch, hERGdof), IC₅₀=>10, >10, >10
Cocktail Cyp-450, IC₅₀=>20 (except CYP3A4, which was not conclusive)
CLint (μl/min/mg prot)=(H) 29.6/(M) 21.5

The following data was generated on Compound 13:
c Log P=3.3/TPSA=120.7
CVS (Na Ch, Ca Ch, hERGdof), IC₅₀=>10, >10, 7.4
Cocktail Cyp-450, IC₅₀=>20 (except CYP3A4 and CY2D6, which were not
conclusive)
CLint (μl/min/mg prot)=(H) 16.3/(M) 13.3

The following data was generated on Compound 20:
c Log P=3.75/TPSA=107.7
CVS (Na Ch, Ca Ch, hERGdof), IC₅₀=>10, >10, >10
Cocktail Cyp-450, IC₅₀=>20 (except CYP3A4, IC₅₀=13.2 μM)
CLint (μl/min/mg prot)=(H) 56.6/(M) 15.9

The following data was generated on Compound 73:

It was tested and showed no measure of GSH
c Log P=3.2/TPSA 140.8
CVS (Ca, Na, Herg), IC₅₀=>10
Cocktail Cyp-450, IC₅₀=>20 (for all)
CLint (μl/min/mg prot)=(H) 18/(M) 93

The following data was generated on Compound 9
c Log P=4.4/TPSA 107.8
CVS (Ca, Na, Herg), IC₅₀=>10
Cocktail Cyp-450, IC₅₀=>20 (for all)
CLint (μl/min/mg prot)=(H) 19/(M) 41

The following data was generated on Compound 26
c Log P=3.1/TPSA 129.9
CVS (Ca, Na, Herg), IC₅₀=>10
Cocktail Cyp-450, IC₅₀=>20 (for all)
CLint (μl/min/mg prot)=(H) 37/(M) 35

The following data was generated on Compound 16
c Log P=4.4/TPSA 107.8
CVS (Ca, Na, Herg), IC₅₀=>10
Cocktail Cyp-450, IC₅₀=>20 (for all)
CLint (μl/min/mg prot)=(H) 24/(M) 18

The following data was generated on Compound 6

It was tested and showed no measure of GSH
c Log P=4.3/TPSA 117
CVS (Ca, Na, Herg), IC₅₀=>10
Cocktail Cyp-450, IC₅₀=>20 (for all)
CLint (μl/min/mg prot)=(H) 37.6/(M) 49

The following further data/results were generated

Compound 1:
Was found to have low mitotoxicity (<3 in the Glu/Gal assay)—hence no mitotoxicity alerts
Had good bioavailability (as shown in rodents)

Compound 6:
Was found to have low mitotoxicity (<3 in the Glu/Gal assay)—hence no mitotoxicity alerts
Did not produce unwanted reactive metabolites (it showed no measure of GSH)

Compound 152:
Found to have low mitotoxicity (<3 in the Glu/Gal assay)—hence no mitotoxicity alerts
Had good bioavailaibility (as shown in rodents)
The formation of reactive metabolites was blocked

Compound 161:
Found to have low mitotoxicity (<3 in the Glu/Gal assay)—hence no mitotoxicity alerts
Had good bioavailaibility (as shown in rodents)
The formation of reactive metabolites was blocked

Specific Data on Compound 161:
TPSA=120.6
HTEq Sol (μg/mL)—pH 2: 33, pH 7: <0.02, FaSSIF: 5, FeSSIF: 16
Cocktail Cyp-450, IC₅₀ (μM)=>20
Cyp 3A4 induction (% control)—at 1 μM=3.0
CLint Hep (ml/min/10⁶ cells)=(M) 0.012/(R) 0.019/(D) 0.0047/(H) 0.0067
PPB (% unbound) (H) 1.5/(M) 2.45
AMES II—negative (Score 1)
Glu/Gal—negative (ratio <3)
GSH/CN—no reactive metabolites
Kinase panel—negative
CTCM (μM)—clean up to 5 μM
CVS (Na Ch, Ca Ch, hERGdof), IC₅₀=>10, >10, 15.85

Oral Bioavailability of Compound 161 in Rat

Compound 161 was administed PO in rat (5 mg/kg, PEG4000 (sol.), 0.5 w/v Methocel (susp.) and the following results were obtained for the solution and suspension.

| | Solution | Suspension |
|---|---|---|
| | (Compound 161) | (Compound 161) |
| Cₘₐₓ (ng/mL) | 1228 ± 406 | 787 ± 226 |
| Tₘₐₓ (h) | 4.0 | 2.0 (1.0-2.0) |
| AUC_{0-inf} (ng · h/mL) | 10880 ± 1715 | 5610 ± 2747 |
| t_{1/2} (h) | 3.55 ± 0.45 | 3.49 ± 0.91 |
| F (%) | 106 ± 17 | 55 ± 27 |

### CONCLUSIONS

Compounds disclosed herein, including reference compounds and/or compounds of the invention/examples (e.g. as exemplified by Compound 161), may therefore have the advantage that:
No in vitro cardiotoxicity is observed (for example either due to the CVS results or due to the Glu/Gal assay results);
No reactive metabolite formation is observed (e.g. GSH); and/or
There is a relatively higher unbound fraction, for instance as compared to other compounds, for instance prior art compounds.

Certain compounds of the invention/examples may also have the additional advantage that they do not form degradants (e.g. that are undesired or may elicit unwanted side-effects).

Compounds (for instance, as represented by Compound 161), may have the advantage that a faster oral absorption and improved bioavailability are displayed (as may be shown by the oral bioavailability data in rat).

Chemical Stability Testing

Compounds disclosed herein may have the advantage that they are chemically more stable than other compounds (e.g. than other known compounds), for instance as tested in the chemical stability assay described below.

Preliminary Protocol
Add 3 μl of a 10 mM DMSO stock solution to 1 ml of the following solvents in a 1.5 ml HPLC vial.
DMSO (reference solution)
H₂O/Acetonitril 1/1 (assay solution)
0.1N HCl/Acetonitril 1/1 (assay solution)
Mix well, store them on the bench for 72h
Analyse the samples with LCMS
Compare the chromatograms of the two assay solutions with the reference solution and report the additional peaks as degradation peaks

For instance, the following chemical stability results (in % by LCMS) were observed:
Compound 257: DMSO (0 hr, rt)=99%; ACN/H₂O (48 hr, rt)=99%; ACN/0.1N HCl (pH 1.6; 48 hr, rt)=99%
Compound 252: DMSO (0 hr, rt)=99%; ACN/H₂O (48 hr, rt)=99%; ACN/0.1N HCl (pH 1.6; 48 hr, rt)=99%
Compound 255: DMSO (0 hr, rt)=100%; ACN/H₂O (48 hr, rt)=100%; ACN/0.1N HCl (pH 1.6; 48 hr, rt)=98%
Compound 251: DMSO (0 hr, rt)=100%; ACN/H₂O (48 hr, rt)=100%; ACN/0.1N HCl (pH 1.6; 48 hr, rt)=100%
Compound 253: DMSO (0 hr, rt)=100%; ACN/H₂O (48 hr, rt)=100%; ACN/0.1N HCl (pH 1.6; 48 hr, rt)=100%
Compound 258: DMSO (0 hr, rt)=99%; ACN/H₂O (48 hr, rt)=99%; ACN/0.1N HCl (pH 1.6; 48 hr, rt)=100%
Compound 260: DMSO (0 hr, rt)=98%; ACN/H₂O (48 hr, rt)=100%; ACN/0.1N HCl (pH 1.6; 48 hr, rt)=100%
Compound 259: DMSO (0 hr, rt)=98%; ACN/H₂O (48 hr, rt)=100%; ACN/0.1N HCl (pH 1.6; 48 hr, rt)=100%
Compound 234: DMSO (0 hr, rt)=96%; ACN/H₂O (48 hr, rt)=95%; ACN/0.1N HCl (pH 1.6; 48 hr, rt)=97%
Compound 233: DMSO (0 hr, rt)=99%; ACN/H₂O (48 hr, rt)=99%; ACN/0.1N HCl (pH 1.6; 48 hr, rt)=99%
Compound 235: DMSO (0 hr, rt)=99%; ACN/H₂O (48 hr, rt)=99%; ACN/0.1N HCl (pH 1.6; 48 hr, rt)=98%
Compound 241: DMSO (0 hr, rt)=100%; ACN/H₂O (48 hr, rt)=100%; ACN/0.1N HCl (pH 1.6; 48 hr, rt)=100%
Compound 240: DMSO (0 hr, rt)=100%; ACN/H₂O (48 hr, rt)=100%; ACN/0.1N HCl (pH 1.6; 48 hr, rt)=100%
Compound 238: DMSO (0 hr, rt)=100%; ACN/H₂O (48 hr, rt)=100%; ACN/0.1N HCl (pH 1.6; 48 hr, rt)=100%
Compound 248: DMSO (0 hr, rt)=92%; ACN/H₂O (48 hr, rt)=100%; ACN/0.1N HCl (pH 1.6; 48 hr, rt)=100%
Compound 247: DMSO (0 hr, rt)=96%; ACN/H₂O (48 hr, rt)=100%; ACN/0.1N HCl (pH 1.6; 48 hr, rt)=100%
Compound 231: DMSO (0 hr, rt)=98%; ACN/H₂O (48 hr, rt)=98%; ACN/0.1N HCl (pH 1.6; 48 hr, rt)=98%

This showed that, under the tested conditions, the compounds were stable, and mostly not susceptible to unwanted degradation in acidic media.

1. A compound of formula (I) 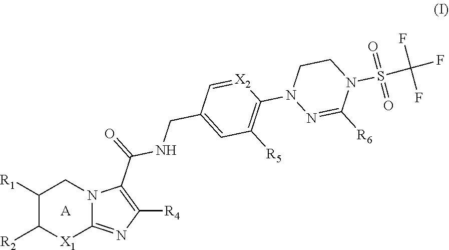
wherein:
A is a 6-membered ring, which is aromatic or non-aromatic;
X₁ is ═N— or ═C(R₃)— (when aromatic) or —CH₂— (when non-aromatic);
X₂ is ═N— or ═CH—;
R₁ is H, —CH₃, F or Cl;
R₂ is H or —CH₃;
R₃ is H or F;
R₄ is —CF₃, —CHF₂ or —C₂H₅;
R₅ is H or F;
R₆ is —CH₃, —C₂H₅, isopropyl, cyclopropyl, cyclobutyl, —C(═O)—OCH₃, —C(═O)—NH₂ or —C(═O)—N(CH₃)₂;
or a pharmaceutically-acceptable salt thereof.

2. The compound of claim 1, that is of formula (II): 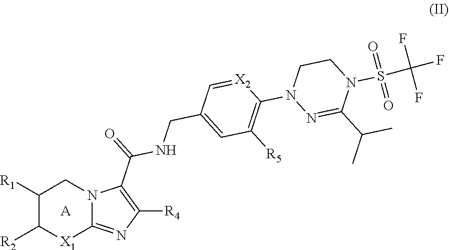
wherein:
X₁ is ═N— or ═C(R₃)—;
R₁ is H, —CH₃, or Cl;
or a pharmaceutically-acceptable salt thereof.

3. The compound of claim 1, that is of formula (III): 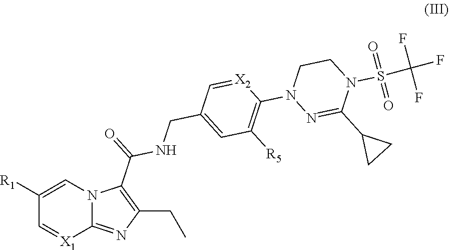
wherein:
X₁ is ═N— or ═C(R₃)—;
R₁ is CH₃, F, or Cl;
or a pharmaceutically-acceptable salt thereof.

4. The compound of claim 1, that is of formula (IV): 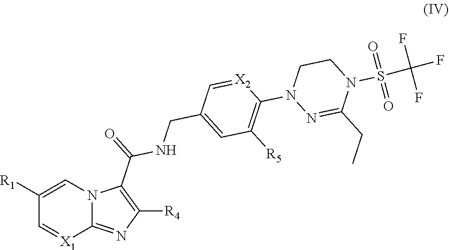
wherein:
X₁ or ═N— or ═CH—;
R₁ is H or —CH₃;
R₄ is —CF₃ or —C₂H₅,
or a pharmaceutically-acceptable salt thereof.

5. The compound of claim 1, that is of formula (V): 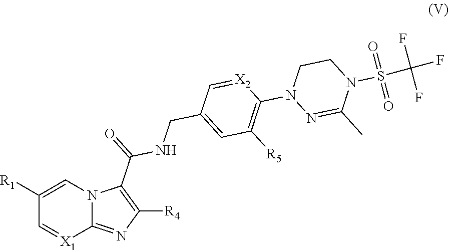
wherein
X₁ or ═N— or ═CH—;
R₁ is H or —CH₃;
or a pharmaceutically-acceptable salt thereof.

6. The compound of claim 1, that is of formula (VI) according to claim 1, 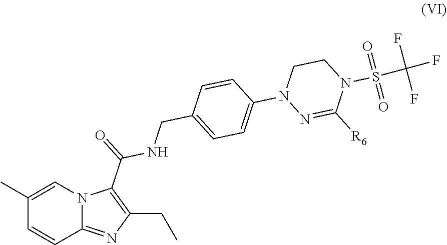
wherein
R₆ is cyclopropyl, cyclobutyl, —C(═O)—OCH₃, —C(═O)—NH₂ or —C(═O)—N(CH₃)₂,
or a pharmaceutically-acceptable salt thereof.

7. (canceled)

8. A pharmaceutical composition comprising a pharmaceutically acceptable carrier and, as active ingredient, a therapeutically effective amount of a compound of claim 1.

9. (canceled)

10. (canceled)

11. A method of treating a mycobacterial infection (e.g. tuberculosis) in a patient, comprising administering a therapeutically effective amount of the compound of claim 1 to the patient.

12. A combination comprising (a) the compound of claim 1, and (b) one or more other anti-mycobacterial agent.

13. A product containing (a) the compound of claim 1, and (b) one or more other anti-mycobacterial agent, as a combined preparation for simultaneous, separate or sequential use of treating a bacterial infection.

14. A process for preparing the compound of formula (II) of claim 2, comprising:
(i) reacting a compound of formula (XIV): 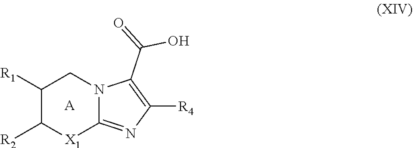
with a compound of formula (XV) or (XVA); 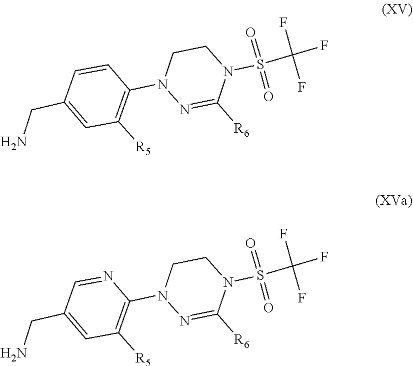
(ii) coupling a compound of formula (XVII) or (XVIIa) from step (i): 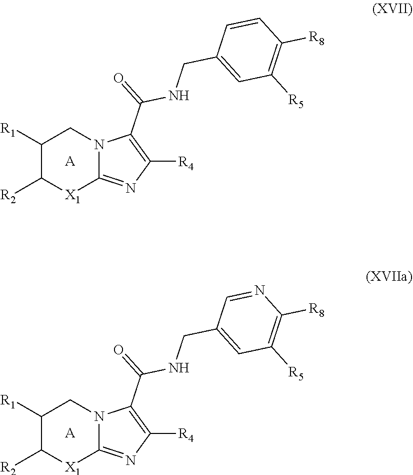
wherein is a leaving group, with a compound of formula (XVI),

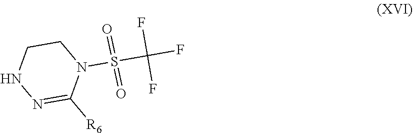

15. The combination of claim 12, wherein the one or more other anti-mycobacterial agent is an anti-tuberculosis agent.

16. The product of claim 13, wherein the one or more other anti-mycobacterial agent is an anti-tuberculosis agent.

17. A process for preparing the compound of formula (I) of claim 1, comprising:
(i) reacting a compound of formula (XIV), 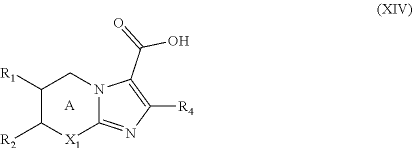
with a compound of formula (XV) or (XVA): 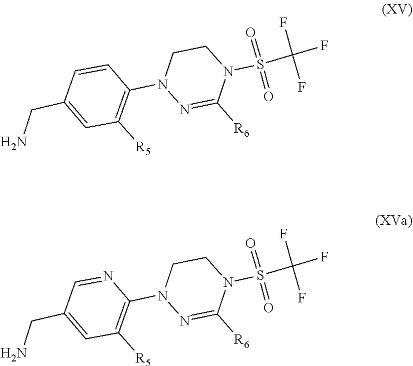
(ii) coupling a compound of formula (XVII) or (XVIIa) from step (i): 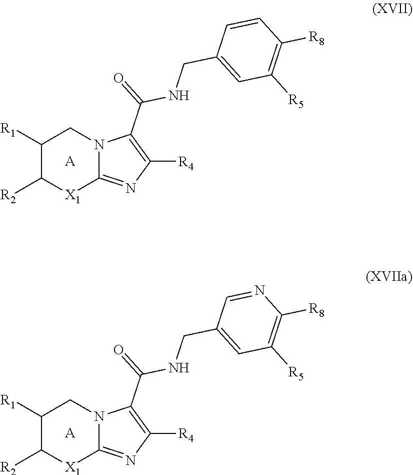
wherein R₈ is a leaving group, with a compound of formula (XVI): 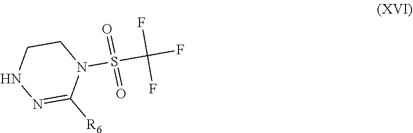
(iii) reacting a compound of formula (XVIII) or (XVIIIA) from step (ii): 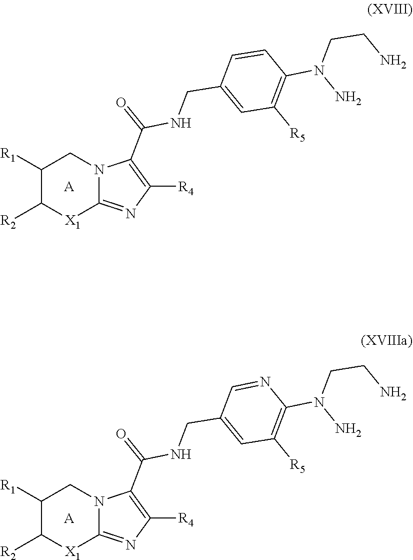
with a compound of formula (XIX):
R₆C(OCH₃)₃ (XIX); and
(iv) reacting the product of step (iii) with a compound of formula (XIXA):
LG¹-S(O)₂CF₃ (XIXA)
wherein LG¹ is a leaving group.
